(19) United States
(12) Patent Application Publication
(10) Pub. No.: US 2023/0293630 A1
(43) Pub. Date: Sep. 21, 2023

(54) Coronavirus T Cell Epitopes and Uses Thereof

(71) Applicant: La Jolla Institute for Immunology, La Jolla, CA (US)

(72) Inventors: Alessandro Sette, La Jolla, CA (US); Shane Crotty, La Jolla, CA (US); Alba Grifoni, La Jolla, CA (US); Daniela Weiskopf, La Jolla, CA (US); Bjoern Peters, La Jolla, CA (US); John Sidney, La Jolla, CA (US)

(21) Appl. No.: 17/760,402

(22) PCT Filed: Feb. 11, 2021
(86) PCT No.: PCT/US2021/017716

§ 371 Date: Aug. 10, 2022

Related U.S. Application Data
(60) Provisional application No. 63/124,164, filed on Dec. 11, 2020.
(60) Provisional application No. 63/124,172, filed on Dec. 11, 2020.
(60) Provisional application No. 63/108,281, filed on Oct. 30, 2020.
(60) Provisional application No. 63/061,145, filed on Aug. 4, 2020.
(60) Provisional application No. 63/050,776, filed on Jul. 11, 2020.
(60) Provisional application No. 63/040,749, filed on Jun. 18, 2020.
(60) Provisional application No. 63/029,336, filed on May 22, 2020.
(60) Provisional application No. 63/024,356, filed on May 13, 2020.
(60) Provisional application No. 63/019,895, filed on May 4, 2020.
(60) Provisional application No. 63/012,902, filed on Apr. 20, 2020.
(60) Provisional application No. 63/003,854, filed on Apr. 1, 2020.
(60) Provisional application No. 62/985,526, filed on Mar. 5, 2020.
(60) Provisional application No. 62/975,740, filed on Feb. 12, 2020.

(51) Int. Cl. A61K 38/16; C07K 14/005; A61K 38/20; A61K 38/18; A61K 38/21; G01N 33/50; G01N 33/569; C12N 15/63; A61P 31/14

(52) U.S. Cl. CPC A61K 38/162; A61K 38/1841; A61K 38/204; A61K 38/2066; A61K 38/217; A61P 31/14; C07K 14/005; C12N 15/63; G01N 33/505; G01N 33/56983; G01N 2333/165

(57) ABSTRACT

The present invention includes compositions and methods for detecting the presence of: a coronavirus or an immune response to a coronavirus infection including T cells responsive to one or more coronavirus peptides or proteins comprising, consisting of, or consisting essentially of: one or more amino acid sequences selected from SEQ ID NO: 1 to 1126, subsequences, portions, homologues, variants or derivatives; a fusion protein comprising one or more amino acid sequences selected from SEQ ID NO: 1 to 1126; a pool of peptides or proteins selected from the amino acid sequences set forth in SEQ ID NO: 1 to 1126; or a polynucleotide that encodes one or more peptides or proteins, comprising, consisting of, or consisting essentially of an amino acid sequence selected from SEQ ID NO: 1 to 1126, subsequences, portions, homologues, variants or derivatives. The invention further provides vaccines, diagnostics, therapies, and kits, comprising such proteins or peptides.

### CROSS-REFERENCE TO RELATED APPLICATIONS

This application claims priority to U.S. Provisional Application Serial No. 62/975,740, filed Feb. 12, 2020; U.S. Provisional Application Serial No. 62/985,526, filed Mar. 5, 2020; U.S. Provisional Application Serial No. 63/003,854, filed Apr. 1, 2020; U.S. Provisional Application Serial No. 63/012,902, filed Apr. 20, 2020; U.S. Provisional Application Serial No. 63/019,895, filed May 4, 2020; U.S. Provisional Application Serial No. 63/024,356, filed May 13, 2020; U.S. Provisional Application Serial No. 63/029,336, filed May 22, 2020; U.S. Provisional Application Serial No. 63/040,749, filed Jun. 18, 2020; U.S. Provisional Application Serial No. 63/050,776, filed Jul. 11, 2020; U.S. Provisional Application Serial No. 63/061,145, filed Aug. 4, 2020; U.S. Provisional Application Serial No. 63/108,281, filed Oct. 30, 2020; U.S. Provisional Application Serial No. 63/124,164, filed Dec. 11, 2020; and U.S. Provisional Application Serial No. 63/124,172, filed Dec. 11, 2020, all applications of which are expressly incorporated herein by reference, including their entire contents.

### TECHNICAL FIELD OF THE INVENTION

The present invention relates in general to the field of peptides that are T cell epitopes for coronavirus, and more particularly, to compositions and methods for the prevention, treatment, diagnosis, kits, and uses of such T cell epitopes.

### STATEMENT OF FEDERALLY FUNDED RESEARCH

This invention was made with government support under grant and contract numbers U19 AI142742, 75N9301900065, 75N93019C00001, and U19 AI118626, awarded by the National Institutes of Health/NIAID. The government has certain rights in the invention.

### SEQUENCE LISTING

The present application includes a Sequence Listing which has been submitted in ASCII format via EFS-Web and is hereby incorporated by reference in its entirety. Said ASCII copy, created on Feb. 11, 2021, is named LJII 2006WO.txt and is 326,622 bytes in size.

### BACKGROUND OF THE INVENTION

Without limiting the scope of the invention, its background is described in connection with coronaviruses.

As of February 2021, SARS-CoV-2 infections are associated with 2.24 million deaths and over 100 million cases worldwide, and over 27 million cases in the United States alone (https://coronavirus.jhu.edu/map.html). The severity of the associated Coronavirus Disease 2019 (COVID-19) ranges from asymptomatic or mild self-limiting disease, to severe pneumonia and acute respiratory distress syndrome (WHO; https://www.who.int/publications/i/item/clinical-management-of-covid-19). The present inventors and others have started to delineate the role of SARS-CoV-2-specific T cell immunity in COVID-19 clinical outcomes (Altmann and Boyton, 2020; Braun et al., 2020; Grifoni et al., 2020; Le Bert et al., 2020; Meckiff et al., 2020; Rydyznski Moderbacher et al., 2020; Sekine et al., 2020; Weiskopf et al., 2020). A growing body of evidence points to a key role for SARS-CoV-2-specific T cell responses in COVID-19 disease resolution and modulation of disease severity (Rydyznski Moderbacher et al., 2020; Schub et al., 2020; Weiskopf et al., 2020). Milder cases of acute COVID-19 were associated with coordinated antibody, CD4+ and CD8+ T cell responses, whereas severe cases correlated with a lack of coordination of cellular and antibody responses, and delayed kinetics of adaptive responses (Rydyznski Moderbacher et al., 2020; Weiskopf et al., 2020).

Despite these advances, a need remains for identifying T cell epitopes for use in diagnostics, treatments, vaccines, kits, etc.

### SUMMARY OF THE INVENTION

In one embodiment, the present invention includes a composition comprising: one or more peptides or proteins, comprising, consisting of, or consisting essentially of an amino acid sequence selected from SEQ ID NO: 1 to 1126, or a subsequence, portion, homologue, variant or derivative thereof; a fusion protein comprising one or more amino acid sequences selected from SEQ ID NO: 1 to 1126; a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 or more peptides comprising, consisting of, or consisting essentially of amino acid sequences selected from SEQ ID NO: 1 to 1126; or a polynucleotide that encodes one or more peptides or proteins, comprising, consisting of, or consisting essentially of an amino acid sequence selected from SEQ ID NO: 1 to 1126, or a subsequence, portion, homologue, variant or derivative thereof. SEQ ID NOS: 1 to 1126 are found in Tables 4 to 9. In one aspect, the one or more peptides or proteins comprises, or wherein the fusion protein comprises 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 or more amino acid sequences selected from SEQ ID NO: 1 to 1126, or a subsequence, portion, homologue, variant or derivative thereof. In another aspect, the amino acid sequence is selected from a coronavirus T cell epitope selected from SEQ ID NO: 874 to 1126. SEQ ID NOS: 874 to 1126 are found in Tables 8 and 9. In another aspect, the composition comprises one or more SARS-CoV-2 peptides amino acid sequences selected from SEQ ID NO: 1 to 873 (SEQ ID NOS:1 to 873 are found in Tables 4 to 7), or a subsequence, portion, homologue, variant or derivative thereof; a fusion protein comprising one or more amino acid sequences selected from SEQ ID NO: 1 to 873; or a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or 873 more peptides selected from SEQ ID NO: 1 to 873. In another aspect, the peptide or protein comprises a coronavirus T cell epitope. In another aspect, the one or more peptides or proteins comprises a coronavirus CD8+ or CD4+ T cell epitope. In another aspect, the coronavirus is SARS-CoV-2 and the SARS-CoV-2 T cell epitope is not conserved in another coronavirus. In another aspect, the coronavirus is SARS-CoV-2 and the SARS-CoV-2 T cell epitope is conserved in another coronavirus. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to a coronavirus. In another aspect, the one or more peptides or proteins that elicits, stimulates, induces, promotes, increases or enhances the T cell response to the coronavirus is a coronavirus spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund’s complete adjuvant, Freund’s incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-y), Transforming growth factor beta (TGF-β), or Interleukin-10 (IL-10), or an agonist or antagonist thereof.

In another embodiment, the present invention includes a composition comprising monomers or multimers of: peptides or proteins comprising, consisting of, or consisting essentially of: one or more amino acid sequences selected from SEQ ID NO: 1 to 1126, concatemers, subsequences, portions, homologues, variants or derivatives thereof; a fusion protein comprising one or more amino acid sequences selected from SEQ ID NO: 1 to 1126; or a polynucleotide that encodes one or more peptides or proteins, comprising, consisting of, or consisting essentially of an amino acid sequence selected from SEQ ID NO: 1 to 1126, or a subsequence, portion, homologue, variant or derivative thereof.

In another embodiment, the present invention includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from SEQ ID NO: 1 to 1126, in a groove of the MHC monomer or multimer.

In another embodiment, the present invention includes a composition comprising: one or more peptides or proteins comprising, consisting of, or consisting essentially of an amino acid sequence selected from SEQ ID NO: 1 to 873, or a subsequence, portion, homologue, variant or derivative thereof; a fusion protein comprising one or more amino acid sequences selected from SEQ ID NO: 1 to 873; a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or more peptides selected from SEQ ID NO: 1 to 873; a polynucleotide that encodes one or more peptides or proteins, comprising, consisting of, or consisting essentially of an amino acid sequence selected from SEQ ID NO: 1 to 873, or a subsequence, portion, homologue, variant or derivative thereof. In one aspect, the one or more peptides or proteins comprises, or wherein the fusion protein comprises, 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or more amino acid sequences selected from SEQ ID NO: 1 to 873, or a subsequence, portion, homologue, variant or derivative thereof. In another aspect, the protein or peptide comprises a SARS-CoV-2 T cell epitope. In another aspect, the one or more peptides or proteins comprises a SARS-CoV-2 CD8+ or CD4+ T cell epitope. In another aspect, the SARS-CoV-2 T cell epitope is not conserved in another coronavirus. In another aspect, the SARS-CoV-2 T cell epitope is conserved in another coronavirus. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to SARS-CoV-2. In another aspect, the one or more peptides or proteins that elicits, stimulates, induces, promotes, increases or enhances the T cell response to SARS-CoV-2 is a SARS-CoV-2 spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund’s complete adjuvant, Freund’s incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLRS ligands, TLR⅞ ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-y), Transforming growth factor beta (TGF-β), or Interleukin-10 (IL-10), or an agonist or antagonist thereof. In another aspect, the one or more peptides or proteins exclude the amino acid sequences selected from SEQ ID NOS: 245-280 and 804-873.

In another embodiment, the present invention includes a composition comprising monomers or multimers of: one or more peptides or proteins comprising, consisting of, or consisting essentially of: one or more SARS-CoV-2 amino acid sequences selected from SEQ ID NO: 1 to 873, concatemers, subsequences, portions, homologues, variants or derivatives thereof; a fusion protein comprising one or more amino acid sequences selected from SEQ ID NO: 1 to 873; or a polynucleotide that encodes one or more peptides or proteins, comprising, consisting of, or consisting essentially of an amino acid sequence selected from SEQ ID NO: 1 to 873, or a subsequence, portion, homologue, variant or derivative thereof.

In another embodiment, the present invention includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from SEQ ID NO: 1 to 873, in a groove of the (MHC) monomer or multimer. In one aspect, the compositions exclude those amino acid sequences selected from SEQ ID NOS: 245-280 and 804-873.

In another embodiment, the present invention includes a method for detecting the presence of: (i) a coronavirus or (ii) an immune response relevant to coronavirus infections, vaccines or therapies, including T cells responsive to one or more coronavirus peptides, comprising: providing one or more proteins or peptides for detection of an amount or a relative amount of, and/or the activity of, and/or the state of antigen-specific T-cells; contacting a biological sample suspected of having coronavirus-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in SEQ ID NO: 1 to 1126, or comprise a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 or more amino acid sequences set forth in SEQ ID NO: 1 to 1126. In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2, 3, 4, 5, 6, 7, 8, 9, 10, 15, 20, 25, 30, 40, 50, 60, 75, 80, 90, 100, 125, 150, 175, 200, 225, 250 or more amino acid sequences selected from SEQ ID NO:874 to 1126. In another aspect, the detecting the amount or a relative amount of, and/or activity of antigen-specific T-cells comprises indirect detection and/or direct detection. In another aspect, the method of detecting an immune response relevant to the coronavirus comprises the following steps: providing an MHC monomer or an MHC multimer; contacting a population T-cells to the MHC monomer or MHC multimer; and measuring the number, activity or state of T-cells specific for the MHC monomer or MHC multimer. In one aspect, the MHC monomer or MHC multimer comprises a protein or peptide of the coronavirus. In another aspect, the protein or peptide comprises a CD8+ or CD4+ T cell epitope. In another aspect, the T cell epitope is not conserved in another coronavirus. In another aspect, the T cell epitope is conserved in another coronavirus. In another aspect, the protein or peptide has a length from about 9-15, 15-20, 20-25, 25-30, 30-40, 40-50, 50-75 or 75-100 amino acids. In another aspect, the proteins or peptides comprise 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 or more amino acid sequences selected from SEQ ID NO: 1 to 1126, or a subsequence, portion, homologue, variant or derivative thereof. In another aspect, the method further comprises detecting the presence or amount of the one or more peptides in a biological sample, or a response thereto, which is diagnostic of a coronavirus infection. In another aspect, the detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present invention includes a method for detecting the presence of: (i) SARS-CoV-2 or (ii) an immune response relevant to SARS-CoV-2 infections, vaccines or therapies, including T cells responsive to one or more SARS-CoV-2 peptides, comprising: providing one or more proteins or peptides for detection of an amount or a relative amount of, and/or the activity of, and/or the state of antigen-specific T-cells; contacting a biological sample suspected of having SARS-CoV-2-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in SEQ ID NO: 1 to 873, or comprise a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or more amino acid sequences set forth in SEQ ID NO: 1 to 873. In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2, 3, 4, 5, 6, 7, 8, 9, 10, 15, 20, 25, 30, 40, 50, 60, 75, 80, 90, 100, 125, 150, 175, 200, 225, 250 or more amino acid sequences selected from SEQ ID NO: 1 to 873. In another aspect, detecting the amount or a relative amount of, and/or activity of antigen-specific T-cells comprises indirect detection and/or direct detection. In another aspect, detecting an immune response relevant to SARS-CoV-2 comprises the following steps: providing an MHC monomer or an MHC multimer; contacting a population T-cells to the MHC monomer or MHC multimer; and measuring the number, activity or state of T-cells specific for the MHC monomer or MHC multimer. In another aspect, the MHC monomer or MHC multimer comprises a protein or peptide of SARS-CoV-2. In another aspect, the protein or peptide comprises a SARS-CoV-2 CD8+ or CD4+ T cell epitope. In another aspect, the SARS-CoV-2 T cell epitope is not conserved in another coronavirus. In another aspect, the SARS-CoV-2 T cell epitope is conserved in another coronavirus. In another aspect, the protein or peptide has a length from about 9-15, 15-20, 20-25, 25-30, 30-40, 40-50, 50-75 or 75-100 amino acids. In another aspect, the proteins or peptides comprise 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or more amino acid sequences selected from SEQ ID NO: 1 to 873, or a subsequence, portion, homologue, variant or derivative thereof. In another aspect, the method further comprises detecting the presence or amount of the one or more peptides in a biological sample, or a response thereto, which is diagnostic of a SARS-CoV-2 infection. In another aspect, detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present invention includes a method detecting a coronavirus infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with coronavirus. In one aspect, the sample comprises T cells. In another aspect, the response comprises inducing, increasing, promoting or stimulating anti-coronavirus activity of T cells. In another aspect, the T cells are CD8+ or CD4+ T cells. In another aspect, the method comprises determining whether the subject has been infected by or exposed to the coronavirus more than once by determining if the subject elicits a secondary T cell immune response profile that is different from a primary T cell immune response profile. In another aspect, the method further comprises diagnosing a coronavirus infection or exposure in a subject, the method comprising contacting a biological sample from a subject with a composition of composition of one or more proteins, peptides or multimers, and determining if the composition elicits a T cell immune response, wherein the T cell immune response identifies that the subject has been infected with or exposed to a coronavirus. In another aspect, the method is conducted three or more days following the date of suspected infection by or exposure to a coronavirus.

In another embodiment, the present invention includes a method detecting SARS-CoV-2 infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with SARS-CoV-2. In another aspect, the sample comprises T cells. In another aspect, the response comprises inducing, increasing, promoting or stimulating anti-SARS-CoV-2 activity of T cells. In another aspect, the T cells are CD8+ or CD4+ T cells. In another aspect, the method comprises determining whether the subject has been infected by or exposed to SARS-CoV-2 more than once by determining if the subject elicits a secondary T cell immune response profile that is different from a primary T cell immune response profile. In another aspect, the method further comprises diagnosing a SARS-CoV-2 infection or exposure in a subject, the method comprising contacting a biological sample from a subject with a composition of one or more proteins, peptides or multimers; and determining if the composition elicits a T cell immune response, wherein the T cell immune response identifies that the subject has been infected with or exposed to SARS-CoV-2. In another aspect, the method is conducted three or more days following the date of suspected infection by or exposure to a coronavirus.

In another embodiment, the present invention includes a kit for the detection of coronavirus or an immune response to coronavirus in a subject comprising, consisting of or consisting essentially of: one or more T cells that specifically detect the presence of: one or more amino acid sequences selected from SEQ ID NO: 1 to 1126, or a subsequence, portion, homologue, variant or derivative thereof; or a fusion protein comprising one or more amino acid sequences selected from SEQ ID NO: 1 to 1126; or a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 or more peptides selected from the amino acid sequences set forth in SEQ ID NO: 1 to 1126. In one aspect, the one or more amino acid sequences are selected from a coronavirus T cell epitope set forth in SEQ ID NO:874 to 1126. In another aspect, the composition comprises: one or more amino acid sequences selected from SEQ ID NO: 1 to 873, or a subsequence, portion, homologue, variant or derivative thereof; a fusion protein comprising one or more amino acid sequences selected from SEQ ID NO: 1 to 873; or a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or 873 more peptides selected from the amino acid sequences set forth in SEQ ID NO: 1 to 873. In another aspect, the amino acid sequence comprises a coronavirus CD8+ or CD4+ T cell epitope. In another aspect, the T cell epitope is not conserved in another coronavirus. In another aspect, the T cell epitope is conserved in another coronavirus. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) coronavirus or (ii) an immune response relevant to coronavirus infections, vaccines or therapies, including T cells responsive to coronavirus. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to coronavirus.

In another embodiment, the present invention includes a kit for the detection of SARS-CoV-2 or an immune response to SARS-CoV-2 in a subject comprising, consisting of or consisting essentially of: one or more T cells that specifically detect the presence of: one or more amino acid sequences selected from SEQ ID NO: 1 to 873, or a subsequence, portion, homologue, variant or derivative thereof; a fusion protein comprising one or more amino acid sequences selected from SEQ ID NO: 1 to 873; or a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or 873 more peptides selected from the amino acid sequences set forth in SEQ ID NO: 1 to 873. In another aspect, the one or more amino acid sequences is selected from a SARS-CoV-2 CD4 T cell epitope selected from SEQ ID NO: 1-280; a SARS-CoV-2 CD8 T cell epitope selected from SEQ ID NO: 281-803; or both. In another aspect, the one or more amino acid sequences exclude amino acid sequences selected from SEQ ID NOS: 245-280 and 804-873. In another aspect, the amino acid sequence comprises a SARS-CoV-2 CD8+ or CD4+ T cell epitope. In another aspect, the SARS-CoV-2 T cell epitope is not conserved in another coronavirus. In another aspect, the SARS-CoV-2 T cell epitope is conserved in another coronavirus. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) SARS-CoV-2 or (ii) an immune response relevant to SARS-CoV-2 infections, vaccines or therapies, including T cells responsive to SARS-CoV-2. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to SARS-CoV-2.

In another embodiment, the present invention includes a method of stimulating, inducing, promoting, increasing, or enhancing an immune response against a coronavirus in a subject, comprising: administering a composition of one or more proteins, peptides, multimers or a polynucleotide that expresses the protein, peptide or multimers, in an amount sufficient to stimulate, induce, promote, increase, or enhance an immune response against the coronavirus in the subject. In another aspect, the immune response provides the subject with protection against a coronavirus infection or pathology, or one or more physiological conditions, disorders, illnesses, diseases or symptoms caused by or associated with coronavirus infection or pathology. In another aspect, the immune response is specific to: one or more SARS-CoV-2 peptides selected from the amino acid sequences set forth in SEQ ID NO: 1 to 1126, or a subsequence, portion, homologue, variant or derivative thereof.

In another embodiment, the present invention includes a method of stimulating, inducing, promoting, increasing, or enhancing an immune response against SARS-CoV-2 in a subject, comprising: administering a composition of proteins, peptides, multimers or a polynucleotide that expresses the protein, peptide or multimers, in an amount sufficient to stimulate, induce, promote, increase, or enhance an immune response against SARS-CoV-2 in the subject. In one aspect, the immune response provides the subject with protection against a SARS-CoV-2 infection or pathology, or one or more physiological conditions, disorders, illnesses, diseases or symptoms caused by or associated with SARS-CoV-2 infection or pathology. In another aspect, the immune response is specific to: one or more SARS-CoV-2 peptides selected from the amino acid sequences set forth in SEQ ID NO: 1 to 873, or a subsequence, portion, homologue, variant or derivative thereof. In another aspect, the one or more SARS-CoV-2 peptides selected from the amino acid sequences set forth in SEQ ID NO: 1 to 873, or a subsequence, portion, homologue, variant or derivative thereof, exclude the amino acid sequences selected from SEQ ID NOS: 245-280 and 804-873.

In another embodiment, the present invention includes a method of stimulating, inducing, promoting, increasing, or enhancing an immune response against SARS-CoV-2 in a subject, comprising: administering to a subject an amount of a protein or peptide comprising, consisting of or consisting essentially of an amino acid sequence of the SARS-CoV-2 spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two peptides selected from the amino acid sequences set forth in SEQ ID NO: 1 to 873 or a subsequence, portion, homologue, variant or derivative thereof, in an amount sufficient to prevent, stimulate, induce, promote, increase, immunize against, or enhance an immune response against SARS-CoV-2 in the subject. In one aspect, the immune response provides the subject with protection against SARS-CoV-2 infection or pathology, or one or more physiological conditions, disorders, illnesses, diseases or symptoms caused by or associated with SARS-CoV-2 infection or pathology.

In another embodiment, the present invention includes a method of treating, preventing, or immunizing a subject against SARS-CoV-2 infection, comprising administering to a subject an amount of a protein or peptide comprising, consisting of, or consisting essentially of an amino acid sequence of a coronavirus spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two amino acid sequences selected from SEQ ID NO: 1 to 1126 or a subsequence, portion, homologue, variant or derivative thereof, in an amount sufficient to treat, prevent, or immunize the subject for SARS-CoV-2 infection, wherein the protein or peptide comprises or consists of a coronavirus T cell epitope that elicits, stimulates, induces, promotes, increases, or enhances an anti-SARS-CoV-2 T cell immune response. In one aspect, the one or more amino acid sequences are selected from SEQ ID NO: 1 to 873, or a subsequence, portion, homologue, variant or derivative thereof; a fusion protein comprising one or more amino acid sequences selected from SEQ ID NO: 1 to 873; or a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or 873 more peptides selected from the amino acid sequences set forth in SEQ ID NO: 1 to 873. In one aspect, the anti-SARS-CoV-2 T cell response is a CD8+, a CD4+ T cell response, or both. In another aspect, the T cell epitope is conserved across two or more clinical isolates of SARS-CoV-2, two or more circulating forms of SARS-CoV-2, or two or more coronaviruses. In another aspect, the SARS-CoV-2 infection is an acute infection. In another aspect, the subject is a mammal or a human. In another aspect, the method reduces SARS-CoV-2 viral titer, increases or stimulates SARS-CoV-2 viral clearance, reduces or inhibits SARS-CoV-2 viral proliferation, reduces or inhibits increases in SARS-CoV-2 viral titer or SARS-CoV-2 viral proliferation, reduces the amount of a SARS-CoV-2 viral protein or the amount of a SARS-CoV-2 viral nucleic acid, or reduces or inhibits synthesis of a SARS-CoV-2 viral protein or a SARS-CoV-2 viral nucleic acid. In another aspect, the method reduces one or more adverse physiological conditions, disorders, illness, diseases, symptoms or complications caused by or associated with SARS-CoV-2 infection or pathology. In another aspect, the method improves one or more adverse physiological conditions, disorders, illness, diseases, symptoms or complications caused by or associated with SARS-CoV-2 infection or pathology. In another aspect, the symptom is fever or chills, cough, shortness of breath or difficulty breathing, fatigue, muscle or body aches, headache, new loss of taste or smell, sore throat, congestion or runny nose, nausea or vomiting, or diarrhea. In another aspect, the method reduces or inhibits susceptibility to SARS-CoV-2 infection or pathology. In another aspect, the protein or peptide, or a subsequence, portion, homologue, variant or derivative thereof, is administered prior to, substantially contemporaneously with or following exposure to or infection of the subject with SARS-CoV-2. In another aspect, a plurality of SARS-CoV-2 T cell epitopes are administered prior to, substantially contemporaneously with or following exposure to or infection of the subject with SARS-CoV-2. In another aspect, the protein or peptide, or a subsequence, portion, homologue, variant or derivative thereof is administered within 2-72 hours, 2-48 hours, 4-24 hours, 4-18 hours, or 6-12 hours after a symptom of SARS-CoV-2 infection or exposure develops. In another aspect, the protein or peptide, or a subsequence, portion, homologue, variant or derivative thereof is administered prior to exposure to or infection of the subject with SARS-CoV-2. In another aspect, the method further comprises administering a modulator of immune response prior to, substantially contemporaneously with or following the administration to the subject of an amount of a protein or peptide. In another aspect, the modulator of immune response is a modulator of the innate immune response. In another aspect, the modulator is IL-6, IFN-γ, TGF-β, or IL-10, or an agonist or antagonist thereof. In another aspect, the one or amino acid sequences exclude amino acid sequences selected from SEQ ID NOS: 245-280 and 804-873.

In another embodiment, the present invention includes a method of treating, preventing, or immunizing a subject against SARS-CoV-2 infection, comprising administering to a subject the composition of one or more proteins, peptides or multimers in an amount sufficient to treat, prevent, or immunize the subject for SARS-CoV-2 infection. In one aspect, the SARS-CoV-2 infection is an acute infection. In another aspect, the method reduces SARS-CoV-2 viral titer, increases or stimulates SARS-CoV-2 viral clearance, reduces or inhibits SARS-CoV-2 viral proliferation, reduces or inhibits increases in SARS-CoV-2 viral titer or SARS-CoV-2 viral proliferation, reduces the amount of a SARS-CoV-2 viral protein or the amount of a SARS-CoV-2 viral nucleic acid, or reduces or inhibits synthesis of a SARS-CoV-2 viral protein or a SARS-CoV-2 viral nucleic acid. In another aspect, the method reduces one or more adverse physiological conditions, disorders, illness, diseases, symptoms or complications caused by or associated with SARS-CoV-2 infection or pathology. In another aspect, the method improves one or more adverse physiological conditions, disorders, illness, diseases, symptoms or complications caused by or associated with SARS-CoV-2 infection or pathology. In another aspect, the symptom is fever or chills, cough, shortness of breath or difficulty breathing, fatigue, muscle or body aches, headache, new loss of taste or smell, sore throat, congestion or runny nose, nausea, vomiting, or diarrhea. In another aspect, the method reduces or inhibits susceptibility to SARS-CoV-2 infection or pathology. In another aspect, the composition is administered prior to, substantially contemporaneously with or following exposure to or infection of the subject with SARS-CoV-2. In another aspect, the composition is administered prior to, substantially contemporaneously with or following exposure to or infection of the subject with SARS-CoV-2. In another aspect, the composition is administered within 2-72 hours, 2-48 hours, 4-24 hours, 4-18 hours, or 6-12 hours after a symptom of SARS-CoV-2 infection or exposure develops. In another aspect, the composition is administered prior to exposure to or infection of the subject with SARS-CoV-2.

In another embodiment, the present invention includes a peptide or peptides that are immunoprevalent or immunodominant in a virus obtained by a method consisting of, or consisting essentially of: obtaining an amino acid sequence of the virus; determining one or more sets of overlapping peptides spanning one or more virus antigen using unbiased selection; synthesizing one or more pools of virus peptides comprising the one or more sets of overlapping peptides; combining the one or more pools of virus peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is a coronavirus. In another aspect, the coronavirus is SARS-CoV-2. In another aspect, the immunodominant peptides are selected from 1, 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 or more peptides selected from the amino acid sequences set forth in SEQ ID NO: 1 to 1126. In another aspect, the immunodominant peptides are selected from 1, 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or more peptides selected from the amino acid sequences set forth in SEQ ID NO: 1 to 873. In another aspect, the peptide or peptides exclude amino acid sequences set forth in SEQ ID NOS: 245-280 and 804-873.

In another embodiment, the present invention includes a method of selecting an immunoprevalent or immunodominant peptide or protein of a virus comprising, consisting of, or consisting essentially of: obtaining an amino acid sequence of the virus; determining one or more sets of overlapping peptides spanning one or more virus antigen using unbiased selection; synthesizing one or more pools of virus peptides comprising the one or more sets of overlapping peptides; combining the one or more pools of virus peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is a coronavirus. In another aspect, the coronavirus is SARS-CoV-2. In another aspect, the immunodominant peptides are selected from 1, 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 or more peptides selected from the amino acid sequences set forth in SEQ ID NO: 1 to 1126. In another aspect, the immunodominant peptides are selected from 1, 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or more peptides selected from the amino acid sequences set forth in SEQ ID NO: 1 to 873. In another aspect, the peptide or peptides exclude amino acid sequences set forth in SEQ ID NOS: 245-280 and 804-873.

In another embodiment, the present invention includes a polynucleotide that expresses one or more peptides or proteins, comprising, consisting of, or consisting essentially of an amino acid sequence selected from SEQ ID NO: 1 to 1126, or a subsequence, portion, homologue, variant or derivative thereof; a fusion protein comprising one or more amino acid sequences selected from SEQ ID NO: 1 to 1126; or a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 or more peptides comprising, consisting of, or consisting essentially of amino acid sequences selected from SEQ ID NO: 1 to 1126. In one aspect, the vector comprises the polynucleotide of claim that expresses one or more peptides or proteins, comprising, consisting of, or consisting essentially of an amino acid sequence selected from SEQ ID NO: 1 to 1126, or a subsequence, portion, homologue, variant or derivative thereof; a fusion protein comprising one or more amino acid sequences selected from SEQ ID NO: 1 to 1126; or a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 or more peptides comprising, consisting of, or consisting essentially of amino acid sequences selected from SEQ ID NO: 1 to 1126, a viral vector, or a host cell the comprises the same.

In another embodiment, the present invention includes a polynucleotide that expresses one or more peptides or proteins comprising, consisting of, or consisting essentially of an amino acid sequence selected from SEQ ID NO: 1 to 873, or a subsequence, portion, homologue, variant or derivative thereof; a fusion protein comprising one or more amino acid sequences selected from SEQ ID NO: 1 to 873; or a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or more peptides selected from SEQ ID NO: 1 to 873. In one aspect, the vector comprises the polynucleotide of claim that expresses one or more peptides or proteins comprising, consisting of, or consisting essentially of an amino acid sequence selected from SEQ ID NO: 1 to 873, or a subsequence, portion, homologue, variant or derivative thereof; a fusion protein comprising one or more amino acid sequences selected from SEQ ID NO: 1 to 873; or a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or more peptides selected from SEQ ID NO: 1 to 873, a viral vector, or a host cell that comprises the same.

### BRIEF DESCRIPTION OF THE DRAWINGS

For a more complete understanding of the features and advantages of the present invention, reference is now made to the detailed description of the invention along with the accompanying figures and in which:

FIGS. 1A to 1E show SARS-CoV-2-specific T cell reactivity per protein. Immunodominance at the ORF/antigen level and breath of T cell responses are shown for CD4⁺ (FIG. 1A) and CD8⁺ (FIG. 1C) T cells. Data are shown as geometric mean ± geometric SD. The numbers of donors recognizing one or more antigens with a response >10%, normalized per donor to account for the differences in magnitude based on days PSO, are shown for CD4⁺ (FIG. 1B) and CD8⁺ (FIG. 1D) T cells. Empty circles represent CD4⁺ and CD8⁺ T cell reactivity per protein, respectively. Filled circles highlight the immunodominant antigens recognized by CD4⁺ and CD8⁺ T cells, respectively. FIG. 1E shows a flow chart of a scheme of experimental strategy selected for HLA class I and class II epitope identification, and representative graphs depicting the flow cytometry gating strategy for defining antigen-specific CD4⁺ and CD8⁺ T cells by OX40⁺CD137⁺ and CD69⁺CD137⁺ expression, respectively.

FIGS. 2A to 2L show SARS-CoV-2-specific CD4⁺ T cell reactivities and their correlations with antibody production and CD8⁺ T cell reactivity. RBD IgG serology is shown for all the donors of this cohort (FIG. 2A). Serology data of panel A are correlated with CD4⁺ T cell reactivities specific against all combined proteins (FIG. 2B), structural proteins S, M, and N (FIG. 2C), non-structural proteins nsp3, nsp4, nsp12, and nsp13 (FIG. 2D), and ORF8 and ORF3a (FIG. 2E). The total CD8⁺ T cell reactivity is correlated with the total CD4⁺ T cell reactivity (FIG. 2F) and the CD4⁺ T cell reactivity against structural proteins S, M, and N (FIG. 2G), non-structural proteins nsp3, nsp4, nsp12, and nsp13 (FIG. 2H), and ORF8 and ORF3a (FIG. 2I). Empty and filled circles represent correlation between CD4⁺ T cell reactivity and serology or CD8⁺ T cell reactivity, respectively. All analyses were performed using Spearman correlation and the p-values shown were not corrected for multiple hypothesis testing. FIGS. 2J to 2L shows the correlations of SARS-CoV-2-specific CD4+ and CD8+ T cell reactivities per protein. CD4+ and CD8+ T cell reactivities are correlated for each of the 9 SARS-CoV-2 antigens that were immunodominant for CD4+ T cells: S, M, and N (FIG. 2J); nsp3, nsp4, nsp12, and nsp13 (FIG. 2K); and ORF8 and ORF3a (FIG. 2L). All analyses were performed using Spearman correlation and the p-values shown were not corrected for multiple hypothesis testing.

FIG. 3A shows SARS-CoV-2 CD4+ T cell epitopes as a function of the number of responding donors recognized and strength of responses (FIG. 3A). These data highlight that 49 immunodominant epitopes account for 45% of the total response. Heat maps of HLA predicted binding patterns in the 27 most frequent HLA class II alleles worldwide (Greenbaum et al., 2011). Predicted binding patterns for the top 49 most immunodominant SARS-CoV-2 CD4+ T cell epitopes are compared with a set of matched non-epitopes. Predicted IC50 were calculated using NetMHCIIpan embedded in Tepitool (Dhanda et al., 2019; Karosiene et al., 2013; Paul et al., 2016) and converted to Log10 scale. Lower values indicate stronger predicted binding affinity, and are highlighted at the red end of the spectrum. Predicted values with an IC50 <1000 nM (Log10 scale <3) are considered positive binders (Paul et al., 2019; Southwood et al., 1998). FIGS. 3B to 3F show SARS-CoV-2 immunodominant epitope HLA class II binding capacity and promiscuity. A comparison of the HLA class II binding capacity of 49 immunodominant epitopes as determined by binding predictions or as measured experimentally (FIG. 3B), suggesting feasibility for using binding predictions to assess HLA-restriction. Predicted HLA class II binding promiscuity is shown for the same 49 epitopes (white circles), and also 49 non-epitopes (black circles), considering the 27 HLA class II alleles most frequent worldwide (FIGS. 3C-3D), or the 58 HLA class II alleles specific to the study cohort (FIGS. 3E-3F). The number of HLA class II alleles predicted to bind epitopes (white circles) and non-epitopes (black circles) are based on a prediction cutoff value of IC50<1000nM. Statistical comparisons were performed using Mann-Whitney.

FIGS. 4A to 4Q show the number of donors tested with their HLA-matched class I peptides for each of the 8 dominant proteins for CD8⁺ is shown in panel (FIG. 4A). The distribution of allele-specific CD8⁺ responses for the 18 class I alleles that were tested in 3 or more donors is shown as function of protein composition (FIG. 4B) or the HLA class I alleles tested (FIG. 4C). Bars to the right represent the total magnitude of AIM⁺ CD8⁺ T cells divided by the number of positive donors. Bars to the left represent the frequency of positive tests. The total number of epitopes identified for each class I allele is shown in panel (FIG. 4D). FIGS. 4E to 4J show HLA phenotype frequency in the COVID-19 cohort analyzed compared with the worldwide phenotype frequencies available in the IEDB-AR population coverage tool (Bui et al., 2006; Dhanda et al., 2019). HLA class I frequency for A and B loci for the top 28 HLA class I with frequency >5% in the worldwide population are shown in panels FIG. 4E and FIG. 4F, respectively. (FIG. 4G) Coverage of class I predicted peptides based on the HLA typing of the population. HLA class II frequency for DRB1, DP and DQ loci for the top HLA class II with frequency >5% in the worldwide population or the studied cohort are shown in panels FIGS. 4H, 4I, and 4J respectively. FIGS. 4K-4Q show analyses of CD4+ and CD8+ T cell epitopes identified compared to non-epitopes within the same proteins. Comparison of sequenced identity between CD4+ T cell epitopes and non-epitopes as a function of sequence identity with the CCC in S, M, and N combined (FIG. 4K), ORF8 and ORF3a (FIG. 4L), and non-structural proteins (FIG. 4M). For CD8+ epitopes and non-epitopes, the sequence identities with CCC are shown for S, M, and N (FIG. 4N), ORF3a (FIG. 4O), and non-structural proteins (FIG. 4P). Statistical analyses were performed using the Kolmogorov-Smirnov test, and data are shown as violin plots. (FIG. 4Q) Overlap of previously identified epitopes in unexposed (Mateus et al., 2020 Science) with the proteins analyzed in this study and the current epitopes identified in COVID-19 donors. The Venn diagram was calculated with the Venn Diagram Plotter (PNNL, OMICS.PNL.gov).

FIGS. 5A to 5L show the immunodominant regions for CD4⁺ T cell reactivity for S (FIG. 5A), N (FIG. 5B) and M (FIG. 5C) proteins as a function of the frequency of positive response (red) and total magnitude (black) in the topmost panel. The dotted red line indicates the cutoff of 20% frequency of positivity used to define the immunodominant regions boxed in red. The x-axis labels in this topmost panel indicate the middle position of the peptide. Binding promiscuity was calculated based on NetMHCIIpan predicted IC₅₀ for the alleles present in the cohort of donors tested and is shown in grey on the upper middle panel. The lower middle panel shows the % homology of SARS-CoV-2 to the four most frequent CCC (229E, NL63, HKU1, and OC43) and the max value. The linear structure of each protein is drawn below the graph of homology (Cai et al., 2020; Zeng et al., 2020; UniProtKB - P59596 (VME1_SARS)). The magnitude of CD8⁺ responses to class I predicted epitopes is shown in the bottom panel, where black dots represent epitopes and grey dots represent non-epitopes, each centered on the middle position of the peptide. FIGS. 5D to 5L show correlations of predicted binding promiscuity to the alleles present in the donor cohort tested with the frequency of positive response for S (FIG. 5D), N (FIG. 5H), and M (FIG. 5J) epitopes. Frequency of positive response is also correlated with the maximum % homology of the SARS-CoV-2 sequence to CCC and plotted for S (FIG. 5E), M (FIG. 5F), and N (FIG. 5K). In the final column of panels, the correlation of frequency of positivity and the cleavage probability percentile rank (calculated using the IEDB MHCII-NP tool) are shown for S (FIG. 5F), N (FIG. 5I), and M (FIG. 5L). Statistics were performed using the Spearman correlation and the line on each graph is a simple linear regression.

FIGS. 6A to 6L show T cell responses to SARS-CoV-2 megapools as measured in AIM (empty circles) and FluoroSpot (filled in circles) assays. Twenty-five unexposed and 31 convalescent COVID-19 donors were tested in the AIM assays (FIG. 6A and FIG. 6C), and all donors were also tested in the FluoroSpot assays (FIG. 6B and FIG. 6D). CD4⁺ T cell responses to CD4-R+S (previously described), CD4-E (280 class II epitopes identified in this study), and EC Class II (Nelde et al 2020) megapools were measured via AIM (FIG. 6A) and FluoroSpot (FIG. 6B). CD8⁺ T cell responses to CD8-A+B (previously described), CD8-E (454 class I epitopes identified in this study), and EC Class I (Nelde et al 2020) megapools were measured via AIM (C) and FluoroSpot (FIG. 6D). Bars represent geometric mean ± geometric SD, and p-values were calculated by Mann-Whitney. Panels FIG. 6E- FIG. 6H show ROC analysis for CD4⁺ and CD8⁺ T cell response data in FluoroSpot (FIG. 6F- FIG. 6H) and AIM (FIG. 6E -FIG. 6G) assays. In each panel, curves are shown for the 3 peptide pools tested. For a given pool, T cell responses were used to classify individuals into predicted exposed’ or ‘predicted unexposed’, at varying thresholds starting with the highest observed response to the lowest. The inventors then compared these data with the actual SARS-CoV-2 exposure status of the individuals and calculated the rate of true positive (predicted exposed / total exposed) and the rate of false positives (predicted exposed / total non-exposed). Additionally, the inventors further tested 17 of these COVID-19 convalescent donors in FluoroSpot with a titration of 200, 50, 25, and 12.5×10³ cells per well with the indicated CD4-MPs (FIG. 6I- FIG. 6J) and CD8-MPs (FIG. 6K- FIG. 6L).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. Numerous variations, changes, and substitutions will now occur to those skilled in the art without departing from the invention. It should be understood that various alternatives to the embodiments of the invention described herein may be employed in practicing the invention. To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless specifically stated or obvious from context, as used herein, the term “or” is understood to be inclusive.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

Proteins and peptides include isolated and purified forms. Proteins and peptides also include those immobilized on a substrate, as well as amino acid sequences, subsequences, portions, homologues, variants, and derivatives immobilized on a substrate.

Proteins and peptides can be included in compositions, for example, a pharmaceutical composition. In particular embodiments, a pharmaceutical composition is suitable for specific or non-specific immunotherapy, or is a vaccine composition.

Isolated nucleic acid (including isolated nucleic acid) encoding the proteins and peptides are also provided. Cells expressing a protein or peptide are further provided. Such cells include eukaryotic and prokaryotic cells, such as mammalian, insect, fungal and bacterial cells.

Methods and uses and medicaments of proteins and peptides of the invention are included. Such methods, uses and medicaments include modulating immune activity of a cell against a pathogen, for example, a bacteria or virus.

The term “peptide mimetic” or “peptidomimetic” refers to protein-like chain designed to mimic a peptide or protein. Peptide mimetics may be generated by modifying an existing peptide or by designing a compound that mimic peptides, including peptoids and β-peptides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

A “percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “multimer” refers to a complex comprising multiple monomers (e.g., a protein complex) associated by noncovalent bonds. The monomers be substantially identical monomers, or the monomers may be different. In embodiments, the multimer is a dimer, a trimer, a tetramer, or a pentamer.

As used herein, the term “Major Histocompatibility Complex” (MHC) is a generic designation meant to encompass the histocompatibility antigen systems described in different species including the human leucocyte antigens (HLA). Typically, MHC Class I or Class II multimers are well known in the art and include but are not limited to dimers, tetramers, pentamers, hexamers, heptamers and octamers.

As used herein, the term “MHC/peptide multimer” refers to a stable multimeric complex composed of MHC protein(s) subunits loaded with a peptide of the present invention. For example, an MHC/peptide multimer (also called herein MHC/peptide complex) include, but are not limited to, an MHC/peptide dimer, trimer, tetramer, pentamer or higher valency multimer. In humans there are three major different genetic loci that encode MHC class I molecules (the MHC molecules of the human are also designated human leukocyte antigens (HLA)): HLA-A, HLA-B, HLA-C, e.g., HLA-A*01, HLA-A*02, and HLA-A*11 are examples of different MHC class I alleles that can be expressed from these loci. Non-classical human MHC class I molecules such as HLA-E (homolog of mice Qa-1b) and MICA/B molecules are also encompassed by the present invention. In some embodiments, the MHC/peptide multimer is an HLA/peptide multimer selected from the group consisting of HLA-A/peptide multimer, HLA-B/peptide multimer, HLA-C/peptide multimer, HLA-E/peptide multimer, MICA/peptide multimer and MICB/peptide multimer.

In humans there are three major different genetic loci that encode MHC class II molecules: HLA-DR, HLA-DP, and HLA-DQ, each formed of two polypeptides, alpha and beta chains (A and B genes). For example, HLA-DQA1*01, HLA-DRB1 *01, and HLA-DRB1*03 are different MHC class II alleles that can be expressed from these loci. It should be further noted that non-classical human MHC class II molecules such as HLA-DM and HL-DOA (homolog in mice is H2-DM and H2-O) are also encompassed by the present invention. In some embodiments, the MHC/peptide multimer is an HLA/peptide multimer selected from the group consisting of HLA-DP/peptide multimer, HLA-DQ/peptide multimer, HLA-DR/peptide multimer, HLA-DM/peptide multimer and HLA-DO/peptide multimer.

An MHC/peptide multimer may be a multimer where the heavy chain of the MHC is biotinylated, which allows combination as a tetramer with streptavidin. MHC-peptide tetramers have increased avidity for the appropriate T cell receptor (TCR) on T lymphocytes. The multimers can also be attached to paramagnetic particles or magnetic beads to facilitate removal of non-specifically bound reporter and cell sorting. Multimer staining does not kill the labelled cells, thus, cell integrity is maintained for further analysis. In some embodiments, the MHC/peptide multimer of the present invention is particularly suitable for isolating and/or identifying a population of CD8+ T cells having specificity for the peptide of the present invention (in a flow cytometry assay).

The peptides or MHC class I or class II multimer as described herein is particularly suitable for detecting T cells specific for one or more peptides of the present invention. The peptide(s) and/or the MHC/multimer complex of the present invention is particularly suitable for diagnosing coronavirus infection in a subject. For example, the method comprises obtaining a blood or PBMC sample obtained from the subject with an amount of a least peptide of the present invention and detecting at least one T cell displaying a specificity for the peptide. Another diagnostic method of the present invention involves the use of a peptide of the present invention that is loaded on multimers as described above, so that the isolated CD8+ or CD4+ T cells from the subject are brought into contact with the multimers, at which the binding, activation and/or expansion of the T cells is measured. For example, following the binding to antigen presenting cells, e.g., those having the MHC class I or class II multimer, the number of CD8+ and/or CD4+ cells binding specifically to the HLA-peptide multimer may be quantified by measuring the secretion of lymphokines/cytokines, division of the T cells, or standard flow cytometry methods, such as, for example, using fluorescence activated cell sorting (FACS). The multimers can also be attached to paramagnetic ferrous or magnetic beads to facilitate removal of non-specifically bound reporter and cell sorting.

The MHC class I or class II peptide multimers as described herein can also be used as therapeutic agents. The peptide and/or the MHC class I or class II peptide multimers of the present invention are suitable for treating or preventing a coronavirus infection in a subject. The MHC Class I or Class II multimers can be administered in soluble form or loaded on nanoparticles.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein or peptide, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies are large, complex molecules (molecular weight of ~150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

As used herein, the term “antigen” and the term “epitope” refers to a molecule or substance capable of stimulating an immune response. In one example, epitopes include but are not limited to a polypeptide and a nucleic acid encoding a polypeptide, wherein expression of the nucleic acid into a polypeptide is capable of stimulating an immune response when the polypeptide is processed and presented on a Major Histocompatibility Complex (MHC) molecule. Generally, epitopes include peptides presented on the surface of cells non-covalently bound to the binding groove of Class I or Class II MHC, such that they can interact with T cell receptors and the respective T cell accessory molecules. However, antigens and epitopes also apply when discussing the antigen binding portion of an antibody, wherein the antibody binds to a specific structure of the antigen.

Proteolytic Processing of Antigens. Epitopes that are displayed by MHC on antigen presenting cells are cleavage peptides or products of larger peptide or protein antigen precursors. For MHC I epitopes, protein antigens are often digested by proteasomes resident in the cell. Intracellular proteasomal digestion produces peptide fragments of about 3 to 23 amino acids in length that are then loaded onto the MHC protein. Additional proteolytic activities within the cell, or in the extracellular milieu, can trim and process these fragments further. Processing of MHC Class II epitopes generally occurs via intracellular proteases from the lysosomal/endosomal compartment. The present invention includes, in one embodiment, pre-processed peptides that are attached to the anti-CD40 antibody (or fragment thereof) that directs the peptides against which an enhanced immune response is sought directly to antigen presenting cells.

The present invention includes methods for specifically identifying the epitopes within antigens most likely to lead to the immune response sought for the specific sources of antigen presenting cells and responder T cells.

As used herein, the term “T cell epitope” refers to a specific amino acid that when present in the context of a Major or Minor Histocompatibility Complex provides a reactive site for a T cell receptor. The T-cell epitopes or peptides that stimulate the cellular arm of a subject’s immune system are short peptides of about 8-25 amino acids. T-cell epitopes are recognized by T cells from animals that are immune to the antigen of interest. These T-cell epitopes or peptides can be used in assays such as the stimulation of cytokine release or secretion or evaluated by constructing major histocompatibility (MHC) proteins containing or “presenting” the peptide. Such immunogenically active fragments are often identified based on their ability to stimulate lymphocyte proliferation in response to stimulation by various fragments from the antigen of interest.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of effector and/or suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term an “immunogenic composition” and “vaccine” refer to a composition that comprises an antigenic molecule where administration of the composition to a subject or patient results in the development in the subject of a humoral and/or a cellular immune response to the antigenic molecule of interest. “Vaccine” refers to a composition that can provide active acquired immunity to and/or therapeutic effect (e.g., treatment) of a particular disease or a pathogen. A vaccine typically contains one or more agents that can induce an immune response in a subject against a pathogen or disease, i.e., a target pathogen or disease. The immunogenic agent stimulates the body’s immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g., preventing or ameliorating the effects of a future infection by any natural or pathogen) or therapeutic (e.g., reducing symptoms or aberrant conditions associated with infection). The administration of vaccines is referred to vaccination. In some examples, a vaccine composition can provide nucleic acid, e.g., mRNA that encodes antigenic molecules (e.g., peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g., one or more peptides that are known to be expressed in the pathogen (e.g., pathogenic bacterium or virus).

The present invention provides nucleic acid molecules, specifically polynucleotides, primary constructs and/or mRNA that encode one or more polynucleotides that express one or more peptides or proteins, comprising, consisting of, or consisting essentially of an amino acid sequence selected from SEQ ID NO: 1 to 1126, or a subsequence, portion, homologue, variant or derivative thereof for use in immune modulation. The term “nucleic acid” refers to any compound and/or substance that comprise a polymer of nucleotides, referred to herein as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs), including diastereomers of LNAs, functionalized LNAs, or hybrids thereof.

One method of immune modulation of the present invention includes direct or indirect gene transfer, i.e., local application of a preparation containing the one or more polynucleotides (DNA, RNA, mRNA, etc.) that expresses the one or more peptides or proteins, comprising, consisting of, or consisting essentially of an amino acid sequence selected from SEQ ID NO: 1 to 1126, or a subsequence, portion, homologue, variant or derivative thereof. A variety of well-known vectors can be used to deliver to cells the one or more polynucleotides or the peptides or proteins expressed by the polynucleotides, including but not limited to adenoviral vectors and adeno-associated vectors. In addition, naked DNA, liposome delivery methods, or other novel vectors developed to deliver the polynucleotides to cells can also be beneficial. Any of a variety of promoters can be used to drive peptide or protein expression, including but not limited to endogenous promoters, constitutive promoters (e.g., cytomegalovirus, adenovirus, or SV40), inducible promoters (e.g., a cytokine promoter such as the interleukin-1, tumor necrosis factor-alpha, or interleukin-6 promoter), and tissue specific promoters to express the immunogenic peptides or proteins of the present invention.

The immunization may include adenovirus, adeno-associated virus, herpes virus, vaccinia virus, retroviruses, or other viral vectors with the appropriate tropism for cells likely to present the antigenic peptide(s) or protein(s) may be used as a gene transfer delivery system for a therapeutic peptide(s) or protein(s), comprising, consisting of, or consisting essentially of an amino acid sequence selected from SEQ ID NO: 1 to 1126, or a subsequence, portion, homologue, variant or derivative thereof, gene expression construct. Viral vectors which do not require that the target cell be actively dividing, such as adenoviral and adeno-associated vectors, are particularly useful when the cells are accumulating, but not proliferative. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; and Miller and Rosman, Bio Techniques 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

The immunization may also include inserting the one or more polynucleotides (DNA, RNA, mRNA, etc.) that express the one or more peptides or proteins, comprising, consisting of, or consisting essentially of an amino acid sequence selected from SEQ ID NO: 1 to 1126, or a subsequence, portion, homologue, variant or derivative thereof into the viral vector, along with another gene which encodes the ligand for a receptor on a specific target cell, for example, such that the vector is now target specific. Viral vectors can be made target specific by attaching, for example, a sugar, a glycolipid, or a protein. Targeting can also be accomplished by using an antibody to target the viral vector. Those of skill in the art will know of, or can readily ascertain without undue experimentation, specific polynucleotide sequences which can be inserted into the viral genome or attached to a viral envelope to allow target specific delivery of the viral vector containing the gene.

Since recombinant viruses are defective, they require assistance in order to produce infectious vector particles. This assistance can be provided, for example, by using helper cell lines that contain plasmids encoding all of the structural genes of the virus under the control of regulatory sequences within the viral genome. These plasmids are missing a nucleotide sequence which enables the packaging mechanism to recognize a polynucleotide transcript for encapsidation. These cell lines produce empty virions, since no genome is packaged. If a viral vector is introduced into such cells in which the packaging signal is intact, but the structural genes are replaced by other genes of interest, the vector can be packaged and vector virion produced.

Viral or non-viral approaches may also be employed for the introduction of one or more therapeutic polynucleotides that express the one or more peptides or proteins, comprising, consisting of, or consisting essentially of an amino acid sequence selected from SEQ ID NO: 1 to 1126, or a subsequence, portion, homologue, variant or derivative thereof, into polynucleotide-encoding polynucleotide into antigen presenting cells. The polynucleotides may be DNA, RNA, mRNA that directly encode the one or more peptides or proteins of the present invention, or may be introduced as part of an expression vector. Another example of an immunization includes colloidal dispersion systems that include macromolecule complexes, nanocapsules, microspheres, beads, and lipid-based systems including oil-in-water emulsions, micelles, mixed micelles, and liposomes and the one or more polynucleotides that express the one or more peptides or proteins, comprising, consisting of, or consisting essentially of an amino acid sequence selected from SEQ ID NO: 1 to 1126, or a subsequence, portion, homologue, variant or derivative thereof. One non-limiting example of a colloidal system for use with the present invention is a liposome. Liposomes are artificial membrane vesicles which are useful as delivery vehicles in vitro and in vivo. It has been shown that large unilamellar vesicles (LUV), which range in size from 0.2-4.0 micrometers that can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (Zakut and Givol, supra) encapsulation of the genes of interest at high efficiency while not compromising their biological activity; (Fearnhead, et al., supra) preferential and substantial binding to a target cell in comparison to non-target cells; (Korsmeyer, S. J., supra) delivery of the aqueous contents of the vesicle to the target cell cytoplasm at high efficiency; and (Kinoshita, et al., supra) accurate and effective expression of genetic information (Mannino, et al., Bio Techniques, 6:682, 1988).

The composition for immunizing the subject or patient may, in certain embodiments comprise a combination of phospholipid, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization, specifically, cells that can become infected with a coronavirus or interact with the proteins, peptides, and/or gene products of a coronavirus, e.g., immune cells.

For any of the above approaches, the immune modulating polynucleotide construct, composition, or formulation is preferably applied to a site that will enhance the immune response. For example, the immunization may be intramuscular, intraperitoneal, enteral, parenteral, intranasal, intrapulmonary, or subcutaneous. In the gene delivery constructs of the instant invention, polynucleotide expression is directed from any suitable promoter (e.g., the human cytomegalovirus, simian virus 40, actin or adenovirus constitutive promoters; or the cytokine or metalloprotease promoters for activated synoviocyte specific expression).

In one example of the immune modifying peptide(s) or protein(s) include polynucleotides, constructs and/or mRNAs that express the one or more polynucleotides that express the one or more peptides or proteins, comprising, consisting of, or consisting essentially of an amino acid sequence selected from SEQ ID NO: 1 to 1126, or a subsequence, portion, homologue, variant or derivative thereof, that are designed to improve one or more of the stability and/or clearance in tissues, uptake and/or kinetics, cellular access by the peptide(s) or protein(s), translational, mRNA half-life, translation efficiency, immune evasion, protein production capacity, accessibility to circulation, peptide(s) or protein(s) half-life and/or presentation in the context of MHC on antigen presenting cells.

The present invention contemplates immunization for use in both active and passive immunization embodiments. Immunogenic compositions, proposed to be suitable for use as a vaccine, may be prepared most readily directly from immunogenic peptides, proteins, monomers, multimers and/or peptide-MHC complexes prepared in a manner disclosed herein. The antigenic material is generally processed to remove undesired contaminants, such as, small molecular weight molecules, incomplete proteins, or when manufactured in plant cells, plant components such as cell walls, plant proteins, and the like. Often, these immunizations are lyophilized for ease of transport and/or to increase shelf-life and can then be more readily dissolved in a desired vehicle, such as saline.

The preparation of immunizations (also referred to as vaccines) that contain the immunogenic proteins of the present invention as active ingredients is generally well understood in the art, as exemplified by U.S. Letters Pats. 4,608,251; 4,601,903; 4,599,231; 4,599,230; 4,596,792; and 4,578,770, all incorporated herein by reference. Typically, such immunizations are prepared as injectables. The immunizations can be a liquid solution or suspension but may also be provided in a solid form suitable for solution in, or suspension in, liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, buffers, or the like and combinations thereof. In addition, if desired, the immunization may contain minor amounts of auxiliary substances such as wetting or emulsifying agents, pH buffering agents, or adjuvants which enhance the effectiveness of the vaccines. The immunization is/are administered in a manner compatible with the dosage formulation, and in such amount as will be therapeutically effective and immunogenic. The quantity to be administered depends on the subject to be treated, including, e.g., the capacity of the individual’s immune system to synthesize antibodies, and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable but are typified by an initial administration followed by subsequent inoculations or other administrations.

The manner of application of the immunization may be varied widely. Any of the conventional methods for administration of a vaccine are applicable. These are believed to also include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 70° to 101° C. for 30 second to 2-minute periods respectively. Aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

In many instances, it will be desirable to have multiple administrations of the vaccine, usually not exceeding six to ten immunizations, more usually not exceeding four immunizations and preferably one or more, usually at least about three immunizations. The immunizations will normally be at from two to twelve-week intervals, more usually from three to five-week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescent agents, and the like. These techniques are well known and may be found in a wide variety of patents, such as Hudson and Cranage, Vaccine Protocols, 2003 Humana Press, relevant portions incorporated herein by reference.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington’s Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

Many suitable expression systems are commercially available, including, for example, the following: baculovirus expression (Reilly, P. R., et al., BACULOVIRUS EXPRESSION VECTORS: A LABORATORY MANUAL (1992); Beames, et al., Biotechniques 11:378 (1991); Pharmingen; Clontech, Palo Alto, Calif.)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif., and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), relevant portion incorporated herein by reference.

As used herein, the term “effective amount” or “effective dose” refers to that amount of the peptide or protein T cell epitopes of the invention sufficient to induce immunity, to prevent and/or ameliorate an infection or to reduce at least one symptom of an infection and/or to enhance the efficacy of another dose of peptide or protein T cell epitopes. An effective dose may refer to the amount of peptide or protein T cell epitopes sufficient to delay or minimize the onset of an infection. An effective dose may also refer to the amount of peptide or protein T cell epitopes that provides a therapeutic benefit in the treatment or management of an infection. Further, an effective dose is the amount with respect to peptide or protein T cell epitopes of the invention alone, or in combination with other therapies, that provides a therapeutic benefit in the treatment or management of an infection. An effective dose may also be the amount sufficient to enhance a subject’s (e.g., a human’s) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms, in this case, an infectious disease, and more particularly, a coronavirus infection. The full prophylactic effect does not necessarily occur by administration of one dose, and may occur only after administration of a series of doses. Thus, a prophylactically effective amount may be administered in one or more administrations. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control.

The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), relevant portions incorporated herein by reference.

As used herein, the term “immune stimulator” refers to a compound that enhances an immune response via the body’s own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as peptide or protein T cell epitopes s of the invention, or can be administered separately. Either the protein or an expression vector encoding the protein can be administered to produce an immunostimulatory effect.

As used herein, in certain embodiments, the term “protective immune response” or “protective response” refers to an immune response mediated by antibodies against an infectious agent, which is exhibited by a vertebrate (e.g., a human), which prevents or ameliorates an infection or reduces at least one symptom thereof. Peptide and protein T cell epitopes of the invention can stimulate the production of antibodies that, for example, neutralize infectious agents, blocks infectious agents from entering cells, blocks replication of said infectious agents, and/or protect host cells from infection and destruction. In other embodiments, the term can also refer to an immune response that is mediated by T-lymphocytes and/or other white blood cells against an infectious agent, exhibited by a vertebrate (e.g., a human), that prevents or ameliorates flavivirus infection or reduces at least one symptom thereof. Peptide and protein T cell epitopes of the invention can stimulate the T cell responses that, for example, neutralize infectious agents, kill virus infected cells, blocks infectious agents from entering cells, blocks replication of said infectious agents, and/or protect host cells from infection and destruction.

The terms “biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The terms “virus” or “virus particle” are used according to its plain ordinary meaning within Virology and refers to a virion including the viral genome (e.g., DNA, RNA, single strand, double strand), viral capsid and associated proteins, and in the case of enveloped viruses (e.g., herpesvirus), an envelope including lipids and optionally components of host cell membranes, and/or viral proteins.

In embodiments, the virus is a coronavirus. Non-limiting examples of coronaviruses (CoV) from which T cell epitopes can be identified include, e.g., SARS-CoV (SARS-CoV-1), MERS-CoV, and SARS-CoV-2, but also betacoronaviruses, e.g., HCoV-OC43, HCoVHKU1, HCoV-229E and alphacoronaviuses such as HCoV-NL63, and/or other coronaviruses endemic in humans. The viral genome of coronaviruses encodes at least the following structure proteins, the spike (S), envelope (E), membrane (M), and nucleocapsid (N) proteins. The S glycoprotein is responsible for binding the host receptor via the receptor-binding domain (RBD) in its S1 subunit, as well as the subsequent membrane fusion and viral entry driven by its S2 subunit. Gene sequencing of SARS-CoV-2 showed that this novel coronavirus, a betacoronavirus, is related to the MERS-CoV and the SARS-CoV. SARS-CoV, MERS-CoV, and SARS-CoV-2 belong to the betacoronavirus genus and are highly pathogenic zoonotic viruses. Thus, the present invention can be used not only to determine antigenic peptides from the three highly pathogenic betacoronaviruses, but also low-pathogenicity betacoronaviruses, such as, HCoV-OC43, HCoVHKU1, HCoV-NL63 and HCoV-229E, are also endemic in humans. In certain specific embodiments, the coronavirus is SARS-CoV-2, including novel mutants of SARS-CoV-2 that include mutants from five clades (19A, 19B, 20A, 20B, and 20C) according to Nextstrain, in GISAID nomenclature which divides them into seven clades (L, O, V, S, G, GH, and GR), and/or PANGOLIN nomenclature which divides them into six major lineages (A, B, B.1, B.1.1, B.1.177, B.1.1.7). Notable mutations of SARS-CoV-2 include, e.g., D614G, P681H, N501Y, 69-70del, P681H, Y453F, 69-70deltaHV, N501Y, K417N, E484K, N501Y, and E484K.

As used herein, a “cell” refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the term “contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be, for example, an amino acid sequence, protein, or peptide as provided herein and an immune cell, such as a T cell.

As used herein, a “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The terms “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The terms “subject” or “subject in need thereof” refers to a living organism who is at risk of or prone to having a disease or condition, or who is suffering from a disease or condition that can be treated by administration of a composition or pharmaceutical composition as provided herein. Non-limiting examples include humans and other primates, but also includes non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

The terms “disease” or “condition” refer to a state of being or health status of a patient or subject capable of being treated with a compound, pharmaceutical composition, or method provided herein.

In embodiments, a patient or subject is human. In embodiments, the disease is coronavirus infection.

In certain alternative embodiments, the disease is SARS-CoV-2 infection. In still other embodiments, the disease is COVID-19.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated or the disorder resulting from viral infection. Also, a therapeutic benefit is achieved with the eradication or amelioration of one or more of the physiological symptoms associated with viral infection or the underlying disorder such that an improvement is observed in the patient, notwithstanding that the patient may still be afflicted with the underlying disorder or may still be infected. For prophylactic benefit, the compositions may be administered to a patient at risk of viral infection, of developing a particular disease, or to a patient reporting one or more of the physiological symptoms of a disease, even though a diagnosis of this disease may not have been made. Treatment includes preventing the infection or disease, that is, causing the clinical symptoms of the disease not to develop by administration of a protective composition prior to infection or the induction of the disease; suppressing the disease, that is, causing the clinical symptoms of the disease or infection not to develop by administration of a protective composition after the inductive event or infection but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. “Treatment” can also refer to any of (i) the prevention of infection or reinfection, as in a traditional vaccine, (ii) the reduction or elimination of symptoms, and (iii) the substantial or complete elimination of the pathogen in question. Treatment may be affected prophylactically (prior to infection) or therapeutically (following infection).

In addition, in certain embodiments, “treatment,” “treat,” or “treating” refers to a method of reducing the effects of one or more symptoms of infection with a coronavirus. Thus, in the disclosed method, treatment can refer to a 10%, 20%, 30%, 40%, 50%, 60%, 70%, 80%, 90%, or 100% reduction in the severity of an established infection, disease, condition, or symptom of the infection, disease or condition.

For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition and/or complete prevention of infection. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

As used herein the terms “diagnose” or “diagnosing” refers to recognition of an infection, disease or condition by signs and symptoms. Diagnosing can refer to determination of whether a subject has an infection or disease. Diagnosis may refer to determination of the type of disease or condition a subject has or the type of virus the subject is infected with.

Diagnostic agents provided herein include any such agent, which are well-known in the relevant art. Among imaging agents are fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. Enzymes that may be used as imaging agents in accordance with the embodiments of the disclosure include, but are not limited to, horseradish peroxidase, alkaline phosphatase, acid phosphatase, glucose oxidase, β-galactosidase, β-glucoronidase or P-lactarnase. Such enzymes may be used in combination with a chromogen, a fluorogenic compound or a luminogenic compound to generate a detectable signal.

The peptide(s) or protein(s) of the present invention can also be used in binding assays including, but are not limited to, immunoassays such as competitive and non-competitive assay systems using techniques such as western blots, radioimmunoassays, ELISA (enzyme linked immunosorbent assay), “sandwich” immunoassays, Meso Scale Discovery (MSD, Gaithersburg, Md.), immunoprecipitation assays, ELISPOT, precipitin reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al., eds, 1994, Current Protocols in Molecular Biology, Vol. 1, John Wiley & Sons, Inc., New York, relevant portions incorporated herein by reference).

Radioactive substances that may be used as imaging agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁷Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71).

These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the imaging agent is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art. Pharmaceutical compositions can also include large, slowly metabolized macromolecules such as proteins, polysaccharides such as chitosan, polylactic acids, polyglycolic acids and copolymers (such as latex functionalized sepharose (TM), agarose, cellulose, and the like), polymeric amino acids, amino acid copolymers, and lipid aggregates (such as oil droplets or liposomes). Additionally, these carriers can function as immunostimulating agents (i.e., adjuvants).

The term “adjuvant” refers to a compound that when administered in conjunction with the compositions provided herein including embodiments thereof, augments the composition’s immune response. Generally, adjuvants are non-toxic, have high-purity, are degradable, and are stable. Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other adjuvants contemplated for the invention are saponin adjuvants, such as Stimulon™ (QS-21, Aquila, Framingham, MA) or particles generated therefrom such as ISCOMs (immunostimulating complexes) and ISCOMATRIX. Other adjuvants include RC-529, GM-CSF and Complete Freund’s Adjuvant (CFA) and Incomplete Freund’s Adjuvant (IFA). Other adjuvants include cytokines, such as interleukins (e.g., IL-1 α and β peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants. Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration. As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer’s, normal sucrose, normal glucose, binders, fillers, disintegrants, lubricants, coatings, sweeteners, flavors, salt solutions (such as Ringer’s solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like., that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

The present invention describes methods utilizing and compositions comprising or expressing T cell epitopes, T cell epitope-containing peptides, and T cell epitope-containing proteins associated with binding to a subset of the naturally occurring MHC Class II and/or MHC Class I molecules within the human population. Compositions comprising or expressing one or more of the disclosed peptides (e.g., the amino acid sequences set forth in any one of Tables 4-9) or polynucleotides encoding the same, covering different HLA Class II and/or MHC Class I alleles, capable of generating a treatment acting broadly on a population level are disclosed herein. As the antigen repertoire of MHC Class I and MHC Class II alleles varies from one individual to another and from one ethnic population to another, it is challenging to provide vaccines or peptide or epitopes-based immunotherapies that can be offered to subjects of any geographic region in the world or provide sufficient protection against infection across a wide segment of the populations unless numerous epitopes or peptides are included (e.g., in a vaccine). Taking into consideration the need for a single vaccine formulation that can provide protection across populations, if it desirable to provide a treatment containing or expressing proteins, peptides or epitopes that will provide protection against infection amongst the majority of the worldwide population. Also, taking into consideration the enormous costs and risks in the clinical development of new treatments and the increasing demands from regulatory bodies to meet high standards for toxicity testing, dose justification, safety and efficacy trials, it is desirable to provide treatments containing or expressing as few peptides as possible, but at the same time to be able to treat the majority of subjects in a worldwide population with a single immunotherapy. Such a product should comprise as a first requirement an expression or inclusion of combination of epitopes or peptides that are able to bind the worldwide MHC Class I and/or MHC Class II allele repertoire, and the resulting peptide-MHC complexes should as a second requirement be recognized by the T cells of the subject so as to induce the desired immunological reactions.

It is an object of claims of the present invention to provide improved epitope or peptide combinations for modulating an immune response, for treating a subject for an infection or aberrant immune response, and for use in diagnostic methods and kits comprising such peptide combinations. It is another object of the invention to provide epitope or peptide combinations exhibiting very good HLA Class I and Class II coverage in a worldwide population and being immunologically potent in a worldwide population. It is another object of the invention to provide epitope or peptide combinations having good cross reactivity to other viral strains, including co-circulating strains (for example, mutants) of coronaviruses, including SARS-CoV-2, common cold coronaviruses, as well as SARS-CoV, MERS, etc. It is another object of the invention to provide epitope or peptide combinations of a relatively small number of epitopes or peptides yet obtaining at least 70%, and more preferably around 90-100% donor coverage in a donor cohort representative of a worldwide population. In certain embodiments, this is achieved by selecting one or more immunodominant and/or immunoprevalent proteins (e.g., a SARS-CoV-2 protein) or subsequences, portions, homologues, variants or derivatives thereof for use in the methods and compositions of the present disclosure, wherein said immunodominant and/or immunoprevalent proteins or subsequences, portions, homologues, variants or derivatives thereof comprise two or more epitopes that are immunodominant and/or immunoprevalant. In some embodiments, the two or more epitopes comprise two to ten epitopes and/or polynucleotides encoding the same. Another object of the invention is to provide epitope combinations which are so immunologically potent that even at very low doses of epitopes, the percentage of responding donors can be retained at a very high level in a donor cohort representative of a worldwide population. Another object of the invention is to provide epitope combinations which have minor risk of inducing IgE-mediated adverse events. An additional object of the invention is to provide proteins, peptides, or nucleic acids containing or expressing epitopes or combinations of such proteins, peptides or nucleic acids which have a sufficient solubility profile for being formulated in a pharmaceutical product, preferably which have acceptable estimated in vivo stability. One further objective of the invention is to select epitopes for use in the compositions and methods described herein, based on one or both of their immunodominance or immunoprevalence. A still further object of the invention is to select such epitopes and epitopes combinations not only in accordance with those embodiments previously described, but also those epitopes and epitope combinations capable of eliciting a B cell response and T cell response (e.g., selecting one or more peptides for use in the methods and compositions described herein capable of generating a T cell and antibody response in a subject).

Provided herein are methods and compositions for diagnosing, treating, and immunizing against a coronavirus, including methods and compositions of detecting an immune response or immune cells relevant to a coronavirus infection. These methods and compositions include vaccines, diagnostics, therapies, reagents and kits, for modulating, eliciting, or detecting T cells responsive to one or more coronavirus peptides or proteins. The proteins and peptides described herein comprise, consist of, or consist essentially of: one or more amino acid sequences selected from SEQ ID NO: 1 to 1126, or a subsequence, portion, homologue, variant or derivative thereof; a fusion protein comprising one or more amino acid sequences selected from SEQ ID NO: 1 to 1126; a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1150 or more peptides selected from the amino acid sequences set forth in SEQ ID NO: 1 to 1126, or a polynucleotide that encodes one or more peptides or proteins, comprising, consisting of, or consisting essentially of an amino acid sequence selected from SEQ ID NO: 1 to 1126, or a subsequence, portion, homologue, variant or derivative thereof. In certain preferred embodiments, the coronavirus is one or more of SARS-CoV-2 or a variant thereof, or SARS, MERS, or a common cold coronavirus strain (e.g., 229E, NL63, HKU1, OC43). Further description and embodiments of such methods and compositions are provided in the definitions provided herein, and a person skilled in the art will recognize that the methods and compositions can be embodied in numerous variations, changes, and substitutions or as may occur to or be understood by one skilled in the art without departing from the invention.

The present inventors recognized that defining a comprehensive set of epitope specificities is important for several reasons. First, it allows the determination of whether within different SARS-CoV-2 antigens certain regions are immunodominant. This will be important for vaccine design, so as to ensure that vaccine constructs include not only regions targeted by neutralizing antibodies, such as the receptor binding domain (RBD) in the spike (S) region, but also include regions capable of delivering sufficient T cell help and are suitable targets of CD4+ T cell activity. Second, a comprehensive set of epitopes helps define the breadth of responses, in terms of the average number of different CD4+ and CD8+ T cell SARS-CoV-2 epitopes generally recognized by each individual. This is key because some reports have described a T cell repertoire focused on few viral epitopes (Ferretti et al., 2020), which would be concerning for potential viral escape from immune recognition via accumulated mutations that can occur during replication or through viral reassortment. Third, a comprehensive survey of epitopes restricted by a set of different HLAs representative of the diversity present in the general population is important to ensure that results obtained are generally applicable across different ethnicities and racial groups, and also to lay the foundations to examine the potential associations of certain HLAs with COVID-19 severity. Finally, the definition of the epitopes recognized in SARS-CoV-2 infection is relevant in the context of the debate on the potential influence of SARS-CoV-2 cross-reactivity with endemic “Common Cold” Coronaviruses (CCC) (Braun et al., 2020; Le Bert et al., 2020). Several studies have defined the repertoire of SARS-CoV-2 epitopes recognized in unexposed individuals (Braun et al., 2020; Mateus et al., 2020; Nelde et al., 2020), but the correspondence between that repertoire and the epitope repertoire elicited by SARS-CoV-2 infection has not been previously evaluated.

The present inventors provide a comprehensive map of epitopes recognized by CD4+ and CD8+ T cell responses across the entire SARS-CoV-2 viral proteome. Importantly, these epitopes have been characterized in the context of a broad set of HLA alleles using a direct ex vivo, cytokine-independent, approach.

Characteristics of the study participants. To broadly define the pattern of immunodominance and epitope recognition associated with SARS-CoV-2 infection, the inventors studied PBMC samples from 99 adult convalescent COVID-19 donors. Their age ranged from 19 to 91 years (median 41), with a gender ratio of about 2M:3F (Male 41%; Female 59%). Ethnic breakdown was reflective of the demographics of the local enrolled population. Samples were obtained 3 to 184 days post-symptom onset (median 67 days). Peak COVID-19 disease severity was representative of the distribution observed in the general population to date (mild 91%, moderate 2%, severe and critical 7%) (Table 1).

SARS-CoV-2 infection was determined by PCR-based testing during the acute phase of infection, if available (79% of the cases), and/or verified by plasma SARS-CoV-2 S protein RBD IgG ELISA (Stadlbauer et al., 2020) using plasma from convalescent phase blood draws. All donors were seropositive at the time of blood donation, with the exception of two mildly symptomatic donors with positive PCR results from the acute phase of illness, but seronegative results at time of blood donation (at 55- and 148-days post-symptom onset (PSO), respectively).

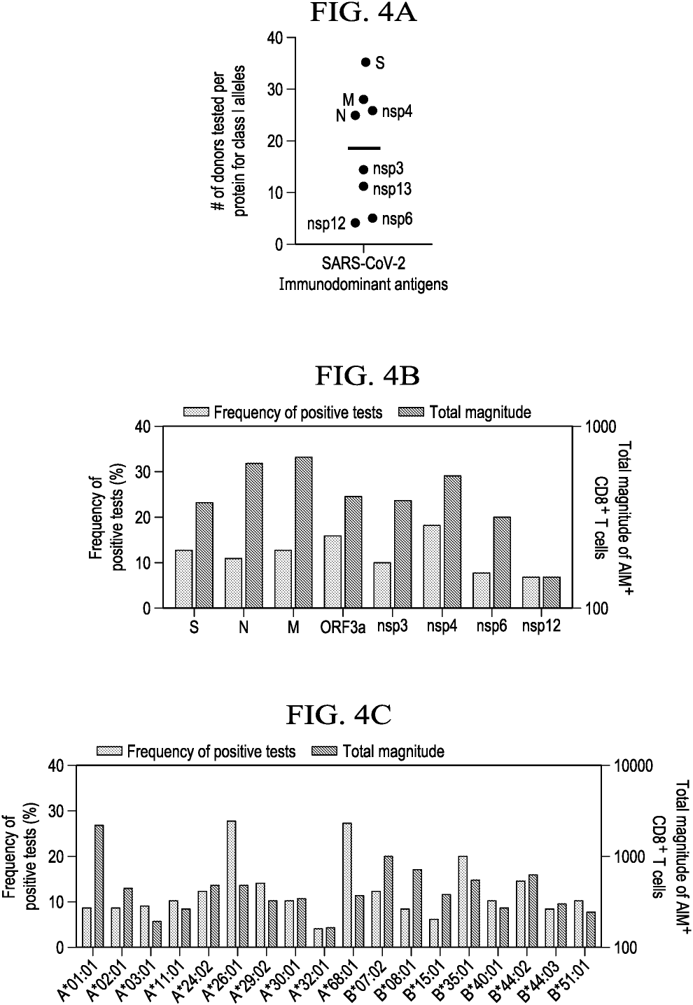

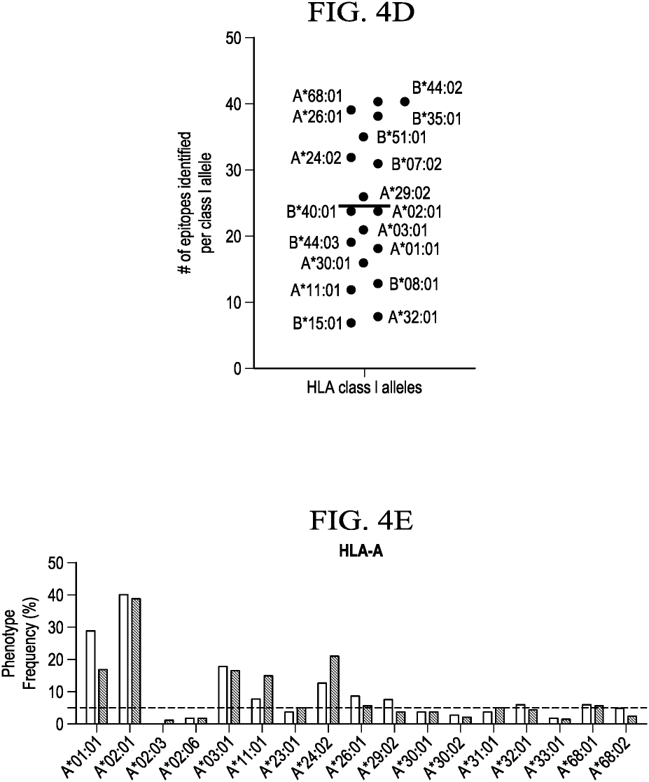

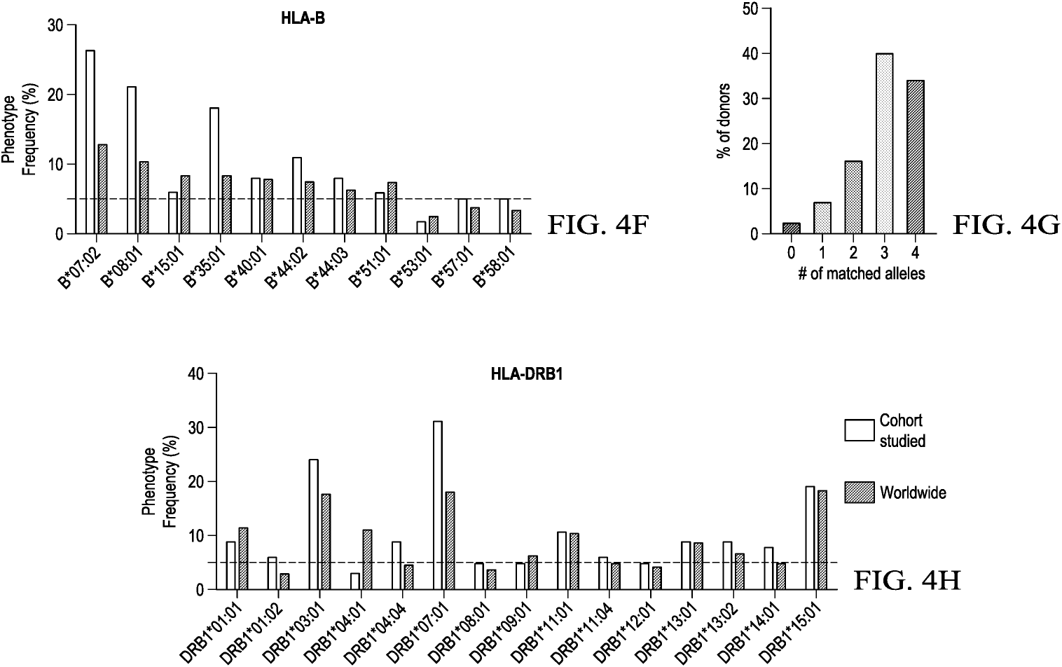

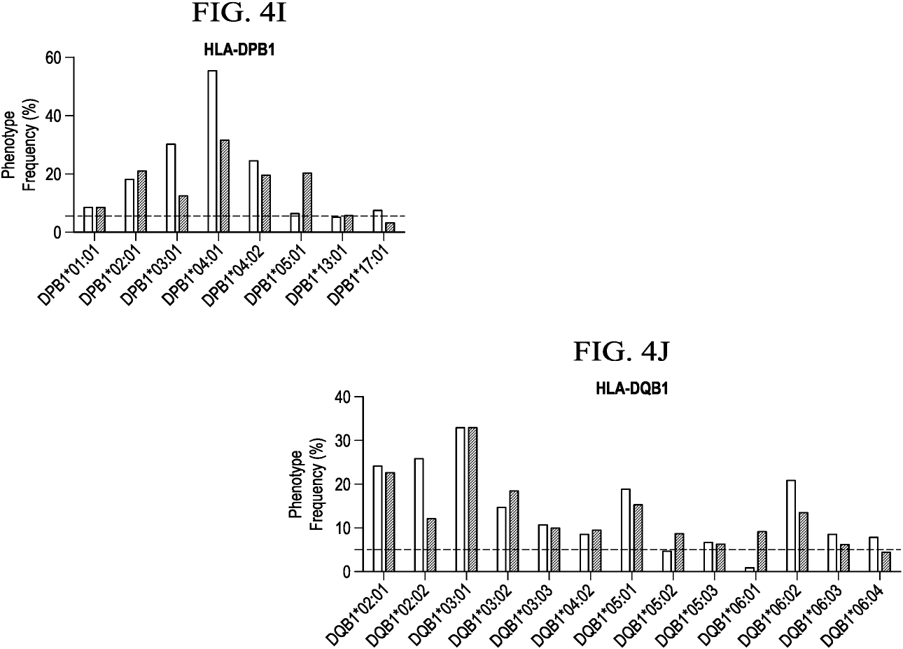

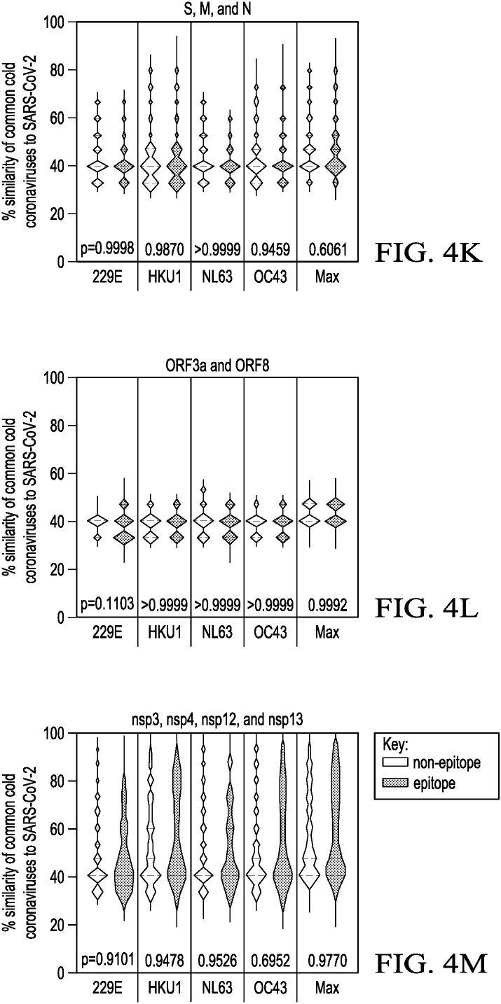

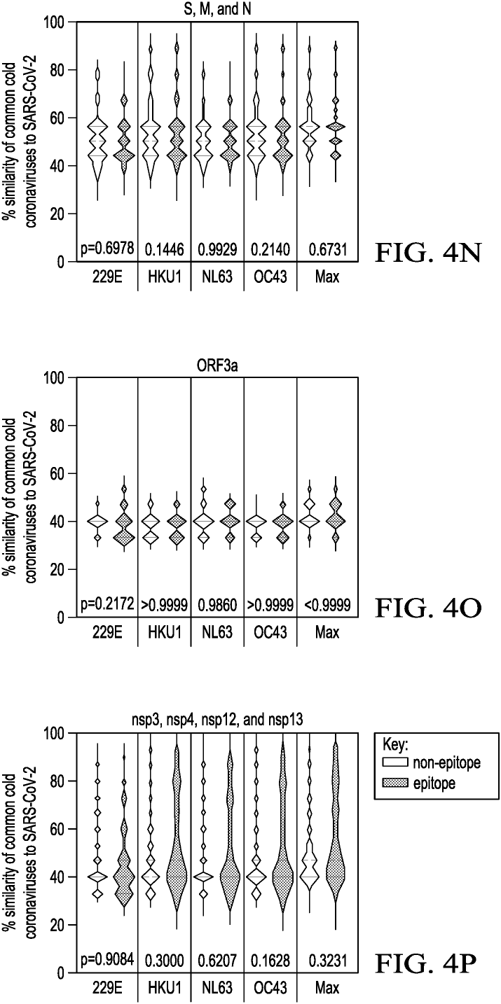

All donors were HLA typed at both class I and class II loci (data not shown). The HLA class I and II alleles frequently observed in the enrolled cohort were largely reflective of what is found in the worldwide population, as reported by the Allele Frequency Net Database (Gonzalez-Galarza et al., 2020), and as retrieved from the Immune Epitope Database’s (IEDB; www.iedb.org) population coverage tool (Bui et al., 2006; Dhanda et al., 2019) (FIGS. 4E-4J). Of the 20 different HLA class I alleles with phenotypic frequencies >5% in this cohort, 15 (75%) are also present in the most common and representative class I alleles in the worldwide population (Paul et al., 2013) (FIGS. 4G-4H). Likewise, of the 34 different HLA class II alleles with phenotypic frequencies >5% in this cohort, 26 (76%) are also present in the worldwide population with frequencies >5%. These alleles correspond to 16 of the 27 (59%) alleles included in a reference panel of the most common and representative class II alleles in the general population (Greenbaum et al., 2011)(FIGS. 4H-4J). In conclusion, this cohort is largely representative of the HLA allelic variants commonly expressed worldwide.

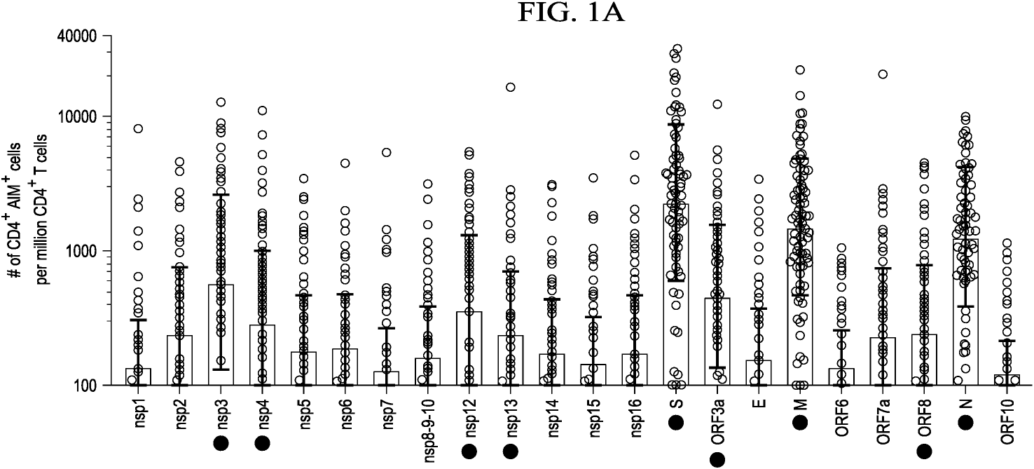

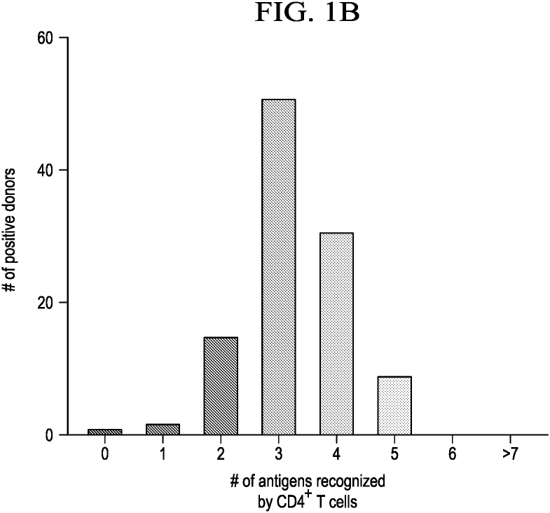

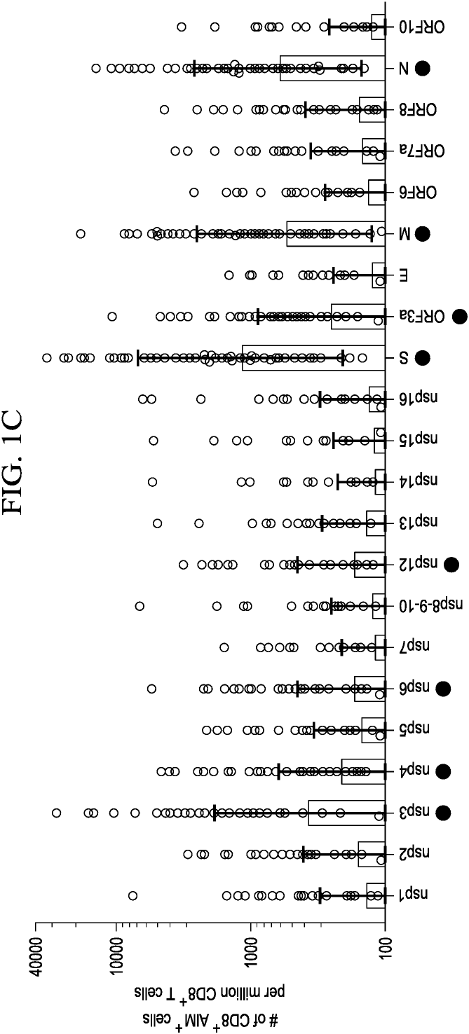

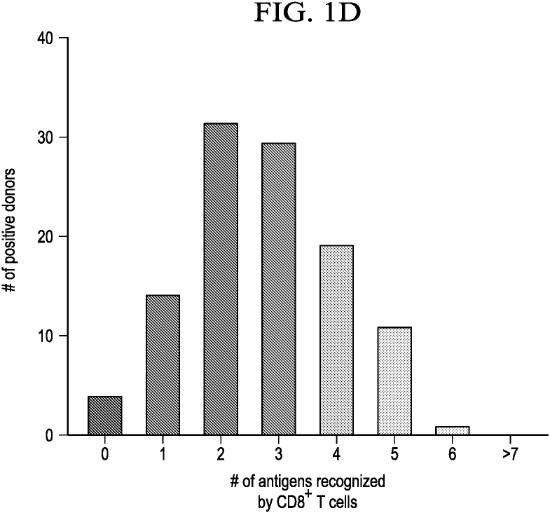

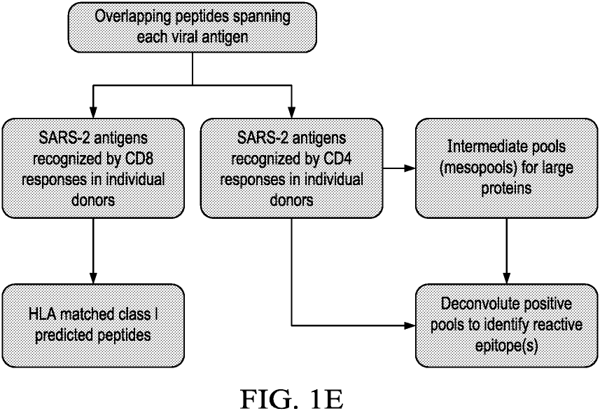

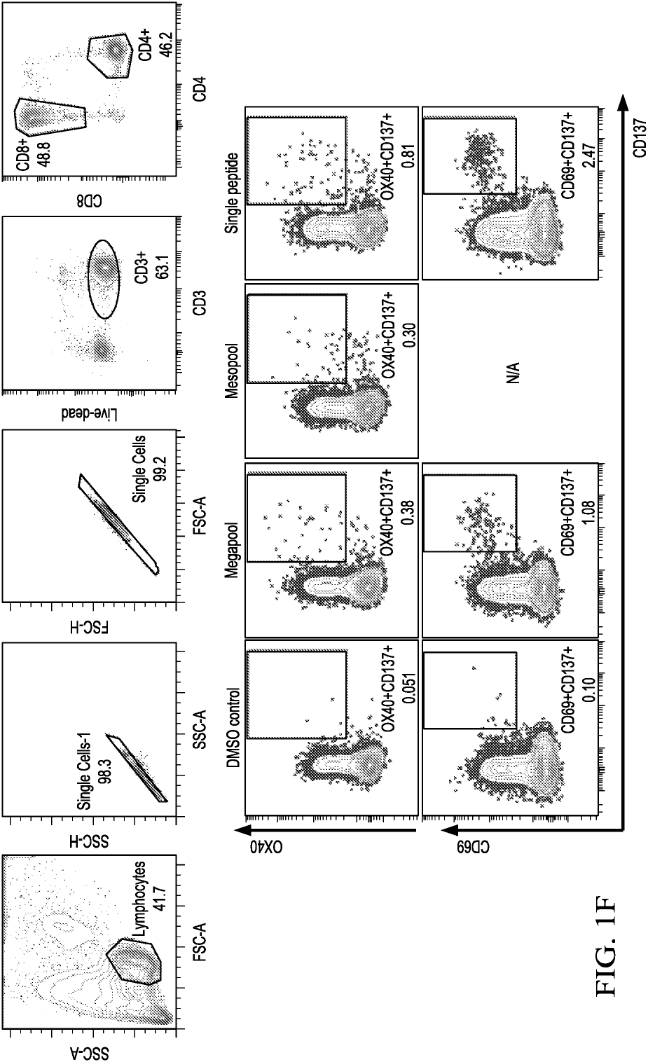

Pattern of antigen immunodominance in CD4+ and CD8+ T cell responses to SARS-CoV-2 antigens. To study adaptive immune responses in COVID-19 convalescent donors, the inventors previously utilized T cell receptor (TCR) dependent Activation Induced Marker (AIM) assays to quantify SARS-CoV-2-specific CD4+ and CD8+ T cells utilizing the combination of markers OX40+CD137+ and CD69+CD137+ for CD4+ and CD8+ T cells, respectively (Grifoni et al., 2020; Mateus et al., 2020; Weiskopf et al., 2020). To define the global pattern of immunodominance in the study cohort, the inventors tested PBMC from each donor with sets of overlapping peptides spanning the various SARS-CoV-2 proteins, as previously described (Grifoni et al., 2020b) (data not shown). These data also defined the specific viral antigens recognized by each donor, and therefore highlight the specific antigens/donor pairs suitable for further epitope identification studies, as shown in FIGS. 1A and C.

For each SARS-CoV-2 protein antigen (Table 2) the inventors recorded the % of donors in which a positive response was detected and the total response counts (positive cells/million detected in the AIM assay). This information was used to tabulate the percentage of the total response ascribed to each protein, and calculate the cumulative coverage provided by the most immunodominant proteins.

For CD4+ T cell responses, 9 viral proteins (non-structural protein (nsp) 3, nsp4, nsp12, nsp13, S, ORF3a, Membrane (M), ORF8, and Nucleocapsid (N)) accounted for 83% of the total response. In the context of CD8+ T cell responses, 8 viral proteins (nsp3, nsp4, nsp6, nsp12, S, ORF3a, M, and N) accounted for 81% of the total response. These results confirmed the pattern previously observed with a more limited (n=20) number of COVID-19 patients (Grifoni et al., 2020b) and highlight a broad pattern of immunodominance, where 8-9 antigens are required to cover 80% of the response.

The inventors further evaluated the number of antigens recognized in each of the individual donors analyzed. To this end, the inventors focused on antigens associated with a sizeable response, arbitrarily defined herein as those antigens individually accounting for at least 10% of the total response. It was found that per donor an average of 3.2 and 2.7 proteins were recognized by 10% or more of the total CD4+ and CD8+ SARS-CoV-2-specific T cells, respectively (FIGS. 1B and 1D). FIG. 1E shows a flow chart of a scheme of experimental strategy selected for HLA class I and class II epitope identification, and representative graphs depicting the flow cytometry gating strategy for defining antigen-specific CD4⁺ and CD8⁺ T cells by OX40⁺CD137⁺ and CD69⁺CD137⁺ expression, respectively.

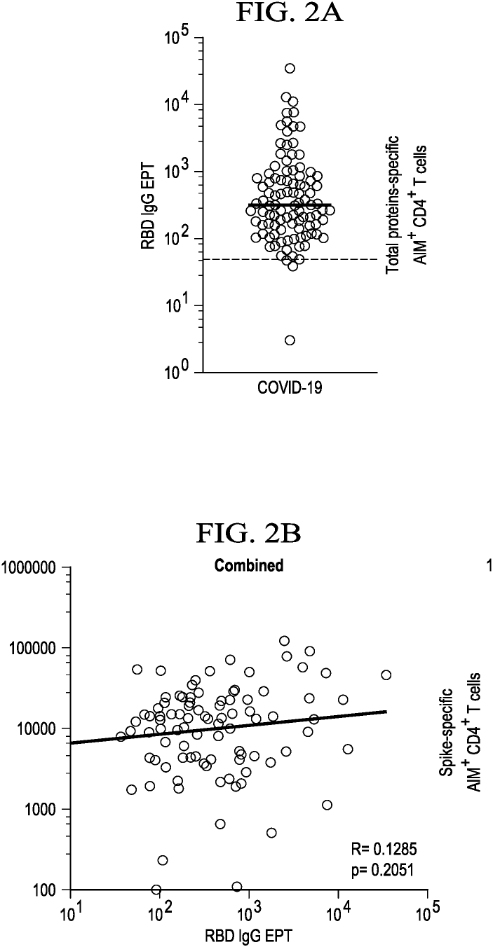

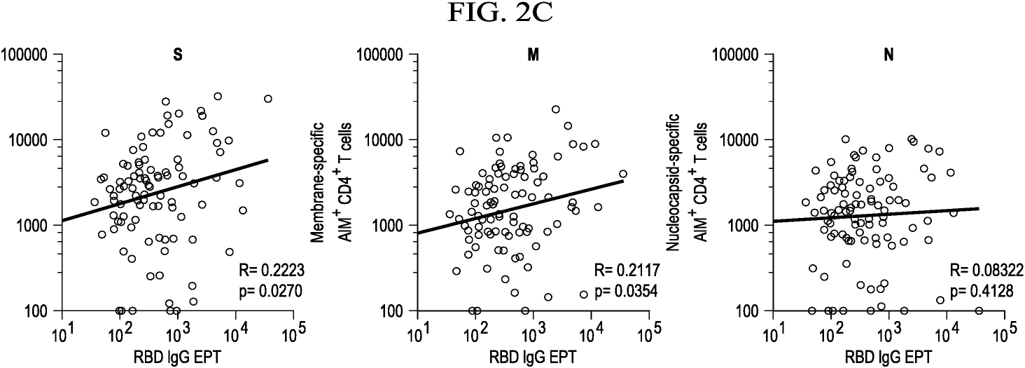

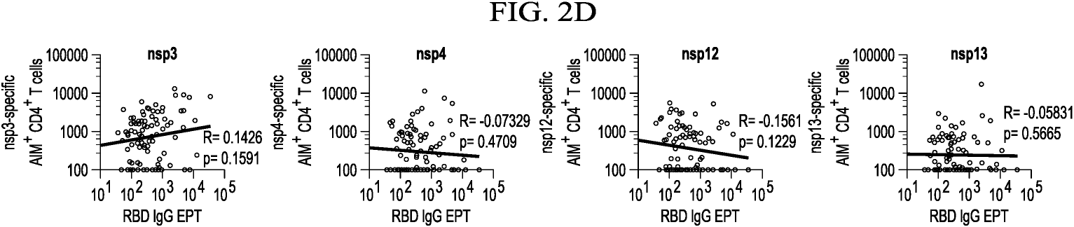

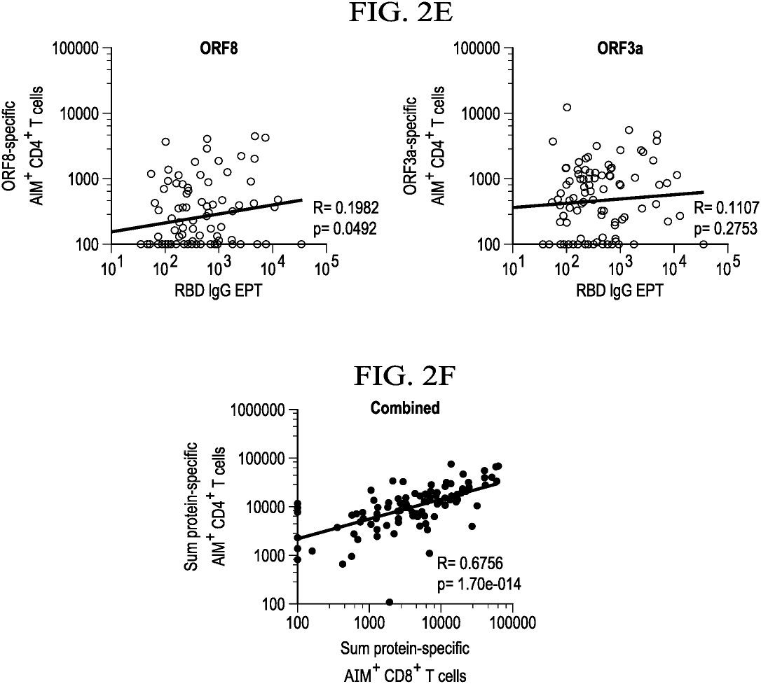

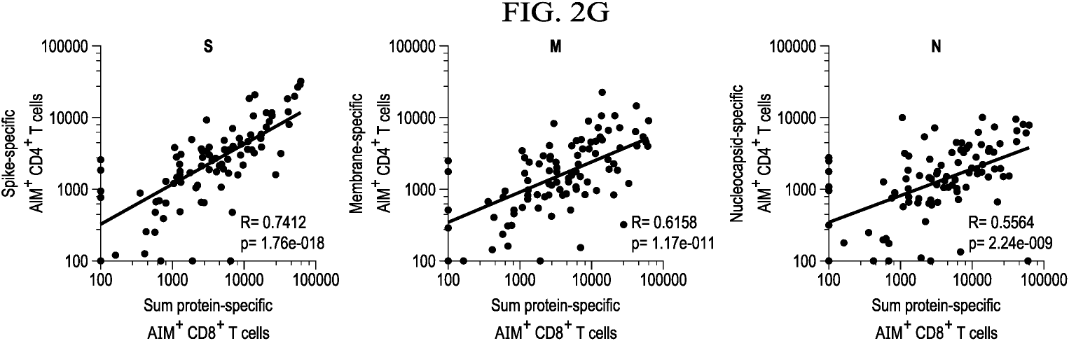

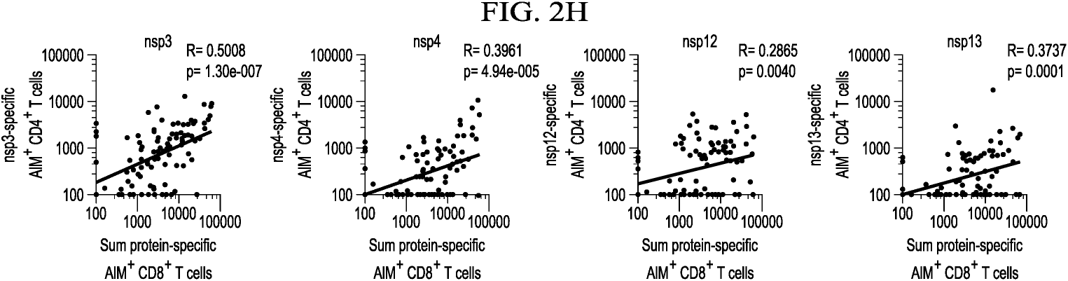

Functional consequences of SARS-CoV-2-specific CD4+ T cell responses directed against different antigens. Next, the inventors investigated whether the recognition of different SARS-CoV-2 antigens by CD4+ T cells correlated with functional antibody and/or CD8+ T cell responses. Consistent with the wide range of blood collection time points (day PSO) and peak disease severity in the COVID-19 donor cohort, a wide range of RBD IgG responses (FIG. 2A) were observed. Combined CD4+ T cell responses did not significantly correlate with the antibody response to RBD (R = 0.1285, p = 0.2051; FIG. 2B). Breaking the correlation down for individual antigens showed that two correlations had p-values <0.05 namely Spike (R = 0.2223, p = 0.0270) and M protein (R = 0.2117, p = 0.0354), but these would not be significant when performing a multiple hypothesis comparison taking all other antigens into account (FIGS. 2C-E). In contrast, the correlation between CD4+ and CD8+ T cell responses was highly significant in aggregate (R = 0.6756, p = 1.70×10-14; FIG. 2F) and was significant for each of the individual antigen comparisons FIGS. 2G-I). The same was observed when the correlations of the matched protein-specific CD4+ and CD8+ T cell responses were considered (FIGS. 2J-2L).

These data show that the CD4+ T cell response against all dominant antigens is relevant in terms of providing helper function for CD8+ T cell-specific responses. These results may reflect that T cell responses correlate with gene expression. S, N, and M may be immunodominant because of the very high gene expression for each (Xie et al., 2020). In this context, it is perhaps surprising that a strong CD4+ and CD8+ T cell response was elicited by nsp3, which is not known to be expressed at high levels (Xie et al., 2020).

SARS-CoV-2 peptides and epitope screening strategy. The analysis of the SARS-CoV-2 proteome summarized above identified the major viral antigens accounting for 80% or more of the total CD4+ and CD8+ T cell response. These antigens were then introduced into the epitope screening pipeline (FIG. 1E). FIG. 1E shows a flow chart of a scheme of experimental strategy selected for HLA class I and class II epitope identification, and representative graphs depicting the flow cytometry gating strategy for defining antigen-specific CD4⁺ and CD8⁺ T cells by OX40⁺CD137⁺ and CD69⁺CD137⁺ expression, respectively. Since class II epitope prediction is not as robust as class I prediction (Peters et al., 2020), and because of the high degree of overlap in binding capacity of different HLA class II alleles, to determine CD4+ T cell reactivity in more detail a comprehensive and unbiased approach based on the use of complete sets of overlapping peptides spanning each antigen, and composition of antigen-specific peptide pools was used. Positivity was defined as net AIM+ counts (background subtracted by the average of triplicate negative controls) >100 and a Stimulation Index (SI) >2, as previously described (da Silva Antunes et al., 2020). Positive peptide pools were deconvoluted to identify the specific 15-mer peptide(s) recognized. For large proteins, such as S, an intermediate “mesopool” step was used to optimize use of reagents.

In parallel, panels of predicted HLA class I binders for the 28 most common allelic variants were synthesized (data not shown), as described in the methods section. The top two hundred predicted peptides were synthesized for each allele, leading to 5,600 predicted HLA binders in total. To identify CD8+ T cell epitopes, the inventors tested individual peptides derived from the specific antigen(s) recognized by CD8+ T cells of individual donors and that were predicted to bind the HLA class I alleles expressed by the respective donor. (FIG. 1C). To quantify the population coverage provided by the HLA class I alleles selected for study, the inventors tabulated the fraction of the donor cohort studied where allele matches were identified for 0, 1, 2, 3 or 4 of the respective HLA A and B alleles expressed by the donor. It was found that 98% of the participants in this cohort were covered by at least one allele, 91% by 2 or more, and 74% were covered by 3 or more of the alleles in this panel (FIG. 4G). As shown in Table 2, focusing on the 8 most dominant SARS-CoV-2 antigens for the purpose of epitope identification allowed mapping of 80% or more of the response, while screening only 35-40% of the total peptides.

To broadly identify T cell epitopes recognized in a cytokine-independent manner, the inventors used the AIM assay mentioned above (Grifoni et al., 2020; Reiss et al., 2017). Examples of gating strategies, pool deconvolution and epitope identification for both CD4+ and CD8+ T cell responses are shown in FIG. 1F. AIM+ cell counts were calculated per million CD4+ or CD8+ T cells, respectively. FIG. 1E shows representative graphs depicting the flow cytometry gating strategy for defining antigen-specific CD4⁺ and CD8⁺ T cells by OX40⁺CD137⁺ and CD69⁺CD137⁺ expression, respectively.

Summary of CD4+ T cell epitope identification results. To identify specific CD4+ T cell epitopes, the inventors deconvoluted peptide pools corresponding to antigens previously identified as positive for CD4+ T cell activity in each specific donor. In instances where not all positive pools could be deconvoluted due to limited cell availability, peptide pools were selected for screening to ensure that each of the 9 major antigens was tested in at least 10 donors. Overall, the inventors were able to test each peptide for these antigens in a median of 13 donors (range 10 to 17). Each donor was previously determined to be positive for CD4+ T cell responses to that specific antigen.

Taken together, a total of 280 SARS-CoV-2 CD4+ T cell epitopes were identified, including 3 nsp16 (this protein was not included in the top proteins studied) epitopes identified in parallel experiments in 2 donors (data not shown). It was found that each donor responded to an average of 3.2 viral antigens (FIG. 1D), and 5.9 CD4+ T cell epitopes were recognized per antigen for the top 80% most immunodominant antigens (data not shown). For each epitope/responding donor combination, potential HLA restrictions were also inferred based on the predicted HLA binding capacity of the epitope for the HLA alleles present in the respective responding donor (data not shown), as previously described (Mateus et al., 2020; Voic et al., 2020).

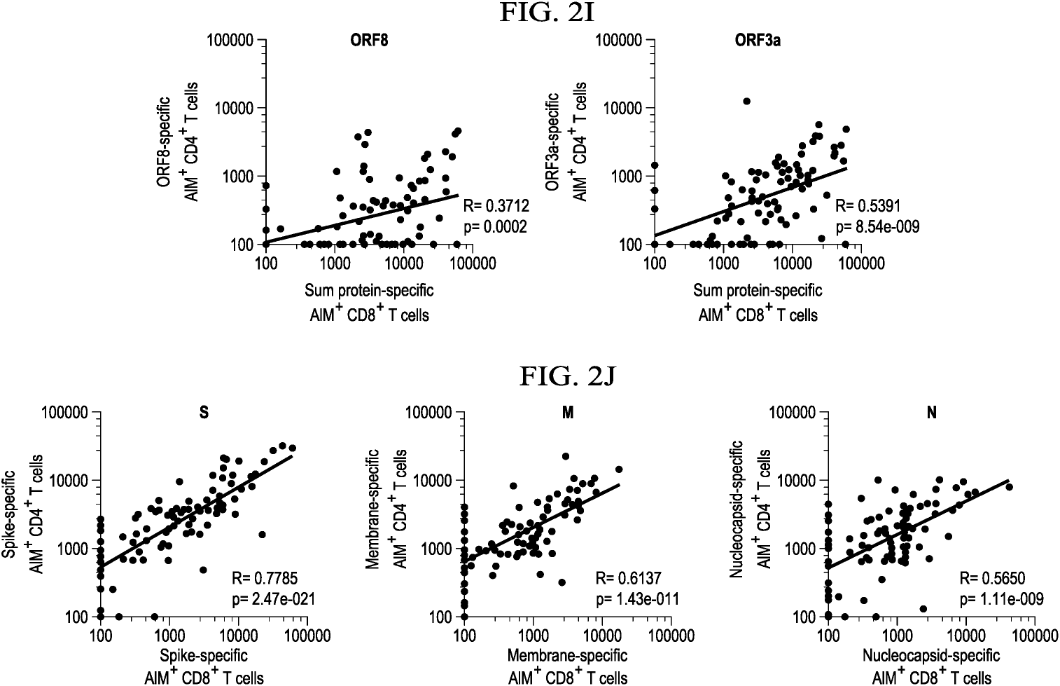

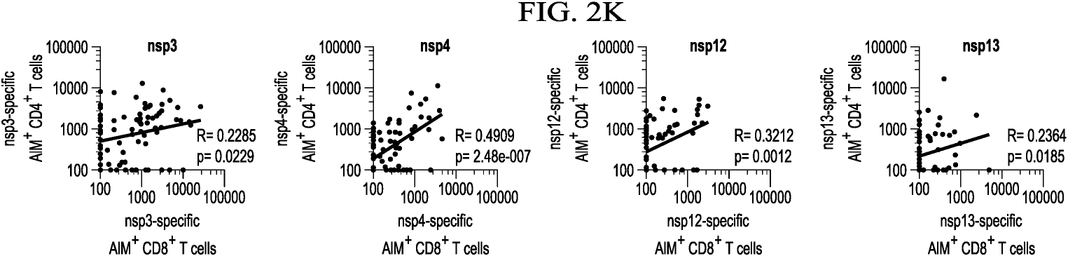

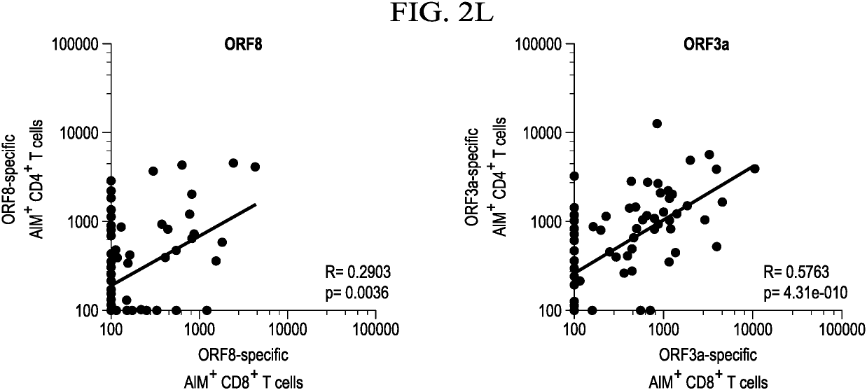

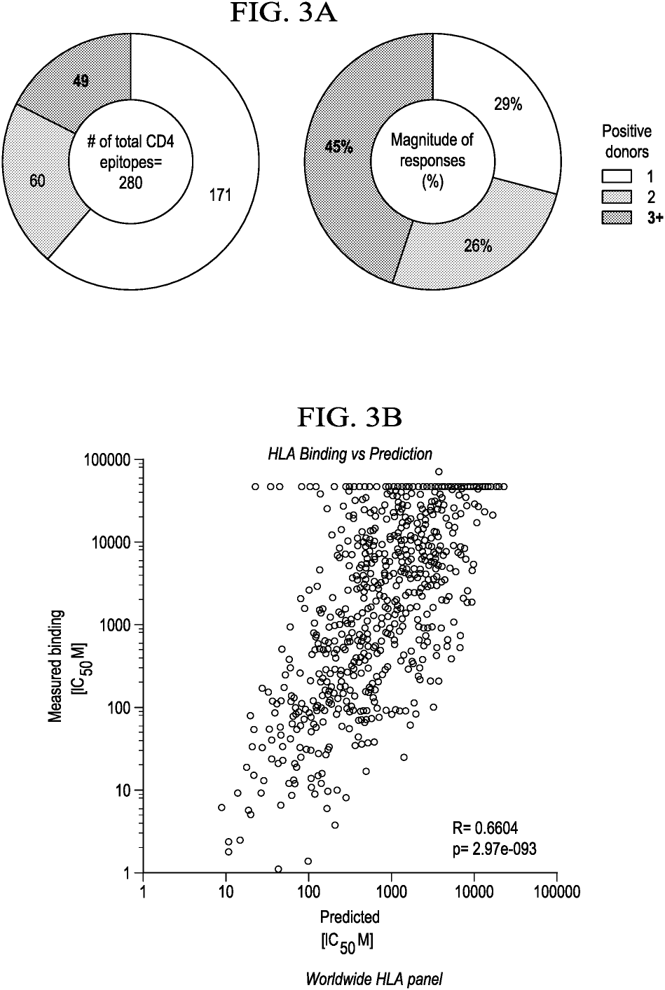

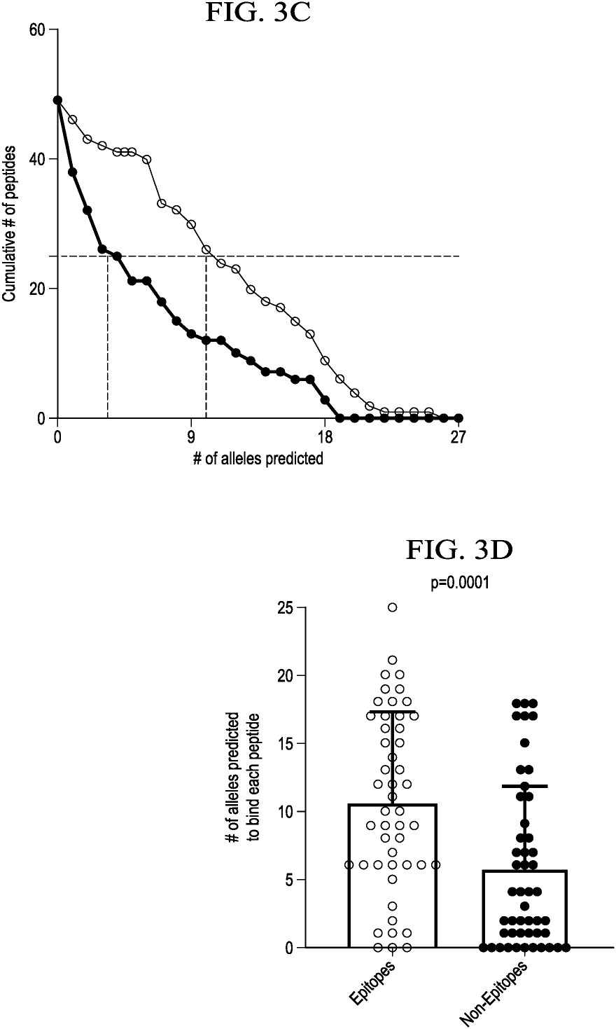

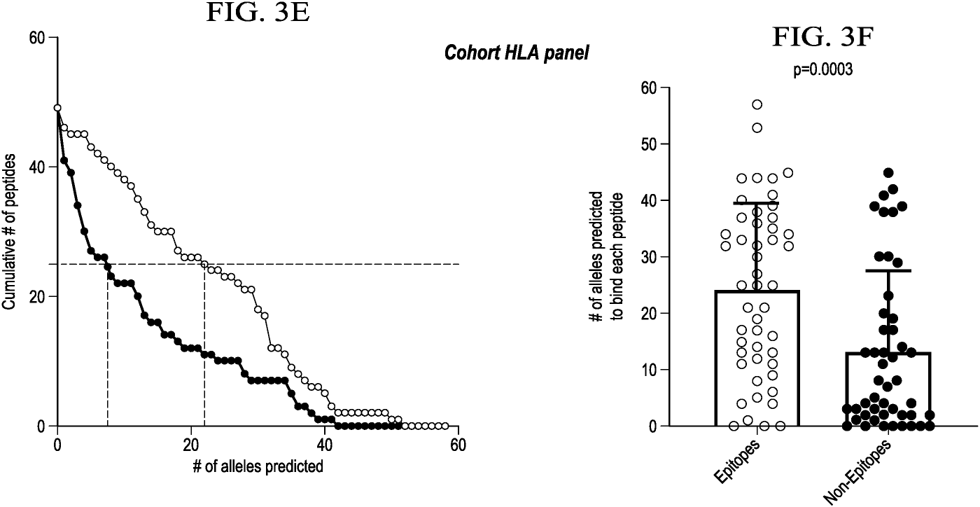

HLA binding capacity of dominant epitopes. A total of 109 of the 280 epitopes were recognized by 2 or more donors, accounting for 71% of the total response. The 49 most dominant epitopes, recognized in 3 or more donors, accounted for 45% of the total response (FIG. 3A).

Since dominant epitopes are associated with promiscuous HLA class II binding (Lindestam Arlehamn et al., 2013; Oseroff et al., 2010), defined as the capacity to bind multiple HLA allelic variants, the inventors investigated the role of HLA binding in determining immunodominant SARS-CoV-2 epitopes. Specifically, the inventors measured the in vitro binding capacity of the 49 most dominant epitopes (positive in 3 or more donors, as mentioned above) for a panel of 15 of the most common DR alleles using individual peptides and purified HLA class II molecules (Sidney et al., 2013). It was noted that, in general, a good correlation was observed between predicted and measured binding (R = 0.6604, p = 2.97×10-93; FIG. 3D). Based on these results, the inventors further characterized those 49 most dominant epitopes using predicted binding for additional HLA class II alleles, including a panel of the 12 most common HLA-DQ and DP allelic variants, and all HLA class II variants (DR, DQ, and DP) expressed in the cohort.

Overall, the 49 most dominant epitopes showed significantly higher binding promiscuity (number of alleles bound at the 1,000 nM or better threshold) (Paul et al., 2019; Southwood et al., 1998) for the panel of common HLA class II than a control group of 49 non-epitopes derived from the same proteins (Average number of HLA predicted to be bind ± SD epitopes = 10.8 ± 6.5; non-epitopes = 5.7 ± 6; p = 0.0001 by Mann-Whitney; FIGS. 3A-3F). The same conclusion was reached when the full set of HLA alleles present in the cohort were considered using the same criteria (Average ± SD epitopes = 24.3 ± 15.2; non-epitopes = 13.2 ± 14.1; p = 0.0003 by Mann-Whitney; FIG. 3). FIGS. 3B to 3F show SARS-CoV-2 immunodominant epitope HLA class II binding capacity and promiscuity. A comparison of the HLA class II binding capacity of 49 immunodominant epitopes as determined by binding predictions or as measured experimentally (FIG. 3B), suggesting feasibility for using binding predictions to assess HLA-restriction. Predicted HLA class II binding promiscuity is shown for the same 49 epitopes (white circles), and also 49 non-epitopes (black circles), considering the 27 HLA class II alleles most frequent worldwide (FIGS. 3C-3D), or the 58 HLA class II alleles specific to the study cohort (FIGS. 3E-3F). The number of HLA class II alleles predicted to bind epitopes (white circles) and non-epitopes (black circles) are based on a prediction cutoff value of IC50<1000nM. Statistical comparisons were performed using Mann-Whitney.

Heat maps of the 49 epitopes and non-epitopes considering the panel of common HLA DR, DP, and DQ are shown in FIGS. 3B-C. These results confirm that broad HLA binding capacity is a key feature of dominant epitopes. It further indicates that, because of their broad binding capacity, these epitopes are likely to be recognized in different geographical settings and different ethnicities.

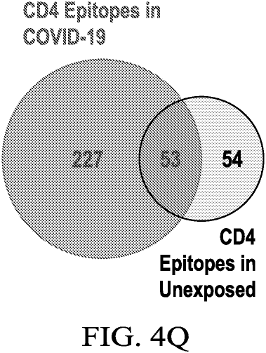

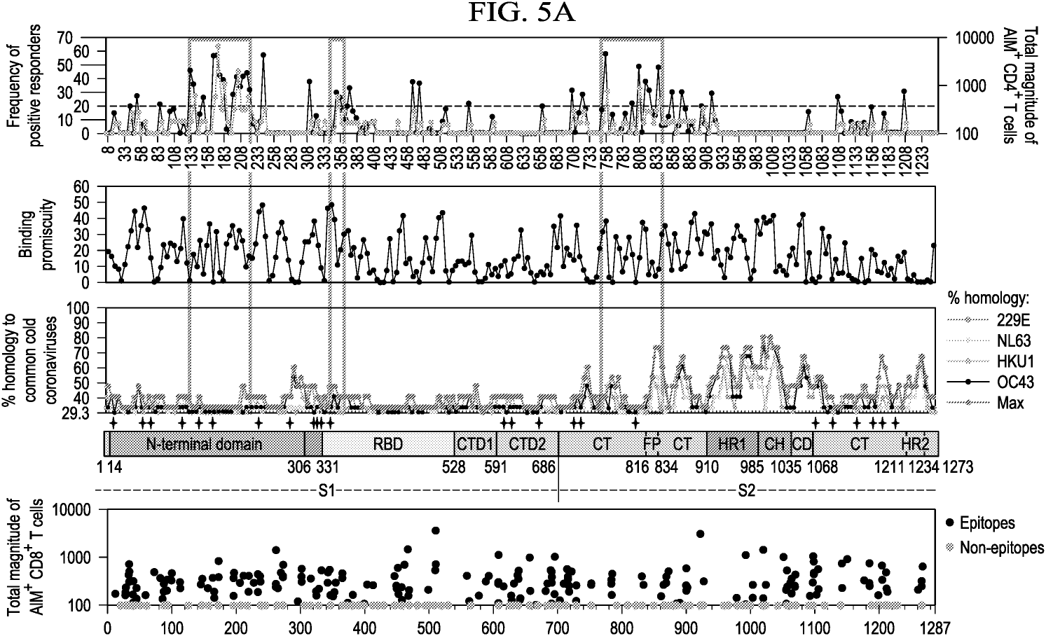

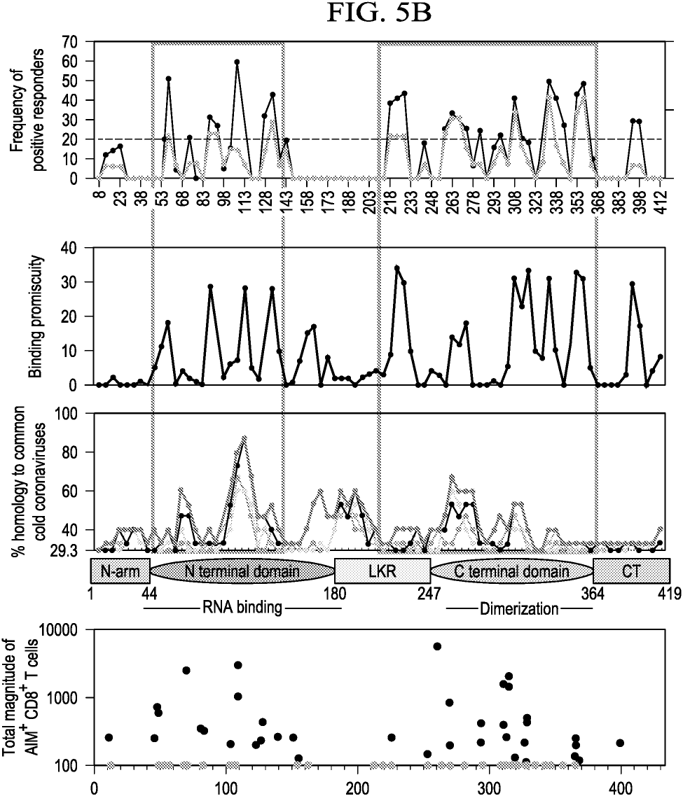

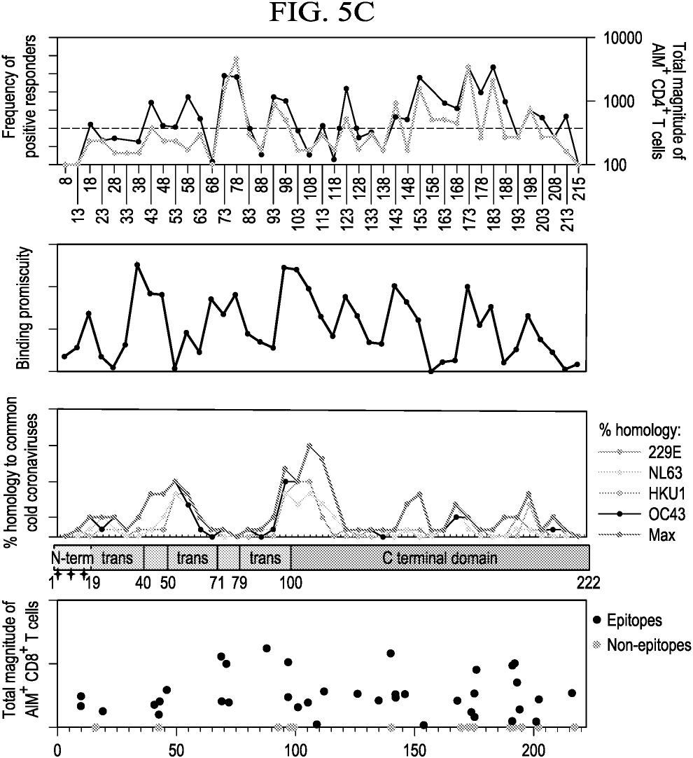

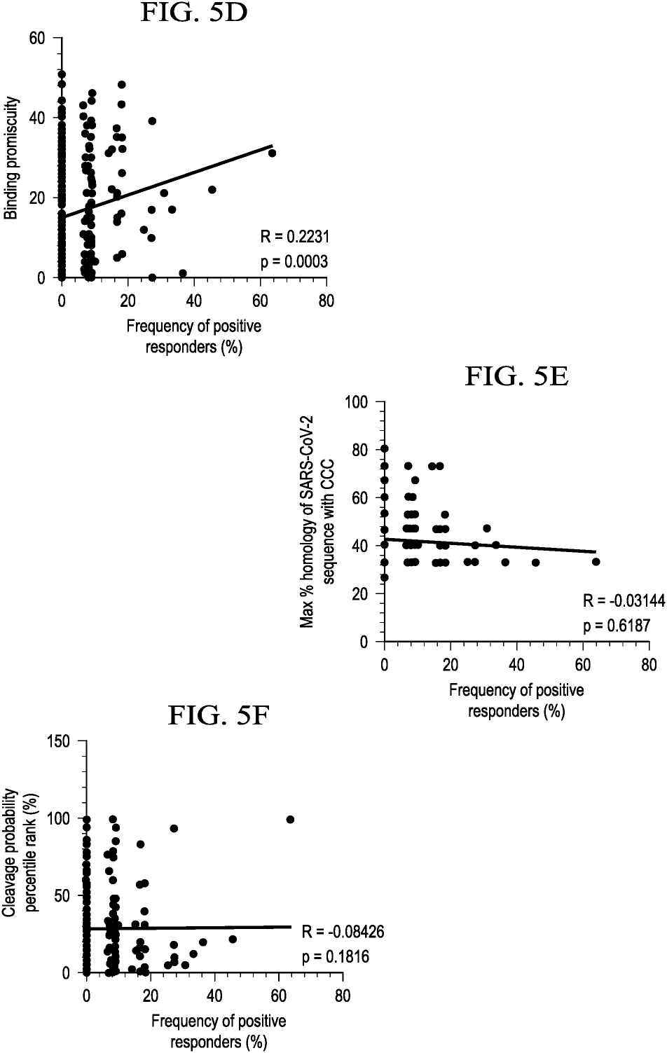

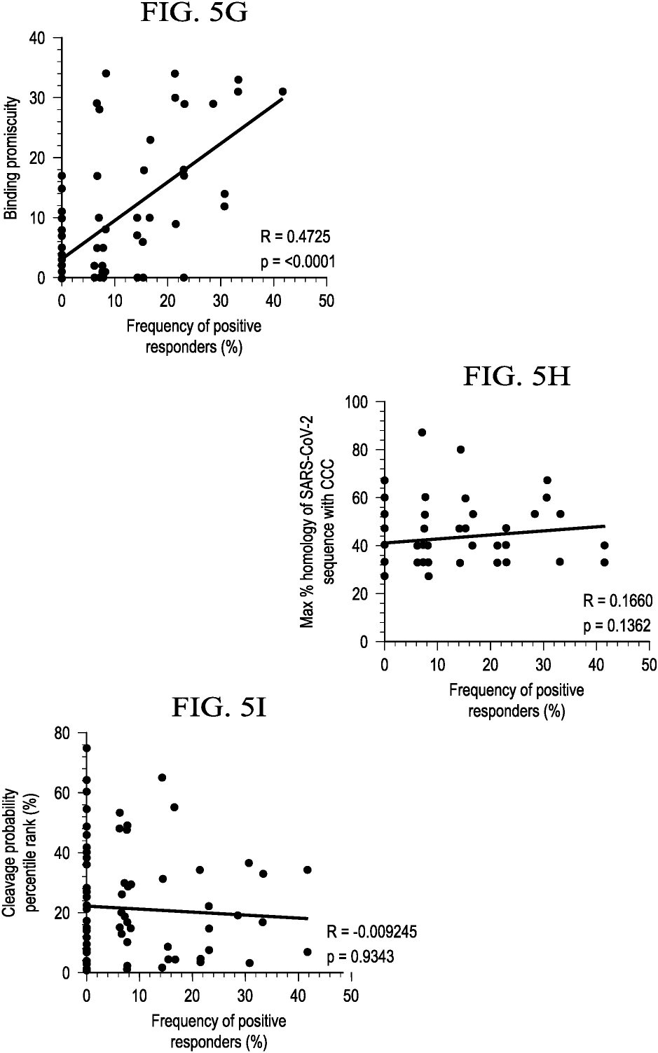

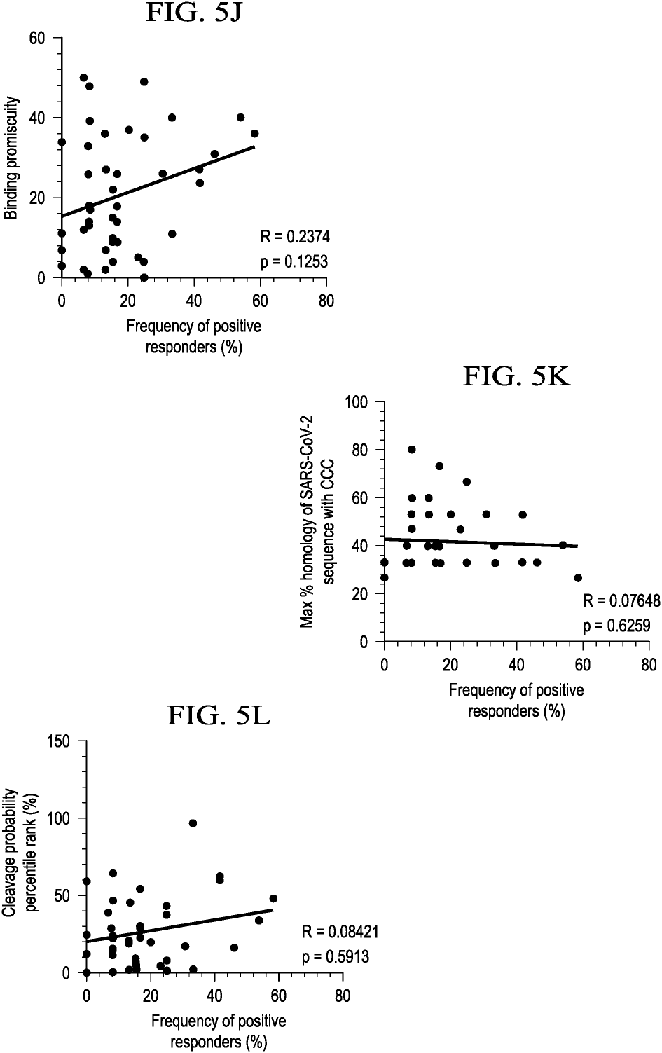

Similarity of SARS-CoV-2 CD4+ T cell epitopes to CCC sequences. Several studies have reported significant preexisting immune memory to SARS-CoV-2 peptides in unexposed donors (Braun et al., 2020; Grifoni et al., 2020; Le Bert et al., 2020; Mateus et al., 2020). This reactivity was shown to be associated, at least in some instances, with memory T cells specific for human common cold coronaviruses (CCC) cross-reactively recognizing SARS-CoV-2 sequences (Braun et al., 2020; Mateus et al., 2020). In particular, it was shown that the SARS-CoV-2 epitopes recognized in unexposed donors had significantly higher homology to CCC than SARS-CoV-2 sequences not recognized in unexposed donors. Here, using the exact same methodology (Mateus et al., 2020), the inventors performed the converse analysis, namely an analysis of the homology between the CD4+ T cell epitopes experimentally identified in COVID-19 donors (FIGS. 5D-L) and sequences of peptides derived from the four widely circulating human CCC (NL63, OC43, HKU1, 229E). No significant differences were observed based on percent sequence identity between epitopes recognized from the COVID-19 cohorts and non-epitope controls in structural proteins S, M, and N (FIGS. 5D-F), and accessory proteins encoded by ORF3a and ORF8 (FIGS. 5G-I) or non-structural proteins (FIGS. 5J-L).

It was found that the pattern of antigen recognition in exposed and unexposed donors was significantly different. Here, having defined the actual epitopes recognized in COVID-19, the inventors compared them to the epitopes previously identified in unexposed donors. The present study re-identified 50% of the epitopes in a prior COVID-19 cohort, but in addition identified 227 novel CD4+ T cell epitopes specific for SARS-CoV-2 infection (Table 4). Thus, more than 80% (227/280) of the epitopes identified herein are novel and were not previously seen in the unexposed cohort. These results are consistent with the notion that while a cross-reactive repertoire is present in unexposed donors, SARS-CoV-2 infection elicits a vast repertoire of novel T cell specificities.

Summary of CD8+ T cell epitope identification results. Following the approach described above, a total of 523 SARS-CoV-2 CD8+ T cell epitopes were identified (Table 5). These epitopes are associated with 26 different HLA restrictions, based on predicted HLA binding capacity matched to the HLA alleles of the responding donor. For eight HLAs, only 1-2 donors expressing the matching HLA could be tested. Predicted binders for the remaining 18 HLAs were tested in a median of 5 donors (range 3 to 9). The 8 most immunodominant proteins were screened in an average of 19 donors (range 4 to 35) (FIG. 4A). Of the 523 CD8+ T cell epitopes identified, 61 were recognized in 2 or 3 different donor-allele combinations, meaning that there were 454 unique peptides recognized. Of these, 101 (22%) were recognized by 2 or more donors, accounting for 49% of the total response. It was found that each donor recognized an average of 2.7 antigens (FIG. 1F) and responded to an average of 1.6 CD8+ T cell epitopes per antigen per HLA allele (data not shown). Considering 4 HLA A and B alleles in each donor, the inventors estimated at least 17 epitopes per donor for class I (2.7 × 1.6 × 4 = 17.3).

FIGS. 4 shows the frequency of positive epitopes (identified epitopes/peptides screened), and the average magnitude of epitope responses (total magnitude of response normalized by the number of positive donors), as a function of protein (FIG. 4B) or HLA class I allele (FIG. 4C) analyzed. Each HLA was associated with an average of 25 epitopes (range 7 to 40, median 24) (FIG. 4D). Interestingly, as also previously detected in other systems (Goulder et al., 1997; Weiskopf et al., 2013), there was a wide variation as a function of HLA allele. Some alleles, such as A*03:01 and A*32:01, were associated with responses that were both infrequent and weak; in other cases (e.g., A*01:01), responses were infrequent, but when observed were of high magnitude. Finally, and conversely, other alleles were associated with relatively frequent but low magnitude responses (e.g., A*68:01). This effect was previously linked to differences in the size of peptide repertoires associated with different HLA motifs (Paul et al., 2013).

In terms of antigen specificity of CD8+ T cell responses, relatively similar epitope-specific response frequencies were observed for the various antigens, with the exception of nsp12, which was associated with responses of low frequency and magnitude (FIG. 4B). These results should be interpreted with the caveat in mind that the donors screened were pre-selected on the basis of association with positive responses to that particular antigen; thus, this data does not directly address protein immunodominance, which is instead addressed in Table 2. These data instead point to the relative frequency and magnitude of responses at the level of individual epitopes associated with a given antigen, which were found to be overall similar.

To address the potential relationship between CD8+ T cell epitope recognition and CCC homology, as performed above in the case of CD4+ T cell epitopes, the inventors analyzed the homology of the CD8+ T cell epitopes to CCC (NL63, OC43, HKU1, 229E), as compared to the homolog to the same CCC viruses detected in the case of peptides that tested negative in all donors tested, regardless of the HLA-restriction (FIGS. 5D-L). Similar to what was observed in the context of CD4+ T cell responses, the CD8+ T cell epitopes recognized in convalescent COVID-19 donors were not associated with higher sequence identity to CCC as compared to non-epitopes, when structural, accessory or non-structural proteins were considered.

Distribution of CD4+ and CD8+ T cell epitopes within dominant SARS-CoV-2 antigens. Next, the inventors analyzed the distribution of CD4+ and CD8+ T cell epitopes within the dominant SARS-CoV-2 S, N, and M antigens (FIGS. 5). For each antigen, the inventors show the frequency (red line) and magnitude (black line) of CD4+ T cell responses along the antigen sequence, considering regions with response frequency above 20% as immunodominant. Based on the results presented above, the inventors also plotted HLA class II binding promiscuity (defined as the number of HLA allelic variants expressed in the donor cohort predicted to be bound by a given peptide), and the degree of homology of each 15-mer peptide for aligned CCC antigen sequences. The bottom panel represents the distribution of CD8+ T cell epitopes (black) and non-epitopes (red) along the antigen sequence.

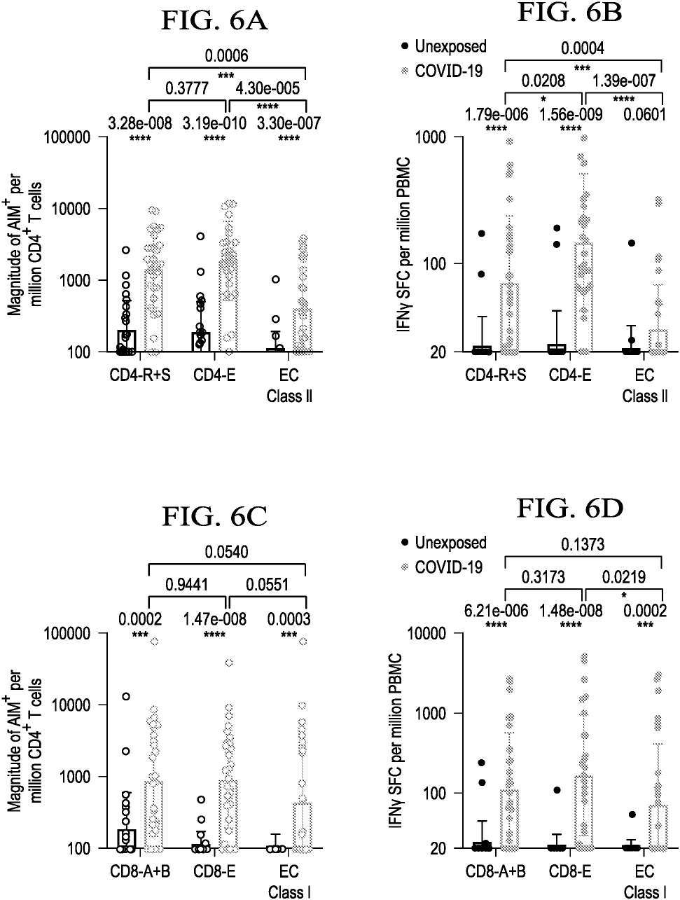

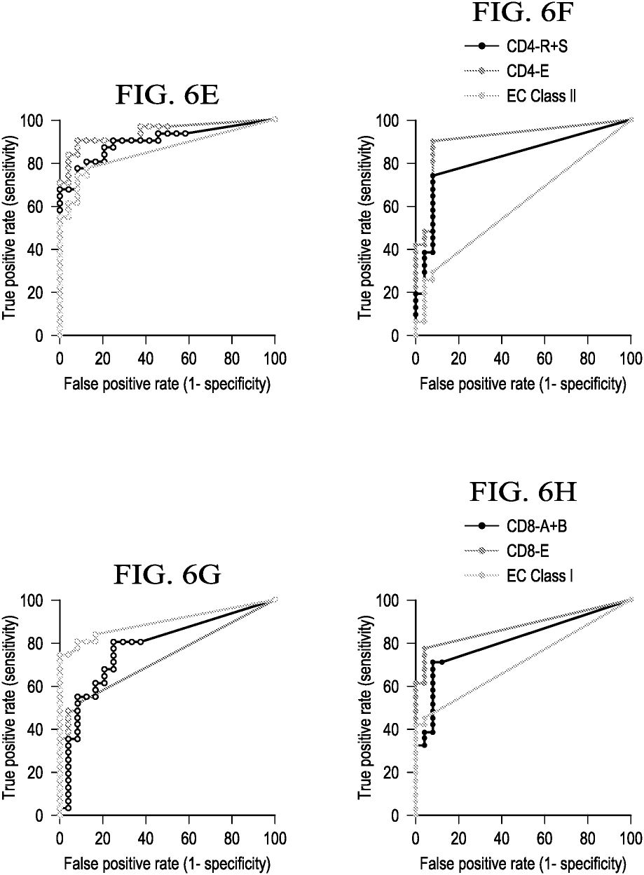

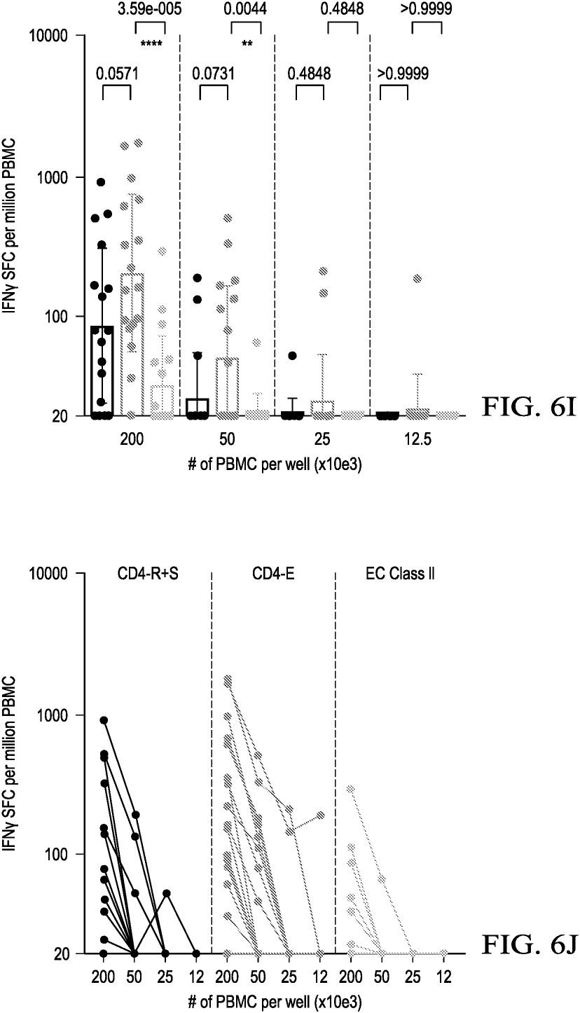

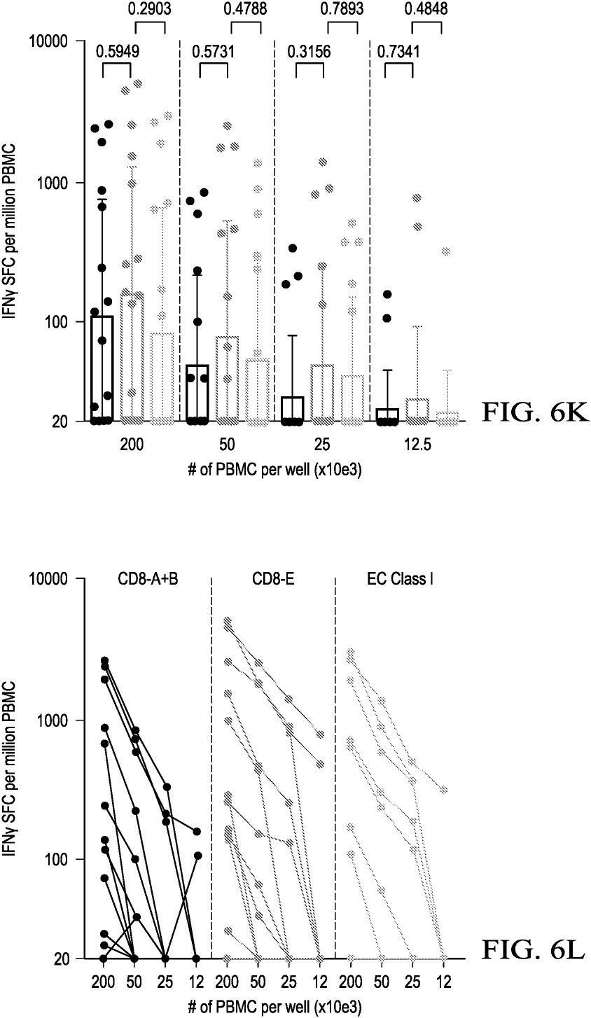

Responses to S peptides with a frequency of 20% or higher were focused on discrete regions of the protein involving the N-terminal domain (NTD), the C-terminal (CT) 686-816 region, and the neighboring fusion protein (FP) region; only a few responses were focused on the RBD. These immunodominant regions are boxed in red in FIG. 5A. Based on these results, it was found that HLA-binding capacity is associated with T cell immunodominant regions, and indeed had a significant positive correlation with the frequency of responses (R = 0.2231, p = 0.0003 by Spearman correlation, FIG. 5D). No significant correlation (R = -0.03144, p = 0.6187 by Spearman correlation, FIG. 5E) was found with sequence homology to CCC (calculated as maximum sequence homology to the four main CCC species). As indicated in the 3D rendering of the S crystal structure (PDB ID: 6XR8), these immunodominant regions were mostly located in the surface-exposed portions of the S monomer, and were not particularly influenced by the glycosylation pattern (shown in FIG. 5A as stars in the linear structure description, and based on experimental identification by Cai and co-authors (Cai et al., 2020)). The glycosylation patterns are also shown in the 3D-rendering of the corresponding crystal structure, based on curation done by the authors of the same manuscript, and shown as grey dots (FIG. 6A). The correlation between CD4+ T cell immunodominance and location of proteolytic cleavage sites, utilizing the MHCII-NP algorithm (Paul et al., 2018) was further explored. The results did not reveal any significant correlation between the predicted cleavage sites and immunodominant regions (Spearman correlation has R = -0.08426 and p = 0.1816, FIG. 5F). This is consistent with previous results that indicated that predicted cleavage sites do not significantly improve epitope predictions (Paul et al., 2018). Finally, CD8+ T cell reactivity did not reveal any particular immunodominant region in S, with epitopes and non-epitopes roughly equally distributed along the sequence (FIG. 5A).

In the same way, the inventors compared responses observed within the N and M proteins as a function of structural protein composition, HLA promiscuity and CCC homology (FIGS. 5B-C and FIGS. 6B-C). For the N protein (FIG. 5B), the majority of the response was focused on the NTD and CTD regions, with lower contributions from the linker region (all outlined in red boxes); segments in the middle and towards the ends of the protein were devoid of any reactivity. The correlation between immunodominance and HLA binding promiscuity was even stronger than observed for S (R = 0.4725, p = 7.41×10-6; FIG. 5G). Similar to what was observed for the S protein, no significant correlation between the frequency of positive responses was observed with CCC similarity (R = 0.1660, p = 0.1362; FIG. 5H) or predicted cleavage sites (R = -0.009245, p = 0.9343; FIG. 5I). The immunodominance of N-specific CD8+ T cell responses mirrors the one observed for the CD4+ T cell counterpart, highlighting that in general the N-terminal and C-terminal domains are the major immunodominant regions of N recognized by both T cell types.

CD4+ T cell immunogenic regions were distributed across the entire span of the M protein (FIG. 5C), including the transmembrane region (FIG. 6C). No significant correlation was observed when investigating HLA binding promiscuity (R = 0.2374, p = 0.1253; FIG. 5J), CCC similarity (R = 0.07648, p = 0.6259; FIG. 5K), or predicted cleavage sites (R = 0.08421, p = 0.5913; FIG. 5L). The lack of correlation between M epitopes and HLA binding is consistent with the interpretation that M is a prominent antigen because it is highly expressed, not because it contains high quality epitopes. No particular immunodominance patterns were observed for the M protein with respect to CD8+ epitopes. Finally, the location of immunodominant T cell regions relative to the main sites identified for antibody reactivity (Shrock et al., 2020) was investigated, the CD4+ T cell immunodominant regions identified in S and N showed minimal overlap with immunodominant linear regions targeted by antibody responses (Shrock et al., 2020) (FIGS. 6A TO 6L). The CD4+ T cell epitope recognition patterns of ORF3a, ORF8, nsp3, nsp4, nsp12, and nsp13 are as follows. The ORF8 protein was similar to M in that epitopes throughout both of these small proteins were recognized. ORF3a had clear regions of response clustered in the middle and at the C-terminus. Nsp3, which was the 4th most immunodominant antigen, was associated with a rather striking immunodominant region centered around residue 1643. Other non-structural proteins were less immunodominant overall, but had discreet regions targeted by CD4+ T cell responses (i.e., residue 5253 for nsp12).

Reactivity of megapools based on the experimentally identified epitopes. The experiments described above identified a total of 280 CD4+ and 454 CD8+ T cell epitopes. These epitopes were arranged into two epitope megapools (MPs), CD4-E and CD8-E, respectively (where the E denotes “experimentally defined”). These MPs were tested in a new cohort of 31 COVID-19 convalescent donors (none of these donors were utilized in the epitope identification experiments) and 25 unexposed controls (Table 3). MP reactivity was assessed for all donors using AIM and IFNγ FluoroSpot assays.

To put the results in context, the inventors also tested peptides contained in the CD4-R and CD4-S, and CD8-A and CD8-B MPs previously utilized to measure SARS-CoV-2 CD4+ and CD8+ T cell responses, respectively (Grifoni et al., 2020; Mateus et al., 2020; Rydyznski Moderbacher et al., 2020; Weiskopf et al., 2020). These MPs are based on either overlapping peptides spanning the entire S sequence (CD4-S) or predicted peptides (all other proteins). While these pools contain a larger total number of peptides (474 for CD4-R + CD4-S, and 628 for the CD8-A + CD8-B) than the corresponding experimentally defined sets, it could be expected that the experimentally defined peptide sets would be able to recapitulate the reactivity observed with the previously utilized MPs. As a further context, the inventors also tested the T cell Epitope Compositions (EC) class I and EC class II pools of experimentally defined CD8+ and CD4+ epitopes described by Nelde et al. (Nelde et al., 2020), encompassing 29 and 20 epitopes each, which prior to this study represented the most comprehensive set of experimentally defined epitopes.

As might be expected, the results showed that the AIM assay was more sensitive than the FluoroSpot assay . On the other hand, as a tradeoff for the lower signal, the FluoroSpot assay showed higher specificity in the responses detected, with fewer unexposed individuals showing any reactivity compared to the AIM assay. For CD4+ T cell responses as detected in the AIM assay (FIG. 6A), the CD4-E MP recapitulated the reactivity observed with the MPs of larger numbers of predicted peptides (CD4-R+S), and showed significantly higher reactivity (p = 4.30×10-6 by Mann-Whitney) as compared to the EC class IIpool. A similar picture was observed when the FluoroSpot assay was utilized (FIG. 6B), with a significantly higher reactivity of the CD4-E MP compared to the CD4-R+S (p = 0.0208 by Mann-Whitney), and to the EC class II pool (p = 1.39×10-7 by Mann-Whitney). In both AIM and FluoroSpot assays, the CD4-E MP showed the highest capacity to discriminate between COVID-19 convalescent and unexposed donors (p = 3.19×10-10 and p = 1.56×10-9, respectively by Mann-Whitney).

A similar picture was noted in the case of CD8+ T cell reactivity (FIGS. 6C-D), where the CD8-E MP recapitulated the reactivity observed with the MPs of larger numbers of predicted peptides (CD8-A+B), with a strong trend (p = 0.0551 by Mann-Whitney) towards more reactivity than the EC class II pool. In the case of the FluoroSpot assay, the inventors noted equivalent reactivity for the CD8-E and CD8-A+B MPs, and significantly higher reactivity (p = 0.0219 by Mann-Whitney) than the EC class II pool (FIG. 6D). In both assays, the CD8-E MP showed highest capacity to discriminate between COVID-19 convalescent and unexposed subjects (p = 1.47×10-8 and p = 1.48×10-8, respectively by Mann-Whitney). To test how well the different T cell responses measured separate individuals that have been exposed to SARS-Cov-2 versus those that do not, the inventors performed ROC analyses (FIGS. 6E-H) which allow us to directly compare the classification success based on true- and false-positive rates. The CD4-E and CD8-E response data were associated with the best performance.

Considering that a potential practical limitation in the characterization of SARS-CoV-2 responses is the number of cells available for study, in selected COVID-19 donors the inventors titrated the number of PBMC/well to determine if a response could be measured with lower cell numbers. As expected, as the cell input was decreased, the magnitude of responses decreased correspondingly. While marginal responses were seen with 25,000 cells/well and below, a sizeable response was still detectable with 50,000 cell/well, with 8 out of 17 donors responding for the CD4-E MP (as compared to 16 out of 17 in the case of 200,000 cell level). Similarly, in the case of the CD8-E MP, with 8 out of 17 donors responding (as compared to 11 out of 17 in the case of 200,000 cell level). The frequency and magnitude of responses of CD4-E were higher compared to the EC class II (p = 3.59×10-5 and p = 0.0044 by Mann-Whitney) (FIGS. 6I-J). The CD8-E MP was also associated with a higher magnitude of response than the EC class I pool (FIGS. 6K-L). In conclusion, these results underline the biological relevance of the more comprehensive CD4-E and CD8-E MPs.

The present invention includes a comprehensive analysis of the patterns of epitope recognition associated with SARS-CoV-2 infection in humans. The analysis was performed using a cohort of approximately 100 different convalescent donors spanning a range of peak COVID-19 disease severity representative of the observed distribution in the San Diego area. SARS-CoV-2 was probed using 1,925 different overlapping peptides spanning the entire viral proteome, ensuring an unbiased coverage of the different HLA class II alleles expressed in the donor cohort. For HLA class I the inventors used an alternative approach, selecting 5,600 predicted binders for 28 prominent HLA class I alleles, representing 61% of the HLA A and B allelic variants in the worldwide population, and affording an overall 98.8% HLA class I coverage at the phenotypic level.

The biological relevance of the epitope characterization studies summarized here is underlined by the use of the ex vivo AIM assay that does not require in vitro stimulation, which potentially skews the results by eliciting responses from naïve cells. The AIM assay is also more agnostic for different types of CD4+ T cells, as it measures all activated cells, regardless of T cell subset or any particular pattern of cytokine secretion.

To date, the repertoire of CD4+ and CD8+ T cell epitopes recognized in SARS-CoV-2 infection with a comparable level of granularity or breadth has not been determined. While several previous reports have described SARS-CoV-2 epitopes, and accordingly represent very useful advances, these studies either utilized in vitro expansion (Nelde et al., 2020), were limited in the number of proteins analyzed (Le Bert et al., 2020), characterized responses in fewer than 10 HLA types (Ferretti et al., 2020; Nelde et al., 2020; Peng et al., 2020), or focused on TCR repertoire after in vitro expansion of small numbers of cells (Snyder et al., 2020). Comparing these results with those obtained in those previous studies, it should be note that of the 20 HLA class II peptides identified by Nelde and co-authors (Nelde et al., 2020), 14 were contained within proteins the inventors mapped here in detail, and independently re-identified 12 (86%) of them (identical or largely overlapping sequences). Of 137 class I peptides reported thus far (Ferretti et al., 2020; Nelde et al., 2020; Peng et al., 2020), 98 were contained within the viral proteins the inventors mapped in detail, and independently re-identified 68 (69%) of them (identical or largely overlapping sequences).

Importantly, because SARS-CoV-2 antigen-specific T cell responses were evaluated in a systematic and unbiased fashion, quantitative estimates of the size of the repertoire of T cell epitope specificities recognized in each donor can be derived. Determining the breadth of responses is of relevance, since previous studies (Ferretti et al., 2020; Snyder et al., 2020) have suggested narrow SARS-CoV-2-specific T cell repertoires in COVID-19 patients; notably, a limited repertoire could favor viral mutation, a particular concern with this RNA virus. Based on these results, it could be estimated that each donor would be able to recognize about 19 CD4+ T cell epitopes, on average. Likewise, for CD8+ T cells, it could be estimated at least 17 epitopes per donor to be recognized. Overall, T cell responses in SARS-CoV-2 are estimated to recognize even more epitopes per donor than seen in the context of other RNA viruses, such as dengue (Grifoni et al., 2017; Weiskopf et al., 2015), where 11.6 and 7 CD4+ and CD8+ T cell epitopes, respectively, were recognized on average. This analysis should allay concerns over the potential for SARS-CoV-2 to escape T cell recognition by mutation of a few key viral epitopes.

The inventors defined the patterns of immunodominance across the various antigens encoded in the SARS-CoV-2 genome recognized in COVID-19 donors. Clear patterns of immunodominance were found, with a limited number of antigens accounting for about 80% of the total response. In general, the same antigens are dominant for both CD4+ and CD8+ responses, with some differences in relative ranking, such as in the case of nsp3, which is relatively more dominant for CD8+ than CD4+ T cell responses. Immunodominance at the protein level correlated with protein abundance/ gene, as previously noted for CD4+ T cell responses (Xie et al., 2020), although accessory proteins and nsps also account for a significant fraction of the response despite their predicted lower abundance in infected cells.

Because of their role in instructing both antibody and CD8+ T cell responses, the inventors correlated CD4+ T cell activity on a per donor and per antigen level with antibody and CD8+ T cell adaptive responses. This enabled establishing which antigens have functional relevance in terms of eliciting CD4+ T cell responses correlated with antibody and CD8+ T cell responses. At the level of antibody responses, S and M were correlated with RBD antibody titers, highlighting their capacity to support antibody responses, presumably by a deterministic linkage (viral antigen bridge) and cognate interactions (Sette et al., 2008). Surprisingly, N-specific CD4+ T cell responses did not correlate with S RBD antibody titers, suggesting unexpected complexity of the N-specific CD4+ T cell response. By contrast with these selective effects, CD4+ T cell activity against any of the antigens correlated with the total CD8+ T cell activity, suggesting that the role of CD4+ T cell responses driven by the different proteins is determinant in its helper function for either RBD-specific antibody production or CD8+ T cell responses. This was particularly true in both contexts when looking specifically at the S and M proteins, which are also the strongest and most frequently recognized antigens for both CD4+ and CD8+ T cells.

After examining relative immunodominance at the level of the different SARS-CoV-2 antigens, the inventors probed for variables that may influence which specific peptides are recognized within a given antigen/ORF. Previously, the inventors have shown that SARS-CoV-2 sequences recognized in unexposed individuals were associated with a higher degree of similarity to sequences encoded in the genome of various CCC. Here, repeating the same analysis with the SARS-CoV-2 epitopes recognized in COVID-19 donors, the inventors found no significant correlation. The inventors further show that while a large fraction of the epitopes previously identified in unexposed donors are re-identified in COVID-19 donors, about 80% of the epitopes are novel (not previously seen in unexposed), suggesting that the SARS-CoV-2-specific T cell repertoire of COVID-19 cases is overlapping, but substantially different from, the SARS-CoV-2-cross-reactive memory T cell repertoire of unexposed donors. This is consistent with the present inventors’ observation of a different pattern of reactivity (Mateus et al., 2020), and consistent with reports from other groups (Le Bert et al., 2020; Nelde et al., 2020).

HLA binding capacity was a major determinant of immunogenicity for CD4+ T cells (the influence of HLA binding was not evaluated for CD8+ T cell, since the tested epitope candidates were picked based of their predicted HLA binding capacity). As found in several previous large-scale pathogen-derived epitope identification studies, immunodominant epitopes were also found to be promiscuous HLA class II binders (Lindestam Arlehamn et al., 2016; Oseroff et al., 2010). Binding to multiple HLA allelic variants is an important mechanism to amplify the potential immunogenicity of peptide epitopes and specific regions within an antigen. It is possible that the dominance of particular regions might further correlate with processing. However, at this juncture, HLA class II processing algorithms do not effectively predict epitope recognition (Barra et al., 2018; Cassotta et al., 2020; Paul et al., 2018).

Further analysis projected the CD4+ T cell dominant regions on known or predicted SARS-CoV-2 protein structures. This established that the dominant epitope regions are different for B and T cells. This is of relevance for vaccine development, as inclusion of antigen sub-regions selected on the basis of dominance for antibody reactivity might result in an immunogen devoid of sufficient CD4+ T cell activity. In this context, it is important to note that the RBD region had very few CD4+ T cell epitopes recognized in COVID-19 donors, but inclusion of regions neighboring the RBD N- and C-termini would be expected to provide sufficient CD4+ T cell help.

In contrast to the clear demarcation of dominant regions for antibody and CD4+ T cell responses, CD8+ T cell epitopes were uniformly dispersed throughout the various antigens, consistent with previous in-depth analyses revealing little positional effect in CD8+ T cell epitope distribution (Kim et al., 2013). In the case of CD8+ T cell responses, these data highlights HLA-allele specific differences in the frequency and magnitude of responses. This effect was noted before in the case of dengue virus (Weiskopf et al., 2013) and related to potential HLA-linked protective versus susceptibility effects. The current study is not powered to test these potential effects, leaving it to future studies to examine this possibility. Regardless, this study provides a roadmap for inclusion of specific regions or discrete epitopes, to allow for CD8+ T cell epitope representation across a variety of different HLAs.

Finally, the functional relevance of this study was highlighted by the generation of novel and improved epitope MPs for measuring T cell responses to SARS-CoV-2; these newer experimentally defined pools are associated with increased activity and lower complexity when compared to the inventors’ previous MPs based on overlapping and predicted peptides. These epitope pools can be used by the scientific community at large and can facilitate further investigation of the role of T cell immunity in SARS-CoV-2 infection and COVID-19.

In conclusion, the present invention includes several hundred different HLA class I and class II restricted SARS-CoV-2-derived epitopes. These HLA class I and class II restricted SARS-CoV-2-derived epitopes can be used for basic investigation of SARS-CoV-2 immune responses and in the development of both multimeric staining reagents and T cell-based diagnostics, as well as in treatments, immunizations, and kits. In addition, the results shed light on the mechanisms of immunodominance of SARS-CoV-2, which have implications for understanding host-virus interactions, as well as for vaccine design.

Human Subjects. Convalescent COVID-19 Donors utilized for epitope identification. Blood donations from the 99 convalescent donors included in this study’s cohort were collected through either the UC San Diego Health Clinic under IRB approved protocols (200236X), or under IRB approval (VD-214) at the La Jolla Institute. Donations obtained through the CROs Sanguine, BioIVT and Stem Express were collected under the same IRB approval (VD-214) at the La Jolla Institute. Details of this cohort can be found in Table 1. All donors were over the age of 18 years and no exclusions were made due to disease severity, race, ethnicity, or gender. All donors were able to provide informed consent, or had a legal guardian or representative able to do so. Study exclusion criteria included lack of willingness or ability to provide informed consent, or lack of an appropriate legal guardian to provide informed consent.

Disease severity was defined as mild, moderate, severe or critical as previously described (Grifoni 2020). In brief, this classification of disease severity is based on a modified version of the WHO interim guidance, “Clinical management of severe acute respiratory infection when COVID-19 is suspected” (WHO Reference Number: WHO/2019-nCoV/clinical/2020.4). At the time of enrollment in the study, 80% of donors had been confirmed positive by swab test viral PCR during the acute phase of infection. Plasma samples from all donors were later tested by IgG ELISA for SARS-CoV-2 S protein RBD to verify previous infection (Table 1 and FIG. 2A).

Healthy Unexposed donors utilized for CD4-E and CD8-E megapool validation. Samples from healthy adult donors were obtained from the San Diego Blood Bank (SDBB). According to the criteria set up by the SDBB if a subject was eligible to donate blood, they were considered eligible for this study. All the donors were tested for SARS-CoV-2 RBD IgG serology and were found negative and therefore considered unexposed. An overview of the characteristics of these donors is provided in Table 3. Convalescent COVID-19 donors utilized for CD4-E and CD8-E megapool validation. The 31 convalescent donors tested in the megapool AIM and FluoroSpot assays (FIGS. 6A-6L) were collected from the same clinics using the same protocols as described above for the donors utilized for epitope identification. Similarly, no donors enrolled were under the age of 18 and none were excluded due to disease severity, race, ethnicity, or gender. All donors, or legal guardians, gave informed consent. Specific characteristics of these donors can be found in Table 3, including the summary of ELISA testing for SARS-CoV-2 S protein RBD.

Peptide Pools. Preparation of 15-mers and subsequent megapools and mesopools. To identify SARS-CoV-2-specific T cell epitopes, 15-mer peptides overlapping by 10 amino acids and spanning entire SARS-CoV-2 proteins were synthesized. All peptides were synthesized as crude material (A&A, San Diego, CA) and individually resuspended in dimethyl sulfoxide (DMSO) at a concentration of 10 mg/mL. Aliquots of these peptides were pooled by antigen of provenance into megapools (MP) (as described in Table 2) and sequentially lyophilized as previously reported (Carrasco Pro et al., 2015). Another portion of the 15-mer peptides were pooled into smaller mesopools of ten peptides each. All pools were resuspended at 1 mg/mL in DMSO.

Class I peptide preparation. Class I predicted peptides were designed using the protein sequences derived from the SARS-CoV-2 reference strain (GenBank: MN908947). Predictions were performed as previously reported using NetMHC pan EL 4.0 algorithm (Jurtz et al., 2017) for 28 HLA A and B alleles that were selected based on frequency in this cohort and also representative of the worldwide population (FIGS. 4E-4F). The top 200 predicted peptides were selected for each allele. In total 5,600 class I peptides were synthesized and resuspended in DMSO at 10 mg/mL.

PBMC isolation and HLA typing. Whole blood was collected from all donors in either Acid Citrate Dextrose (ACD) tubes or heparin coated blood bags. Whole blood was then centrifuged at room temperature for 15 minutes at 1850 rpm to separate the cellular fraction and plasma. The plasma was then carefully removed from the cell pellet and stored at -20 C. Peripheral blood mononuclear cells (PBMC) were isolated by density-gradient sedimentation using Ficoll-Paque (Lymphoprep, Nycomed Pharma) as previously described (Weiskopf et al., 2013). Isolated PBMC were cryopreserved in cell recovery media containing 10% DMSO (Gibco), supplemented with 90% heat-inactivated fetal bovine serum, depending on the processing laboratory, (FBS; Hyclone Laboratories, Logan UT) and stored in liquid nitrogen until used in the assays. Each sample was HLA typed by Murdoch University in Western Australia, an ASHI-Accredited laboratory (Voic 2020, Madden 1995, Gorse 2010). Typing was performed for the class I HLA A and B loci and class II DRBI, DQB1, and DPB1 loci.

SARS-CoV-2 RBD ELISA. The SARS-CoV-2 RBD ELISA has been described in detail elsewhere (Grifoni 2020, Amanat 2020). All convalescent COVID-19 donors had their serology determined by ELISA. Briefly, 96-well half-area plates (ThermoFisher 3690) were coated with 1 ug/mL SARS-CoV-2 Spike (S) Receptor Binding Domain (RBD) and incubated at 4° C. overnight. On the following day plates were blocked at room temperature for 2 hours with 3% milk in phosphate buffered saline (PBS) containing 0.05% Tween-20. Then, heat-inactivated plasma was added to the plates for another 90-minute incubation at room temperature followed by incubation with conjugated secondary antibody, detection, and subsequent data analysis by reading the plates on Spectramax Plate Reader at 450 nm using SoftMax Pro. Limit of detection (LOD) was defined as 1:3. Limit of sensitivity (LOS) for SARS-CoV-2 infected individuals was established based on uninfected subjects, using plasma from normal healthy donors not exposed to SARS-CoV-2.

Flow Cytometry. Activation induced cell marker (AIM) assay. The AIM assay was performed as previously described (Dan et al., 2016; Reiss et al., 2017). Cryopreserved PBMCs were thawed by diluting the cells in 10 mL complete RPMI 1640 with 5% human AB serum (Gemini Bioproducts) in the presence of benzonase [20 µl/10 ml]. Cells were cultured for 20 to 24 hours in the presence of SARS-CoV-2 specific MPs [1 µg/ml], mesopools [1 µg/ml], 15-mers [10 µg/ml], or class I predicted peptides [10 µg/ml] in 96-wells U bottom plates with 1×106 PBMC per well. As a negative control, an equimolar amount of DMSO was used to stimulate the cells as a negative control in triplicate wells, and phytohemagglutinin (PHA, Roche, 1 µg/ml) was included as the positive control. The cells were stained with CD3 AF700 (4:100; Life Technologies Cat# 56-0038-42), CD4 BV605 (4:100; BD Biosciences Cat# 562658), CD8 BV650 (2:100; Biolegend Cat# 301042), and Live/Dead Aqua (1:1000; eBioscience Cat# 65-0866-14). Activation was measured by the following markers: CD137 APC (4:100; Biolegend Cat# 309810), OX40 PE-Cy7 (2:100; Biolegend Cat#350012), and CD69 PE (10:100; BD Biosciences Cat# 555531). All samples were acquired on either a ZE5 cell analyzer (Bio-rad laboratories) or an Aurora flow cytometry system (Cytek), and analyzed with FlowJo software (Tree Star).

\\HLA binding assays. The binding of selected SARS-CoV-2 15-mer epitopes to HLA class II MHC molecules was measured as previously described (Sidney 2013, Voic 2020). In brief, the binding is quantified by each peptide’s capacity to inhibit the binding of a radiolabeled peptide probe to purified MHC in classical competition assays. The probe was incubated with purified MHC, a mixture of protease inhibitors, and different concentrations of unlabeled inhibitor peptide at room temperature or 37° C. for 2 days. MHC molecules were subsequently captured on HLA-DR-specific monoclonal antibody (L243) coated Lumitrac 600 plates (Greiner Bio-one, Frickenhausen, Germany) and radioactivity was measured using the TopCount microscintillation counter (Packard Instrument Co., Meriden, CT). Each peptide was tested at 6 concentrations to cover a 100,000-fold dose range, and an unlabeled version of the radiolabeled probe was included in each experiment as a positive control for inhibition. To analyze the results, the inventors calculated the concentration of peptide at which the binding was inhibited by 50% (IC50 nM). For these values to approximate true Kd values, the following conditions were met: 1) the concentration of radiolabelled probe is less than the concentration of MHC, and 2) the measured IC50 is greater than or equal to the concentration of MHC.

FluoroSpot. PBMCs derived from 25 unexposed donors were stimulated in triplicate at a single density of 200×103 cells/well (one donor was tested at 50×103 due to limitation in cell numbers). PBMCs from a cohort of 31 convalescent COVID-19 donors were stimulated in triplicates of 200×103 cells/well, with the exception of 5 donors tested at 50-100×103 cells/well due to cell limitations (FIGS. 6B, D, F, and H). Seventeen of these convalescent donors were further titrated at 200, 50, 25, and 12.5×103 cells/well (FIGS. 6I-L). The cells were stimulated with the different MPs analyzed (1 µg/mL), PHA (10 µg/mL), and DMSO (0.1%) in 96-well plates previously coated with anti-cytokine antibodies for IFNγ, (mAbs 1-D1K; Mabtech, Stockholm, Sweden) at a concentration of 10 µg/mL. After 20 hours of incubation at 37° C., 5% CO2, cells were discarded and FluoroSpot plates were washed and further incubated for 2 hours with cytokine antibodies (mAbs 7-B6-1-BAM; Mabtech, Stockholm, Sweden). Subsequently, plates were washed again with PBS/0.05% Tween20 and incubated for 1 hour with fluorophore-conjugated antibodies (Anti-BAM-490). Computer-assisted image analysis was performed by counting fluorescent spots using an AID iSPOT FluoroSpot reader (AIS-diagnostika, Germany). Each megapool was considered positive compared to the background based on the following criteria: 20 or more spot forming cells (SFC) per 106 PBMC after background subtraction for each cytokine analyzed, a stimulation index (S.I.) greater than 2, and statistically different from the background (p <0.05) in either a Poisson or T test. Bioinformatic and statistical analysis. FlowJo 10 and GraphPad Prism 8.4 were used to perform data and statistical analyses, unless otherwise stated. Statistical details of the experiments are provided in the respective figure legends. Data plotted in linear scale are expressed as mean + standard deviation (SD). Data plotted in logarithmic scales are expressed as median + 95% confidence interval (CI) or geometric mean + geometric SD. Statistical analyses were performed using Spearman correlation and Mann-Whitney or Kolmogorov-Smirnov tests for unpaired comparisons. Details pertaining to significance are also noted in the respective figure legends.

AIM assay analysis. In analyzing data from the AIM assays, the counts of AIM+ CD4+ and CD8+ T cells were normalized based on the counts of CD4+ and CD8+ T cells in each well to be equivalent to 1×106 total CD8+ or CD4+ T cells. The background was removed from the data by subtracting the single or the average of the counts of AIM+ cells plated as single or triplicate wells stimulated with DMSO. The inventors included the triplicate wells stimulated with DMSO in the mesopools and epitope identification steps to take into account the variability of the weaker signals observed in those two respect to the original MP reactivity (da Silva Antunes et al., 2020). The Stimulation Index (SI) was calculated by dividing the count of AIM+ cells after SARS-CoV-2 stimulation with the ones in the negative control. A positive response had an SI greater than 2 and a minimum of 100 AIM+ cells after background subtraction. The gates for AIM+ cells were drawn relative to the negative and positive controls for each donor. A representative example of the gating strategy is depicted in FIG. 1.

HLA class I nested epitopes. For some alleles and proteins, multiple nested class I predicted peptides were tested in the AIM assay. In cases where a specific donor responded to multiple nested epitopes corresponding to the same allele and protein, the epitope with the highest magnitude of response was classified as the optimal epitope. If multiple nested epitopes had the same response (within a range of 50 AIM+ cells), the epitope with the shortest length was selected. Nested epitopes corresponding to different donors or different alleles were conserved as separate epitopes.

CCC homology analysis. SARS-CoV-2-derived 15-mer peptides were analyzed for their identity with the common cold coronaviruses (CCC) 229E, NL63, HKU1, and OC43, as previously described (Mateus et al., 2020). In brief, every SARS-CoV-2 15-mer peptide tested for immunogenicity was compared against every position in the corresponding protein sequences of common coronaviruses obtained from GenBank. The region that best matched the respective SARS-CoV-2 peptide was used to calculate percent sequence identity for each of the four CCC viruses individually, as well as the maximum across all four (FIGS. 4K-4Q). The same methodology was also used to calculate sequence identity for SARS-CoV-2 class I peptides (FIGS. 4K-4Q). Using the same set of common coronavirus reference sequences, an alternative analysis was performed by mapping each SARS-CoV-2 peptide with the S, M and N protein sequences corresponding to the four common coronavirus using Immunobrowser tool (Dhanda et al., 2018). The values resulted from this specific analysis are plotted in FIGS. 5.

T cell epitope restriction predictions. Putative HLA class II restrictions for individual 15-mer CD4+ T cell epitopes were inferred using the IEDB’s TepiTool resource (Paul 2016). All CD4+ T cell prediction analyses were performed applying the NetMHCIIpan algorithm (Karosiene et al., 2013). Prediction analyses were performed to either infer HLA restriction based on the HLA typing of the cohort or to assess potential binding promiscuity of experimentally defined epitopes, considering the 27 most frequent class II alleles in the worldwide population (Greenbaum et al., 2011). In both types of prediction analyses, a 20th percentile threshold was applied, as previously described (Mateus et al., 2020). Assigning regions within the linear structure. Simple diagrams were created to describe the linear structures of S, N, and M proteins (FIGS. 4). The different regions of the S protein were defined based on the works of Cai et al. 2020. The structure of the N protein was divided into 3 main regions, the N- and C-terminal domains, and the linker region in between (Zeng et al., 2020). For the M protein, the regions of the structure were extracted from UniProt (UniProtKB - P59596 (VME1_SARS).

3D-rendering and model design. Three different approaches have been used to map T and B cell immunodominant regions on the 3D-structures for SARS-CoV-2 S, M and N proteins. The S protein model was based on the crystal structure described in Cai et al. 2020 (PDB ID: 6XR8) and using the glycosylation sites annotated in the submitted PDB. The M protein model has been previously described by Heo et al., 2020. The model for the N protein was run on four different homology prediction servers (SWISS-MODEL, RaptorX, iTasser and Phyre2). In order to have a complete N sequence, Phyre2 server was subsequently selected using the intensive mode (Kelley and Sternberg, 2009). The resulting model showed a variable level of confidence with higher percentages (>90%) in the C-Terminal domain (CTD) and N-terminal domain (NTD) regions and low confidence percentages (>10%) in the linker domain. The N model was superimposable with both the crystal structures for the CTD (PDB ID: 6WZO) and NTD (PDB ID: 6M3M). The current N model has the only purpose of visualization for mapping immunodominant regions. All the mapping analyses have been performed using the free version of YASARA (Land and Humble, 2018).

TABLE 1

Characteristics of donor cohort utilized in the protein screen

| | |
|---|---|
| | COVID-19 (n = 99) |
| Age (years) | 19-91 [median = 41, IQR = 23] |
| Gender | |
| Male (%) | 41% (42/99) |
| Female (%) | 59% (58/99) |
| Sample Collection Date | Mar-September 2020 |
| SARS-CoV-2 PCR | Positive = 100% (78/78) |
| | Not tested= 21% (21/99) |
| S RBD IgG | 98% (97/99), |
| Peak disease Severity^{a} | |
| Mild | 91% (90/99) |
| Moderate | 2% (2/99) |
| Severe | 1% (1/99) |
| Critical | 6% (6/99) |
| Race-Ethnicity | |
| White- not Hispanic or Latino | 76% (76/99) |
| Hispanic or Latino | 12% (12/99) |
| Asian | 5% (5/99) |
| American Indian/Alaska Native | 1% (1/99), |
| Native Hawaiian or other Pacific ......... Islander | 0% (0/99) |
| Black or African American | 2%(2/99) |
| More than one race | 3% (3/99) |
| Not reported | 0% (0/99) |
| Days Post Symptom Onset at Collection^{b} | 3-184 (108/108) [Median = 67, IQR = 48] |

^{a}According to WHO criteria.
^{b}Multiple visits for the same donor have been analyzed.

TABLE 2

Summary of SARS-CoV-2-specific T cell reactivity as a function of the most immunodominant proteins^{a}

| CD4⁺ T cells s (n= 99) | | | | | | | | CD8⁺ T cell s (n=9 9) | | | | | | | |
|---|---|---|---|---|---|---|---|---|---|---|---|---|---|---|---|
| proteins | Frequency (%) | Total counts | Average counts | % of responses per protein | Cumulative (%) | # of peptides tested | Cumulative (%) | proteins | Frequency (%) | Total counts | Average counts | % of responses per protein | Cumulative (%) | # of peptides tested | Cumulative (%) |
| S | 95 | 4718 73 | 4766 | 26 | 25 | 253 | 13 | S | 80 | 4409 27 | 4454 | 26 | 26 | 253 | 13 |
| M | 96 | 2688 83 | 2716 | 17 | 43 | 43 | 15 | nsp3 | 49 | 1741 04 | 1759 | 17 | 43 | 388 | 33 |
| N | 93 | 2204 25 | 2227 | 15 | 58 | 82 | 20 | N | 75 | 1801 91 | 1820 | 15 | 58 | 82 | 38 |
| nsp3 | 70 | 1605 67 | 1622 | 8 | 66 | 388 | 40 | M | 69 | 1490 71 | 1506 | 11 | 69 | 43 | 40 |
| ORF 3a | 76 | 9402 5 | 950 | 5 | 70 | 53 | 43 | ORF 3a | 47 | 5843 9 | 590 | 4 | 73 | 53 | 43 |
| nsp1 2 | 64 | 7515 7 | 759 | 4 | 74 | 186 | 52 | nsp4 | 45 | 3994 5 | 403 | 3 | 76 | 100 | 48 |
| nsp4 | 58 | 6877 8 | 695 | 3 | 77 | 100 | 57 | nsp1 2 | 35 | 2676 4 | 270 | 3 | 79 | 186 | 57 |
| nsp1 3 | 58 | 5168 1 | 522 | 3 | 80 | 120 | 64 | nsp6 | 34 | 2661 9 | 269 | 2 | 81 | 58 | 60 |
| ORF 8 | 58 | 5021 7 | 507 | 2 | 83 | 23 | 65 | nsp2 | 30 | 2232 4 | 225 | 2 | 83 | 127 | 67 |
| nsp1 6 | 34 | 2917 5 | 295 | 2 | 85 | 59 | 68 | nsp1 | 24 | 1839 3 | 186 | 2 | 85 | 36 | 69 |
| nsp2 | 54 | 4867 4 | 492 | 2 | 87 | 127 | 74 | nsp5 | 31 | 1713 6 | 173 | 2 | 87 | 61 | 72 |
| ORF 7a | 48 | 5927 0 | 599 | 2 | 89 | 23 | 76 | nsp1 6 | 20 | 2022 1 | 204 | 2 | 89 | 59 | 75 |
| Nsp6 | 49 | 2878 3 | 291 | 2 | 91 | 58 | 79 | nsp1 3 | 25 | 1600 4 | 162 | 2 | 91 | 120 | 81 |
| nsp1 4 | 44 | 2605 9 | 263 | 2 | 93 | 105 | 84 | ORF 8 | 32 | 2238 9 | 226 | 1 | 92 | 23 | 83 |
| nsp5 | 46 | 2913 9 | 294 | 2 | 94 | 61 | 87 | ORF 7a | 28 | 1961 2 | 198 | 1 | 93 | 23 | 84 |
| E | 33 | 2116 1 | 214 | 1 | 95 | 13 | 88 | nsp1 4 | 19 | 1113 5 | 112 | 1 | 94 | 105 | 89 |
| nsp1 | 22 | 2035 7 | 206 | 1 | 97 | 36 | 90 | nsp7 | 18 | 7480 | 76 | 1 | 95 | 17 | 90 |
| nsp8 -9-10 | 37 | 2141 9 | 216 | 1 | 98 | 91 | 95 | nsp8 -9-10 | 18 | 1406 7 | 142 | 1 | 97 | 91 | 95 |
| nsp1 5 | 29 | 1715 4 | 173 | 1 | 98 | 70 | 98 | ORF 6 | 24 | 1294 7 | 131 | 1 | 98 | 11 | 95 |
| ORF 6 | 27 | 1063 2 | 107 | 1 | 99 | 11 | 99 | nsp1 5 | 14 | 1220 8 | 123 | 1 | 99 | 70 | 99 |
| ORF 10 | 19 | 7551 | 76 | 0 | 100 | 6 | 99 | ORF 10 | 20 | 1217 8 | 123 | 1 | 99 | 6 | 99 |
| nsp7 | 19 | 1424 8 | 144 | 0 | 100 | 17 | 100 | E | 20 | 954296 | | 1 | 100 | 13 | 100 |

^{a}Bold font indicates the top SARS-CoV-2 proteins accounting for a cumulative response >80% for CD4⁺ and CD8⁺ T cells.

TABLE 3

Characteristics of donor cohorts utilized to validate megapools in FIGS. 6A-6L

| | | |
|---|---|---|
| | Unexposed (n = 25) | COVID-19 (n = 31) |
| Age (years) | 24-82 [Median = 36, IQR = 31] | 21-81 [Median = 38, IQR = 29] |
| Gender | | |
| Male (%) | 52% (13/25) | 35% (11/31) |
| Female (%) | 48% (12/25) | 65% (20/31) |
| Sample Collection Date | March - May 2020 | April - September 2020 |
| SARS-CoV-2 PCR | N/A | Positive 100% (18/18) |
| | | Not tested= 42% (13/31) |
| S RBD IgG | 0% (0/25) | 100% (31/31) |
| Peak disease Severity^{a} | | |
| Mild | N/A | 58% (18/31) |
| Moderate | N/A | 36% (36/31) |
| Severe | N/A | 6% (2/31) |
| Critical | N/A | 0% (0/31) |
| Race-Ethnicity | | |
| White- not Hispanic or Latino | 44% (11/25) | 81% (25/31) |
| Hispanic or Latino | 8% (2/25) | 13% (4/31) |
| Asian | 12% (3/25) | 3% (1/31) |
| American Indian/Alaska Native | 0% (0/25) | 0% (0/31) |
| Native Hawaiian or Other Pacific ......... Islander | 4% (1/25) | 0% (0/31) |
| Black or African American | 0% (0/25) | 0% (0/31) |
| More than one race | 0% (0/25) | 0% (0/31) |
| Not reported | 32% (8/25) | 3% (1/31) |
| Days Post Symptom Onset at Collection | N/A | 22-128 [Median = 69, IQR = 50] |

^{a}According to WHO criteria.

TABLE 4

List of CD4+ T cell epitopes identified and their predicted HLA restriction(s). A total of 280 15-mer epitopes were identified by AIM assay and encompassed the 9 dominant SARS-CoV-2 antigens for CD4+ T cells

| Protein | sequence | HLA restriction | Frequency of response (%) | Sum of response | Magnitude by # of responding donors | SEQ ID NO |
|---|---|---|---|---|---|---|
| M | YRINWITGGIAIAMA | DQB1*02:02, DQB1*03:01, DQB1*05:01, DQB1*05:02, DQB1*05:03, DQB1*06:02, DQB1*06:03, DRB1*07:01, DRB1*10:01, DRB1*12:01, DRB1*13:01, DRB1*14:01, DRB1*15:01, DRB1*16:01 | 58 | 2417 | 345 | 1 |
| ORF8 | PCPIHFYSKWYIRVG | DRB1*08:02, DRB1*13:01, DRB1*15:01, DRB1*15:02, DRB1*16:01 | 55 | 2948 | 491 | 2 |
| M | KEITVATSRTLSYYK | DQB1*06:03, DRB1*03:01, DRB1*07:01, DRB1*14:01, DRB1*14:06, DRB1*15:01, DRB1*16:02 | 54 | 3615 | 516 | 3 |
| M | LSYYKLGASQRVAGD | DQB1*03:01, DQB1*06:02, DQB1*06:03, DRB1*01:01, DRB1*07:01, DRB1*14:06, DRB1*15:01, DRB1*16:01, DRB1*16:02 | 46 | 3465 | 578 | 4 |
| ORF8 | IGNYTVSCLPFTINC | DQB1*03:03, DRB1*15:02 | 45 | 1223 | 245 | 5 |
| S | NIDGYFKIYSKHTPI | DRB1*07:01, DRB1*15:01, DRB1*16:01 | 45 | 1475 | 295 | 6 |
| M | VLAAVYRINWITGGI | DQB1*05:01, DQB1*05:03, DRB1*12:02, DRB1*14:01 | 42 | 2519 | 504 | 7 |
| M | RGHLRIAGHHLGRCD | DRB1*07:01, DRB1*13:01, | 42 | 2359 | 472 | 8 |
| | | DRB1*14:01, DRB1*15:01 | | | | |
| N | ILLNKHIDAYKTFPP | DRB1*14:06, DRB1*15:01 | 42 | 2416 | 483 | 9 |
| ORF8 | TQHQPYVVDDPCPIH | DRB1*13:02 | 36 | 1352 | 338 | 10 |
| ORF8 | YVVDDPCPIHFYSKW | DRB1*13:02, DRB1*15:02 | 36 | 3488 | 872 | 11 |
| ORF8 | RCSFYEDFLEYHDVR | DQB1*05:02 | 36 | 696 | 174 | 12 |
| S | LMDLEGKQGNFKNLR | HLA class II | 36 | 1386 | 347 | 13 |
| M | CLVGLMWLSYFIASF | DQB1*05:01, DQB1*05:03, DRB1*12:01, DRB1*12:02 | 33 | 1181 | 295 | 14 |
| M | SELVIGAVILRGHLR | DQB1*06:02, DQB1*06:03, DRB1*01:02, DRB1*13:01, DRB1*14:01, DRB1*15:01, DRB1*16:01 | 33 | 563 | 141 | 15 |
| N | WPQIAQFAPSASAFF | DQB1*02:02, DQB1*05:03, DRB1*04:04, DRB1*07:01, DRB1*15:01 | 33 | 1503 | 376 | 16 |
| N | FKDQVILLNKHIDAY | DRB1*03:01, DRB1*14:01, DRB1*14:06, DRB1*15:01, DRB1*16:02 | 33 | 1785 | 446 | 17 |
| nsp16 | AVMSLKEGQINDMIL | HLA class II | 33 | 104 | 104 | 18 |
| nsp16 | KEGQINDMILSLLSK | HLA class II | 33 | 391 | 391 | 19 |
| nsp16 | RENNRVVISSDVLVN | DQB1*02:02, DRB1*07:01 | 33 | 170 | 170 | 20 |
| M | SGFAAYSRYRIGNYK | DRB1*15:01 | 31 | 739 | 185 | 21 |
| N | KKPRQKRTATKAYNV | HLA class II | 31 | 907 | 227 | 22 |
| N | KRTATKAYNVTQAFG | DQB1*06:02, DQB1*06:03 | 31 | 752 | 188 | 23 |
| N | NKDGIIWVATEGALN | DQB1*02:01, DQB1*02:02, DQB1*03:02, DQB1*05:03, DRB1*04:04, DRB1*07:01 | 29 | 1747 | 437 | 24 |
| ORF8 | FTINCQEPKLGSLVV | HLA class II | 27 | 502 | 167 | 25 |
| ORF8 | GSLVVRCSFYEDFLE | DQB1*05:02, DQB1*05:03 | 27 | 694 | 231 | 26 |
| S | SSANNCTFEYVSQPF | HLA class II | 27 | 4279 | 1426 | 27 |
| S | NLVRDLPQGFSALEP | DRB1*03:01 | 27 | 1822 | 607 | 28 |
| S | RFASVYAWNRKRISN | DRB1*07:01, DRB1*13:01, DRB1*14:01 | 27 | 722 | 241 | 29 |
| M | MWLSYFIASFRLFAR | DQB1*02:01, DQB1*05:01, DQB1*05:02, DQB1*05:03, DRB1*03:01, DRB1*14:01, DRB1*14:06, DRB1*16:01, DRB1*16:02 | 25 | 1030 | 343 | 30 |
| M | TNILLNVPLHGTILT | DRB1*10:01, DRB1*12:02, DRB1*14:01, DRB1*14:06, DRB1*15:01, DRB1*16:02 | 25 | 1628 | 543 | 31 |
| M | IAGHHLGRCDIKDLP | HLA class II | 25 | 1565 | 522 | 32 |
| M | LGRCDIKDLPKEITV | HLA class II | 25 | 938 | 313 | 33 |
| S | AGFIKQYGDCLGDIA | DQB1*05:03 | 25 | 2450 | 817 | 34 |
| N | SWFTALTQHGKEDLK | HLA class II | 23 | 3265 | 1088 | 35 |
| N | YYRRATRRIRGGDGK | DRB1*13:01, DRB1*14:06 | 23 | 592 | 197 | 36 |
| M | IKDLPKEITVATSRT | HLA class II | 23 | 774 | 258 | 37 |
| N | AAEASKKPRQKRTAT | HLA class II | 23 | 537 | 179 | 38 |
| ORF3a | LYLYALVYFLQSINF | DQB1*02:01, DQB1*03:02, DQB1*05:03, DRB1*11:01, DRB1*12:01 | 21 | 371 | 124 | 39 |
| N | AGNGGDAALALLLLD | DQB1*03:01, DQB1*06:02 | 21 | 1249 | 416 | 40 |
| M | QFAYANRNRFLYIIK | DRB1*14:01 | 20 | 978 | 326 | 41 |
| ORF8 | TTVAAFHQECSLQSC | DQB1*05:02 | 18 | 2016 | 1008 | 42 |
| ORF8 | FHQECSLQSCTQHQP | HLA class II | 18 | 826 | 413 | 43 |
| ORF8 | ARKSAPLIELCVDEA | HLA class II | 18 | 658 | 329 | 44 |
| ORF8 | PLIELCVDEAGSKSP | HLA class II | 18 | 341 | 171 | 45 |
| ORF3a | CNLLLLFVTVYSHLL | DQB1*02:01, DRB1*07:01, DRB1*11:01 | 18 | 424 | 212 | 46 |
| ORF8 | EDFLEYHDVRVVLDF | DQB1*05:02, DQB1*06:04, DRB1*13:02, DRB1*15:02 | 18 | 1141 | 571 | 47 |
| ORF8 | DFLEYHDVRVVLDFI | DQB1*05:02, DQB1*06:04, DRB1*15:02 | 18 | 681 | 341 | 48 |
| S | VSQPFLMDLEGKQGN | DRB1*03:01 | 18 | 1694 | 847 | 49 |
| S | EFVFKNIDGYFKIYS | DQB1*05:03, DRB1*14:01, DRB1*15:01 | 18 | 672 | 336 | 50 |
| S | FKIYSKHTPINLVRD | DRB1*07:01, DRB1*13:01 | 18 | 967 | 484 | 51 |
| S | KHTPINLVRDLPQGF | DRB1*03:01 | 18 | 1641 | 821 | 52 |
| S | LPQGFSALEPLVDLP | DQB1*02:02, DQB1*03:03, DQB1*05:03 | 18 | 836 | 418 | 53 |
| S | IPFAMQMAYRFNGIG | DQB1*04:02, DQB1*05:03, DRB1*12:01, DRB1*14:01, DRB1*15:01 | 18 | 373 | 187 | 54 |
| nsp3 | TLRVEAFEYYHTTDP | DQB1*05:03 | 18 | 635 | 318 | 55 |
| nsp3 | HTTDPSFLGRYMSAL | HLA class II | 18 | 4921 | 2461 | 56 |
| M | LLWPVTLACFVLAAV | DQB1*05:01, DQB1*05:03, DQB1*06:03 | 17 | 520 | 260 | 57 |
| M | ITGGIAIAMACLVGL | DQB1*03:01 | 17 | 414 | 207 | 58 |
| M | TRSMWSFNPETNILL | DQB1*05:01, DQB1*05:02, DQB1*05:03, DRB1*01:02 | 17 | 410 | 205 | 59 |
| M | GTILTRPLLESELVI | DRB1*13:01 | 17 | 322 | 161 | 60 |
| N | QFAPSASAFFGMSRI | DRB1*07:01 | 17 | 381 | 191 | 61 |
| N | LTYTGAIKLDDKDPN | DRB1*07:01 | 17 | 1484 | 742 | 62 |
| S | KRISNCVADYSVLYN | DQB1*02:01, DQB1*02:02, DRB1*03:01 | 17 | 327 | 164 | 63 |
| S | LKPFERDISTEIYQA | HLA class II | 17 | 1188 | 594 | 64 |
| S | AENSVAYSNNSIAIP | DQB1*03:01, DRB1*15:01 | 17 | 805 | 403 | 65 |
| S | TNFTISVTTEILPVS | DQB1*02:02, DQB1*06:03, DRB1*07:01, DRB1*14:01 | 17 | 663 | 332 | 66 |
| S | VTLADAGFIKQYGDC | HLA class II | 17 | 237 | 119 | 67 |
| nsp4 | YLTFYLTNDVSFLAH | DQB1*05:02, DRB1*14:01, DRB1*15:01, DRB1*16:01 | 17 | 715 | 358 | 68 |
| S | VYYPDKVFRSSVLHS | DRB1*03:01, DRB1*13:01, DRB1*14:01 | 15 | 395 | 198 | 69 |
| S | SVLHSTQDLFLPFFS | DQB1*02:02, DRB1*07:01 | 15 | 609 | 305 | 70 |
| N | GGDGKMKDLSPRWYF | HLA class II | 15 | 295 | 148 | 71 |
| M | ATSRTLSYYKLGASQ | DQB1*05:01, DQB1*05:03, DRB1*16:01 | 15 | 1386 | 693 | 72 |
| M | LGASQRVAGDSGFAA | HLA class II | 15 | 981 | 491 | 73 |
| M | RVAGDSGFAAYSRYR | HLA class II | 15 | 278 | 139 | 74 |
| M | YSRYRIGNYKLNTDH | HLA class II | 15 | 551 | 276 | 75 |
| M | IGNYKLNTDHSSSSD | DRB1*03:01 | 15 | 267 | 134 | 76 |
| N | KAYNVTQAFGRRGPE | HLA class II | 15 | 530 | 265 | 77 |
| N | LIRQGTDYKHWPQIA | HLA class II | 15 | 431 | 216 | 78 |
| nsp3 | GYKKPASRELKVTFF | HLA class II | 15 | 491 | 246 | 79 |
| nsp3 | ASRELKVTFFPDLNG | DQB1*05:03 | 15 | 324 | 162 | 80 |
| nsp3 | PDLNGDVVAIDYKHY | HLA class II | 15 | 430 | 215 | 81 |
| nsp4 | RYLALYNKYKYFSGA | DRB1*15:01 | 15 | 1415 | 708 | 82 |
| N | MKDLSPRWYFYYLGT | HLA class II | 14 | 5072 | 2536 | 83 |
| N | LPYGANKDGIIWVAT | HLA class II | 14 | 835 | 418 | 84 |
| ORF3a | VRIIMRLWLCWKCRS | DRB1*11:01 | 14 | 261 | 131 | 85 |
| N | EGALNTPKDHIGTRN | HLA class II | 14 | 369 | 185 | 86 |
| ORF3a | EHVTFFIYNKIVDEP | DQB1*02:01, DQB1*05:01, DRB1*08:01, DRB1*11:01 | 14 | 277 | 139 | 87 |
| ORF3a | FIYNKIVDEPEEHVQ | DQB1*05:01, DRB1*08:01 | 14 | 486 | 243 | 88 |
| S | VTQNVLYENQKLIAN | DRB1*03:01, DRB1*12:01 | 14 | 705 | 353 | 89 |
| nsp12 | FVSLAIDAYPLTKHP | DQB1*02:02, DQB1*03:02, DQB1*06:04, DRB1*15:01 | 14 | 230 | 115 | 90 |
| nsp12 | IDAYPLTKHPNQEYA | HLA class II | 14 | 229 | 115 | 91 |
| M | EELKKLLEQWNLVIG | DQB1*05:01, DQB1*05:02 | 13 | 446 | 223 | 92 |
| M | LLEQWNLVIGFLFLT | DQB1*02:01, DQB1*05:03 | 13 | 243 | 122 | 93 |
| M | NRNRFLYIIKLIFLW | DQB1*02:01, DQB1*05:03, DRB1*03:01, DRB1*12:01 | 13 | 420 | 210 | 94 |
| M | LYIIKLIFLWLLWPV | DQB1*02:01, DRB1*12:01 | 13 | 397 | 199 | 95 |
| S | VNNSYECDIPIGAGI | HLA class II | 11 | 398 | 398 | 96 |
| ORF8 | SLQSCTQHQPYVVDD | HLA class II | 9 | 1222 | 1222 | 97 |
| S | LPFFSNVTWFHAIHV | DQB1*05:02, DRB1*15:01, DRB1*16:01 | 9 | 156 | 156 | 98 |
| ORF8 | CVDEAGSKSPIQYID | HLA class II | 9 | 327 | 327 | 99 |
| ORF3a | KIITLKKRWQLALSK | DRB1*11:01 | 9 | 120 | 120 | 100 |
| ORF8 | IQYIDIGNYTVSCLP | DRB1*11:04, DRB1*13:02 | 9 | 1805 | 1805 | 101 |
| S | NPVLPFNDGVYFAST | HLA class II | 9 | 413 | 413 | 102 |
| ORF8 | QEPKLGSLVVRCSFY | DRB1*11:04 | 9 | 124 | 124 | 103 |
| ORF3a | LVAAGLEAPFLYLYA | DQB1*02:01, DQB1*02:02 | 9 | 133 | 133 | 104 |
| S | EKSNIIRGWIFGTTL | DRB1*15:01 | 9 | 299 | 299 | 105 |
| S | IRGWIFGTTLDSKTQ | HLA class II | 9 | 327 | 327 | 106 |
| S | FGTTLDSKTQSLLIV | HLA class II | 9 | 174 | 174 | 107 |
| S | DSKTQSLLIVNNATN | HLA class II | 9 | 108 | 108 | 108 |
| S | VVIKVCEFQFCNDPF | HLA class II | 9 | 2088 | 2088 | 109 |
| S | LGVYYHKNNKSWMES | HLA class II | 9 | 259 | 259 | 110 |
| S | HKNNKSWMESEFRVY | DQB1*05:03 | 9 | 572 | 572 | 111 |
| S | GKQGNFKNLREFVFK | HLA class II | 9 | 129 | 129 | 112 |
| S | SALEPLVDLPIGINI | HLA class II | 9 | 167 | 167 | 113 |
| S | VIRGDEVRQIAPGQT | DRB1*13:01 | 9 | 180 | 180 | 114 |
| S | WNSNNLDSKVGGNYN | HLA class II | 9 | 101 | 101 | 115 |
| S | LPPLLTDEMIAQYTS | DQB1*04:02 | 9 | 153 | 153 | 116 |
| S | TDEMIAQYTSALLAG | DQB1*06:02, DRB1*04:04, DRB1*15:01 | 9 | 745 | 745 | 117 |
| S | ALLAGTITSGWTFGA | DQB1*06:02 | 9 | 116 | 116 | 118 |
| S | TITSGWTFGAGAALQ | DQB1*04:02, DQB1*06:02 | 9 | 164 | 164 | 119 |
| nsp3 | LPNDDTLRVEAFEYY | DQB1*03:02 | 9 | 162 | 162 | 120 |
| nsp3 | SFLGRYMSALNHTKK | DRB1*04:04 | 9 | 219 | 219 | 121 |
| nsp3 | YMSALNHTKKWKYPQ | HLA class II | 9 | 109 | 109 | 122 |
| nsp3 | NHTKKWKYPQVNGLT | HLA class II | 9 | 123 | 123 | 123 |
| nsp4 | AVITREVGFVVPGLP | HLA class II | 9 | 682 | 682 | 124 |
| nsp4 | VPGLPGTILRTTNGD | HLA class II | 9 | 439 | 439 | 125 |
| nsp4 | FLHFLPRVFSAVGNI | DRB1*15:01 | 9 | 491 | 491 | 126 |
| nsp4 | SIVAGGIVAIVVTCL | DQB1*03:02, DQB1*04:02 | 9 | 109 | 109 | 127 |
| S | VSSQCVNLTTRTQLP | HLA class II | 8 | 274 | 274 | 128 |
| S | VNLTTRTQLPPAYTN | HLA class II | 8 | 170 | 170 | 129 |
| M | LIFLWLLWPVTLACF | DQB1*05:01, DQB1*05:03, DRB1*01:02 | 8 | 1194 | 1194 | 130 |
| N | LTQHGKEDLKFPRGQ | HLA class II | 8 | 127 | 127 | 131 |
| N | FPRGQGVPINTNSSP | HLA class II | 8 | 397 | 397 | 132 |
| N | GVPINTNSSPDDQIG | HLA class II | 8 | 108 | 108 | 133 |
| M | AIAMACLVGLMWLSY | HLA class II | 8 | 151 | 151 | 134 |
| N | TRRIRGGDGKMKDLS | HLA class II | 8 | 151 | 151 | 135 |
| M | FIASFRLFARTRSMW | DQB1*05:03, DRB1*03:01, DRB1*14:01 | 8 | 353 | 353 | 136 |
| M | RLFARTRSMWSFNPE | DQB1*05:03, DRB1*14:01 | 8 | 141 | 141 | 137 |
| M | SFNPETNILLNVPLH | DRB1*01:02 | 8 | 123 | 123 | 138 |
| M | NVPLHGTILTRPLLE | HLA class II | 8 | 263 | 263 | 139 |
| M | RPLLESELVIGAVIL | DQB1*02:02 | 8 | 183 | 183 | 140 |
| M | GAVILRGHLRIAGHH | DRB1*13:01, DRB1*15:01 | 8 | 512 | 512 | 141 |
| M | LNTDHSSSSDNIALL | HLA class II | 8 | 568 | 568 | 142 |
| ORF3a | YFTSDYYQLYSTQLS | DRB1*16:02 | 8 | 198 | 198 | 143 |
| N | TQAFGRRGPEQTQGN | HLA class II | 8 | 151 | 151 | 144 |
| N | RRGPEQTQGNFGDQE | HLA class II | 8 | 518 | 518 | 145 |
| N | FGDQELIRQGTDYKH | HLA class II | 8 | 281 | 281 | 146 |
| N | TDYKHWPQIAQFAPS | HLA class II | 8 | 180 | 180 | 147 |
| N | ASAFFGMSRIGMEVT | DQB1*05:03, DRB1*01:02, DRB1*14:01 | 8 | 337 | 337 | 148 |
| N | GMEVTPSGTWLTYTG | HLA class II | 8 | 380 | 380 | 149 |
| N | AIKLDDKDPNFKDQV | DRB1*03:01 | 8 | 585 | 585 | 150 |
| S | YAWNRKRISNCVADY | DRB1*13:01 | 8 | 582 | 582 | 151 |
| S | CVADYSVLYNSASFS | DQB1*05:03, DQB1*06:02 | 8 | 210 | 210 | 152 |
| S | SVLYNSASFSTFKCY | DRB1*15:01 | 8 | 892 | 892 | 153 |
| S | SASFSTFKCYGVSPT | DRB1*15:01 | 8 | 294 | 294 | 154 |
| S | TFKCYGVSPTKLNDL | HLA class II | 8 | 221 | 221 | 155 |
| S | KLNDLCFTNVYADSF | DQB1*02:01, DQB1*02:02, DRB1*07:01 | 8 | 130 | 130 | 156 |
| S | YADSFVIRGDEVRQI | DQB1*02:02, DRB1*13:01 | 8 | 160 | 160 | 157 |
| S | EIYQAGSTPCNGVEG | HLA class II | 8 | 1121 | 1121 | 158 |
| S | FNCYFPLQSYGFQPT | DQB1*05:03, DRB1*12:01 | 8 | 130 | 130 | 159 |
| S | TLEILDITPCSFGGV | HLA class II | 8 | 227 | 227 | 160 |
| S | SIAIPTNFTISVTTE | HLA class II | 8 | 274 | 274 | 161 |
| S | SVTTEILPVSMTKTS | HLA class II | 8 | 341 | 341 | 162 |
| S | STECSNLLLQYGSFC | DQB1*06:02 | 8 | 314 | 314 | 163 |
| S | KNTQEVFAQVKQIYK | HLA class II | 8 | 125 | 125 | 164 |
| S | TPPIKDFGGFNFSQI | DRB1*15:01 | 8 | 421 | 421 | 165 |
| S | LPDPSKPSKRSFIED | HLA class II | 8 | 109 | 109 | 166 |
| S | LLFNKVTLADAGFIK | DQB1*06:02 | 8 | 297 | 297 | 167 |
| S | QYGDCLGDIAARDLI | HLA class II | 8 | 152 | 152 | 168 |
| S | LGDIAARDLICAQKF | HLA class II | 8 | 153 | 153 | 169 |
| S | ARDLICAQKFNGLTV | HLA class II | 8 | 232 | 232 | 170 |
| nsp3 | ADAVIKTLQPVSELL | DRB1*14:01 | 8 | 436 | 436 | 171 |
| nsp3 | ESDDYIATNGPLKVG | HLA class II | 8 | 133 | 133 | 172 |
| nsp3 | IATNGPLKVGGSCVL | DRB1*13:02 | 8 | 112 | 112 | 173 |
| nsp3 | SGHNLAKHCLHVVGP | HLA class II | 8 | 148 | 148 | 174 |
| nsp3 | NLYDKLVSSFLEMKS | DQB1*05:02, DRB1*14:01 | 8 | 150 | 150 | 175 |
| S | QELGKYEQYIKWPWY | HLA class II | 8 | 768 | 768 | 176 |
| nsp3 | RFYFYTSKTTVASLI | DQB1*06:01, DRB1*14:01, DRB1*15:02 | 8 | 126 | 126 | 177 |
| nsp3 | LNQLTGYKKPASREL | HLA class II | 8 | 168 | 168 | 178 |
| nsp3 | TPSFKKGAKLLHKPI | HLA class II | 8 | 103 | 103 | 179 |
| nsp3 | VWHVNNATNKATYKP | HLA class II | 8 | 158 | 158 | 180 |
| nsp3 | TFTRSTNSRIKASMP | DRB1*14:01 | 8 | 120 | 120 | 181 |
| nsp3 | TNSRIKASMPTTIAK | DRB1*04:04, DRB1*07:01 | 8 | 360 | 360 | 182 |
| nsp3 | NTVKSVGKFCLEASF | HLA class II | 8 | 113 | 113 | 183 |
| nsp3 | PNFSKLINIIIWFLL | DRB1*07:01, DRB1*15:01 | 8 | 3802 | 3802 | 184 |
| nsp3 | WDLTAFGLVAEWFLA | DQB1*05:03 | 8 | 134 | 134 | 185 |
| nsp3 | FGLVAEWFLAYILFT | DQB1*05:03, DRB1*12:01 | 8 | 219 | 219 | 186 |
| nsp4 | PVYSFLPGVYSVIYL | DRB1*15:01 | 8 | 650 | 650 | 187 |
| nsp4 | SFLAHIQWMVMFTPL | DQB1*06:02, DRB1*15:01 | 8 | 554 | 554 | 188 |
| nsp4 | IQWMVMFTPLVPFWI | DQB1*02:02, DRB1*04:04, DRB1*07:01 | 8 | 603 | 603 | 189 |
| nsp4 | MFTPLVPFWITIAYI | DRB1*15:01 | 8 | 602 | 602 | 190 |
| nsp4 | LTQYNRYLALYNKYK | DRB1*15:01 | 8 | 638 | 638 | 191 |
| nsp4 | YREAACCHLAKALND | HLA class II | 8 | 109 | 109 | 192 |
| nsp4 | CCHLAKALNDFSNSG | HLA class II | 8 | 111 | 111 | 193 |
| nsp4 | FSNSGSDVLYQPPQT | DQB1*06:02 | 8 | 149 | 149 | 194 |
| nsp4 | SDVLYQPPQTSITSA | HLA class II | 8 | 394 | 394 | 195 |
| nsp13 | ELHLSWEVGKPRPPL | HLA class II | 8 | 103 | 103 | 196 |
| nsp13 | PRPPLNRNYVFTGYR | HLA class II | 8 | 101 | 101 | 197 |
| nsp13 | FTGYRVTKNSKVQIG | DRB1*07:01 | 8 | 116 | 116 | 198 |
| nsp13 | VTKNSKVQIGEYTFE | HLA class II | 8 | 118 | 118 | 199 |
| nsp13 | VNARLRAKHYVYIGD | HLA class II | 8 | 116 | 116 | 200 |
| M | NLVIGFLFLTWICLL | DQB1*05:01, DRB1*01:02 | 7 | 261 | 261 | 201 |
| M | FLFLTWICLLQFAYA | DQB1*05:01, DQB1*05:03, DRB1*01:02 | 7 | 255 | 255 | 202 |
| M | WICLLQFAYANRNRF | DQB1*05:03, DRB1*03:01, DRB1*14:01 | 7 | 234 | 234 | 203 |
| ORF3a | LEAPFLYLYALVYFL | DQB1*02:01, DRB1*11:01 | 7 | 272 | 272 | 204 |
| N | PRWYFYYLGTGPEAG | DQB1*02:01, DQB1*05:03 | 7 | 750 | 750 | 205 |
| ORF3a | RLWLCWKCRSKNPLL | HLA class II | 7 | 290 | 290 | 206 |
| N | IWVATEGALNTPKDH | HLA class II | 7 | 200 | 200 | 207 |
| ORF3a | YDANYFLCWHTNCYD | HLA class II | 7 | 209 | 209 | 208 |
| ORF3a | FLCWHTNCYDYCIPY | DQB1*05:03 | 7 | 209 | 209 | 209 |
| ORF3a | TNCYDYCIPYNSVTS | DQB1*05:01 | 7 | 402 | 402 | 210 |
| ORF3a | TDTGVEHVTFFIYNK | HLA class II | 7 | 118 | 118 | 211 |
| N | GKGQQQQGQTVTKKS | HLA class II | 7 | 318 | 318 | 212 |
| ORF3a | GSSGVVNPVMEPIYD | DQB1*04:02 | 7 | 186 | 186 | 213 |
| N | HIDAYKTFPPTEPKK | HLA class II | 7 | 223 | 223 | 214 |
| N | QKKQQTVTLLPAADL | DRB1*01:02 | 7 | 712 | 712 | 215 |
| N | TVTLLPAADLDDFSK | DQB1*05:03, DRB1*01:02 | 7 | 648 | 648 | 216 |
| S | QPYRVVVLSFELLHA | DQB1*05:03, DRB1*14:01 | 7 | 183 | 183 | 217 |
| S | VVLSFELLHAPATVC | DQB1*05:03, DRB1*12:01, DRB1*14:01 | 7 | 317 | 317 | 218 |
| S | LTGTGVLTESNKKFL | HLA class II | 7 | 427 | 427 | 219 |
| S | QMAYRFNGIGVTQNV | DQB1*03:01 | 7 | 160 | 160 | 220 |
| S | LYENQKLIANQFNSA | DRB1*12:01 | 7 | 195 | 195 | 221 |
| S | HWFVTQRNFYEPQII | HLA class II | 7 | 598 | 598 | 222 |
| S | QRNFYEPQIITTDNT | HLA class II | 7 | 302 | 302 | 223 |
| S | FVSGNCDVVIGIVNN | HLA class II | 7 | 180 | 180 | 224 |
| S | GIVNNTVYDPLQPEL | DQB1*02:02, DQB1*03:02 | 7 | 164 | 164 | 225 |
| S | LQPELDSFKEELDKY | HLA class II | 7 | 173 | 173 | 226 |
| nsp3 | YCIDGALLTKSSEYK | DRB1*15:02 | 7 | 119 | 119 | 227 |
| nsp3 | MAAYVDNSSLTIKKP | DQB1*05:03 | 7 | 185 | 185 | 228 |
| nsp12 | HCANFNVLFSTVFPP | DQB1*02:02, DQB1*06:02, DRB1*07:01 | 7 | 211 | 211 | 229 |
| nsp12 | NVLFSTVFPPTSFGP | DQB1*02:02 | 7 | 109 | 109 | 230 |
| nsp12 | YPKCDRAMPNMLRIM | HLA class II | 7 | 153 | 153 | 231 |
| nsp12 | RAMPNMLRIMASLVL | DQB1*06:02, DRB1*15:01 | 7 | 144 | 144 | 232 |
| nsp12 | AVTANVNALLSTDGN | HLA class II | 7 | 201 | 201 | 233 |
| nsp12 | KTDGTLMIERFVSLA | HLA class II | 7 | 150 | 150 | 234 |
| nsp12 | DVFHLYLQYIRKLHD | DRB1*04:04, DRB1*13:02 | 7 | 247 | 247 | 235 |
| N | PQNQRNAPRITFGGP | HLA class II | 6 | 222 | 222 | 236 |
| N | NAPRITFGGPSDSTG | DQB1*03:01 | 6 | 259 | 259 | 237 |
| N | TFGGPSDSTGSNQNG | DQB1*03:01 | 6 | 291 | 291 | 238 |
| nsp12 | QDALFAYTKRNVIPT | HLA class II | 6 | 132 | 132 | 239 |
| nsp12 | IAATRGATVVIGTSK | DQB1*03:03, DRB1*07:01 | 6 | 117 | 117 | 240 |
| nsp12 | SHRFYRLANECAQVL | DRB1*01:01, DRB1*08:01 | 6 | 535 | 535 | 241 |
| nsp12 | SEMVMCGGSLYVKPG | HLA class II | 6 | 217 | 217 | 242 |
| nsp12 | FNICQAVTANVNALL | DQB1*03:01, DRB1*14:01 | 6 | 220 | 220 | 243 |
| nsp4 | KHFYWFFSNYLKRRV | DRB1*15:01 | 8 | 959 | 959 | 244 |
| N | DDQIGYYRRATRRIR | DRB1*13:01, DRB1*14:01, DRB1*14:06, DRB1*15:01, DRB1*16:02 | 23 | 803 | 268 | 245 |
| N | DAALALLLLDRLNQL | DQB1*05:03, DQB1*06:02, DRB1*03:01, DRB1*12:01, DRB1*14:01, DRB1*15:01 | 21 | 1463 | 488 | 246 |
| N | LLLLDRLNQLESKMS | DRB1*03:01, DRB1*12:01, DRB1*14:01, DRB1*15:01 | 21 | 1742 | 581 | 247 |
| N | PSGTWLTYTGAIKLD | DQB1*06:03, DRB1*01:02, DRB1*07:01, DRB1*15:01 | 42 | 2583 | 517 | 248 |
| ORF3a | ALLAVFQSASKIITL | DQB1*03:01, DRB1*11:01 | 9 | 178 | 178 | 249 |
| ORF3a | KKRWQLALSKGVHFV | DRB1*07:01 | 18 | 287 | 144 | 250 |
| ORF3a | LVYFLQSINFVRIIM | DQB1*05:03, DRB1*12:01, DRB1*14:01 | 7 | 218 | 218 | 251 |
| ORF3a | QSINFVRIIMRLWLC | DQB1*06:02, DRB1*03:01, DRB1*11:01, DRB1*12:01, DRB1*14:01, DRB1*15:01 | 21 | 505 | 168 | 252 |
| ORF3a | KNPLLYDANYFLCWH | DQB1*05:03, DRB1*15:01 | 7 | 112 | 112 | 253 |
| ORF8 | FLGIITTVAAFHQEC | DQB1*03:02, DQB1*06:03, DRB1*04:02 | 9 | 185 | 185 | 254 |
| ORF8 | FYSKWYIRVGARKSA | DQB1*06:01, DRB1*04:02, DRB1*07:01, DRB1*11:01, DRB1*11:04, DRB1*13:03, DRB1*14:01, DRB1*14:06, DRB1*15:01, DRB1*15:02, DRB1*16:01, DRB1*16:02 | 55 | 2805 | 468 | 255 |
| ORF8 | YIRVGARKSAPLIEL | DQB1*03:01, DRB1*11:04, DRB1*13:02, DRB1*15:01, DRB1*16:01 | 18 | 731 | 366 | 256 |
| nsp3 | KVTFFPDLNGDVVAI | DQB1*05:03 | 15 | 293 | 147 | 257 |
| nsp12 | KLLKSIAATRGATVV | DQB1*03:03, DRB1*07:01 | 6 | 230 | 230 | 258 |
| nsp12 | LMIERFVSLAIDAYP | DQB1*02:02, DQB1*03:02, DQB1*04:02, DQB1*05:01, DQB1*06:04, DRB1*01:01, DRB1*04:04, DRB1*07:01, DRB1*10:01, DRB1*13:02, DRB1*15:01 | 36 | 1941 | 388 | 259 |
| S | SLLIVNNATNVVIKV | DRB1*12:01, DRB1*14:01 | 9 | 164 | 164 | 260 |
| S | CEFQFCNDPFLGVYY | DQB1*05:02, DQB1*05:03 | 27 | 1077 | 359 | 261 |
| S | CTFEYVSQPFLMDLE | DQB1*02:01, DQB1*02:02, DQB1*05:02, | 64 | 4332 | 619 | 262 |
| | | DQB1*05:03, DRB1*07:01, DRB1*16:01 | | | | |
| S | IGINITRFQTLLALH | DRB1*07:01 | 9 | 177 | 177 | 263 |
| S | TRFQTLLALHRSYLT | DQB1*05:03, DRB1*12:01, DRB1*14:01 | 18 | 4343 | 2172 | 264 |
| S | FTVEKGIYQTSNFRV | DRB1*07:01 | 9 | 1202 | 1202 | 265 |
| S | SNFRVQPTESIVRFP | DRB1*07:01 | 9 | 243 | 243 | 266 |
| S | IVRFPNITNLCPFGE | DRB1*15:01 | 9 | 107 | 107 | 267 |
| S | CPFGEVFNATRFASV | DQB1*06:03, DRB1*07:01, DRB1*13:01 | 9 | 108 | 108 | 268 |
| S | AYSNNSIAIPTNFTI | DQB1*03:01 | 8 | 112 | 112 | 269 |
| S | NLLLQYGSFCTQLNR | DQB1*05:03, DRB1*04:04, DRB1*15:01 | 31 | 4560 | 1140 | 270 |
| S | TQLNRALTGIAVEQD | DQB1*04:02, DQB1*06:02 | 8 | 260 | 260 | 271 |
| S | VFAQVKQIYKTPPIK | DRB1*15:01 | 8 | 268 | 268 | 272 |
| S | NFSQILPDPSKPSKR | DRB1*03:01 | 33 | 2589 | 647 | 273 |
| S | KPSKRSFIEDLLFNK | DQB1*02:02, DQB1*05:03 | 17 | 1243 | 622 | 274 |
| S | SFIEDLLFNKVTLAD | DQB1*05:03, DRB1*03:01, DRB1*12:01, DRB1*14:01 | 17 | 812 | 406 | 275 |
| S | CAQKFNGLTVLPPLL | DQB1*05:02, DQB1*06:02, DRB1*16:01 | 9 | 741 | 741 | 276 |
| S | AQYTSALLAGTITSG | DQB1*06:02 | 9 | 336 | 336 | 277 |
| S | APHGVVFLHVTYVPA | DRB1*12:01 | 7 | 293 | 293 | 278 |
| S | ELDKYFKNHTSPDVD | HLA class II | 7 | 361 | 361 | 279 |
| S | GINASVVNIQKEIDR | HLA class II | 8 | 267 | 267 | 280 |

TABLE 5

List of CD8+ T cell epitopes identified and the HLA restrictions. A total of 523 class I epitopes were identified by AIM assay and encompassed the 8 dominant SARS-CoV-2 antigens for CD8+ T cells

| Protein | Allele | Start | Length | Sequence | Frequency of positive response | Sum of response | Magnitude of response | SEQ ID NO: |
|---|---|---|---|---|---|---|---|---|
| nsp3 | A*01:01 | 1636 | 11 | HTTDPSFLGRY | 100 | 9005 | 4503 | 281 |
| nsp3 | A*01:01 | 1637 | 11 | TTDPSFLGRYM | 100 | 22755 | 11378 | 282 |
| N | A*30:02 | 324 | 10 | VTPSGTWLTY | 100 | 902 | 451 | 283 |
| M | A*68:01 | 142 | 9 | AVILRGHLR | 100 | 666 | 333 | 284 |
| S | A*68:01 | 30 | 9 | NSFTRGVYY | 100 | 600 | 300 | 285 |
| S | A*68:01 | 69 | 9 | HVSGTNGTK | 100 | 972 | 486 | 286 |
| S | A*68:01 | 409 | 9 | QIAPGQTGK | 100 | 518 | 259 | 287 |
| N | B*57:01 | 43 | 10 | QGLPNNTASW | 100 | 1466 | 733 | 288 |
| N | B*57:01 | 100 | 9 | KMKDLSPRW | 100 | 426 | 213 | 289 |
| ORF3a | B*57:01 | 33 | 9 | ATIPIQASL | 100 | 363 | 182 | 290 |
| nsp3 | B*57:01 | 1551 | 9 | ITFDNLKTL | 100 | 400 | 200 | 291 |
| S | A*24:02 | 265 | 11 | YYVGYLQPRTF | 75 | 1452 | 484 | 292 |
| S | A*24:02 | 1137 | 12 | VYDPLQPELDSF | 75 | 2244 | 748 | 293 |
| S | A*26:01 | 258 | 9 | WTAGAAAYY | 75 | 815 | 272 | 294 |
| S | A*26:01 | 686 | 10 | SVASQSIIAY | 75 | 862 | 287 | 295 |
| S | B*35:01 | 687 | 9 | VASQSIIAY | 75 | 1444 | 481 | 296 |
| nsp3 | A*01:01 | 1322 | 8 | TDNYITTY | 67 | 2748 | 1374 | 297 |
| N | A*11:01 | 361 | 10 | KTFPPTEPKK | 67 | 411 | 206 | 298 |
| N | B*07:02 | 105 | 9 | SPRWYFYYL | 67 | 5911 | 2956 | 299 |
| S | B*15:01 | 634 | 10 | RVYSTGSNVF | 67 | 1045 | 523 | 300 |
| ORF3a | B*35:01 | 35 | 9 | IPIQASLPF | 67 | 1617 | 809 | 301 |
| nsp4 | B*35:01 | 2898 | 8 | TPSKLIEY | 67 | 1438 | 719 | 302 |
| nsp4 | B*35:01 | 3136 | 9 | VPFWITIAY | 67 | 1039 | 520 | 303 |
| N | B*44:02 | 289 | 10 | QELIRQGTDY | 67 | 456 | 228 | 304 |
| S | B*08:01 | 506 | 8 | QPYRVVVL | 60 | 1616 | 539 | 305 |
| S | B*44:02 | 747 | 10 | TECSNLLLQY | 60 | 794 | 265 | 306 |
| S | A*03:01 | 41 | 9 | KVFRSSVLH | 50 | 272 | 136 | 307 |
| S | A*24:02 | 159 | 10 | VYSSANNCTF | 50 | 719 | 360 | 308 |
| S | A*24:02 | 312 | 9 | IYQTSNFRV | 50 | 710 | 355 | 309 |
| S | A*26:01 | 718 | 9 | FTISVTTEI | 50 | 997 | 499 | 310 |
| S | A*29:02 | 162 | 9 | SANNCTFEY | 50 | 282 | 141 | 311 |
| S | A*29:02 | 192 | 9 | FVFKNIDGY | 50 | 314 | 157 | 312 |
| S | A*29:02 | 361 | 9 | CVADYSVLY | 50 | 931 | 466 | 313 |
| S | A*29:02 | 445 | 9 | VGGNYNYLY | 50 | 439 | 220 | 314 |
| M | B*07:02 | 131 | 8 | RPLLESEL | 50 | 537 | 269 | 315 |
| M | B*07:02 | 164 | 9 | LPKEITVAT | 50 | 515 | 258 | 316 |
| S | B*35:01 | 79 | 14 | FDNPVLPFNDGVYF | 50 | 270 | 135 | 317 |
| S | B*35:01 | 895 | 10 | QIPFAMQMAY | 50 | 404 | 202 | 318 |
| nsp12 | B*51:01 | 4586 | 9 | DAMRNAGIV | 50 | 508 | 254 | 319 |
| nsp12 | B*51:01 | 5220 | 10 | YLPYPDPSRI | 50 | 519 | 260 | 320 |
| S | B*07:02 | 462 | 11 | KPFERDISTEI | 40 | 2921 | 1461 | 321 |
| S | B*07:02 | 1052 | 9 | FPQSAPHGV | 40 | 413 | 207 | 322 |
| S | B*08:01 | 216 | 8 | LPQGFSAL | 40 | 816 | 408 | 323 |
| S | B*08:01 | 869 | 9 | MIAQYTSAL | 40 | 1025 | 513 | 324 |
| N | B*40:01 | 322 | 10 | MEVTPSGTWL | 40 | 452 | 226 | 325 |
| S | B*40:01 | 168 | 10 | FEYVSQPFLM | 40 | 748 | 374 | 326 |
| S | B*40:01 | 323 | 13 | TESIVRFPNITNL | 40 | 433 | 217 | 327 |
| S | B*40:01 | 1256 | 10 | FDEDDSEPVL | 40 | 430 | 215 | 328 |
| S | B*40:01 | 1261 | 10 | SEPVLKGVKL | 40 | 501 | 251 | 329 |
| S | B*44:02 | 297 | 10 | SETKCTLKSF | 40 | 619 | 310 | 330 |
| S | B*44:02 | 553 | 13 | TESNKKFLPFQQF | 40 | 813 | 407 | 331 |
| S | B*44:02 | 653 | 8 | AEHVNNSY | 40 | 1949 | 975 | 332 |
| M | A*02:01 | 65 | 9 | FVLAAVYRI | 38 | 751 | 250 | 333 |
| N | B*35:01 | 79 | 9 | SPDDQIGYY | 33 | 682 | 341 | 334 |
| N | B*35:01 | 266 | 9 | KAYNVTQAF | 33 | 1694 | 847 | 335 |
| N | B*35:01 | 307 | 9 | FAPSASAFF | 33 | 809 | 405 | 336 |
| N | B*35:01 | 325 | 9 | TPSGTWLTY | 33 | 975 | 488 | 337 |
| N | B*35:01 | 395 | 9 | LPAADLDDF | 33 | 440 | 220 | 338 |
| M | A*02:01 | 15 | 9 | KLLEQWNLV | 25 | 357 | 179 | 339 |
| M | A*02:01 | 108 | 9 | SMWSFNPET | 25 | 727 | 364 | 340 |
| N | B*07:02 | 257 | 9 | KPRQKRTAT | 33 | 5475 | 5475 | 341 |
| S | B*07:02 | 506 | 8 | QPYRVVVL | 25 | 3589 | 3589 | 342 |
| S | B*44:02 | 917 | 11 | YENQKLIANQF | 20 | 3070 | 3070 | 343 |
| N | B*07:02 | 66 | 9 | FPRGQGVPI | 33 | 2472 | 2472 | 344 |
| nsp4 | A*31:01 | 3155 | 9 | WFFSNYLKR | 100 | 2244 | 2244 | 345 |
| nsp16 | B*57:01 | 6875 | 12 | GVAPGTAVLRQW | 100 | 2223 | 2223 | 346 |
| N | A*68:01 | 311 | 9 | ASAFFGMSR | 50 | 2067 | 2067 | 347 |
| M | B*57:01 | 84 | 9 | MACLVGLMW | 50 | 1711 | 1711 | 348 |
| nsp16 | B*57:01 | 6913 | 10 | CATVHTANKW | 100 | 1697 | 1697 | 349 |
| N | A*24:02 | 306 | 10 | QFAPSASAFF | 25 | 1560 | 1560 | 350 |
| nsp4 | B*35:01 | 3199 | 9 | LPLTQYNRY | 33 | 1552 | 1552 | 351 |
| M | B*44:02 | 136 | 9 | SELVIGAVI | 33 | 1472 | 1472 | 352 |
| S | B*44:02 | 1016 | 9 | AEIRASANL | 20 | 1427 | 1427 | 353 |
| N | A*68:01 | 310 | 10 | SASAFFGMSR | 50 | 1407 | 1407 | 354 |
| S | B*35:01 | 258 | 9 | WTAGAAAYY | 25 | 1401 | 1401 | 355 |
| M | A*68:02 | 65 | 9 | FVLAAVYRI | 100 | 1303 | 1303 | 356 |
| nsp4 | A*02:01 | 2788 | 8 | YLITPVHV | 100 | 1151 | 1151 | 357 |
| nsp3 | B*07:02 | 2017 | 12 | KPVETSNSFDVL | 33 | 1129 | 1129 | 358 |
| nsp12 | B*44:02 | 4645 | 13 | AESHVDTDLTKPY | 33 | 1125 | 1125 | 359 |
| S | B*57:01 | 604 | 9 | TSNQVAVLY | 100 | 1099 | 1099 | 360 |
| S | B*44:02 | 989 | 9 | AEVQIDRLI | 20 | 1098 | 1098 | 361 |
| ORF3a | A*02:01 | 100 | 9 | GLEAPFLYL | 33 | 1086 | 1086 | 362 |
| S | B*57:01 | 1093 | 10 | GVFVSNGTHW | 100 | 1057 | 1057 | 363 |
| M | A*31:01 | 93 | 9 | LSYFIASFR | 100 | 1045 | 1045 | 364 |
| N | B*08:01 | 105 | 9 | SPRWYFYYL | 33 | 1034 | 1034 | 365 |
| S | B*08:01 | 691 | 9 | SIIAYTMSL | 20 | 1028 | 1028 | 366 |
| M | A*01:01 | 188 | 9 | AGDSGFAAY | 25 | 1020 | 1020 | 367 |
| S | A*02:01 | 1048 | 9 | HLMSFPQSA | 17 | 1011 | 1011 | 368 |
| ORF3a | B*08:01 | 57 | 9 | QSASKIITL | 33 | 986 | 986 | 369 |
| nsp3 | A*01:01 | 1637 | 10 | TTDPSFLGRY | 100 | 978 | 978 | 370 |
| M | B*57:01 | 67 | 9 | LAAVYRINW | 50 | 973 | 973 | 371 |
| nsp12 | B*57:01 | 4892 | 10 | KSAGFPFNKW | 100 | 950 | 950 | 372 |
| ORF3a | A*24:02 | 106 | 9 | LYLYALVYF | 50 | 943 | 943 | 373 |
| nsp3 | A*26:01 | 2495 | 10 | TVKNGSIHLY | 50 | 941 | 941 | 374 |
| nsp3 | B*44:02 | 1723 | 14 | GELGDVRETMSYLF | 100 | 939 | 939 | 375 |
| M | B*15:01 | 186 | 11 | RVAGDSGFAAY | 50 | 924 | 924 | 376 |
| S | A*29:02 | 1147 | 9 | SFKEELDKY | 25 | 898 | 898 | 377 |
| nsp4 | A*31:01 | 2941 | 9 | GSVAYESLR | 100 | 896 | 896 | 378 |
| nsp3 | A*26:01 | 2305 | 10 | ETIQITISSF | 50 | 893 | 893 | 379 |
| ORF3a | B*57:01 | 120 | 9 | FVRIIMRLW | 50 | 883 | 883 | 380 |
| nsp4 | A*31:01 | 3219 | 9 | GAMDTTSYR | 100 | 846 | 846 | 381 |
| nsp12 | B*44:02 | 5001 | 10 | VENPHLMGWD | 33 | 830 | 830 | 382 |
| S | A*24:02 | 169 | 9 | EYVSQPFLM | 25 | 815 | 815 | 383 |
| S | A*26:01 | 1095 | 9 | FVSNGTHWF | 25 | 812 | 812 | 384 |
| nsp4 | B*35:01 | 2960 | 9 | SIIQFPNTY | 33 | 803 | 803 | 385 |
| nsp3 | B*07:02 | 2109 | 9 | KPNELSRVL | 33 | 801 | 801 | 386 |
| M | A*30:01 | 171 | 10 | ATSRTLSYYK | 100 | 800 | 800 | 387 |
| nsp16 | B*07:02 | 7048 | 9 | FPLKLRGTA | 50 | 792 | 792 | 388 |
| nsp3 | A*26:01 | 2664 | 14 | EVTGDSCNNYMLTY | 50 | 789 | 789 | 389 |
| nsp3 | A*26:01 | 2089 | 11 | EVGHTDLMAAY | 50 | 785 | 785 | 390 |
| nsp3 | A*01:01 | 2086 | 14 | ITEEVGHTDLMAAY | 50 | 774 | 774 | 391 |
| nsp3 | B*44:02 | 941 | 9 | FEPSTQYEY | 100 | 771 | 771 | 392 |
| nsp12 | B*07:02 | 4503 | 9 | VPHISRQRL | 100 | 759 | 759 | 393 |
| nsp3 | A*02:01 | 2332 | 9 | ILFTRFFYV | 50 | 752 | 752 | 394 |
| S | B*44:03 | 1181 | 9 | KEIDRLNEV | 20 | 746 | 746 | 395 |
| nsp3 | B*44:02 | 1364 | 10 | KQEILGTVSW | 100 | 739 | 739 | 396 |
| nsp4 | A*02:01 | 3115 | 10 | YLTNDVSFLA | 100 | 737 | 737 | 397 |
| S | B*07:02 | 506 | 10 | QPYRVVVLSF | 25 | 721 | 721 | 398 |
| S | A*01:01 | 28 | 10 | YTNSFTRGVY | 33 | 711 | 711 | 399 |
| nsp12 | B*57:01 | 5299 | 10 | LTNDNTSRYW | 100 | 704 | 704 | 400 |
| S | A*31:01 | 458 | 9 | KSNLKPFER | 50 | 701 | 701 | 401 |
| S | B*08:01 | 269 | 9 | YLQPRTFLL | 20 | 691 | 691 | 402 |
| S | B*44:02 | 1201 | 9 | QELGKYEQY | 20 | 685 | 685 | 403 |
| nsp3 | A*26:01 | 1873 | 10 | YTTTIKPVTY | 50 | 683 | 683 | 404 |
| nsp4 | A*31:01 | 3004 | 9 | WVLNNDYYR | 100 | 678 | 678 | 405 |
| nsp3 | A*29:02 | 2389 | 9 | SFYYVWKSY | 50 | 677 | 677 | 406 |
| nsp4 | A*31:01 | 3060 | 9 | LAYYFMRFR | 100 | 676 | 676 | 407 |
| ORF3a | B*08:01 | 63 | 9 | ITLKKRWQL | 33 | 669 | 669 | 408 |
| nsp3 | A*26:01 | 2272 | 10 | NSTNVTIATY | 50 | 660 | 660 | 409 |
| nsp3 | A*26:01 | 2594 | 9 | YVNTFSSTF | 50 | 656 | 656 | 410 |
| S | A*29:02 | 1264 | 9 | VLKGVKLHY | 25 | 631 | 631 | 411 |
| nsp4 | A*02:01 | 3100 | 8 | FLPGVYSV | 100 | 626 | 626 | 412 |
| N | B*07:02 | 45 | 9 | LPNNTASWF | 33 | 609 | 609 | 413 |
| S | A*24:02 | 448 | 9 | NYNYLYRLF | 25 | 606 | 606 | 414 |
| nsp3 | A*26:01 | 2435 | 10 | YVYANGGKGF | 50 | 593 | 593 | 415 |
| S | B*35:01 | 896 | 9 | IPFAMQMAY | 25 | 588 | 588 | 416 |
| nsp3 | A*26:01 | 2585 | 10 | EVAVKMFDAY | 50 | 587 | 587 | 417 |
| S | B*44:03 | 297 | 10 | SETKCTLKSF | 20 | 559 | 559 | 418 |
| S | B*57:01 | 710 | 9 | NSIAIPTNF | 100 | 559 | 559 | 419 |
| nsp4 | B*35:01 | 3196 | 9 | DVLLPLTQY | 33 | 557 | 557 | 420 |
| S | A*26:01 | 340 | 12 | EVFNATRFASVY | 25 | 556 | 556 | 421 |
| S | A*24:02 | 1101 | 9 | HWFVTQRNF | 25 | 551 | 551 | 422 |
| nsp3 | B*44:02 | 2111 | 9 | NELSRVLGL | 33 | 551 | 551 | 423 |
| nsp4 | B*35:01 | 2949 | 9 | RPDTRYVLM | 33 | 542 | 542 | 424 |
| nsp3 | B*44:03 | 2680 | 9 | VENMTPRDL | 50 | 541 | 541 | 425 |
| nsp3 | B*44:02 | 1857 | 12 | SEYKGPITDVFY | 33 | 540 | 540 | 426 |
| S | A*24:02 | 328 | 11 | RFPNITNLCPF | 25 | 539 | 539 | 427 |
| S | B*35:01 | 1052 | 11 | FPQSAPHGVVF | 25 | 537 | 537 | 428 |
| S | B*57:01 | 685 | 9 | RSVASQSII | 100 | 534 | 534 | 429 |
| nsp3 | A*68:01 | 828 | 10 | DTVIEVQGYK | 50 | 533 | 533 | 430 |
| nsp3 | A*26:01 | 1966 | 10 | DVVAIDYKHY | 50 | 530 | 530 | 431 |
| S | A*29:02 | 30 | 9 | NSFTRGVYY | 25 | 507 | 507 | 432 |
| M | A*30:02 | 186 | 14 | RVAGDSGFAAYSRY | 100 | 505 | 505 | 433 |
| ORF3a | B*51:01 | 35 | 9 | IPIQASLPF | 50 | 491 | 491 | 434 |
| S | B*44:02 | 1206 | 9 | YEQYIKWPW | 20 | 489 | 489 | 435 |
| nsp3 | A*30:01 | 1647 | 9 | MSALNHTKK | 100 | 486 | 486 | 436 |
| S | B*44:02 | 339 | 9 | GEVFNATRF | 20 | 482 | 482 | 437 |
| S | A*24:02 | 193 | 9 | VFKNIDGYF | 25 | 467 | 467 | 438 |
| S | A*24:02 | 1094 | 10 | VFVSNGTHWF | 25 | 465 | 465 | 439 |
| nsp3 | B*44:02 | 1023 | 9 | IEVNSFSGY | 100 | 464 | 464 | 440 |
| nsp3 | A*26:01 | 2132 | 10 | SVPWDTIANY | 50 | 461 | 461 | 441 |
| nsp3 | B*44:02 | 2325 | 9 | AEWFLAYIL | 33 | 461 | 461 | 442 |
| S | B*44:02 | 95 | 10 | TEKSNIIRGW | 20 | 459 | 459 | 443 |
| nsp16 | B*07:02 | 7033 | 10 | NPIQLSSYSL | 50 | 453 | 453 | 444 |
| nsp3 | A*26:01 | 2582 | 9 | DSAEVAVKM | 50 | 452 | 452 | 445 |
| nsp4 | A*02:01 | 2884 | 9 | FLPRVFSAV | 100 | 447 | 447 | 446 |
| nsp3 | A*26:01 | 2201 | 9 | NTVKSVGKF | 50 | 443 | 443 | 447 |
| nsp3 | B*57:01 | 2254 | 9 | MSNLGMPSY | 100 | 443 | 443 | 448 |
| S | A*29:02 | 781 | 9 | VFAQVKQIY | 25 | 442 | 442 | 449 |
| S | B*07:02 | 229 | 10 | LPIGINITRF | 20 | 442 | 442 | 450 |
| N | B*57:01 | 123 | 10 | YGANKDGIIW | 50 | 441 | 441 | 451 |
| S | B*35:01 | 1095 | 9 | FVSNGTHWF | 25 | 436 | 436 | 452 |
| S | A*30:01 | 1065 | 9 | VTYVPAQEK | 33 | 435 | 435 | 453 |
| nsp12 | A*11:01 | 4733 | 9 | VVSTGYHFR | 100 | 434 | 434 | 454 |
| nsp3 | A*68:01 | 1504 | 9 | TISLAGSYK | 50 | 433 | 433 | 455 |
| S | A*26:01 | 28 | 10 | YTNSFTRGVY | 25 | 432 | 432 | 456 |
| S | A*30:01 | 634 | 9 | RVYSTGSNV | 33 | 428 | 428 | 457 |
| N | A*26:01 | 290 | 9 | ELIRQGTDY | 33 | 424 | 424 | 458 |
| nsp3 | B*57:01 | 1505 | 10 | ISLAGSYKDW | 50 | 424 | 424 | 459 |
| nsp3 | A*26:01 | 1891 | 9 | EIDPKLDNY | 50 | 422 | 422 | 460 |
| nsp3 | B*44:02 | 2618 | 9 | AELAKNVSL | 33 | 417 | 417 | 461 |
| S | A*26:01 | 442 | 10 | DSKVGGNYNY | 25 | 407 | 407 | 462 |
| nsp3 | B*44:02 | 1825 | 12 | SEYTGNYQCGHY | 33 | 407 | 407 | 463 |
| S | B*07:02 | 208 | 9 | TPINLVRDL | 20 | 406 | 406 | 464 |
| S | B*07:02 | 588 | 10 | TPCSFGGVSV | 20 | 405 | 405 | 465 |
| S | A*02:01 | 269 | 9 | YLQPRTFLL | 17 | 404 | 404 | 466 |
| S | B*08:01 | 233 | 9 | INITRFQTL | 20 | 402 | 402 | 467 |
| S | A*03:01 | 827 | 9 | TLADAGFIK | 25 | 398 | 398 | 468 |
| nsp3 | A*26:01 | 2323 | 9 | LVAEWFLAY | 50 | 394 | 394 | 469 |
| S | A*29:02 | 865 | 9 | LTDEMIAQY | 25 | 393 | 393 | 470 |
| S | B*53:01 | 712 | 9 | IAIPTNFTI | 100 | 393 | 393 | 471 |
| nsp3 | B*44:02 | 1869 | 14 | KENSYTTTIKPVTY | 33 | 392 | 392 | 472 |
| nsp3 | A*30:01 | 2189 | 9 | RIKASMPTT | 100 | 390 | 390 | 473 |
| S | A*26:01 | 298 | 9 | ETKCTLKSF | 25 | 387 | 387 | 474 |
| nsp3 | B*57:01 | 2318 | 10 | LTAFGLVAEW | 100 | 384 | 384 | 475 |
| nsp3 | A*30:01 | 2191 | 9 | KASMPTTIA | 100 | 382 | 382 | 476 |
| M | A*30:01 | 42 | 9 | RNRFLYIIK | 50 | 380 | 380 | 477 |
| S | A*24:02 | 268 | 10 | GYLQPRTFLL | 25 | 378 | 378 | 478 |
| S | A*29:02 | 687 | 9 | VASQSIIAY | 25 | 377 | 377 | 479 |
| nsp3 | A*26:01 | 2246 | 9 | STAALGVLM | 50 | 376 | 376 | 480 |
| nsp3 | B*40:01 | 1006 | 9 | VEVQPQLEM | 50 | 374 | 374 | 481 |
| S | A*02:01 | 1060 | 9 | VVFLHVTYV | 17 | 365 | 365 | 482 |
| S | A*24:02 | 78 | 9 | RFDNPVLPF | 25 | 365 | 365 | 483 |
| S | A*26:01 | 192 | 9 | FVFKNIDGY | 25 | 365 | 365 | 484 |
| S | A*26:01 | 361 | 9 | CVADYSVLY | 25 | 362 | 362 | 485 |
| N | A*01:01 | 75 | 13 | NTNSSPDDQIGYY | 33 | 359 | 359 | 486 |
| nsp3 | B*44:02 | 2513 | 9 | YERHSLSHF | 33 | 359 | 359 | 487 |
| nsp12 | B*44:02 | 4998 | 12 | YSDVENPHLMGW | 33 | 359 | 359 | 488 |
| S | B*51:01 | 712 | 9 | IAIPTNFTI | 25 | 355 | 355 | 489 |
| nsp3 | A*26:01 | 2456 | 9 | DTFCAGSTF | 50 | 351 | 351 | 490 |
| nsp3 | B*44:02 | 2550 | 11 | EESSAKSASVY | 33 | 350 | 350 | 491 |
| S | A*24:02 | 634 | 10 | RVYSTGSNVF | 25 | 349 | 349 | 492 |
| S | A*24:02 | 144 | 9 | YYHKNNKSW | 25 | 348 | 348 | 493 |
| nsp12 | B*51:01 | 4971 | 9 | IAATRGATV | 25 | 343 | 343 | 494 |
| S | A*68:01 | 89 | 9 | GVYFASTEK | 50 | 340 | 340 | 495 |
| S | B*44:03 | 1201 | 9 | QELGKYEQY | 20 | 339 | 339 | 496 |
| M | A*68:02 | 212 | 9 | SSSDNIALL | 100 | 337 | 337 | 497 |
| M | A*11:01 | 171 | 9 | ATSRTLSYY | 100 | 336 | 336 | 498 |
| S | A*26:01 | 780 | 10 | EVFAQVKQIY | 25 | 336 | 336 | 499 |
| nsp12 | B*51:01 | 5221 | 10 | LPYPDPSRIL | 25 | 335 | 335 | 500 |
| M | B*07:02 | 122 | 8 | VPLHGTIL | 25 | 334 | 334 | 501 |
| S | B*44:02 | 779 | 11 | QEVFAQVKQIY | 20 | 333 | 333 | 502 |
| nsp12 | A*68:01 | 4938 | 10 | YAISAKNRAR | 100 | 331 | 331 | 503 |
| nsp3 | B*40:01 | 843 | 11 | FELDERIDKVL | 50 | 329 | 329 | 504 |
| M | A*68:01 | 137 | 10 | ELVIGAVILR | 50 | 328 | 328 | 505 |
| nsp3 | A*30:01 | 2192 | 9 | ASMPTTIAK | 100 | 327 | 327 | 506 |
| S | B*44:03 | 95 | 10 | TEKSNIIRGW | 25 | 327 | 327 | 507 |
| S | A*68:01 | 1173 | 9 | NASVVNIQK | 50 | 326 | 326 | 508 |
| nsp3 | A*11:01 | 1582 | 9 | QVVDMSMTY | 100 | 325 | 325 | 509 |
| nsp3 | A*68:01 | 1378 | 9 | MLAHAEETR | 50 | 324 | 324 | 510 |
| nsp3 | A*26:01 | 1865 | 9 | DVFYKENSY | 50 | 322 | 322 | 511 |
| S | A*68:01 | 35 | 10 | GVYYPDKVFR | 50 | 314 | 314 | 512 |
| S | A*26:01 | 30 | 9 | NSFTRGVYY | 25 | 313 | 313 | 513 |
| S | B*07:02 | 1262 | 9 | EPVLKGVKL | 20 | 313 | 313 | 514 |
| nsp3 | A*68:01 | 1636 | 10 | HTTDPSFLGR | 50 | 312 | 312 | 515 |
| S | B*08:01 | 234 | 8 | NITRFQTL | 20 | 312 | 312 | 516 |
| nsp3 | B*44:02 | 2616 | 9 | AEAELAKNV | 33 | 312 | 312 | 517 |
| nsp12 | B*44:03 | 4558 | 9 | VENPDILRV | 50 | 312 | 312 | 518 |
| nsp3 | B*40:01 | 1135 | 9 | YENFNQHEV | 50 | 310 | 310 | 519 |
| nsp3 | B*51:01 | 1048 | 9 | EAKKVKPTV | 33 | 308 | 308 | 520 |
| S | B*51:01 | 923 | 9 | IANQFNSAI | 25 | 307 | 307 | 521 |
| S | A*26:01 | 603 | 10 | NTSNQVAVLY | 25 | 304 | 304 | 522 |
| S | A*26:01 | 583 | 10 | EILDITPCSF | 25 | 302 | 302 | 523 |
| M | A*68:01 | 6 | 9 | GTITVEELK | 50 | 302 | 302 | 524 |
| S | A*32:01 | 1059 | 9 | GVVFLHVTY | 50 | 301 | 301 | 525 |
| S | B*07:02 | 84 | 9 | LPFNDGVYF | 20 | 299 | 299 | 526 |
| nsp12 | B*44:02 | 5193 | 12 | TETDLTKGPHEF | 33 | 299 | 299 | 527 |
| S | B*44:03 | 1091 | 12 | REGVFVSNGTHW | 20 | 299 | 299 | 528 |
| S | B*08:01 | 202 | 9 | KIYSKHTPI | 20 | 298 | 298 | 529 |
| S | A*02:01 | 109 | 9 | TLDSKTQSL | 17 | 296 | 296 | 530 |
| M | A*68:01 | 93 | 9 | LSYFIASFR | 50 | 294 | 294 | 531 |
| S | B*35:01 | 865 | 9 | LTDEMIAQY | 25 | 294 | 294 | 532 |
| nsp3 | B*35:01 | 1270 | 9 | LVSDIDITF | 100 | 294 | 294 | 533 |
| S | B*40:01 | 339 | 9 | GEVFNATRF | 20 | 294 | 294 | 534 |
| ORF3a | B*44:02 | 98 | 12 | AAGLEAPFLYLY | 33 | 294 | 294 | 535 |
| S | B*51:01 | 224 | 8 | EPLVDLPI | 25 | 294 | 294 | 536 |
| S | B*51:01 | 351 | 8 | YAWNRKRI | 25 | 293 | 293 | 537 |
| M | A*68:01 | 138 | 9 | LVIGAVILR | 50 | 292 | 292 | 538 |
| S | B*44:03 | 339 | 9 | GEVFNATRF | 20 | 292 | 292 | 539 |
| nsp12 | A*68:01 | 4840 | 10 | AAISDYDYYR | 100 | 290 | 290 | 540 |
| S | B*44:02 | 1181 | 9 | KEIDRLNEV | 20 | 286 | 286 | 541 |
| nsp3 | B*44:02 | 2584 | 8 | AEVAVKMF | 33 | 284 | 284 | 542 |
| S | B*44:03 | 747 | 10 | TECSNLLLQY | 20 | 284 | 284 | 543 |
| nsp3 | B*44:03 | 1364 | 10 | KQEILGTVSW | 100 | 284 | 284 | 544 |
| nsp3 | A*30:01 | 1951 | 9 | ASRELKVTF | 100 | 283 | 283 | 545 |
| nsp3 | B*44:03 | 2088 | 12 | EEVGHTDLMAAY | 50 | 283 | 283 | 546 |
| S | B*08:01 | 680 | 8 | SPRRARSV | 20 | 282 | 282 | 547 |
| S | A*30:02 | 628 | 9 | QLTPTWRVY | 100 | 280 | 280 | 548 |
| S | B*51:01 | 780 | 9 | EVFAQVKQI | 25 | 280 | 280 | 549 |
| S | A*24:02 | 706 | 9 | AYSNNSIAI | 25 | 278 | 278 | 550 |
| S | B*35:01 | 604 | 9 | TSNQVAVLY | 25 | 278 | 278 | 551 |
| nsp3 | A*26:01 | 2468 | 10 | EVARDLSLQF | 50 | 275 | 275 | 552 |
| ORF3a | B*35:01 | 97 | 9 | VAAGLEAPF | 33 | 275 | 275 | 553 |
| nsp3 | A*68:01 | 1155 | 9 | GADPIHSLR | 50 | 274 | 274 | 554 |
| nsp3 | A*68:01 | 1296 | 9 | LTAV V IPTK | 50 | 274 | 274 | 555 |
| nsp3 | B*35:01 | 1056 | 9 | VVVNAANVY | 100 | 273 | 273 | 556 |
| ORF3a | A*02:01 | 89 | 9 | TVYSHLLLV | 33 | 272 | 272 | 557 |
| N | A*11:01 | 134 | 10 | ATEGALNTPK | 33 | 272 | 272 | 558 |
| S | B*35:01 | 84 | 9 | LPFNDGVYF | 25 | 272 | 272 | 559 |
| S | A*03:01 | 142 | 9 | GVYYHKNNK | 25 | 270 | 270 | 560 |
| M | A*30:01 | 198 | 9 | RYRIGNYKL | 100 | 270 | 270 | 561 |
| S | A*68:01 | 400 | 9 | FVIRGDEVR | 50 | 269 | 269 | 562 |
| N | B*07:02 | 308 | 10 | APSASAFFGM | 33 | 269 | 269 | 563 |
| S | A*03:01 | 1019 | 10 | RASANLAATK | 25 | 268 | 268 | 564 |
| ORF3a | A*68:01 | 8 | 9 | FTIGTVTLK | 100 | 268 | 268 | 565 |
| N | A*02:01 | 222 | 9 | LLLDRLNQL | 20 | 266 | 266 | 566 |
| N | A*30:01 | 147 | 9 | GTRNPANNA | 50 | 265 | 265 | 567 |
| S | B*44:02 | 829 | 9 | ADAGFIKQY | 20 | 265 | 265 | 568 |
| S | A*02:01 | 1000 | 9 | RLQSLQTYV | 17 | 264 | 264 | 569 |
| S | B*35:01 | 861 | 9 | LPPLLTDEM | 25 | 264 | 264 | 570 |
| nsp3 | B*40:01 | 1012 | 9 | LEMELTPVV | 50 | 262 | 262 | 571 |
| nsp3 | A*03:01 | 2749 | 9 | QVVNVVTTK | 100 | 260 | 260 | 572 |
| nsp12 | A*68:01 | 5025 | 9 | MASLVLARK | 100 | 260 | 260 | 573 |
| N | B*07:02 | 5 | 13 | GPQNQRNAPRITF | 33 | 260 | 260 | 574 |
| nsp12 | A*32:01 | 5315 | 9 | AMYTPHTVL | 50 | 258 | 258 | 575 |
| N | B*07:02 | 41 | 10 | RPQGLPNNTA | 33 | 258 | 258 | 576 |
| nsp3 | A*26:01 | 2136 | 10 | DTIANYAKPF | 50 | 256 | 256 | 577 |
| S | B*40:01 | 1181 | 9 | KEIDRLNEV | 20 | 256 | 256 | 578 |
| N | A*03:01 | 361 | 10 | KTFPPTEPKK | 33 | 255 | 255 | 579 |
| nsp12 | A*11:01 | 4814 | 9 | FAVSKGFFK | 100 | 255 | 255 | 580 |
| S | B*51:01 | 896 | 9 | IPFAMQMAY | 25 | 255 | 255 | 581 |
| S | B*07:02 | 714 | 9 | IPTNFTISV | 20 | 254 | 254 | 582 |
| M | B*35:01 | 39 | 9 | YANRNRFLY | 25 | 254 | 254 | 583 |
| S | A*03:01 | 725 | 9 | EILPVSMTK | 25 | 253 | 253 | 584 |
| S | A*29:02 | 444 | 10 | KVGGNYNYLY | 25 | 253 | 253 | 585 |
| ORF3a | A*68:01 | 227 | 9 | HVTFFIYNK | 100 | 252 | 252 | 586 |
| nsp12 | B*40:01 | 5308 | 9 | WEPEFYEAM | 33 | 252 | 252 | 587 |
| S | A*68:01 | 1064 | 10 | HVTYVPAQEK | 50 | 250 | 250 | 588 |
| nsp12 | A*11:01 | 4800 | 10 | QTVKPGNFNK | 100 | 249 | 249 | 589 |
| nsp3 | B*44:02 | 862 | 10 | VELGTEVNEF | 100 | 249 | 249 | 590 |
| nsp3 | B*40:01 | 887 | 9 | SELLTPLGI | 50 | 245 | 245 | 591 |
| ORF3a | B*51:01 | 158 | 12 | IPYNSVTSSIVI | 50 | 245 | 245 | 592 |
| nsp3 | B*51:01 | 2268 | 9 | EGYLNSTNV | 33 | 245 | 245 | 593 |
| N | B*51:01 | 121 | 10 | LPYGANKDGI | 33 | 244 | 244 | 594 |
| nsp3 | B*57:01 | 1999 | 10 | NKATYKPNTW | 100 | 244 | 244 | 595 |
| S | A*02:06 | 612 | 9 | YQDVNCTEV | 50 | 243 | 243 | 596 |
| S | A*29:02 | 258 | 9 | WTAGAAAYY | 25 | 243 | 243 | 597 |
| nsp12 | B*51:01 | 5217 | 13 | DYVYLPYPDPSRI | 25 | 243 | 243 | 598 |
| N | A*68:01 | 361 | 9 | KTFPPTEPK | 50 | 242 | 242 | 599 |
| M | B*57:01 | 68 | 8 | AAVYRINW | 50 | 242 | 242 | 600 |
| M | A*30:01 | 101 | 9 | RLFARTRSM | 100 | 241 | 241 | 601 |
| nsp12 | A*33:01 | 4810 | 13 | DFYDFAVSKGFFK | 100 | 239 | 239 | 602 |
| nsp3 | B*51:01 | 1283 | 9 | APYIVGDVV | 33 | 238 | 238 | 603 |
| S | B*35:01 | 898 | 9 | FAMQMAYRF | 25 | 236 | 236 | 604 |
| S | A*03:01 | 1065 | 9 | VTYVPAQEK | 25 | 235 | 235 | 605 |
| S | A*24:02 | 1051 | 12 | SFPQSAPHGVVF | 25 | 234 | 234 | 606 |
| S | A*31:01 | 454 | 9 | RLFRKSNLK | 50 | 233 | 233 | 607 |
| S | B*53:01 | 24 | 9 | LPPAYTNSF | 100 | 232 | 232 | 608 |
| nsp3 | A*24:02 | 2593 | 10 | AYVNTFSSTF | 50 | 231 | 231 | 609 |
| S | A*29:02 | 261 | 9 | GAAAYYVGY | 25 | 231 | 231 | 610 |
| nsp3 | B*44:03 | 1365 | 9 | QEILGTVSW | 100 | 230 | 230 | 611 |
| nsp3 | A*68:01 | 1217 | 9 | ESKPSVEQR | 50 | 229 | 229 | 612 |
| ORF3a | B*51:01 | 41 | 8 | LPFGWLIV | 50 | 229 | 229 | 613 |
| nsp3 | B*51:01 | 2194 | 10 | MPTTIAKNTV | 33 | 228 | 228 | 614 |
| S | B*08:01 | 109 | 9 | TLDSKTQSL | 20 | 227 | 227 | 615 |
| nsp4 | B*40:01 | 2804 | 9 | SEIIGYKAI | 100 | 227 | 227 | 616 |
| S | A*03:01 | 1136 | 14 | TVYDPLQPELDSFK | 25 | 225 | 225 | 617 |
| S | A*29:02 | 151 | 10 | SWMESEFRVY | 25 | 225 | 225 | 618 |
| nsp3 | B*44:03 | 2616 | 9 | AEAELAKNV | 50 | 225 | 225 | 619 |
| S | A*30:01 | 41 | 9 | KVFRSSVLH | 33 | 223 | 223 | 620 |
| nsp3 | B*51:01 | 2604 | 9 | VPMEKLKTL | 33 | 223 | 223 | 621 |
| M | B*35:01 | 37 | 9 | FAYANRNRF | 25 | 222 | 222 | 622 |
| nsp12 | B*51:01 | 5219 | 11 | VYLPYPDPSRI | 25 | 222 | 222 | 623 |
| nsp4 | A*24:02 | 3131 | 9 | MFTPLVPFW | 50 | 220 | 220 | 624 |
| nsp12 | B*51:01 | 4920 | 9 | FAYTKRNVI | 25 | 219 | 219 | 625 |
| nsp3 | B*57:01 | 1170 | 9 | RTNVYLAVF | 50 | 219 | 219 | 626 |
| nsp12 | A*68:01 | 4732 | 10 | FVVSTGYHFR | 100 | 217 | 217 | 627 |
| nsp12 | A*33:01 | 5024 | 9 | IMASLVLAR | 100 | 216 | 216 | 628 |
| nsp3 | B*40:01 | 2030 | 10 | SEDAQGMDNL | 50 | 216 | 216 | 629 |
| nsp3 | B*57:01 | 2704 | 10 | AKSHNIALIW | 100 | 216 | 216 | 630 |
| S | A*29:02 | 1102 | 9 | WFVTQRNFY | 25 | 215 | 215 | 631 |
| nsp12 | A*33:01 | 4953 | 9 | SICSTMTNR | 100 | 215 | 215 | 632 |
| S | A*24:02 | 1208 | 9 | QYIKWPWYI | 25 | 214 | 214 | 633 |
| M | A*68:01 | 5 | 10 | NGTITVEELK | 50 | 214 | 214 | 634 |
| nsp4 | A*02:06 | 3122 | 9 | FLAHIQWMV | 100 | 212 | 212 | 635 |
| nsp3 | A*24:02 | 2167 | 9 | NYMPYFFTL | 50 | 209 | 209 | 636 |
| nsp12 | A*29:02 | 4533 | 9 | TLKEILVTY | 100 | 208 | 208 | 637 |
| S | B*51:01 | 495 | 9 | YGFQPTNGV | 25 | 208 | 208 | 638 |
| nsp12 | B*51:01 | 5318 | 9 | TPHTVLQAV | 25 | 206 | 206 | 639 |
| S | A*68:01 | 88 | 10 | DGVYFASTEK | 50 | 205 | 205 | 640 |
| nsp16 | A*32:01 | 7020 | 9 | YVMHANYIF | 100 | 204 | 204 | 641 |
| S | B*44:03 | 464 | 10 | FERDISTEIY | 20 | 204 | 204 | 642 |
| nsp3 | B*44:03 | 2049 | 9 | EEVVENPTI | 50 | 204 | 204 | 643 |
| N | B*57:01 | 266 | 9 | KAYNVTQAF | 50 | 204 | 204 | 644 |
| nsp6 | A*02:01 | 3666 | 9 | WLDMVDTSL | 100 | 203 | 203 | 645 |
| N | A*30:01 | 119 | 9 | AGLPYGANK | 50 | 203 | 203 | 646 |
| M | A*33:01 | 97 | 9 | IASFRLFAR | 100 | 203 | 203 | 647 |
| nsp12 | A*11:01 | 4433 | 10 | KVAGFAKFLK | 100 | 202 | 202 | 648 |
| S | A*31:01 | 677 | 9 | QTNSPRRAR | 50 | 200 | 200 | 649 |
| S | B*44:03 | 653 | 8 | AEHVNNSY | 25 | 199 | 199 | 650 |
| nsp3 | A*24:02 | 2098 | 10 | AYVDNSSLTI | 50 | 197 | 197 | 651 |
| nsp3 | A*11:01 | 1090 | 9 | YIATNGPLK | 100 | 196 | 196 | 652 |
| nsp4 | B*57:01 | 2875 | 10 | RTTNGDFLHF | 100 | 196 | 196 | 653 |
| S | A*23:01 | 1208 | 10 | QYIKWPWYIW | 50 | 193 | 193 | 654 |
| S | B*35:01 | 321 | 9 | QPTESIVRF | 25 | 193 | 193 | 655 |
| nsp3 | A*01:01 | 1863 | 11 | ITDVFYKENSY | 50 | 192 | 192 | 656 |
| S | B*35:01 | 162 | 9 | SANNCTFEY | 25 | 192 | 192 | 657 |
| S | A*03:01 | 349 | 9 | SVYAWNRKR | 25 | 190 | 190 | 658 |
| nsp3 | A*03:01 | 1646 | 9 | YMSALNHTK | 100 | 190 | 190 | 659 |
| nsp3 | B*44:02 | 2325 | 10 | AEWFLAYILF | 33 | 190 | 190 | 660 |
| S | B*51:01 | 714 | 9 | IPTNFTISV | 25 | 190 | 190 | 661 |
| S | B*07:02 | 216 | 8 | LPQGFSAL | 20 | 189 | 189 | 662 |
| S | B*51:01 | 84 | 9 | LPFNDGVYF | 25 | 189 | 189 | 663 |
| nsp3 | B* 15:01 | 2254 | 9 | MSNLGMPSY | 100 | 188 | 188 | 664 |
| nsp3 | B*40:01 | 1014 | 10 | MELTPVVQTI | 50 | 188 | 188 | 665 |
| M | A*68:01 | 190 | 9 | DSGFAAYSR | 50 | 187 | 187 | 666 |
| ORF3a | A*29:02 | 99 | 9 | AGLEAPFLY | 50 | 185 | 185 | 667 |
| S | B*35:01 | 229 | 10 | LPIGINITRF | 25 | 185 | 185 | 668 |
| nsp12 | A*11:01 | 4977 | 9 | ATVVIGTSK | 100 | 184 | 184 | 669 |
| nsp12 | B*44:02 | 4558 | 10 | VENPDILRVY | 33 | 183 | 183 | 670 |
| nsp3 | B*40:01 | 960 | 10 | LEFGATSAAL | 50 | 182 | 182 | 671 |
| nsp3 | B*40:01 | 2111 | 9 | NELSRVLGL | 50 | 182 | 182 | 672 |
| S | B*07:02 | 24 | 9 | LPPAYTNSF | 20 | 181 | 181 | 673 |
| nsp3 | A*29:02 | 2323 | 9 | LVAEWFLAY | 50 | 175 | 175 | 674 |
| S | B*51:01 | 8 | 9 | LPLVSSQCV | 25 | 175 | 175 | 675 |
| nsp3 | B*57:01 | 1648 | 9 | SALNHTKKW | 50 | 175 | 175 | 676 |
| S | A*02:06 | 1060 | 9 | VVFLHVTYV | 50 | 174 | 174 | 677 |
| nsp12 | A*11:01 | 4841 | 9 | AISDYDYYR | 100 | 173 | 173 | 678 |
| nsp3 | B*51:01 | 2169 | 8 | MPYFFTLL | 33 | 173 | 173 | 679 |
| ORF3a | B*53:01 | 137 | 9 | NPLLYDANY | 100 | 172 | 172 | 680 |
| nsp3 | B*57:01 | 2469 | 9 | VARDLSLQF | 100 | 172 | 172 | 681 |
| nsp4 | A*02:06 | 2864 | 9 | FVVPGLPGT | 100 | 170 | 170 | 682 |
| S | A*31:01 | 449 | 9 | YNYLYRLFR | 50 | 170 | 170 | 683 |
| M | B*35:01 | 170 | 9 | VATSRTLSY | 25 | 169 | 169 | 684 |
| nsp3 | B*51:01 | 1658 | 10 | YPQVNGLTSI | 33 | 169 | 169 | 685 |
| nsp3 | B*44:02 | 1890 | 11 | TEIDPKLDNYY | 33 | 168 | 168 | 686 |
| S | A*24:02 | 36 | 8 | VYYPDKVF | 25 | 167 | 167 | 687 |
| nsp4 | B*51:01 | 2854 | 9 | IAAVITREV | 100 | 167 | 167 | 688 |
| S | A*03:01 | 378 | 9 | KCYGVSPTK | 25 | 166 | 166 | 689 |
| nsp3 | A*68:01 | 1210 | 10 | EVKPFITESK | 50 | 166 | 166 | 690 |
| S | A*02:01 | 691 | 9 | SIIAYTMSL | 17 | 165 | 165 | 691 |
| S | B*40:01 | 1194 | 10 | NESLIDLQEL | 20 | 165 | 165 | 692 |
| S | A*31:01 | 1099 | 9 | GTHWFVTQR | 50 | 164 | 164 | 693 |
| S | A*68:01 | 347 | 9 | FASVYAWNR | 50 | 164 | 164 | 694 |
| S | B*35:01 | 24 | 9 | LPPAYTNSF | 25 | 163 | 163 | 695 |
| S | B*44:03 | 779 | 11 | QEVFAQVKQIY | 20 | 163 | 163 | 696 |
| S | B*53:01 | 55 | 10 | FLPFFSNVTW | 100 | 163 | 163 | 697 |
| S | B*35:01 | 343 | 9 | NATRFASVY | 25 | 162 | 162 | 698 |
| S | B*07:02 | 321 | 9 | QPTESIVRF | 20 | 160 | 160 | 699 |
| nsp3 | B*57:01 | 1951 | 9 | ASRELKVTF | 100 | 159 | 159 | 700 |
| nsp3 | A*02:06 | 1923 | 9 | ASFDNFKFV | 100 | 158 | 158 | 701 |
| nsp3 | B*57:01 | 1477 | 10 | VSSPDAVTAY | 50 | 158 | 158 | 702 |
| M | A*01:01 | 39 | 9 | YANRNRFLY | 25 | 157 | 157 | 703 |
| nsp16 | B*44:02 | 7014 | 13 | REQIDGYVMHANY | 50 | 157 | 157 | 704 |
| S | B*51:01 | 574 | 9 | DAVRDPQTL | 25 | 157 | 157 | 705 |
| S | B*51:01 | 1089 | 8 | FPREGVFV | 25 | 156 | 156 | 706 |
| nsp6 | A*24:02 | 3653 | 9 | VYMPASWVM | 100 | 155 | 155 | 707 |
| S | B*57:01 | 622 | 12 | VAIHADQLTPTW | 100 | 155 | 155 | 708 |
| nsp6 | A*02:01 | 3605 | 9 | FLYENAFLP | 100 | 154 | 154 | 709 |
| S | A*31:01 | 346 | 10 | RFASVYAWNR | 50 | 154 | 154 | 710 |
| S | B*40:01 | 464 | 9 | FERDISTEI | 20 | 154 | 154 | 711 |
| nsp3 | B*57:01 | 2355 | 9 | AVHFISNSW | 100 | 154 | 154 | 712 |
| nsp3 | A*29:02 | 2384 | 9 | YIFFASFYY | 50 | 153 | 153 | 713 |
| S | A*02:01 | 857 | 9 | GLTVLPPLL | 17 | 151 | 151 | 714 |
| N | A*11:01 | 249 | 9 | KSAAEASKK | 33 | 151 | 151 | 715 |
| nsp4 | A*24:02 | 3108 | 9 | IYLYLTFYL | 50 | 151 | 151 | 716 |
| nsp3 | A*03:01 | 2753 | 9 | VVTTKIALK | 100 | 149 | 149 | 717 |
| nsp3 | A*24:02 | 1971 | 9 | DYKHYTPSF | 50 | 149 | 149 | 718 |
| ORF3a | A*01:01 | 207 | 9 | FTSDYYQLY | 33 | 147 | 147 | 719 |
| nsp12 | A*02:06 | 4557 | 10 | FVENPDILRV | 100 | 147 | 147 | 720 |
| nsp3 | A*24:02 | 1899 | 9 | YYKKDNSYF | 50 | 147 | 147 | 721 |
| nsp3 | A*24:02 | 2330 | 8 | AYILFTRF | 50 | 147 | 147 | 722 |
| nsp3 | B* 15:01 | 1967 | 9 | VVAIDYKHY | 100 | 147 | 147 | 723 |
| nsp3 | B*57:01 | 1647 | 10 | MSALNHTKKW | 50 | 147 | 147 | 724 |
| nsp3 | A*30:01 | 2196 | 9 | TTIAKNTVK | 100 | 146 | 146 | 725 |
| M | B*57:01 | 171 | 9 | ATSRTLSYY | 50 | 145 | 145 | 726 |
| S | A*68:01 | 975 | 9 | SVLNDILSR | 50 | 142 | 142 | 727 |
| S | B* 15:01 | 1000 | 8 | RLQSLQTY | 33 | 142 | 142 | 728 |
| ORF3a | A*29:02 | 206 | 10 | YFTSDYYQLY | 50 | 140 | 140 | 729 |
| ORF3a | B*57:01 | 58 | 12 | SASKIITLKKRW | 50 | 140 | 140 | 730 |
| ORF3a | B*57:01 | 63 | 9 | ITLKKRWQL | 50 | 140 | 140 | 731 |
| N | A*68:01 | 360 | 10 | YKTFPPTEPK | 50 | 139 | 139 | 732 |
| S | B*44:03 | 1016 | 9 | AEIRASANL | 20 | 139 | 139 | 733 |
| nsp3 | B*57:01 | 1586 | 9 | MSMTYGQQF | 50 | 139 | 139 | 734 |
| nsp3 | A*01:01 | 1415 | 9 | VVDYGARFY | 33 | 138 | 138 | 735 |
| nsp12 | A*29:02 | 5267 | 10 | QEYADVFHLY | 100 | 138 | 138 | 736 |
| nsp3 | B*57:01 | 2553 | 9 | SAKSASVYY | 100 | 138 | 138 | 737 |
| nsp6 | B*07:02 | 3655 | 9 | MPASWVMRI | 50 | 137 | 137 | 738 |
| S | A*31:01 | 637 | 10 | STGSNVFQTR | 50 | 136 | 136 | 739 |
| N | A*02:01 | 316 | 9 | GMSRIGMEV | 20 | 135 | 135 | 740 |
| nsp3 | A*24:02 | 2589 | 10 | KMFDAYVNTF | 50 | 135 | 135 | 741 |
| nsp3 | A*01:01 | 1937 | 11 | FADDLNQLTGY | 50 | 134 | 134 | 742 |
| nsp3 | A*02:01 | 2242 | 9 | SLIYSTAAL | 50 | 134 | 134 | 743 |
| nsp12 | A*03:01 | 4892 | 9 | KSAGFPFNK | 100 | 133 | 133 | 744 |
| nsp3 | A*01:01 | 2494 | 11 | VTVKNGSIHLY | 50 | 131 | 131 | 745 |
| nsp3 | A*32:01 | 2625 | 9 | SLDNVLSTF | 50 | 131 | 131 | 746 |
| N | B*07:02 | 150 | 10 | NPANNAAIVL | 33 | 131 | 131 | 747 |
| nsp4 | B*57:01 | 3148 | 8 | ISTKHFYW | 100 | 131 | 131 | 748 |
| nsp3 | A*01:01 | 2273 | 9 | STNVTIATY | 50 | 129 | 129 | 749 |
| S | A*03:01 | 454 | 9 | RLFRKSNLK | 25 | 128 | 128 | 750 |
| S | A*68:01 | 637 | 10 | STGSNVFQTR | 50 | 128 | 128 | 751 |
| nsp3 | A*29:02 | 2208 | 10 | KFCLEASFNY | 50 | 126 | 126 | 752 |
| nsp3 | B*57:01 | 1538 | 9 | TSNPTTFHL | 50 | 126 | 126 | 753 |
| S | B*35:01 | 699 | 9 | LGAENSVAY | 25 | 125 | 125 | 754 |
| nsp3 | A*03:01 | 1768 | 9 | VMYMGTLSY | 100 | 124 | 124 | 755 |
| nsp4 | B*51:01 | 2885 | 8 | LPRVFSAV | 100 | 124 | 124 | 756 |
| nsp4 | B*51:01 | 3101 | 8 | LPGVYSVI | 100 | 124 | 124 | 757 |
| nsp3 | A*01:01 | 2660 | 14 | QSDIEVTGDSCNNY | 50 | 123 | 123 | 758 |
| S | A*03:01 | 292 | 9 | ALDPLSETK | 25 | 123 | 123 | 759 |
| nsp4 | B*40:01 | 3071 | 10 | FGEYSHVVAF | 100 | 123 | 123 | 760 |
| N | A*03:01 | 361 | 14 | KTFPPTEPKKDKKK | 50 | 122 | 122 | 761 |
| S | B*53:01 | 624 | 10 | IHADQLTPTW | 100 | 122 | 122 | 762 |
| nsp3 | A*03:01 | 1397 | 9 | AIVSTIQRK | 100 | 121 | 121 | 763 |
| M | A*30:01 | 196 | 10 | YSRYRIGNYK | 100 | 121 | 121 | 764 |
| nsp16 | A*32:01 | 6980 | 9 | KLMGHFAWW | 100 | 121 | 121 | 765 |
| M | A*01:01 | 186 | 11 | RVAGDSGFAAY | 33 | 120 | 120 | 766 |
| S | A*68:01 | 725 | 9 | EILPVSMTK | 50 | 120 | 120 | 767 |
| nsp3 | B* 15:01 | 2552 | 9 | SSAKSASVY | 100 | 120 | 120 | 768 |
| nsp3 | A*02:01 | 1675 | 9 | YLATALLTL | 33 | 119 | 119 | 769 |
| S | A*31:01 | 558 | 10 | KFLPFQQFGR | 50 | 119 | 119 | 770 |
| nsp3 | A*32:01 | 2273 | 9 | STNVTIATY | 50 | 119 | 119 | 771 |
| nsp3 | B* 15:01 | 2435 | 10 | YVYANGGKGF | 100 | 117 | 117 | 772 |
| nsp12 | B*57:01 | 4710 | 9 | STVFPPTSF | 100 | 117 | 117 | 773 |
| nsp3 | A*32:01 | 2225 | 9 | KLINIIIWF | 50 | 116 | 116 | 774 |
| ORF3a | A*29:02 | 204 | 9 | HSYFTSDYY | 50 | 115 | 115 | 775 |
| nsp4 | B*35:01 | 2936 | 10 | TNVLEGSVAY | 33 | 115 | 115 | 776 |
| nsp3 | A*01:01 | 1891 | 9 | EIDPKLDNY | 50 | 114 | 114 | 777 |
| S | A*03:01 | 35 | 10 | GVYYPDKVFR | 25 | 114 | 114 | 778 |
| N | A*26:01 | 323 | 11 | EVTPSGTWLTY | 33 | 114 | 114 | 779 |
| nsp12 | A*32:01 | 4433 | 9 | KVAGFAKFL | 50 | 113 | 113 | 780 |
| nsp4 | B*35:01 | 2924 | 9 | DASGKPVPY | 33 | 113 | 113 | 781 |
| S | B*40:01 | 1016 | 9 | AEIRASANL | 20 | 113 | 113 | 782 |
| nsp3 | A*24:02 | 1182 | 9 | LYDKLVSSF | 100 | 112 | 112 | 783 |
| ORF3a | A*29:02 | 101 | 9 | LEAPFLYLY | 50 | 112 | 112 | 784 |
| S | A*68:01 | 370 | 9 | NSASFSTFK | 50 | 112 | 112 | 785 |
| S | A*26:01 | 886 | 9 | WTFGAGAAL | 25 | 111 | 111 | 786 |
| ORF3a | A*24:02 | 211 | 9 | YYQLYSTQL | 50 | 109 | 109 | 787 |
| S | A*26:01 | 604 | 9 | TSNQVAVLY | 25 | 109 | 109 | 788 |
| M | B*57:01 | 105 | 8 | RTRSMWSF | 50 | 109 | 109 | 789 |
| nsp12 | A*33:01 | 5133 | 10 | FVNEFYAYLR | 100 | 108 | 108 | 790 |
| S | A*68:01 | 394 | 10 | NVYADSFVIR | 50 | 108 | 108 | 791 |
| nsp4 | B*51:01 | 3022 | 9 | DAVNLLTNM | 100 | 108 | 108 | 792 |
| nsp16 | B*57:01 | 6807 | 9 | VAMPNLYKM | 100 | 108 | 108 | 793 |
| S | A*29:02 | 686 | 10 | SVASQSIIAY | 33 | 107 | 107 | 794 |
| nsp6 | B*07:02 | 3612 | 10 | LPFAMGIIAM | 50 | 107 | 107 | 795 |
| M | A*03:01 | 150 | 9 | RIAGHHLGR | 50 | 106 | 106 | 796 |
| S | A*26:01 | 695 | 13 | YTMSLGAENSVAY | 25 | 106 | 106 | 797 |
| nsp3 | A*02:01 | 843 | 10 | FELDERIDKV | 33 | 105 | 105 | 798 |
| S | B*44:03 | 989 | 9 | AEVQIDRLI | 20 | 105 | 105 | 799 |
| S | B*57:01 | 880 | 9 | GTITSGWTF | 100 | 105 | 105 | 800 |
| S | B*53:01 | 1052 | 9 | FPQSAPHGV | 100 | 103 | 103 | 801 |
| S | B*40:01 | 660 | 11 | YECDIPIGAGI | 20 | 101 | 101 | 802 |
| S | B*40:01 | 989 | 8 | AEVQIDRL | 20 | 101 | 101 | 803 |

TABLE 6

Cross-reactive CD4 non-spike SARS-CoV-2 Epitopes

| Sequence | Protein | Total SFC | IFNg | IL13 | IL5 | SEQ ID NO |
|---|---|---|---|---|---|---|
| NHNFLVQAGNVQLRV | 3CL | 51387 | 33520 | 1373 | 10640 | 804 |
| QNCVLKLKVDTANPK | 3CL | 1133 | 147 | 0 | 240 | 805 |
| SEETGTLIVNSVLLF | envelope protein | 267 | 267 | 0 | 0 | 806 |
| LAILTALRLCAYCCN | envelope protein | 1800 | 1760 | 0 | 0 | 807 |
| TSHKLVLSVNPYVCN | Hel | 147 | 133 | 0 | 0 | 808 |
| ISPYNSQNAVASKIL | Hel | 4267 | 4093 | 0 | 107 | 809 |
| NVNRFNVAITRAKVG | Hel | 827 | 707 | 0 | 0 | 810 |
| QPYVFIKRSDARTAP | nsp1 | 493 | 360 | 107 | 0 | 811 |
| VLSFCAFAVDAAKAY | nsp10 | 373 | 160 | 0 | 0 | 812 |
| NMFITREEAIRHVRA | nsp14 | 627 | 613 | 0 | 0 | 813 |
| REEAIRHVRAWIGFD | nsp14 | 1467 | 173 | 0 | 0 | 814 |
| PLMYKGLPWNVVRIK | nsp14 | 127 | 0 | 0 | 0 | 815 |
| EIIKSQDLSVVSKVV | nsp15 | 613 | 0 | 0 | 280 | 816 |
| TQLCQYLNTLTLAVP | nsp16 | 1227 | 1027 | 0 | 133 | 817 |
| QIDGYVMHANYIFWR | nsp16 | 773 | 440 | 160 | 0 | 818 |
| PLNSIIKTIQPRVEK | nsp2 | 2653 | 1280 | 0 | 920 | 819 |
| EEIAIILASFSASTS | nsp2 | 13813 | 7773 | 1507 | 1587 | 820 |
| SPLYAFASEAARVVR | nsp2 | 16987 | 7173 | 1160 | 5320 | 821 |
| AITILDGISQYSLRL | nsp2 | 307 | 0 | 0 | 0 | 822 |
| QTFFKLVNKFLALCA | nsp2 | 1173 | 640 | 107 | 133 | 823 |
| GETFVTHSKGLYRKC | nsp2 | 307 | 0 | 0 | 0 | 824 |
| KGGKIVNNWLKQLIK | nsp4 | 253 | 0 | 0 | 253 | 825 |
| LFVAAIFYLITPVHV | nsp4 | 880 | 427 | 0 | 0 | 826 |
| DTRYVLMDGSIIQFP | nsp4 | 213 | 213 | 0 | 0 | 827 |
| FGEYSHVVAFNTLLF | nsp4 | 960 | 0 | 0 | 0 | 828 |
| NTLLFLMSFTVLCLT | nsp4 | 333 | 213 | 0 | 0 | 829 |
| TIAYIICISTKHFYW | nsp4 | 773 | 533 | 0 | 0 | 830 |
| CTFLLNKEMYLKLRS | nsp4 | 547 | 493 | 0 | 0 | 831 |
| LLVLVQSTQWSLFFF | nsp6 | 387 | 253 | 0 | 0 | 832 |
| SLFFFLYENAFLPFA | nsp6 | 240 | 213 | 0 | 0 | 833 |
| LCLFLLPSLATVAYF | nsp6 | 147 | 120 | 0 | 0 | 834 |
| TLVYKVYYGNALDQA | nsp6 | 120 | 0 | 0 | 0 | 835 |
| NRYFRLTLGVYDYLV | nsp6 | 4413 | 2627 | 0 | 200 | 836 |
| DAFKLNIKLLGVGGK | nsp6 | 400 | 400 | 0 | 0 | 837 |
| VLKKLKKSLNVAKSE | nsp8 | 2937 | 2357 | 0 | 420 | 838 |
| LIVTALRANSAVKLQ | nsp8 | 800 | 773 | 0 | 0 | 839 |
| SDFVRATATIPIQAS | ORF3a protein | 19453 | 1453 | 2827 | 8853 | 840 |
| MFHLVDFQVTIAEIL | ORF6 protein | 11073 | 9167 | 340 | 1220 | 841 |
| IAEILLIIMRTFKVS | ORF6 protein | 387 | 0 | 0 | 0 | 842 |
| LIIMRTFKVSIWNLD | ORF6 protein | 733 | 587 | 0 | 0 | 843 |
| TFKVSIWNLDYIINL | ORF6 protein | 2547 | 2360 | 0 | 0 | 844 |
| IWNLDYIINLIIKNL | ORF6 protein | 800 | 760 | 0 | 0 | 845 |
| YIINLIIKNLSKSLT | ORF6 protein | 1393 | 1283 | 0 | 0 | 846 |
| MKIILFLALITLATC | ORF7a protein | 373 | 293 | 0 | 0 | 847 |
| VKHVYQLRARSVSPK | ORF7a protein | 3640 | 2947 | 0 | 227 | 848 |
| LYSPIFLIVAAIVFI | ORF7a protein | 520 | 400 | 0 | 0 | 849 |
| ENLLLYIDINGNLHP | PLpro | 493 | 267 | 0 | 0 | 850 |
| EAARYMRSLKVPATV | PLpro | 227 | 0 | 0 | 0 | 851 |
| DNFKFVCDNIKFADD | PLpro | 173 | 0 | 0 | 0 | 852 |
| NELSRVLGLKTLATH | PLpro | 547 | 213 | 0 | 0 | 853 |
| LEASFNYLKSPNFSK | PLpro | 933 | 627 | 0 | 0 | 854 |
| GSLIYSTAALGVLMS | PLpro | 733 | 733 | 0 | 0 | 855 |
| ISSFKWDLTAFGLVA | PLpro | 267 | 173 | 0 | 0 | 856 |
| FDAYVNTFSSTFNVP | PLpro | 400 | 347 | 0 | 0 | 857 |
| SHNIALIWNVKDFMS | PLpro | 1133 | 320 | 0 | 347 | 858 |
| PDILRVYANLGERVR | RdRpol | 133 | 117 | 0 | 0 | 859 |
| SLLMPILTLTRALTA | RdRpol | 333 | 267 | 0 | 0 | 860 |
| EFYAYLRKHFSMMIL | RdRpol | 880 | 320 | 0 | 0 | 861 |
| LRKHFSMMILSDDAV | RdRpol | 373 | 0 | 0 | 0 | 862 |
| GLVASIKNFKSVLYY | RdRpol | 533 | 240 | 0 | 277 | 863 |
| KHFYWFFSNYLKRRV | nsp4 | 1853 | 0 | 173 | 347 | 244 |
| DDQIGYYRRATRRIR | nucleocapsid phosphoprotein | 787 | 400 | 120 | 0 | 245 |
| DAALALLLLDRLNQL | nucleocapsid phosphoprotein | 3813 | 3520 | 0 | 0 | 246 |
| LLLLDRLNQLESKMS | nucleocapsid phosphoprotein | 1053 | 1053 | 0 | 0 | 247 |
| PSGTWLTYTGAIKLD | nucleocapsid phosphoprotein | 1227 | 947 | 0 | 0 | 248 |
| ALLAVFQSASKIITL | ORF3a protein | 1293 | 360 | 147 | 467 | 249 |
| KKRWQLALSKGVHFV | ORF3a protein | 907 | 227 | 0 | 0 | 250 |
| LVYFLQSINFVRIIM | ORF3a protein | 240 | 0 | 0 | 107 | 251 |
| QSINFVRIIMRLWLC | ORF3a protein | 413 | 213 | 0 | 0 | 252 |
| KNPLLYDANYFLCWH | ORF3a protein | 427 | 307 | 0 | 0 | 253 |
| FLGIITTVAAFHQEC | ORF8 protein | 560 | 0 | 0 | 0 | 254 |
| FYSKWYIRVGARKSA | ORF8 protein | 13327 | 10597 | 1020 | 1097 | 255 |
| YIRVGARKSAPLIEL | ORF8 protein | 713 | 590 | 0 | 0 | 256 |
| KVTFFPDLNGDVVAI | PLpro | 2453 | 2120 | 0 | 253 | 257 |
| KLLKSIAATRGATVV | RdRpol | 200 | 173 | 0 | 0 | 258 |
| LMIERFVSLAIDAYP | RdRpol | 2113 | 1650 | 0 | 140 | 259 |

TABLE 7

31 Best Cross-reactive CD4 Spike SARS-CoV-2 Epitopes

| peptide sequence | Peptide start | Peptide end | Protein specific name | Organism | category | SEQ ID NO |
|---|---|---|---|---|---|---|
| NNATNVVIKVCEFQF | 121 | 135 | S | SARS-Cov-2 | preferred spike epitopes | 864 |
| QPTESIVRFPNITNL | 321 | 335 | S | SARS-Cov-2 | preferred spike epitopes | 865 |
| VFNATRFASVYAWNR | 341 | 355 | S | SARS-Cov-2 | preferred spike epitopes | 866 |
| SVASQSIIAYTMSLG | 686 | 700 | S | SARS-Cov-2 | preferred spike epitopes | 867 |
| WTFGAGAALQIPFAM | 886 | 900 | S | SARS-Cov-2 | preferred spike epitopes | 868 |
| AQALNTLVKQLSSNF | 956 | 970 | S | SARS-Cov-2 | preferred spike epitopes | 869 |
| GAISSVLNDILSRLD | 971 | 985 | S | SARS-Cov-2 | preferred spike epitopes | 870 |
| VQIDRLITGRLQSLQ | 991 | 1005 | S | SARS-Cov-2 | preferred spike epitopes | 871 |
| LNEVAKNLNESLIDL | 1186 | 1200 | S | SARS-Cov-2 | preferred spike epitopes | 872 |
| YEQYIKWPWYIWLGF | 1206 | 1220 | S | SARS-Cov-2 | preferred spike epitopes | 873 |
| SLLIVNNATNVVIKV | 116 | 130 | S | SARS-Cov-2 | preferred spike epitopes | 260 |
| CEFQFCNDPFLGVYY | 131 | 145 | S | SARS-Cov-2 | preferred spike epitopes | 261 |
| CTFEYVSQPFLMDLE | 166 | 180 | S | SARS-Cov-2 | preferred spike epitopes | 262 |
| IGINITRFQTLLALH | 231 | 245 | S | SARS-Cov-2 | preferred spike epitopes | 263 |
| TRFQTLLALHRSYLT | 236 | 250 | S | SARS-Cov-2 | preferred spike epitopes | 264 |
| FTVEKGIYQTSNFRV | 306 | 320 | S | SARS-Cov-2 | preferred spike epitopes | 265 |
| SNFRVQPTESIVRFP | 316 | 330 | S | SARS-Cov-2 | preferred spike epitopes | 266 |
| IVRFPNITNLCPFGE | 326 | 340 | S | SARS-Cov-2 | preferred spike epitopes | 267 |
| CPFGEVFNATRFASV | 336 | 350 | S | SARS-Cov-2 | preferred spike epitopes | 268 |
| AYSNNSIAIPTNFTI | 706 | 720 | S | SARS-Cov-2 | preferred spike epitopes | 269 |
| NLLLQYGSFCTQLNR | 751 | 765 | S | SARS-Cov-2 | preferred spike epitopes | 270 |
| TQLNRALTGIAVEQD | 761 | 775 | S | SARS-Cov-2 | preferred spike epitopes | 271 |
| VFAQVKQIYKTPPIK | 781 | 795 | S | SARS-Cov-2 | preferred spike epitopes | 272 |
| NFSQILPDPSKPSKR | 801 | 815 | S | SARS-Cov-2 | preferred spike epitopes | 273 |
| KPSKRSFIEDLLFNK | 811 | 825 | S | SARS-Cov-2 | preferred spike epitopes | 274 |
| SFIEDLLFNKVTLAD | 816 | 830 | S | SARS-Cov-2 | preferred spike epitopes | 275 |
| CAQKFNGLTVLPPLL | 851 | 865 | S | SARS-Cov-2 | preferred spike epitopes | 276 |
| AQYTSALLAGTITSG | 871 | 885 | S | SARS-Cov-2 | preferred spike epitopes | 277 |
| APHGVVFLHVTYVPA | 1056 | 1070 | S | SARS-Cov-2 | preferred spike epitopes | 278 |
| ELDKYFKNHTSPDVD | 1151 | 1165 | S | SARS-Cov-2 | preferred spike epitopes | 279 |
| GINASVVNIQKEIDR | 1171 | 1185 | S | SARS-Cov-2 | preferred spike epitopes | 280 |

TABLE 8

129 Common Cold Coronavirus Epitopes homologous to C30 subset

| Peptide ID | Sequence | Organism | SEQ ID NO: |
|---|---|---|---|
| 5 | NSLFRMPLGVYNYKI | OC43 | 874 |
| 10 | NHAFWVFSYCRKLGT | OC43 | 875 |
| 15 | PLLENIDYFNMRRAK | OC43 | 876 |
| 25 | EYYEFLNKHFSMMIL | OC43 | 877 |
| 31 | NDVAFVSTFNVLQDV | OC43 | 878 |
| 36 | KEEAVKRVRAWVGFD | OC43 | 879 |
| 42 | YQKVFRVYLAYIKKL | OC43 | 880 |
| 52 | LNKHFSMMILSDDGV | OC43 | 881 |
| 61 | VPLNAIPSLAANTLN | OC43 | 882 |
| 66 | MRFYIIIASFIKLFS | OC43 | 883 |
| 71 | QLKQLEKACNIAKSA | OC43 | 884 |
| 83 | ISPYNSQNFAAKRVL | OC43 | 885 |
| 88 | TDHKYVLSVSPYVCN | OC43 | 886 |
| 94 | KITEWPTATGDVVLA | OC43 | 887 |
| 99 | QYSFKLVMNGLVFGL | OC43 | 888 |
| 115 | TDLTVTSAGQPCVAS | OC43 | 889 |
| 119 | ALIATAHSSIKQGTQ | OC43 | 890 |
| 124 | LQSAATIRSVAYVAN | OC43 | 891 |
| 129 | YENKLKAKNESSSLC | OC43 | 892 |
| 134 | MPFLLDDLVPRAYYL | OC43 | 893 |
| 139 | NSSILSLCAFSVDPK | OC43 | 894 |
| 144 | SPCKELEGVGAKVSA | OC43 | 895 |
| 149 | DLALKLKGLDAMFFY | OC43 | 896 |
| 154 | KDVYELRYNGAIRFD | OC43 | 897 |
| 159 | SLFVDYSNLLHSKVK | OC43 | 898 |
| 4 | NRFFKCTMGVYDFKV | NL63 | 899 |
| 9 | GHFNEEFYNFLRLRG | NL63 | 900 |
| 14 | LLSSLTLTVKFVVES | NL63 | 901 |
| 19 | VLLERYVSLAIDAYP | NL63 | 902 |
| 24 | DYYGYLRKHFSMMIL | NL63 | 903 |
| 30 | LHNFSVSHNGVFLGV | NL63 | 904 |
| 35 | RDFAIRNVRGWLGMD | NL63 | 905 |
| 41 | LFTNSILMLDKQGQL | NL63 | 906 |
| 51 | LRKHFSMMILSDDGV | NL63 | 907 |
| 60 | SDLSTLAVTAIVVVG | NL63 | 908 |
| 65 | KAINNIVASFSSVND | NL63 | 909 |
| 70 | LIKQLKRAMNIAKSE | NL63 | 910 |
| 75 | TQLCQYLNSTTMCVP | NL63 | 911 |
| 82 | ISPYNSQNYVASRFL | NL63 | 912 |
| 93 | KDGFFTYLNGVIREK | NL63 | 913 |
| 98 | VPFDVLCNEFLATFI | NL63 | 914 |
| 103 | KHLKSIVNTRNATVV | NL63 | 915 |
| 108 | FIAGNTVHANYIFWR | NL63 | 916 |
| 114 | GVFVQDPAPIDIDAF | NL63 | 917 |
| 123 | STILQAAGLCVVCGS | NL63 | 918 |
| 128 | YVTNEIGLNASVTLK | NL63 | 919 |
| 133 | MDLLLDDFVTILKSL | NL63 | 920 |
| 138 | VDISYLNRARGSSAA | NL63 | 921 |
| 143 | ANGFFYIDVGNHRSA | NL63 | 922 |
| 148 | IIAVELLLLDFKTAV | NL63 | 923 |
| 153 | PSVAVRTYSEAAAQG | NL63 | 924 |
| 158 | LNLSSELKQLEAKTA | NL63 | 925 |
| 56 | LNKHFSMIILSDDGV | MM3-5 | 926 |
| 55 | LNKHFNMMILSDDGV | MM3-4 | 927 |
| 78 | TQLCQYLSTTTLAVP | MM3-4 | 928 |
| 47 | NVNRFNVAITRARKG | MM3-3 | 929 |
| 54 | LFFHFSMMILSDDGV | MM3-3 | 930 |
| 77 | TQLCQYLSTTTIAVP | MM3-3 | 931 |
| 110 | EIDGSVMHANYLFWR | MM3-3 | 932 |
| 20 | LLIKRFVSLAIHAYP | MM3-2 | 933 |
| 26 | EFYEFLNKHFSMMIL | MM3-2 | 934 |
| 104 | KCLKSIAATRGVSVV | MM3-2 | 935 |
| 37 | RDEAIRRVRAWVGFD | MM3-1 | 936 |
| 46 | NVNRFNLAITRAKKG | MM3-1 | 937 |
| 53 | FNKHFSMMILSDDGV | MM3-1 | 938 |
| 76 | TQLCQYLNTTTIAVP | MM3-1 | 939 |
| 84 | ISPYNSQNYVAKRIL | MM3-1 | 940 |
| 89 | TDHKYVLSVAPYVCN | MM3-1 | 941 |
| 109 | DIDGNVMHANYLFWR | MM3-1 | 942 |
| 3 | NSVFRMPMGVYNYKI | HKU1 | 943 |
| 8 | NHVLWLFSYCRKIGV | HKU1 | 944 |
| 13 | PSNSIVCRFDTRVLN | HKU1 | 945 |
| 18 | LLIERFVSLAIDAYP | HKU1 | 946 |
| 23 | EYYEFLCKHFSMMIL | HKU1 | 947 |
| 29 | FYGPYRDAQVVQLPV | HKU1 | 948 |
| 34 | KDEAIKRVRGWVGFD | HKU1 | 949 |
| 40 | LERVSLWNYGKP1NL | HKU1 | 950 |
| 45 | NVNRFNVAITRAKKG | HKU1 | 951 |
| 50 | LCKHFSMMILSDDGV | HKU1 | 952 |
| 59 | SKLFITKDEAIKRVR | HKU1 | 953 |
| 64 | LEYPIISNEVSINTS | HKU1 | 954 |
| 69 | QIKQLEKACNIAKSV | HKU1 | 955 |
| 74 | TQLCQYLNTTTLAVP | HKU1 | 956 |
| 81 | ISPYNSONYVAKRVL | HKU1 | 957 |
| 87 | TNHKYVLSVSPYVCN | HKU1 | 958 |
| 92 | KVTVWPVATGDVVLA | HKU1 | 959 |
| 97 | ETFGKPVIWFCHDEA | HKU1 | 960 |
| 102 | KCLKSIAATRGVPVV | HKU1 | 961 |
| 107 | EIDGNVMHANYLFWR | HKU1 | 962 |
| 113 | SRFVMRLQTIATICG | HKU1 | 963 |
| 118 | VLLTVDGVNFKSISL | HKU1 | 964 |
| 122 | LALLYRNLKCSYVLN | HKU1 | 965 |
| 127 | KIEDLSIRNLQKRLY | HKU1 | 966 |
| 132 | MHWLIRFIVFVANML | HKU1 | 967 |
| 137 | VDNVYVTYAGSVWHI | HKU1 | 968 |
| 142 | TRSMTYCRVGACEYA | HKU1 | 969 |
| 147 | APATGWLLYQLLNGL | HKU1 | 970 |
| 152 | PGNTFITVEAAIELS | HKU1 | 971 |
| 157 | WLLPDAAEELASPMK | HKU1 | 972 |
| 2 | NRFCKCTLGVYDFCV | 229E | 973 |
| 7 | KSFSTFESAYMPIAD | 229E | 974 |
| 12 | PCPSILKVIDGGKIW | 229E | 975 |
| 17 | ILLERYVSLAIDAYP | 229E | 976 |
| 22 | DFYGYLQKHFSMMIL | 229E | 977 |
| 28 | LHNFSIISGTAFLGV | 229E | 978 |
| 33 | RDFAMRHVRGWLGMD | 229E | 979 |
| 39 | NDKITEFQLDYSIDV | 229E | 980 |
| 44 | NANRFNVAITRAKKG | 229E | 981 |
| 49 | LQKHFSMMILSDDSV | 229E | 982 |
| 58 | VDHSAFAYESAVVNG | 229E | 983 |
| 63 | NLVFNILSMFSSSFS | 229E | 984 |
| 68 | IIKQLKKAMNVAKAE | 229E | 985 |
| 73 | TQLCQYFNSTTLCVP | 229E | 986 |
| 80 | ISPYNSQNYVAARLL | 229E | 987 |
| 86 | TDHKFILAITPYVCN | 229E | 988 |
| 91 | QEATLPDIAEDVVDQ | 229E | 989 |
| 96 | QTLFCNIMKFSDRPF | 229E | 990 |
| 101 | KCLKSIVATRNATVV | 229E | 991 |
| 106 | FIDGNIIHANYVFWR | 229E | 992 |
| 112 | SAVAVVGGTIQILAS | 229E | 993 |
| 117 | ALLAFFLSKHSDFGL | 229E | 994 |
| 121 | LAKFTKLLLLIYTLY | 229E | 995 |
| 126 | VIDNEIIVKPNISLC | 229E | 996 |
| 131 | NVHLKDVTKENQEIL | 229E | 997 |
| 136 | IKNVNSVRDWLKSLK | 229E | 998 |
| 141 | FISVLDITDAAVKAA | 229E | 999 |
| 146 | DAGHSLTWLWLLCGL | 229E | 1000 |
| 151 | PEGCVLTNTGSVVKP | 229E | 1001 |
| 156 | ELLLALLAFFLSKHS | 229E | 1002 |

TABLE 9

124 Common Cold Coronavirus Epitopes homologous to C31 subset

| peptide sequence | Organism | category | SEQ ID NO: |
|---|---|---|---|
| ALDKLYKVFGSPVMT | 229E | common cold corona analogs | 1003 |
| APEGLVFLHTVLLPT | 229E | common cold corona analogs | 1004 |
| AWYFLAMLTGLLPSL | 229E | common cold corona analogs | 1005 |
| CAQYYNGIMVLPGVA | 229E | common cold corona analogs | 1006 |
| CVEMHNKINLCDDPE | 229E | common cold corona analogs | 1007 |
| EEIEYVHGDALHTLR | 229E | common cold corona analogs | 1008 |
| ELLQFVTDPTLIVAS | 229E | common cold corona analogs | 1009 |
| ESAYMPIADPTHFDI | 229E | common cold corona analogs | 1010 |
| FAVESGGYIPSDFAF | 229E | common cold corona analogs | 1011 |
| FSFGKVNNFVKFGSV | 229E | common cold corona analogs | 1012 |
| GLNKVKYATVVVGST | 229E | common cold corona analogs | 1013 |
| GNQTLFCNIMKFSDR | 229E | common cold corona analogs | 1014 |
| GNSLNHLTSQLRQNF | 229E | common cold corona analogs | 1015 |
| LLLWESGKAKPPLNR | 229E | common cold corona analogs | 1016 |
| NGSNILEAFTKPVFI | 229E | common cold corona analogs | 1017 |
| NPVSFVVKPVCSSIF | 229E | common cold corona analogs | 1018 |
| QAISSSIQAIYDRLD | 229E | common cold corona analogs | 1019 |
| QPVEGVSSFMNVTLD | 229E | common cold corona analogs | 1020 |
| QQVDRLITGRLAALN | 229E | common cold corona analogs | 1021 |
| RLHNFSIISGTAFLG | 229E | common cold corona analogs | 1022 |
| RVAGRSAIEDILFSK | 229E | common cold corona analogs | 1023 |
| SAIEDILFSKLVTSG | 229E | common cold corona analogs | 1024 |
| SNSNYLLEE FFFDVVFG | 229E | common cold corona analogs | 1025 |
| TGVNDAITQTSQALQ | 229E | common cold corona analogs | 1026 |
| TPFMILLVALSLCLT | 229E | common cold corona analogs | 1027 |
| TTQQAGAGIKYFCGM | 229E | common cold corona analogs | 1028 |
| VETYIKWPWWVWLCI | 229E | common cold corona analogs | 1029 |
| VMLQIQLTGILDGDY | 229E | common cold corona analogs | 1030 |
| VVGGTIQILASVPEK | 229E | common cold corona analogs | 1031 |
| VWHAKDFNSLSAEGR | 229E | common cold corona analogs | 1032 |
| WNGVIKNVNSVRDWL | 229E | common cold corona analogs | 1033 |
| APYGLLFMHFSYKPI | HKU1 | common cold corona analogs | 1034 |
| AQALNSLLQQLFNKF | HKU1 | common cold corona analogs | 1035 |
| AWRFPCAGRKVNFNE | HKU1 | common cold corona analogs | 1036 |
| CGIKYVAQPTEDVVD | HKU1 | common cold corona analogs | 1037 |
| CKFAVCGDGFVPFLL | HKU1 | common cold corona analogs | 1038 |
| CNAQEQQIYFFEGVA | HKU1 | common cold corona analogs | 1039 |
| CVQSFNGIKVLPPIL | HKU1 | common cold corona analogs | 1040 |
| DLLSEYGTFCDNINS | HKU1 | common cold corona analogs | 1041 |
| EDAIIVNDENSSIKV | HKU1 | common cold corona analogs | 1042 |
| ELSHWFKNQTSIAPN | HKU1 | common cold corona analogs | 1043 |
| ESQGNVVTSVMESQI | HKU1 | common cold corona analogs | 1044 |
| FEVEKGVTVDDFVAV | HKU1 | common cold corona analogs | 1045 |
| FKFDEPSDATDFIRV | HKU1 | common cold corona analogs | 1046 |
| FPKGYVMGLFRSYKT | HKU1 | common cold corona analogs | 1047 |
| GAISSSLQEILSRLD | HKU1 | common cold corona analogs | 1048 |
| GSSSRSFFEDLLFDK | HKU1 | common cold corona analogs | 1049 |
| IFKNNTSFPTNIAVE | HKU1 | common cold corona analogs | 1050 |
| IQESIKSLNNSYINL | HKU1 | common cold corona analogs | 1051 |
| KLITTACNGISVTQT | HKU1 | common cold corona analogs | 1052 |
| LIPRSYYLIQSGIFF | HKU1 | common cold corona analogs | 1053 |
| LLCRVTLGDFTIMSG | HKU1 | common cold corona analogs | 1054 |
| MLHGGGVAKAIAVAA | HKU1 | common cold corona analogs | 1055 |
| NFVALIPDYAKILVN | HKU1 | common cold corona analogs | 1056 |
| QPTEDVVDGDVVIRE | HKU1 | common cold corona analogs | 1057 |
| SFFEDLLFDKVKLSD | HKU1 | common cold corona analogs | 1058 |
| SVATFYIEHYVNRLV | HKU1 | common cold corona analogs | 1059 |
| VGGLYEIKIPTNFTI | HKU1 | common cold corona analogs | 1060 |
| VQIDRLINGRLTALN | HKU1 | common cold corona analogs | 1061 |
| WFNTMLDASAPATGW | HKU1 | common cold corona analogs | 1062 |
| YEMYVKWPWYVWLLI | HKU1 | common cold corona analogs | 1063 |
| YSFGRCPTSSIIKHP | HKU1 | common cold corona analogs | 1064 |
| APDGLLFLHTVLLPT | NL63 | common cold corona analogs | 1065 |
| ASGVFGVNLRTNFTI | NL63 | common cold corona analogs | 1066 |
| CAQYYNGIMVLPGVA | NL63 | common cold corona analogs | 1067 |
| CVDLHNKINLCDDPE | NL63 | common cold corona analogs | 1068 |
| DTCFGVSKPNAIDVE | NL63 | common cold corona analogs | 1069 |
| EFRDYFNNNTDSIVI | NL63 | common cold corona analogs | 1070 |
| FDVVFGHGAGSVVFV | NL63 | common cold corona analogs | 1071 |
| FENYIKWPWWVWLII | NL63 | common cold corona analogs | 1072 |
| FSPFNSLLCGDIVSG | NL63 | common cold corona analogs | 1073 |
| GLNASVTLKICKFSR | NL63 | common cold corona analogs | 1074 |
| GSALNHLTSQLRHNF | NL63 | common cold corona analogs | 1075 |
| GTFESAAAGTFVLDM | NL63 | common cold corona analogs | 1076 |
| GVFGVNLRTNFTIKG | NL63 | common cold corona analogs | 1077 |
| LFVVALFIGVSFIDY | NL63 | common cold corona analogs | 1078 |
| NIAFNVVKKGCFTGV | NL63 | common cold corona analogs | 1079 |
| NLLKQYTSACKTIED | NL63 | common cold corona analogs | 1080 |
| NNTDSIVIGGVTYQL | NL63 | common cold corona analogs | 1081 |
| NQLRLAFLGASVTED | NL63 | common cold corona analogs | 1082 |
| QPFSFSFRDELGVRV | NL63 | common cold corona analogs | 1083 |
| QQVDRLITGRLAALN | NL63 | common cold corona analogs | 1084 |
| RIAGRSALEDLLFSK | NL63 | common cold corona analogs | 1085 |
| SALEDLLFSKVVTSG | NL63 | common cold corona analogs | 1086 |
| SAMHSLLFGMLRRLD | NL63 | common cold corona analogs | 1087 |
| SLWRVTAVHSDGMFV | NL63 | common cold corona analogs | 1088 |
| SSATDAIIAVFTLLL | NL63 | common cold corona analogs | 1089 |
| STVVEVKSAIVCASV | NL63 | common cold corona analogs | 1090 |
| TLFKFLLLLYAIYAL | NL63 | common cold corona analogs | 1091 |
| VMNNIVLFLTWLLSM | NL63 | common cold corona analogs | 1092 |
| VTEDVKFAASTGVID | NL63 | common cold corona analogs | 1093 |
| WFCANQSTSVYSANG | NL63 | common cold corona analogs | 1094 |
| YFSQLLCEPIKLVNS | NL63 | common cold corona analogs | 1095 |
| AEALNNLLQQLSNRF | OC43 | common cold corona analogs | 1096 |
| APYGLYFIHFSYVPT | OC43 | common cold corona analogs | 1097 |
| AQIDRLINGRLTALN | OC43 | common cold corona analogs | 1098 |
| CQFKDKNLQDLWVLY | OC43 | common cold corona analogs | 1099 |
| CVQSYKGIKVLPPLL | OC43 | common cold corona analogs | 1100 |
| ELDQWFKNQTSVAPD | OC43 | common cold corona analogs | 1101 |
| ETFTVCADGFMPFLL | OC43 | common cold corona analogs | 1102 |
| GAISASLQEILSRLD | OC43 | common cold corona analogs | 1103 |
| GIFAKVKNTKVIKDR | OC43 | common cold corona analogs | 1104 |
| ICSASDMTNPDYTNL | OC43 | common cold corona analogs | 1105 |
| IFINNTTYPTNVAVE | OC43 | common cold corona analogs | 1106 |
| ISSTVRLQAGTATEY | OC43 | common cold corona analogs | 1107 |
| KASSRSAIEDLLFDK | OC43 | common cold corona analogs | 1108 |
| KRGEKGAYNKDHGRG | OC43 | common cold corona analogs | 1109 |
| LIREIVMNASPYDLE | OC43 | common cold corona analogs | 1110 |
| LLFDVIVAWHVVRDP | OC43 | common cold corona analogs | 1111 |
| LQEAIKVLNQSYINL | OC43 | common cold corona analogs | 1112 |
| LVPQENYSSIRFASV | OC43 | common cold corona analogs | 1113 |
| NIEAWLNDKSVPSPL | OC43 | common cold corona analogs | 1114 |
| NPPTNVVSHVNGDWF | OC43 | common cold corona analogs | 1115 |
| SAIEDLLFDKVKLSD | OC43 | common cold corona analogs | 1116 |
| SLGFYNPPTNVVSHV | OC43 | common cold corona analogs | 1117 |
| SQLVEYGSFCDNINA | OC43 | common cold corona analogs | 1118 |
| SVKSYDSLVYTGVLG | OC43 | common cold corona analogs | 1119 |
| SVVEVVTTSLTPCGY | OC43 | common cold corona analogs | 1120 |
| TIFNCVYALNNVYLG | OC43 | common cold corona analogs | 1121 |
| TILNTACGVFEVDDT | OC43 | common cold corona analogs | 1122 |
| VGGLYEIQIPSEFTI | OC43 | common cold corona analogs | 1123 |
| VKNIPRYASAVAQAF | OC43 | common cold corona analogs | 1124 |
| VSATVLQNNELMPAK | OC43 | common cold corona analogs | 1125 |
| YEYYVKWPWYVWLLI | OC43 | common cold corona analogs | 1126 |

### Vaccine Composition Embodiments

In certain embodiments described herein, constructs and compositions designed to induce optimal Neutralizing antibody and T cell activity against COVID targets are provided. These constructs and compositions are provided to elicit maximal focused neutralizing antibodies, plus CD4 and CD8 T cells.

### Example 1

In some embodiments, these constructs may encompass, by way of example and not by way of limitation, two components; component A and component B (E.g., SARS-COV-2 mRBD + nsp6).

Examples of component A are:

Example A.1: Membrane tethered RBD (mRBD). RBD-linker-spikeTM-dCT (spikeTM = spike transmembrane domain, dCT = deletion of the cytoplasmic tail as described in https://science.sciencemag.org/content/early/2020/05/19/science.abc6284.full). Example construct formula: RBD-short linker-PADRE-short linker-spikeTMdCT (underlined).

A.1

RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELL
HAPATVCGPKKSTNLVKNGGSGGGSGYEQYIKWPWYIWLGFIAGLIAIVM VTIMLCCMTSCCSCLK (SEQ ID NO: 1127)

Example A.2: Same as above, but with an added PADRE sequence (PADRE stands for synthetic Pan DR epitope, example sequence AKFVAAWTLKAAA (SEQ ID NO: 1128)(bold, as described at least in (www.ncbi.nlm.nih.gov/pmc/articles/PMC46405401) to make sure RBD has T cell help.

A.2

RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDI
STEIYQAGSTPCNGVEGFNCYFPLQSYGFQPTNGVGYQPYRVVVLSFELL
HAPATVCGPKKSTNLVKNGGSGGGSGAKFVAAWTLKAAAGGSGGGSGYEO YIKWPWYIWLGFIAGLIAIVMVTIMLCCMTSCCSCLK (SEQ ID NO:1
129)

Examples of component B are:

### 1.B.1.

N, M, ORF3a, or nsp6, or any combination thereof, either under the control of a second promoter or physically associated via a linker. The linker could have a 2A-protease-type sequence or not, to elicit an enhanced CD4 and CD8 T cell response.

Designs: the sequences for each protein - protein sequences derived from the SARS-CoV-2 reference (GenBank: MN908947). Example construct sequence to be provided. See protein sequences in “SARS-CoV-2 aa seq” below.

### 1.B.2

Same as above but including SARS-CoV-2 proteins modified to contain any of the 4 ‘common cold’ coronavirus sequences homologous to the identified SARS-COV-2 epitopes identified (selected from Table 8 and/or Table 9), from which the corresponding common cold corona virus sequences can be identified (e.g., taking an identified SARS-COV-2 epitope and modifying it to a homolog found in one of the common cold corona strains (229E, HKU1, NL63, OC43) - examples of such SARS-COV-2 peptides appear in Table 6 and Table 7, and their corresponding homologs appear in Table 8 and Table 9. An example is NRYFRLTLGVYDYLV (SEQ ID NO:836) selected from Table 6.

### Example 2. SARS-CoV-2 Spike Protein Sequence With Enhanced Activity by Incorporating Common Cold Corona T Cell Epitopes

In certain embodiments, the SARS-COV-2 spike protein is provided, with incorporated common cold corona epitopes that are cross-reactive with SARS-CoV-2, for example, those amino acid sequences provided in either Table 8 or Table 9. These compositions 1) to maximally recruit memory CD4 T cells to help the antibody responses, and 2) induce CD8 T cell responses.

Two general classes of constructs:

### 2.1

Constructs which include SARS-CoV-2 spike and additional T cell epitopes to the N- and C-termini of S. In the most preferred application 2-5 epitopes will be selected, from Table 6 or Table 8.

SARS-COV-2 S protein with 3CL epitope conjugated to C-term (SEQ ID NO:1130):

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQGSGYIPEAPRDGQAYVRKDGEWVLLSTFLGGSGGSNHNFLVQ AGNVQLRV

### 2.2.

Constructs which include SARS-COV-2 spike (Table 7) and additional ‘common cold’ epitope homologs to SARS-COV-2 spike epitopes (selected from Table 9) to recruit memory T cells.

S protein with OC43 and HKU1 T cell epitope replacement. (SEQ ID NO:1131)

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSAACK SQLVEYGSFCDNINRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNSLLQQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQGSGYIPEAPRDGQAYVRKDGEWVLLSTFL

### 2.3

Same as either 2.1 or 2.2 but incorporating additional CD4 and CD8 epitopes selected from Table 4 and/or Table 5.

### Example 3: SARS-CoV-2 Spike RBD With Enhanced T Cell Epitopes. A Construct Encompassing The Minimal SARS-COV-2 RBD Domain and Additional Cross-Reactive Epitopes Added

The RBD domain is the dominant target of neutralizing antibodies against SARS-COV-2 and is a relatively unique domain. However, it has limited T cell help.

As above, disclosed are two general classes of constructs;

### 3.1

Constructs with additional T cell epitopes to the N- and C-termini of RBD. (Same as above) Top 2-5 epitopes might be selected, from Table 6 and/or Table 8.

RBD with N-term ORF3a epitope, C-term 3CL epitope. (SEQ ID NO:1132)

SDFVRATATIPIQASRVQPTESIVRFPNITNLCPFGEVFNATRFASVYAW
NRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLY
RLFRKSNLKPFERDISTEIYQAGSTPCNGVEGFNCYFPLQSYGFQPTNGV
GYQPYRVVVLSFELLHAPATVCGPKKSTNLVKNGGSGGGSGNHNFLVOAG NVOLRV

### 3.2

Constructs which incorporate ‘common cold’ epitope homologs within the RBD sequence, to recruit memory T cells. (SEQ ID NO:1133)

RVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGKI
ADYNYRIDTTATSCQIAWNSNNLDSKVGGNYNYLYRLFRKSNLKPFERDI
STEIYQAGSTPCNGVEGFNCYFPLQSYGFQPTNGVGYQPYRVVVLSFELL
HAPATVCGPKKSTNLVKN

It is demonstrated herein that CD4 and CD8 T cell responses are present to many SARS-COV-2 proteins (Cell 2020). Most importantly, it is also shown that epitopes from Table 6 and Table 8 and the spike epitopes set forth in Tables 7 have all been shown to be recognized by human T cells as a pool or in isolation.

### 3.3

Same as either 3.1 or 3.2, but incorporating additional CD4 or CD8 epitopes selected from Table 4 and/or Table 5.

### Example 4. Constructs Incorporating Spike and Additional CD4 and CD8 Epitopes

### 4.1.

Constructs which encompass spike protein or RDB of spike protein from SARS-CoV-2, as described above, and a number of CD4 or CD8 epitopes derived from the remainder of the genome (e.g., those epitopes set forth in Table 4 and/or Table 5). The epitopes are delivered as minigenes, string of beads or other convenient modalities to deliver multiple identified epitopes described in the art.

### Example 5

Further embodiments comprise the prior embodiments (Examples 1-4), but with additional help. In some embodiments, the signal peptide MFVFLVLLPLVSSQ (SEQ ID NO:1134) is added to the C or N terminal end of the construct. In alternative embodiments, pSer (GGSGHHHHHHC) (SEQ ID NO:1135) is added to the C or N terminal end of the construct. In other embodiments, PADRE (AKFVAAWTLKAA) (SEQ ID NO:1136) is added to either or both the N and C terminal ends of any of the above embodiments. In further examples, the construct comprises an RBD trimer by trimerizing RBD with a foldon trimer domain.

SARS-CoV-2 aa seq:

### Membrane (M): 222 AA

NCBI Reference Sequence: YP_009724393.1 (SEQ ID NO:1137)

1 MADSNGTITV EELKKLLEQW NLVIGFLFLT WICLLQFAYA NRNRFLYIIK LIFLWLLWPV
61 TLACFVLAAV YRINWITGGI AIAMACLVGL MWLSYFIASF RLFARTRSMW SFNPETNILL
121 NVPLHGTILT RPLLESELVI GAVILRGHLR IAGHHLGRCD IKDLPKEITV ATSRTLSYYK
181 LGASQRVAGD SGFAAYSRYR IGNYKLNTDH SSSSDNIALL VQ

### Nucleocapsid (N): 419 AA

NCBI Reference Sequence: YP_009724397.2 (SEQ ID NO:1138)

1 MSDNGPQNQR NAPRITFGGP SDSTGSNQNG ERSGARSKQR RPQGLPNNTA SWFTALTQHG
61 KEDLKFPRGQ GVPINTNSSP DDQIGYYRRA TRRIRGGDGK MKDLSPRWYF YYLGTGPEAG
121 LPYGANKDGI IWVATEGALN TPKDHIGTRN PANNAAIVLQ LPQGTTLPKG FYAEGSRGGS
181 QASSRSSSRS RNSSRNSTPG SSRGTSPARM AGNGGDAALA LLLLDRLNQL
ESKMSGKGQQ
241 QQGQTVTKKS AAEASKKPRQ KRTATKAYNV TQAFGRRGPE QTQGNFGDQE
LIRQGTDYKH
301 WPQIAQFAPS ASAFFGMSRI GMEVTPSGTW LTYTGAIKLD DKDPNFKDQV ILLNKHIDAY
361 KTFPPTEPKK DKKKKADETQ ALPQRQKKQQ TVTLLPAADL DDFSKQLQQS MSSADSTQA

### Nsp6: 209 Aa; Processed From ORF1a and ORFlab

NCBI Reference Sequence: YP_009725302.1 (orf1ab) & NCBI Reference Sequence: YP_009742613.1 (orfla) (SEQ ID NO:1139)

1 SAVKRTIKGT HHWLLLTILT SLLVLVQSTQ WSLFFFLYEN AFLPFAMGII AMSAFAMMFV
61 KHKHAFLCLF LLPSLATVAY FNMVYMPASW VMRIMTWLDM VDTSLSGFKL
KDCVMYASAV
121 VLLILMTART VYDDGARRVW TLMNVLTLVY KVYYGNALDQ AISMWALIIS
VTSNYSGVVT
181 TVMFLARGIV FMCVEYCPIF FITGNTLQCI MLVYCFLGYF CTCYFGLFCL LNRYFRLTLG
241 VYDYLVSTQE FRYMNSQGLL PPKNSIDAFK LNIKLLGVGG KPCIKVATVQ

### ORF3a: 275 AA

NCBI Reference Sequence: YP_009724391.1 (SEQ ID NO:1140)

1 MDLFMRIFTI GTVTLKQGEI KDATPSDFVR ATATIPIQAS LPFGWLIVGV ALLAVFQSAS
61 KIITLKKRWQ LALSKGVHFV CNLLLLFVTV YSHLLLVAAG LEAPFLYLYA LVYFLQSINF
121 VRIIMRLWLC WKCRSKNPLL YDANYFLCWH TNCYDYCIPY NSVTSSIVIT SGDGTTSPIS
181 EHDYQIGGYT EKWESGVKDC VVLHSYFTSD YYQLYSTQLS TDTGVEHVTF FIYNKIVDEP
241 EEHVQIHTID GSSGVVNPVM EPIYDEPTTT TSVPL

### Common Cold HCoV Proteins (Amino Acid Sequences)

### Membrane (M):

### HCoV-229E

NCBI Reference Sequence: NP_073555.1 (225 aa) (SEQ ID NO:1141)

1 MSNDNCTGDI VTHLKNWNFG WNVILTIFIV ILQFGHYKYS RLFYGLKMLV LWLLWPLVLA
61 LSIFDTWANW DSNWAFVAFS FFMAVSTLVM WVMYFANSFR LFRRARTFWA
WNPEVNAITV
121 TTVLGQTYYQ PIQQAPTGIT VTLLSGVLYV DGHRLASGVQ VHNLPEYMTV AVPSTTIIYS
181 RVGRSVNSQN STGWVFYVRV KHGDFSAVSS PMSNMTENER LLHFF

### HCoV-HKU1

NCBI Reference Sequence: YP_173241.1 (223 aa) (SEQ ID NO:1142)

1 MNKSFLPQFT SDQAVTFLKE WNFSLGVILL FITIILQFGY TSRSMFVYLI KMIILWLMWP
61 LTITLTIFNC FYALNNAFLA FSIVFTIISI VIWILYFVNS IRLFIRTGSW WSFNPETNNL
121 MCIDMKGKMF VRPVIEDYHT LTATVIRGHL YIQGVKLGTG YTLSDLPVYV
TVAKVQVLCT
181 YKRAFLDKLD VNSGFAVFVK SKVGNYRLPS SKPSGMDTAL LRA

### HCoV-NL63

NCBI Reference Sequence: YP_003770.1 (226 aa) (SEQ ID NO:1143)

1 MSNSSVPLLE VYVHLRNWNF SWNLILTLFI VVLQYGHYKY SRLLYGLKMS
VLWCLWPLVL
61 ALSIFDCFVN FNVDWVFFGF SILMSIITLC LWVMYFVNSF RLWRRVKTFW AFNPETNAII
121 SLQVYGHNYY LPVMAAPTGV TLTLLSGVLL VDGHKIATRV QVGQLPKYVI VATPSTTIVC
181 DRVGRSVNET SQTGWAFYVR AKHGDFSGVA SQEGVLSERE KLLHLI

### HCoV-OC43

NCBI Reference Sequence: YP_009555244.1 (230 aa) (SEQ ID NO:1144)

1 MSSKTTPAPV YIWTADEAIK FLKEWNFSLG IILLFITIIL QFGYTSRSMF VYVIKMIILW
61 LMWPLTIILT IFNCVYALNN VYLGLSIVFT IVAIIMWIVY FVNSIRLFIR TGSFWSFNPE
121 TNNLMCIDMK GTMYVRPIIE DYHTLTVTII RGHLYIQGIK LGTGYSLADL PAYMTVAKVT
181 HLCTYKRGFL DRISDTSGFA VYVKSKVGNY RLPSTQKGSG MDTALLRNNI

### Nucleocapsid (N):

### HCoV-229E

NCBI Reference Sequence: NP_073556.1 (389 aa) (SEQ ID NO:1145)

1 MATVKWADAS EPQRGRQGRI PYSLYSPLLV DSEQPWKVIP RNLVPINKKD KNKLIGYWNV
61 QKRFRTRKGK RVDLSPKLHF YYLGTGPHKD AKFRERVEGV VWVAVDGAKT
EPTGYGVRRK
121 NSEPEIPHFN QKLPNGVTVV EEPDSRAPSR SQSRSQSRGR GESKPQSRNP SSDRNHNSQD
181 DIMKAVAAAL KSLGFDKPQE KDKKSAKTGT PKPSRNQSPA SSQTSAKSLA RSQSSETKEQ
241 KHEMQKPRWK RQPNDDVTSN VTQCFGPRDL DHNFGSAGVV ANGVKAKGYP
QFAELVPSTA
301 AMLFDSHIVS KESGNTVVLT FTTRVTVPKD HPHLGKFLEE LNAFTREMQQ HPLLNPSALE
361 FNPSQTSPAT AEPVRDEVSI ETDIIDEVN

### HCoV-HKU1

NCBI Reference Sequence: YP_173242.1 (441 aa) (SEQ ID NO:1146)

1 MSYTPGHYAG SRSSSGNRSG ILKKTSWADQ SERNYQTFNR GRKTQPKFTV STQPQGNTIP
61 HYSWFSGITQ FQKGRDFKFS DGQGVPIAFG VPPSEAKGYW YRHSRRSFKT ADGQQKQLLP
121 RWYFYYLGTG PYANASYGES LEGVFWVANH QADTSTPSDV SSRDPTTQEA IPTRFPPGTI
181 LPQGYYVEGS GRSASNSRPG SRSQSRGPNN RSLSRSNSNF RHSDSIVKPD MADEIANLVL
241 AKLGKDSKPQ QVTKQNAKEI RHKILTKPRQ KRTPNKHCNV QQCFGKRGPS
QNFGNAEMLK
301 LGTNDPQFPI LAELAPTPGA FFFGSKLDLV KRDSEADSPV KDVFELHYSG SIRFDSTLPG
361 FETIMKVLEE NLNAYVNSNQ NTDSDSLSSK PQRKRGVKQL PEQFDSLNLS AGTQHISNDF
421 TPEDHSLLAT LDDPYVEDSV A

### CoV-NL63

NCBI Reference Sequence: YP_003771.1 (377 aa) (SEQ ID NO:1147)

1 MASVNWADDR AARKKFPPPS FYMPLLVSSD KAPYRVIPRN LVPIGKGNKD
EQIGYWNVQE
61 RWRMRRGQRV DLPPKVHFYY LGTGPHKDLK FRQRSDGVVW VAKEGAKTVN
TSLGNRKRNQ
121 KPLEPKFSIA LPPELSVVEF EDRSNNSSRA SSRSSTRNNS RDSSRSTSRQ QSRTRSDSNQ
181 SSSDLVAAVT LALKNLGFDN QSKSPSSSGT STPKKPNKPL SQPRADKPSQ LKKPRWKRVP
241 TREENVIQCF GPRDFNHNMG DSDLVQNGVD AKGFPQLAEL IPNQAALFFD
SEVSTDEVGD
301 NVQITYTYKM LVAKDNKNLP KFIEQISAFT KPSSIKEMQS QSSHVAQNTV LNASIPESKP
361 LADDDSAIIE IVNEVLH

### HCoV-OC43

NCBI Reference Sequence: YP_009555245.1 (448 aa) (SEQ ID NO:1148)

1 MSFTPGKQSS SRASSGNRSG NGILKWADQS DQFRNVQTRG RRAQPKQTAT
SQQPSGGNVV
61 PYYSWFSGIT QFQKGKEFEF VEGQGVPIAP GVPATEAKGY WYRHNRRSFK
TADGNQRQLL
121 PRWYFYYLGT GPHAKDQYGT DIDGVYWVAS NQADVNTPAD IVDRDPSSDE
AIPTRFPPGT
181 VLPQGYYIEG SGRSAPNSRS TSRTSSRASS AGSRSRANSG NRTPTSGVTP DMADQIASLV
241 LAKLGKDATK PQQVTKHTAK EVRQKILNKP RQKRSPNKQC TVQQCFGKRG
PNQNFGGGEM
301 LKLGTSDPQF PILAELAPTA GAFFFGSRLE LAKVQNLSGN PDEPQKDVYE LRYNGAIRFD
361 STLSGFETIM KVLNENLNAY QQQDGMMNMS PKPQRQRGHK NGQGENDNIS
VAVPKSRVQQ
421 NKSRELTAED ISLLKKMDEP YTEDTSEI

### Non-Structural Protein 6 (nsp6):

### HCoV-229E

NCBI Reference Sequence: NP_073549.1 (6758 aa) polyprotein 1ab (SEQ ID NO:1149)

UniProt (Predicted Nsp6; 279 AA)

>sp|P0C6X1|3268-3546

SGKTTSMFKSISLFAGFFVMFWAELFVYTTTIWVNPGFLTPFMILLVALS
LCLTFVVKHKVLFLQVFLLPSIIVAAIQNCAWDYHVTKVLAEKFDYNVSV
MQMDIQGFVNIFICLFVALLHTWRFAKERCTHWCTYLFSLIAVLYTALYS
YDYVSLLVMLLCAISNEWYIGAIIFRICRFGVAFLPVEYVSYFDGVKTVL
LFYMLLGFVSCMYYGLLYWINRFCKCTLGVYDFCVSPAEFKYMVANGLNA
PNGPFDALFLSFKLMGIGGPRTIKVSTVQ

### HCoV-HKU1

NCBI Reference Sequence: YP_009742613.1 (287 aa) hydrophobic domain (SEQ ID NO:1150)

1 SKTKRFIKET IYWILISTFL FSCIISAFVK WTIFMYINTH MIGVTLCVLC FVSFMMLLVK
61 HKHFYLTMYI IPVLCTLFYV NYLVVYKEGF RGFTYVWLSY FVPAVNFTYV YEVFYGCILC
121 VFAIFITMHS INHDIFSLMF LVGRIVTLIS MWYFGSNLEE DVLLFITAFL GTYTWTTILS
181 LAIAKIVANW LSVNIFYFTD VPYIKLILLS YLFIGYILSC YWGFFSLLNS VFRMPMGVYN
241 YKISVQELRY MNANGLRPPRNSFEAILLNL KLLGIGGVPV IEVSQIQ

### HCoV-NL63

NCBI Reference Sequence: YP_003766.2 (6729 aa) polyprotein 1ab (SEQ ID NO:1151)

### UniProt (predicted Nsp6; 279 Aa)

>sp|P0C6X5|3243-3521

SGKVIFGLKTMFLFSVFFTMFWAELFIYTNTIWINPVILTPIFCLLLFLS
LVLTMFLKHKFLFLQVFLLPTVIATALYNCVLDYYIVKFLADHFNYNVSV
LQMDVQGLVNVLVCLFVVFLHTWRFSKERFTHWFTYVCSLIAVAYTYFYS
GDFLSLLVMFLCAISSDWYIGAIVFRLSRLIVFFSPESVFSVFGDVKLTL
VVYLICGYLVCTYWGILYWFNRFFKCTMGVYDFKVSAAEFKYMVANGLHA
PHGPFDALWLSFKLLGIGGDRCIKISTVQ

### HCoV-OC43

NCBI Reference Sequence: YP_009555252.1 (110 aa) (SEQ ID NO:1152)

1 NNELMPAKLK IQVVNSGPDQ TCNTPTQCYY NNSNNGKIVY AILSDVDGLK YTKILKDDGN
61 FVVLELDPPC KFTVQDAKGL KIKYLYFVKG CNTLARGWVV GTISSTVRLQ

### ORF3a:

### HCoV-229E

ORF4a* NCBI Reference Sequence: NP_073552.1 (133 aa) (SEQ ID NO:1153)

1 MALGLFTLQL VSAVNQSLSN AKVSAEVSRQ VIQDVKDGTV TFNLLAYTLM SLFVVYFALF
61 KARSHRGRAA LIVFKILILF VYVPLLYWSQ AYIYATLIAV ILLGRFFHTA WHCWLYKTWD
121 FIVFNVTTLC YAR

### HCoV-HKU1

NCBI Reference Sequence: (aa) NONE

### HCoV-NL63

NCBI Reference Sequence: YP_003768.1 (225 aa) (SEQ ID NO:1154)

1 MPFGGLFQLT LESTINKSVA NLKLPPHDVT VLRDNLKPVT TLSTITAYLL VSLFVTYFAL
61 FKPLTARGRV ACFVLKLLTL FVYVPLLVLF GMYLDSFIIF STLLFRFIHV GYYAYLYKNF
121 SFVLFNVTKL CFVSGKCWYL EQSFYENRFA AIYGGDHYVV LGGETITFVS FDDLYVAIRG
181 SCEKNLQLMR KVDLYNGAVI YIFAEEPVVG IVYSSQLYED VPSIN

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Altmann, D.M., and Boyton, R.J. (2020). SARS-CoV-2 T cell immunity: Specificity, function, durability, and role in protection. Science immunology 5.

Barra, C., Alvarez, B., Paul, S., Sette, A., Peters, B., Andreatta, M., Buus, S., and Nielsen, M. (2018). Footprints of antigen processing boost MHC class II natural ligand predictions. Genome medicine 10, 84.

Braun, J., Loyal, L., Frentsch, M., Wendisch, D., Georg, P., Kurth, F., Hippenstiel, S., Dingeldey, M., Kruse, B., Fauchere, F., et al. (2020). SARS-CoV-2-reactive T cells in healthy donors and patients with COVID-19. Nature.

Bui, H.H., Sidney, J., Dinh, K., Southwood, S., Newman, M.J., and Sette, A. (2006). Predicting population coverage of T-cell epitope-based diagnostics and vaccines. BMC Bioinformatics 7, 153.

Cai, Y., Zhang, J., Xiao, T., Peng, H., Sterling, S.M., Walsh, R.M., Jr., Rawson, S., Rits-Volloch, S., and Chen, B. (2020). Distinct conformational states of SARS-CoV-2 spike protein. Science 369, 1586-1592.

Carrasco Pro, S., Sidney, J., Paul, S., Lindestam Arlehamn, C., Weiskopf, D., Peters, B., and Sette, A. (2015). Automatic Generation of Validated Specific Epitope Sets. Journal of immunology research 2015, 763461.

Cassotta, A., Paparoditis, P., Geiger, R., Mettu, R.R., Landry, S.J., Donati, A., Benevento, M., Foglierini, M., Lewis, D.J.M., Lanzavecchia, A., et al. (2020). Deciphering and predicting CD4+ T cell immunodominance of influenza virus hemagglutinin. J Exp Med 217.

da Silva Antunes, R., Quiambao, L.G., Sutherland, A., Soldevila, F., Dhanda, S.K., Armstrong, S.K., Brickman, T.J., Merkel, T., Peters, B., and Sette, A. (2020). Development and Validation of a Bordetella pertussis Whole-Genome Screening Strategy. Journal of immunology research 2020, 8202067.

Dan, J.M., Lindestam Arlehamn, C.S., Weiskopf, D., da Silva Antunes, R., Havenar-Daughton, C., Reiss, S.M., Brigger, M., Bothwell, M., Sette, A., and Crotty, S. (2016). A Cytokine-Independent Approach To Identify Antigen-Specific Human Germinal Center T Follicular Helper Cells and Rare Antigen-Specific CD4+ T Cells in Blood. J Immunol 197, 983-993.

Dhanda, S.K., Mahajan, S., Paul, S., Yan, Z., Kim, H., Jespersen, M.C., Jurtz, V., Andreatta, M., Greenbaum, J.A., Marcatili, P., et al. (2019). IEDB-AR: immune epitope database-analysis resource in 2019. Nucleic Acids Res 47, W502-W506.

Dhanda, S.K., Vita, R., Ha, B., Grifoni, A., Peters, B., and Sette, A. (2018). ImmunomeBrowser: a tool to aggregate and visualize complex and heterogeneous epitopes in reference proteins. Bioinformatics 34, 3931-3933.

Ferretti, A.P., Kula, T., Wang, Y., Nguyen, D.M.V., Weinheimer, A., Dunlap, G.S., Xu, Q., Nabilsi, N., Perullo, C.R., Cristofaro, A.W., et al. (2020). Unbiased Screens Show CD8(+) T Cells of COVID-19 Patients Recognize Shared Epitopes in SARS-CoV-2 that Largely Reside outside the Spike Protein. Immunity.

Gonzalez-Galarza, F.F., McCabe, A., Santos, E., Jones, J., Takeshita, L., Ortega-Rivera, N.D., Cid-Pavon, G.M.D., Ramsbottom, K., Ghattaoraya, G., Alfirevic, A., et al. (2020). Allele frequency net database (AFND) 2020 update: gold-standard data classification, open access genotype data and new query tools. Nucleic Acids Res 48, D783-D788.

Goulder, P.J., Phillips, R.E., Colbert, R.A., McAdam, S., Ogg, G., Nowak, M.A., Giangrande, P., Luzzi, G., Morgan, B., Edwards, A., et al. (1997). Late escape from an immunodominant cytotoxic T-lymphocyte response associated with progression to AIDS. Nat Med 3, 212-217.

Greenbaum, J., Sidney, J., Chung, J., Brander, C., Peters, B., and Sette, A. (2011). Functional classification of class II human leukocyte antigen (HLA) molecules reveals seven different supertypes and a surprising degree of repertoire sharing across supertypes. Immunogenetics 63, 325-335.

Grifoni, A., Angelo, M.A., Lopez, B., O’Rourke, P.H., Sidney, J., Cerpas, C., Balmaseda, A., Silveira, C.G.T., Maestri, A., Costa, P.R., et al. (2017). Global Assessment of Dengue Virus-Specific CD4+ T Cell Responses in Dengue-Endemic Areas. Front Immunol 8, 1309.

Grifoni, A., Weiskopf, D., Ramirez, S.I., Mateus, J., Dan, J.M., Moderbacher, C.R., Rawlings, S.A., Sutherland, A., Premkumar, L., Jadi, R.S., et al. (2020). Targets of T Cell Responses to SARS-CoV-2 Coronavirus in Humans with COVID-19 Disease and Unexposed Individuals. Cell.

Jurtz, V., Paul, S., Andreatta, M., Marcatili, P., Peters, B., and Nielsen, M. (2017). NetMHCpan-4.0: Improved Peptide-MHC Class I Interaction Predictions Integrating Eluted Ligand and Peptide Binding Affinity Data. J Immunol 199, 3360-3368.

Karosiene, E., Rasmussen, M., Blicher, T., Lund, O., Buus, S., and Nielsen, M. (2013). NetMHCIIpan-3.0, a common pan-specific MHC class II prediction method including all three human MHC class II isotypes, HLA-DR, HLA-DP and HLA-DQ. Immunogenetics 65, 711-724.

Keller, M.D., Harris, K.M., Jensen-Wachspress, M.A., Kankate, V., Lang, H., Lazarski, C.A., Durkee-Shock, J.R., Lee, P.H., Chaudhry, K., Webber, K., et al. (2020). SARS-CoV-2 specific T-cells Are Rapidly Expanded for Therapeutic Use and Target Conserved Regions of Membrane Protein. Blood.

Kelley, L.A., and Sternberg, M.J. (2009). Protein structure prediction on the Web: a case study using the Phyre server. Nature protocols 4, 363-371.

Kim, Y., Yewdell, J.W., Sette, A., and Peters, B. (2013). Positional bias of MHC class I restricted T-cell epitopes in viral antigens is likely due to a bias in conservation. PLoS Comput Biol 9, e1002884.

Land, H., and Humble, M.S. (2018). YASARA: A Tool to Obtain Structural Guidance in Biocatalytic Investigations. Methods Mol Biol 1685, 43-67.

Le Bert, N., Tan, A.T., Kunasegaran, K., Tham, C.Y.L., Hafezi, M., Chia, A., Chng, M.H.Y., Lin, M., Tan, N., Linster, M., et al. (2020). SARS-CoV-2-specific T cell immunity in cases of COVID-19 and SARS, and uninfected controls. Nature.

Lindestam Arlehamn, C.S., Gerasimova, A., Mele, F., Henderson, R., Swann, J., Greenbaum, J.A., Kim, Y., Sidney, J., James, E.A., Taplitz, R., et al. (2013). Memory T cells in latent Mycobacterium tuberculosis infection are directed against three antigenic islands and largely contained in a CXCR3+CCR6+ Th1 subset. PLoS Pathog 9, e1003130.

Lindestam Arlehamn, C.S., McKinney, D.M., Carpenter, C., Paul, S., Rozot, V., Makgotlho, E., Gregg, Y., van Rooyen, M., Ernst, J.D., Hatherill, M., et al. (2016). A Quantitative Analysis of Complexity of Human Pathogen-Specific CD4 T Cell Responses in Healthy M. tuberculosis Infected South Africans. PLoS Pathog 12, e1005760.

Mateus, J., Grifoni, A., Tarke, A., Sidney, J., Ramirez, S.I., Dan, J.M., Burger, Z.C., Rawlings, S.A., Smith, D.M., Phillips, E., et al. (2020). Selective and cross-reactive SARS-CoV-2 T cell epitopes in unexposed humans. Science 370, 89-94.

Meckiff, B.J., Ramirez-Suastegui, C., Fajardo, V., Chee, S.J., Kusnadi, A., Simon, H., Eschweiler, S., Grifoni, A., Pelosi, E., Weiskopf, D., et al. (2020). Imbalance of Regulatory and Cytotoxic SARS-CoV-2-Reactive CD4(+) T Cells in COVID-19. Cell.

Nelde, A., Bilich, T., Heitmann, J.S., Maringer, Y., Salih, H.R., Roerden, M., Lubke, M., Bauer, J., Rieth, J., Wacker, M., et al. (2020). SARS-CoV-2-derived peptides define heterologous and COVID-19-induced T cell recognition. Nat Immunol.

Oseroff, C., Sidney, J., Kotturi, M.F., Kolla, R., Alam, R., Broide, D.H., Wasserman, S.I., Weiskopf, D., McKinney, D.M., Chung, J.L., et al. (2010). Molecular determinants of T cell epitope recognition to the common Timothy grass allergen. J Immunol 185, 943-955.

Paul, S., Grifoni, A., Peters, B., and Sette, A. (2019). Major Histocompatibility Complex Binding, Eluted Ligands, and Immunogenicity: Benchmark Testing and Predictions. Frontiers in immunology 10, 3151.

Paul, S., Karosiene, E., Dhanda, S.K., Jurtz, V., Edwards, L., Nielsen, M., Sette, A., and Peters, B. (2018). Determination of a Predictive Cleavage Motif for Eluted Major Histocompatibility Complex Class II Ligands. Front Immunol 9, 1795.

Paul, S., Weiskopf, D., Angelo, M.A., Sidney, J., Peters, B., and Sette, A. (2013). HLA class I alleles are associated with peptide-binding repertoires of different size, affinity, and immunogenicity. J Immunol 191, 5831-5839.

Peng, Y., Mentzer, A.J., Liu, G., Yao, X., Yin, Z., Dong, D., Dejnirattisai, W., Rostron, T., Supasa, P., Liu, C., et al. (2020). Broad and strong memory CD4(+) and CD8(+) T cells induced by SARS-CoV-2 in UK convalescent individuals following COVID-19. Nat Immunol 21, 1336-1345.

Peters, B., Nielsen, M., and Sette, A. (2020). T Cell Epitope Predictions. Annu Rev Immunol 38, 123-145.

Reiss, S., Baxter, A.E., Cirelli, K.M., Dan, J.M., Morou, A., Daigneault, A., Brassard, N., Silvestri, G., Routy, J.P., Havenar-Daughton, C., et al. (2017). Comparative analysis of activation induced marker (AIM) assays for sensitive identification of antigen-specific CD4 T cells. PLoS One 12, e0186998.

Rydyznski Moderbacher, C., Ramirez, S.I., Dan, J.M., Grifoni, A., Hastie, K.M., Weiskopf, D., Belanger, S., Abbott, R.K., Kim, C., Choi, J., et al. (2020). Antigen-Specific Adaptive Immunity to SARS-CoV-2 in Acute COVID-19 and Associations with Age and Disease Severity. Cell.

Schub, D., Klemis, V., Schneitler, S., Mihm, J., Lepper, P.M., Wilkens, H., Bals, R., Eichler, H., Gartner, B.C., Becker, S.L., et al. (2020). High levels of SARS-CoV-2-specific T cells with restricted functionality in severe courses of COVID-19. JCI insight 5.

Sekine, T., Perez-Potti, A., Rivera-Ballesteros, O., Stralin, K., Gorin, J.B., Olsson, A., Llewellyn-Lacey, S., Kamal, H., Bogdanovic, G., Muschiol, S., et al. (2020). Robust T Cell Immunity in Convalescent Individuals with Asymptomatic or Mild COVID-19. Cell 183, 158-168 e114.

Sette, A., Moutaftsi, M., Moyron-Quiroz, J., McCausland, M.M., Davies, D.H., Johnston, R.J., Peters, B., Rafii-El-Idrissi Benhnia, M., Hoffmann, J., Su, H.P., et al. (2008). Selective CD4+ T cell help for antibody responses to a large viral pathogen: deterministic linkage of specificities. Immunity 28, 847-858.

Shrock, E., Fujimura, E., Kula, T., Timms, R.T., Lee, I.H., Leng, Y., Robinson, M.L., Sie, B.M., Li, M.Z., Chen, Y., et al. (2020). Viral epitope profiling of COVID-19 patients reveals cross-reactivity and correlates of severity. Science.

Sidney, J., Southwood, S., Moore, C., Oseroff, C., Pinilla, C., Grey, H.M., and Sette, A. (2013). Measurement of MHC/peptide interactions by gel filtration or monoclonal antibody capture. Curr Protoc Immunol Chapter 18, Unit 18 13.

Snyder, T.M., Gittelman, R.M., Klinger, M., May, D.H., Osborne, E.J., Taniguchi, R., Zahid, H.J., Kaplan, I.M., Dines, J.N., Noakes, M.N., et al. (2020). Magnitude and Dynamics of the T-Cell Response to SARS-CoV-2 Infection at Both Individual and Population Levels. medRxiv.

Southwood, S., Sidney, J., Kondo, A., del Guercio, M.F., Appella, E., Hoffman, S., Kubo, R.T., Chesnut, R.W., Grey, H.M., and Sette, A. (1998). Several common HLA-DR types share largely overlapping peptide binding repertoires. J Immunol 160, 3363-3373.

Stadlbauer, D., Amanat, F., Chromikova, V., Jiang, K., Strohmeier, S., Arunkumar, G.A., Tan, J., Bhavsar, D., Capuano, C., Kirkpatrick, E., et al. (2020). SARS-CoV-2 Seroconversion in Humans: A Detailed Protocol for a Serological Assay, Antigen Production, and Test Setup. Curr Protoc Microbiol 57, e100.

Weiskopf, D., Angelo, M.A., de Azeredo, E.L., Sidney, J., Greenbaum, J.A., Fernando, A.N., Broadwater, A., Kolla, R.V., De Silva, A.D., de Silva, A.M., et al. (2013). Comprehensive analysis of dengue virus-specific responses supports an HLA-linked protective role for CD8+ T cells. Proc Natl Acad Sci U S A 110, E2046-2053.

Weiskopf, D., Cerpas, C., Angelo, M.A., Bangs, D.J., Sidney, J., Paul, S., Peters, B., Sanches, F.P., Silvera, C.G., Costa, P.R., et al. (2015). Human CD8+ T-Cell Responses Against the 4 Dengue Virus Serotypes Are Associated With Distinct Patterns of Protein Targets. J Infect Dis 212, 1743-1751.

Weiskopf, D., Schmitz, K.S., Raadsen, M.P., Grifoni, A., Okba, N.M.A., Endeman, H., van den Akker, J.P.C., Molenkamp, R., Koopmans, M.P.G., van Gorp, E.C.M., et al. (2020). Phenotype and kinetics of SARS-CoV-2-specific T cells in COVID-19 patients with acute respiratory distress syndrome. Science immunology 5.

Xie, X., Muruato, A., Lokugamage, K.G., Narayanan, K., Zhang, X., Zou, J., Liu, J., Schindewolf, C., Bopp, N.E., Aguilar, P.V., et al. (2020). An Infectious cDNA Clone of SARS-CoV-2. Cell host & microbe 27, 841-848 e843.

Zeng, W., Liu, G., Ma, H., Zhao, D., Yang, Y., Liu, M., Mohammed, A., Zhao, C., Yang, Y., Xie, J., et al. (2020). Biochemical characterization of SARS-CoV-2 nucleocapsid protein. Biochem Biophys Res Commun 527, 618-623.

What is claimed is:

1-157. (canceled)

158. A composition comprising monomers or multimers of:
one or more peptides or proteins comprising, consisting of, or consisting essentially of:
one or more SARS-CoV-2 amino acid sequences selected from SEQ ID NO: 1 to 1126, concatemers, subsequences, portions, homologues, variants or derivatives thereof;
a fusion protein comprising one or more amino acid sequences selected from SEQ ID NO: 1 to 1126.

159. The composition of claim 158, wherein the one or more peptides or proteins or the fusion protein comprises, 22, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125, or more amino acid sequences selected from SEQ ID NO: 1 to 1126, or a subsequence, portion, homologue, variant or derivative thereof.

160. The composition of claim 158, wherein the one or more protein or peptide comprises at least one of: a SARS-CoV-2 T cell epitope, a SARS-CoV-2 CD8+ or CD4+ T cell epitope, the SARS-CoV-2 T cell epitope is not conserved in another coronavirus, the SARS-CoV-2 T cell epitope is conserved in another coronavirus, or the one or more peptides or proteins exclude the amino acid sequences selected from SEQ ID NOS: 245-280 and 804-873.

161. The composition of claim 158, wherein one or more peptides or proteins has a length from about 9-15, 15-20, 20-25, 25-30, 30-40, 40-50, 50-75 or 75-100 amino acids.

162. The composition of claim 158, wherein the one or more peptides or proteins elicits, stimulates, induces, promotes, increases or enhances a T cell response to SARS-CoV-2, or wherein the one or more peptides or proteins that elicits, stimulates, induces, promotes, increases or enhances the T cell response to SARS-CoV-2 is a SARS-CoV-2 spike, nucleoprotein, membrane, replicase polyprotein 1ab, protein 3a, envelope small membrane protein, non-structural protein 3b, protein 7a, protein 9b, non-structural protein 6, or non-structural protein 8a protein or peptide, or a variant, homologue, derivative or subsequence thereof.

163. The composition of claim 158, further comprising formulating the one or more peptides or proteins into an immunogenic formulation with an adjuvant, wherein the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund’s complete adjuvant, Freund’s incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR⅞ ligands, and TLR9 ligands.

164. The composition of claim 158, wherein the composition further comprises a modulator of an immune response, an innate immune response, or the modulator is Interleukin-6 (IL-6), Interferon-gamma (IFN-g), Transforming growth factor beta (TGF-B), Interleukin-10 (IL-10), or an agonist or antagonist thereof.

165. A method for detecting the presence of: (i) SARS-CoV-2 or (ii) an immune response relevant to SARS-CoV-2 infections, vaccines or therapies, including T cells responsive to one or more SARS-CoV-2 peptides, comprising:
providing one or more proteins or peptides for detection of an amount or a relative amount of, and/or the activity of, and/or the state of antigen-specific T-cells;
contacting a biological sample suspected of having SARS-CoV-2-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in SEQ ID NO: 1 to 873, or comprise a pool of 22, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125, or more amino acid sequences set forth in SEQ ID NO: 1 to 1126.

166. A kit for the detection of SARS-CoV-2 or an immune response to SARS-CoV-2 in a subject comprising:
one or more T cells that specifically detect the presence of:
one or more amino acid sequences selected from SEQ ID NO: 1 to 1126, or a subsequence, portion, homologue, variant or derivative thereof;
a fusion protein comprising one or more amino acid sequences selected from SEQ ID NO: 1 to 1126; or
a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 more peptides selected from the amino acid sequences set forth in SEQ ID NO: 1 to 1126.

167. The kit of claim 166, wherein the one or more amino acid sequences are selected from at least one of:
a coronavirus T cell epitope set forth in SEQ ID NO:874 to 1126; wherein the one or more amino acid sequences comprises:
one or more amino acid sequences selected from SEQ ID NO: 1 to 873, or a subsequence, portion, homologue, variant or derivative thereof;
a fusion protein comprising one or more amino acid sequences selected from SEQ ID NO: 1 to 873;
a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, or 873 more peptides selected from the amino acid sequences set forth in SEQ ID NO: 1 to 873; or
the fusion protein has a length from about 9-15, 15-20, 20-25, 25-30, 30-40, 40-50, 50-75 or 75-100 amino acids
a coronavirus CD8+ or CD4+ T cell epitope;
the T cell epitope is not conserved in another coronavirus; or
the T cell epitope is conserved in another coronavirus.

168. The kit of claim 166, wherein the kit includes instruction for a diagnostic method, a process, a composition, a product, a service or component part thereof for the detection of: (i) coronavirus or (ii) an immune response relevant to coronavirus infections, vaccines or therapies, including T cells responsive to coronavirus.

169. The kit of claim 166, wherein the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

170. The kit of claim 166, wherein the kit includes reagents for determining a Human Leukocyte Antigen (HLA) profile of a subject, and selecting peptides that are presented by the HLA profile of the subject for detecting an immune response to coronavirus.

171. A method of stimulating, inducing, promoting, increasing, or enhancing an immune response against SARS-CoV-2 in a subject, comprising:
administering to a subject an amount of a protein or peptide or a polynucleotide that expresses the protein or peptide comprising an amino acid sequence of the SARS-CoV-2 spike, nucleoprotein, membrane, replicase polyprotein 1ab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two peptides selected from the amino acid sequences set forth in SEQ ID NO:1 to 1126 or a subsequence, portion, homologue, variant or derivative thereof, in an amount sufficient to prevent, stimulate, induce, promote, increase, immunize against, or enhance an immune response against SARS-CoV-2 in the subject.

172. The method of claim 171, wherein the immune response provides the subject with protection against SARS-CoV-2 infection or pathology, or one or more physiological conditions, disorders, illnesses, diseases or symptoms caused by or associated with SARS-CoV-2 infection or pathology, reduces SARS-CoV-2 viral titer, increases or stimulates SARS-CoV-2 viral clearance, reduces or inhibits SARS-CoV-2 viral proliferation, reduces or inhibits increases in SARS-CoV-2 viral titer or SARS-CoV-2 viral proliferation, reduces the amount of a SARS-CoV-2 viral protein or the amount of a SARS-CoV-2 viral nucleic acid, or reduces or inhibits synthesis of a SARS-CoV-2 viral protein or a SARS-CoV-2 viral nucleic acid the SARS-CoV-2 infection is an acute infection, or the subject is a mammal or a human, or the immune response is to two or more circulating forms of SARS-CoV-2 or two or more coronaviruses.

173. The method of claim 171, wherein the one or more amino acid sequences are selected from at least one of:
the one or more peptides selected from SEQ ID NO: 1 to 1126, or a subsequence, portion, homologue, variant or derivative thereof;
a fusion protein comprising one or more amino acid sequences selected from SEQ ID NO: 1 to 1126; or
a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 more peptides selected from the amino acid sequences set forth in SEQ ID NO: 1 to 1126.
the anti-SARS-CoV-2 T cell response is a CD8+, a CD4+ T cell response, or both; or
the T cell epitope is conserved across two or more clinical isolates of SARS-CoV-2.

174. The method of claim 171, wherein the composition further comprising formulating the one or more peptides or proteins into an immunogenic formulation with an adjuvant, wherein the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund’s complete adjuvant, Freund’s incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR⅞ ligands, and TLR9 ligands, or a modulator of an immune response, an innate immune response, or the modulator is Interleukin-6 (IL-6), Interferon-gamma (IFN-y), Transforming growth factor beta (TGF-β), Interleukin-10 (IL-10), or an agonist or antagonist thereof.

175. A polynucleotide that expresses one or more peptides or proteins, comprising, an amino acid sequence selected from SEQ ID NO: 1 to 1126, or a subsequence, portion, homologue, variant or derivative thereof;
a fusion protein comprising one or more amino acid sequences selected from SEQ ID NO: 1 to 1126; or
a pool of 2, 3, 4, 5, 6, 7, 8, 9, 10, 15, 20, 25, 30, 40, 50, 60, 75, 80, 90, 100, 125, 150, 175, 200, 225, 250, 275, 300, 350, 400, 450, 500, 600, 700, 800, 900, 1000, 1100, 1125 or more peptides comprising, consisting of, or consisting essentially of amino acid sequences selected from SEQ ID NO: 1 to 1126.

176. A vector that comprises the polynucleotide of claim 175.

177. The vector of claim 176, wherein the vector is a viral vector.

178. A host cell that comprises the vector of claim 176.


SEQUENCE LISTING (1154 sequences)

SEQ ID NO: 1 - protein, 15 aa, Artificial Sequence
  Synthetic peptide
YRINWITGGIAIAMA

SEQ ID NO: 2 - protein, 15 aa, Artificial Sequence
  Synthetic peptide
PCPIHFYSKWYIRVG

SEQ ID NO: 3 - protein, 15 aa, Artificial Sequence
  Synthetic peptide
KEITVATSRTLSYYK

SEQ ID NO: 4 - protein, 15 aa, Artificial Sequence
  Synthetic peptide
LSYYKLGASQRVAGD

SEQ ID NO: 5 - protein, 15 aa, Artificial Sequence
  Synthetic peptide
IGNYTVSCLPFTINC

SEQ ID NO: 6 - protein, 15 aa, Artificial Sequence
  Synthetic peptide
NIDGYFKIYSKHTPI

SEQ ID NO: 7 - protein, 15 aa, Artificial Sequence
  Synthetic peptide
VLAAVYRINWITGGI

SEQ ID NO: 8 - protein, 15 aa, Artificial Sequence
  Synthetic peptide
RGHLRIAGHHLGRCD

SEQ ID NO: 9 - protein, 15 aa, Artificial Sequence
  Synthetic peptide
ILLNKHIDAYKTFPP

SEQ ID NO: 10 - protein, 15 aa, Artificial Sequence
  Synthetic peptide
TQHQPYVVDDPCPIH

SEQ ID NO: 11 - protein, 15 aa, Artificial Sequence
  Synthetic peptide
YVVDDPCPIHFYSKW

SEQ ID NO: 12 - protein, 15 aa, Artificial Sequence
  Synthetic peptide
RCSFYEDFLEYHDVR

SEQ ID NO: 13 - protein, 15 aa, Artificial Sequence
  Synthetic peptide
LMDLEGKQGNFKNLR

SEQ ID NO: 14 - protein, 15 aa, Artificial Sequence
  Synthetic peptide
CLVGLMWLSYFIASF

SEQ ID NO: 15 - protein, 15 aa, Artificial Sequence
  Synthetic peptide
SELVIGAVILRGHLR

SEQ ID NO: 16 - protein, 15 aa, Artificial Sequence
  Synthetic peptide
WPQIAQFAPSASAFF

SEQ ID NO: 17 - protein, 15 aa, Artificial Sequence
  Synthetic peptide
FKDQVILLNKHIDAY

SEQ ID NO: 18 - protein, 15 aa, Artificial Sequence
  Synthetic peptide
AVMSLKEGQINDMIL

SEQ ID NO: 19 - protein, 15 aa, Artificial Sequence
  Synthetic peptide
KEGQINDMILSLLSK

SEQ ID NO: 20 - protein, 15 aa, Artificial Sequence
  Synthetic peptide
RENNRVVISSDVLVN

SEQ ID NO: 21 - protein, 15 aa, Artificial Sequence
  Synthetic peptide
SGFAAYSRYRIGNYK

SEQ ID NO: 22 - protein, 15 aa, Artificial Sequence
  Synthetic peptide
KKPRQKRTATKAYNV

SEQ ID NO: 23 - protein, 15 aa, Artificial Sequence
  Synthetic peptide
KRTATKAYNVTQAFG

SEQ ID NO: 24 - protein, 15 aa, Artificial Sequence
  Synthetic peptide
NKDGIIWVATEGALN

SEQ ID NO: 25 - protein, 15 aa, Artificial Sequence
  Synthetic peptide
FTINCQEPKLGSLVV

SEQ ID NO: 26 - protein, 15 aa, Artificial Sequence
  Synthetic peptide
GSLVVRCSFYEDFLE

SEQ ID NO: 27 - protein, 15 aa, Artificial Sequence
  Synthetic peptide
SSANNCTFEYVSQPF

SEQ ID NO: 28 - protein, 15 aa, Artificial Sequence
  Synthetic peptide
NLVRDLPQGFSALEP

SEQ ID NO: 29 - protein, 15 aa, Artificial Sequence
  Synthetic peptide
RFASVYAWNRKRISN

SEQ ID NO: 30 - protein, 15 aa, Artificial Sequence
  Synthetic peptide
MWLSYFIASFRLFAR

SEQ ID NO: 31 - protein, 15 aa, Artificial Sequence
  Synthetic peptide
TNILLNVPLHGTILT

SEQ ID NO: 32 - protein, 15 aa, Artificial Sequence
  Synthetic peptide
IAGHHLGRCDIKDLP

SEQ ID NO: 33 - protein, 15 aa, Artificial Sequence
  Synthetic peptide
LGRCDIKDLPKEITV

SEQ ID NO: 34 - protein, 15 aa, Artificial Sequence
  Synthetic peptide
AGFIKQYGDCLGDIA

SEQ ID NO: 35 - protein, 15 aa, Artificial Sequence
  Synthetic peptide
SWFTALTQHGKEDLK

SEQ ID NO: 36 - protein, 15 aa, Artificial Sequence
  Synthetic peptide
YYRRATRRIRGGDGK

SEQ ID NO: 37 - protein, 15 aa, Artificial Sequence
  Synthetic peptide
IKDLPKEITVATSRT

SEQ ID NO: 38 - protein, 15 aa, Artificial Sequence
  Synthetic peptide
AAEASKKPRQKRTAT

SEQ ID NO: 39 - protein, 15 aa, Artificial Sequence
  Synthetic peptide
LYLYALVYFLQSINF

SEQ ID NO: 40 - protein, 15 aa, Artificial Sequence
  Synthetic peptide
AGNGGDAALALLLLD

SEQ ID NO: 41 - protein, 15 aa, Artificial Sequence
  Synthetic peptide
QFAYANRNRFLYIIK

SEQ ID NO: 42 - protein, 15 aa, Artificial Sequence
  Synthetic peptide
TTVAAFHQECSLQSC

SEQ ID NO: 43 - protein, 15 aa, Artificial Sequence
  Synthetic peptide
FHQECSLQSCTQHQP

SEQ ID NO: 44 - protein, 15 aa, Artificial Sequence
  Synthetic peptide
ARKSAPLIELCVDEA

SEQ ID NO: 45 - protein, 15 aa, Artificial Sequence
  Synthetic peptide
PLIELCVDEAGSKSP

SEQ ID NO: 46 - protein, 15 aa, Artificial Sequence
  Synthetic peptide
CNLLLLFVTVYSHLL

SEQ ID NO: 47 - protein, 15 aa, Artificial Sequence
  Synthetic peptide
EDFLEYHDVRVVLDF

SEQ ID NO: 48 - protein, 15 aa, Artificial Sequence
  Synthetic peptide
DFLEYHDVRVVLDFI

SEQ ID NO: 49 - protein, 15 aa, Artificial Sequence
  Synthetic peptide
VSQPFLMDLEGKQGN

SEQ ID NO: 50 - protein, 15 aa, Artificial Sequence
  Synthetic peptide
EFVFKNIDGYFKIYS

SEQ ID NO: 51 - protein, 15 aa, Artificial Sequence
  Synthetic peptide
FKIYSKHTPINLVRD

SEQ ID NO: 52 - protein, 15 aa, Artificial Sequence
  Synthetic peptide
KHTPINLVRDLPQGF

SEQ ID NO: 53 - protein, 15 aa, Artificial Sequence
  Synthetic peptide
LPQGFSALEPLVDLP

SEQ ID NO: 54 - protein, 15 aa, Artificial Sequence
  Synthetic peptide
IPFAMQMAYRFNGIG

SEQ ID NO: 55 - protein, 15 aa, Artificial Sequence
  Synthetic peptide
TLRVEAFEYYHTTDP

SEQ ID NO: 56 - protein, 15 aa, Artificial Sequence
  Synthetic peptide
HTTDPSFLGRYMSAL

SEQ ID NO: 57 - protein, 15 aa, Artificial Sequence
  Synthetic peptide
LLWPVTLACFVLAAV

SEQ ID NO: 58 - protein, 15 aa, Artificial Sequence
  Synthetic peptide
ITGGIAIAMACLVGL

SEQ ID NO: 59 - protein, 15 aa, Artificial Sequence
  Synthetic peptide
TRSMWSFNPETNILL

SEQ ID NO: 60 - protein, 15 aa, Artificial Sequence
  Synthetic peptide
GTILTRPLLESELVI

SEQ ID NO: 61 - protein, 15 aa, Artificial Sequence
  Synthetic peptide
QFAPSASAFFGMSRI

SEQ ID NO: 62 - protein, 15 aa, Artificial Sequence
  Synthetic peptide
LTYTGAIKLDDKDPN

SEQ ID NO: 63 - protein, 15 aa, Artificial Sequence
  Synthetic peptide
KRISNCVADYSVLYN

SEQ ID NO: 64 - protein, 15 aa, Artificial Sequence
  Synthetic peptide
LKPFERDISTEIYQA

SEQ ID NO: 65 - protein, 15 aa, Artificial Sequence
  Synthetic peptide
AENSVAYSNNSIAIP

SEQ ID NO: 66 - protein, 15 aa, Artificial Sequence
  Synthetic peptide
TNFTISVTTEILPVS

SEQ ID NO: 67 - protein, 15 aa, Artificial Sequence
  Synthetic peptide
VTLADAGFIKQYGDC

SEQ ID NO: 68 - protein, 15 aa, Artificial Sequence
  Synthetic peptide
YLTFYLTNDVSFLAH

SEQ ID NO: 69 - protein, 15 aa, Artificial Sequence
  Synthetic peptide
VYYPDKVFRSSVLHS

SEQ ID NO: 70 - protein, 15 aa, Artificial Sequence
  Synthetic peptide
SVLHSTQDLFLPFFS

SEQ ID NO: 71 - protein, 15 aa, Artificial Sequence
  Synthetic peptide
GGDGKMKDLSPRWYF

SEQ ID NO: 72 - protein, 15 aa, Artificial Sequence
  Synthetic peptide
ATSRTLSYYKLGASQ

SEQ ID NO: 73 - protein, 15 aa, Artificial Sequence
  Synthetic peptide
LGASQRVAGDSGFAA

SEQ ID NO: 74 - protein, 15 aa, Artificial Sequence
  Synthetic peptide
RVAGDSGFAAYSRYR

SEQ ID NO: 75 - protein, 15 aa, Artificial Sequence
  Synthetic peptide
YSRYRIGNYKLNTDH

SEQ ID NO: 76 - protein, 15 aa, Artificial Sequence
  Synthetic peptide
IGNYKLNTDHSSSSD

SEQ ID NO: 77 - protein, 15 aa, Artificial Sequence
  Synthetic peptide
KAYNVTQAFGRRGPE

SEQ ID NO: 78 - protein, 15 aa, Artificial Sequence
  Synthetic peptide
LIRQGTDYKHWPQIA

SEQ ID NO: 79 - protein, 15 aa, Artificial Sequence
  Synthetic peptide
GYKKPASRELKVTFF

SEQ ID NO: 80 - protein, 15 aa, Artificial Sequence
  Synthetic peptide
ASRELKVTFFPDLNG

SEQ ID NO: 81 - protein, 15 aa, Artificial Sequence
  Synthetic peptide
PDLNGDVVAIDYKHY

SEQ ID NO: 82 - protein, 15 aa, Artificial Sequence
  Synthetic peptide
RYLALYNKYKYFSGA

SEQ ID NO: 83 - protein, 15 aa, Artificial Sequence
  Synthetic peptide
MKDLSPRWYFYYLGT

SEQ ID NO: 84 - protein, 15 aa, Artificial Sequence
  Synthetic peptide
LPYGANKDGIIWVAT

SEQ ID NO: 85 - protein, 15 aa, Artificial Sequence
  Synthetic peptide
VRIIMRLWLCWKCRS

SEQ ID NO: 86 - protein, 15 aa, Artificial Sequence
  Synthetic peptide
EGALNTPKDHIGTRN

SEQ ID NO: 87 - protein, 15 aa, Artificial Sequence
  Synthetic peptide
EHVTFFIYNKIVDEP

SEQ ID NO: 88 - protein, 15 aa, Artificial Sequence
  Synthetic peptide
FIYNKIVDEPEEHVQ

SEQ ID NO: 89 - protein, 15 aa, Artificial Sequence
  Synthetic peptide
VTQNVLYENQKLIAN

SEQ ID NO: 90 - protein, 15 aa, Artificial Sequence
  Synthetic peptide
FVSLAIDAYPLTKHP

SEQ ID NO: 91 - protein, 15 aa, Artificial Sequence
  Synthetic peptide
IDAYPLTKHPNQEYA

SEQ ID NO: 92 - protein, 15 aa, Artificial Sequence
  Synthetic peptide
EELKKLLEQWNLVIG

SEQ ID NO: 93 - protein, 15 aa, Artificial Sequence
  Synthetic peptide
LLEQWNLVIGFLFLT

SEQ ID NO: 94 - protein, 15 aa, Artificial Sequence
  Synthetic peptide
NRNRFLYIIKLIFLW

SEQ ID NO: 95 - protein, 15 aa, Artificial Sequence
  Synthetic peptide
LYIIKLIFLWLLWPV

SEQ ID NO: 96 - protein, 15 aa, Artificial Sequence
  Synthetic peptide
VNNSYECDIPIGAGI

SEQ ID NO: 97 - protein, 15 aa, Artificial Sequence
  Synthetic peptide
SLQSCTQHQPYVVDD

SEQ ID NO: 98 - protein, 15 aa, Artificial Sequence
  Synthetic peptide
LPFFSNVTWFHAIHV

SEQ ID NO: 99 - protein, 15 aa, Artificial Sequence
  Synthetic peptide
CVDEAGSKSPIQYID

SEQ ID NO: 100 - protein, 15 aa, Artificial Sequence
  Synthetic peptide
KIITLKKRWQLALSK

SEQ ID NO: 101 - protein, 15 aa, Artificial Sequence
  Synthetic peptide
IQYIDIGNYTVSCLP

SEQ ID NO: 102 - protein, 15 aa, Artificial Sequence
  Synthetic peptide
NPVLPFNDGVYFAST

SEQ ID NO: 103 - protein, 15 aa, Artificial Sequence
  Synthetic peptide
QEPKLGSLVVRCSFY

SEQ ID NO: 104 - protein, 15 aa, Artificial Sequence
  Synthetic peptide
LVAAGLEAPFLYLYA

SEQ ID NO: 105 - protein, 15 aa, Artificial Sequence
  Synthetic peptide
EKSNIIRGWIFGTTL

SEQ ID NO: 106 - protein, 15 aa, Artificial Sequence
  Synthetic peptide
IRGWIFGTTLDSKTQ

SEQ ID NO: 107 - protein, 15 aa, Artificial Sequence
  Synthetic peptide
FGTTLDSKTQSLLIV

SEQ ID NO: 108 - protein, 15 aa, Artificial Sequence
  Synthetic peptide
DSKTQSLLIVNNATN

SEQ ID NO: 109 - protein, 15 aa, Artificial Sequence
  Synthetic peptide
VVIKVCEFQFCNDPF

SEQ ID NO: 110 - protein, 15 aa, Artificial Sequence
  Synthetic peptide
LGVYYHKNNKSWMES

SEQ ID NO: 111 - protein, 15 aa, Artificial Sequence
  Synthetic peptide
HKNNKSWMESEFRVY

SEQ ID NO: 112 - protein, 15 aa, Artificial Sequence
  Synthetic peptide
GKQGNFKNLREFVFK

SEQ ID NO: 113 - protein, 15 aa, Artificial Sequence
  Synthetic peptide
SALEPLVDLPIGINI

SEQ ID NO: 114 - protein, 15 aa, Artificial Sequence
  Synthetic peptide
VIRGDEVRQIAPGQT

SEQ ID NO: 115 - protein, 15 aa, Artificial Sequence
  Synthetic peptide
WNSNNLDSKVGGNYN

SEQ ID NO: 116 - protein, 15 aa, Artificial Sequence
  Synthetic peptide
LPPLLTDEMIAQYTS

SEQ ID NO: 117 - protein, 15 aa, Artificial Sequence
  Synthetic peptide
TDEMIAQYTSALLAG

SEQ ID NO: 118 - protein, 15 aa, Artificial Sequence
  Synthetic peptide
ALLAGTITSGWTFGA

SEQ ID NO: 119 - protein, 15 aa, Artificial Sequence
  Synthetic peptide
TITSGWTFGAGAALQ

SEQ ID NO: 120 - protein, 15 aa, Artificial Sequence
  Synthetic peptide
LPNDDTLRVEAFEYY

SEQ ID NO: 121 - protein, 15 aa, Artificial Sequence
  Synthetic peptide
SFLGRYMSALNHTKK

SEQ ID NO: 122 - protein, 15 aa, Artificial Sequence
  Synthetic peptide
YMSALNHTKKWKYPQ

SEQ ID NO: 123 - protein, 15 aa, Artificial Sequence
  Synthetic peptide
NHTKKWKYPQVNGLT

SEQ ID NO: 124 - protein, 15 aa, Artificial Sequence
  Synthetic peptide
AVITREVGFVVPGLP

SEQ ID NO: 125 - protein, 15 aa, Artificial Sequence
  Synthetic peptide
VPGLPGTILRTTNGD

SEQ ID NO: 126 - protein, 15 aa, Artificial Sequence
  Synthetic peptide
FLHFLPRVFSAVGNI

SEQ ID NO: 127 - protein, 15 aa, Artificial Sequence
  Synthetic peptide
SIVAGGIVAIVVTCL

SEQ ID NO: 128 - protein, 15 aa, Artificial Sequence
  Synthetic peptide
VSSQCVNLTTRTQLP

SEQ ID NO: 129 - protein, 15 aa, Artificial Sequence
  Synthetic peptide
VNLTTRTQLPPAYTN

SEQ ID NO: 130 - protein, 15 aa, Artificial Sequence
  Synthetic peptide
LIFLWLLWPVTLACF

SEQ ID NO: 131 - protein, 15 aa, Artificial Sequence
  Synthetic peptide
LTQHGKEDLKFPRGQ

SEQ ID NO: 132 - protein, 15 aa, Artificial Sequence
  Synthetic peptide
FPRGQGVPINTNSSP

SEQ ID NO: 133 - protein, 15 aa, Artificial Sequence
  Synthetic peptide
GVPINTNSSPDDQIG

SEQ ID NO: 134 - protein, 15 aa, Artificial Sequence
  Synthetic peptide
AIAMACLVGLMWLSY

SEQ ID NO: 135 - protein, 15 aa, Artificial Sequence
  Synthetic peptide
TRRIRGGDGKMKDLS

SEQ ID NO: 136 - protein, 15 aa, Artificial Sequence
  Synthetic peptide
FIASFRLFARTRSMW

SEQ ID NO: 137 - protein, 15 aa, Artificial Sequence
  Synthetic peptide
RLFARTRSMWSFNPE

SEQ ID NO: 138 - protein, 15 aa, Artificial Sequence
  Synthetic peptide
SFNPETNILLNVPLH

SEQ ID NO: 139 - protein, 15 aa, Artificial Sequence
  Synthetic peptide
NVPLHGTILTRPLLE

SEQ ID NO: 140 - protein, 15 aa, Artificial Sequence
  Synthetic peptide
RPLLESELVIGAVIL

SEQ ID NO: 141 - protein, 15 aa, Artificial Sequence
  Synthetic peptide
GAVILRGHLRIAGHH

SEQ ID NO: 142 - protein, 15 aa, Artificial Sequence
  Synthetic peptide
LNTDHSSSSDNIALL

SEQ ID NO: 143 - protein, 15 aa, Artificial Sequence
  Synthetic peptide
YFTSDYYQLYSTQLS

SEQ ID NO: 144 - protein, 15 aa, Artificial Sequence
  Synthetic peptide
TQAFGRRGPEQTQGN

SEQ ID NO: 145 - protein, 15 aa, Artificial Sequence
  Synthetic peptide
RRGPEQTQGNFGDQE

SEQ ID NO: 146 - protein, 15 aa, Artificial Sequence
  Synthetic peptide
FGDQELIRQGTDYKH

SEQ ID NO: 147 - protein, 15 aa, Artificial Sequence
  Synthetic peptide
TDYKHWPQIAQFAPS

SEQ ID NO: 148 - protein, 15 aa, Artificial Sequence
  Synthetic peptide
ASAFFGMSRIGMEVT

SEQ ID NO: 149 - protein, 15 aa, Artificial Sequence
  Synthetic peptide
GMEVTPSGTWLTYTG

SEQ ID NO: 150 - protein, 15 aa, Artificial Sequence
  Synthetic peptide
AIKLDDKDPNFKDQV

SEQ ID NO: 151 - protein, 15 aa, Artificial Sequence
  Synthetic peptide
YAWNRKRISNCVADY

SEQ ID NO: 152 - protein, 15 aa, Artificial Sequence
  Synthetic peptide
CVADYSVLYNSASFS

SEQ ID NO: 153 - protein, 15 aa, Artificial Sequence
  Synthetic peptide
SVLYNSASFSTFKCY

SEQ ID NO: 154 - protein, 15 aa, Artificial Sequence
  Synthetic peptide
SASFSTFKCYGVSPT

SEQ ID NO: 155 - protein, 15 aa, Artificial Sequence
  Synthetic peptide
TFKCYGVSPTKLNDL

SEQ ID NO: 156 - protein, 15 aa, Artificial Sequence
  Synthetic peptide
KLNDLCFTNVYADSF

SEQ ID NO: 157 - protein, 15 aa, Artificial Sequence
  Synthetic peptide
YADSFVIRGDEVRQI

SEQ ID NO: 158 - protein, 15 aa, Artificial Sequence
  Synthetic peptide
EIYQAGSTPCNGVEG

SEQ ID NO: 159 - protein, 15 aa, Artificial Sequence
  Synthetic peptide
FNCYFPLQSYGFQPT

SEQ ID NO: 160 - protein, 15 aa, Artificial Sequence
  Synthetic peptide
TLEILDITPCSFGGV

SEQ ID NO: 161 - protein, 15 aa, Artificial Sequence
  Synthetic peptide
SIAIPTNFTISVTTE

SEQ ID NO: 162 - protein, 15 aa, Artificial Sequence
  Synthetic peptide
SVTTEILPVSMTKTS

SEQ ID NO: 163 - protein, 15 aa, Artificial Sequence
  Synthetic peptide
STECSNLLLQYGSFC

SEQ ID NO: 164 - protein, 15 aa, Artificial Sequence
  Synthetic peptide
KNTQEVFAQVKQIYK

SEQ ID NO: 165 - protein, 15 aa, Artificial Sequence
  Synthetic peptide
TPPIKDFGGFNFSQI

SEQ ID NO: 166 - protein, 15 aa, Artificial Sequence
  Synthetic peptide
LPDPSKPSKRSFIED

SEQ ID NO: 167 - protein, 15 aa, Artificial Sequence
  Synthetic peptide
LLFNKVTLADAGFIK

SEQ ID NO: 168 - protein, 15 aa, Artificial Sequence
  Synthetic peptide
QYGDCLGDIAARDLI

SEQ ID NO: 169 - protein, 15 aa, Artificial Sequence
  Synthetic peptide
LGDIAARDLICAQKF

SEQ ID NO: 170 - protein, 15 aa, Artificial Sequence
  Synthetic peptide
ARDLICAQKFNGLTV

SEQ ID NO: 171 - protein, 15 aa, Artificial Sequence
  Synthetic peptide
ADAVIKTLQPVSELL

SEQ ID NO: 172 - protein, 15 aa, Artificial Sequence
  Synthetic peptide
ESDDYIATNGPLKVG

SEQ ID NO: 173 - protein, 15 aa, Artificial Sequence
  Synthetic peptide
IATNGPLKVGGSCVL

SEQ ID NO: 174 - protein, 15 aa, Artificial Sequence
  Synthetic peptide
SGHNLAKHCLHVVGP

SEQ ID NO: 175 - protein, 15 aa, Artificial Sequence
  Synthetic peptide
NLYDKLVSSFLEMKS

SEQ ID NO: 176 - protein, 15 aa, Artificial Sequence
  Synthetic peptide
QELGKYEQYIKWPWY

SEQ ID NO: 177 - protein, 15 aa, Artificial Sequence
  Synthetic peptide
RFYFYTSKTTVASLI

SEQ ID NO: 178 - protein, 15 aa, Artificial Sequence
  Synthetic peptide
LNQLTGYKKPASREL

SEQ ID NO: 179 - protein, 15 aa, Artificial Sequence
  Synthetic peptide
TPSFKKGAKLLHKPI

SEQ ID NO: 180 - protein, 15 aa, Artificial Sequence
  Synthetic peptide
VWHVNNATNKATYKP

SEQ ID NO: 181 - protein, 15 aa, Artificial Sequence
  Synthetic peptide
TFTRSTNSRIKASMP

SEQ ID NO: 182 - protein, 15 aa, Artificial Sequence
  Synthetic peptide
TNSRIKASMPTTIAK

SEQ ID NO: 183 - protein, 15 aa, Artificial Sequence
  Synthetic peptide
NTVKSVGKFCLEASF

SEQ ID NO: 184 - protein, 15 aa, Artificial Sequence
  Synthetic peptide
PNFSKLINIIIWFLL

SEQ ID NO: 185 - protein, 15 aa, Artificial Sequence
  Synthetic peptide
WDLTAFGLVAEWFLA

SEQ ID NO: 186 - protein, 15 aa, Artificial Sequence
  Synthetic peptide
FGLVAEWFLAYILFT

SEQ ID NO: 187 - protein, 15 aa, Artificial Sequence
  Synthetic peptide
PVYSFLPGVYSVIYL

SEQ ID NO: 188 - protein, 15 aa, Artificial Sequence
  Synthetic peptide
SFLAHIQWMVMFTPL

SEQ ID NO: 189 - protein, 15 aa, Artificial Sequence
  Synthetic peptide
IQWMVMFTPLVPFWI

SEQ ID NO: 190 - protein, 15 aa, Artificial Sequence
  Synthetic peptide
MFTPLVPFWITIAYI

SEQ ID NO: 191 - protein, 15 aa, Artificial Sequence
  Synthetic peptide
LTQYNRYLALYNKYK

SEQ ID NO: 192 - protein, 15 aa, Artificial Sequence
  Synthetic peptide
YREAACCHLAKALND

SEQ ID NO: 193 - protein, 15 aa, Artificial Sequence
  Synthetic peptide
CCHLAKALNDFSNSG

SEQ ID NO: 194 - protein, 15 aa, Artificial Sequence
  Synthetic peptide
FSNSGSDVLYQPPQT

SEQ ID NO: 195 - protein, 15 aa, Artificial Sequence
  Synthetic peptide
SDVLYQPPQTSITSA

SEQ ID NO: 196 - protein, 15 aa, Artificial Sequence
  Synthetic peptide
ELHLSWEVGKPRPPL

SEQ ID NO: 197 - protein, 15 aa, Artificial Sequence
  Synthetic peptide
PRPPLNRNYVFTGYR

SEQ ID NO: 198 - protein, 15 aa, Artificial Sequence
  Synthetic peptide
FTGYRVTKNSKVQIG

SEQ ID NO: 199 - protein, 15 aa, Artificial Sequence
  Synthetic peptide
VTKNSKVQIGEYTFE

SEQ ID NO: 200 - protein, 15 aa, Artificial Sequence
  Synthetic peptide
VNARLRAKHYVYIGD

SEQ ID NO: 201 - protein, 15 aa, Artificial Sequence
  Synthetic peptide
NLVIGFLFLTWICLL

SEQ ID NO: 202 - protein, 15 aa, Artificial Sequence
  Synthetic peptide
FLFLTWICLLQFAYA

SEQ ID NO: 203 - protein, 15 aa, Artificial Sequence
  Synthetic peptide
WICLLQFAYANRNRF

SEQ ID NO: 204 - protein, 15 aa, Artificial Sequence
  Synthetic peptide
LEAPFLYLYALVYFL

SEQ ID NO: 205 - protein, 15 aa, Artificial Sequence
  Synthetic peptide
PRWYFYYLGTGPEAG

SEQ ID NO: 206 - protein, 15 aa, Artificial Sequence
  Synthetic peptide
RLWLCWKCRSKNPLL

SEQ ID NO: 207 - protein, 15 aa, Artificial Sequence
  Synthetic peptide
IWVATEGALNTPKDH

SEQ ID NO: 208 - protein, 15 aa, Artificial Sequence
  Synthetic peptide
YDANYFLCWHTNCYD

SEQ ID NO: 209 - protein, 15 aa, Artificial Sequence
  Synthetic peptide
FLCWHTNCYDYCIPY

SEQ ID NO: 210 - protein, 15 aa, Artificial Sequence
  Synthetic peptide
TNCYDYCIPYNSVTS

SEQ ID NO: 211 - protein, 15 aa, Artificial Sequence
  Synthetic peptide
TDTGVEHVTFFIYNK

SEQ ID NO: 212 - protein, 15 aa, Artificial Sequence
  Synthetic peptide
TDTGVEHVTFFIYNK

SEQ ID NO: 213 - protein, 15 aa, Artificial Sequence
  Synthetic peptide
GSSGVVNPVMEPIYD

SEQ ID NO: 214 - protein, 15 aa, Artificial Sequence
  Synthetic peptide
HIDAYKTFPPTEPKK

SEQ ID NO: 215 - protein, 15 aa, Artificial Sequence
  Synthetic peptide
QKKQQTVTLLPAADL

SEQ ID NO: 216 - protein, 15 aa, Artificial Sequence
  Synthetic peptide
TVTLLPAADLDDFSK

SEQ ID NO: 217 - protein, 15 aa, Artificial Sequence
  Synthetic peptide
QPYRVVVLSFELLHA

SEQ ID NO: 218 - protein, 15 aa, Artificial Sequence
  Synthetic peptide
VVLSFELLHAPATVC

SEQ ID NO: 219 - protein, 15 aa, Artificial Sequence
  Synthetic peptide
LTGTGVLTESNKKFL

SEQ ID NO: 220 - protein, 15 aa, Artificial Sequence
  Synthetic peptide
QMAYRFNGIGVTQNV

SEQ ID NO: 221 - protein, 15 aa, Artificial Sequence
  Synthetic peptide
LYENQKLIANQFNSA

SEQ ID NO: 222 - protein, 15 aa, Artificial Sequence
  Synthetic peptide
HWFVTQRNFYEPQII

SEQ ID NO: 223 - protein, 15 aa, Artificial Sequence
  Synthetic peptide
QRNFYEPQIITTDNT

SEQ ID NO: 224 - protein, 15 aa, Artificial Sequence
  Synthetic peptide
FVSGNCDVVIGIVNN

SEQ ID NO: 225 - protein, 15 aa, Artificial Sequence
  Synthetic peptide
GIVNNTVYDPLQPEL

SEQ ID NO: 226 - protein, 15 aa, Artificial Sequence
  Synthetic peptide
LQPELDSFKEELDKY

SEQ ID NO: 227 - protein, 15 aa, Artificial Sequence
  Synthetic peptide
YCIDGALLTKSSEYK

SEQ ID NO: 228 - protein, 15 aa, Artificial Sequence
  Synthetic peptide
MAAYVDNSSLTIKKP

SEQ ID NO: 229 - protein, 15 aa, Artificial Sequence
  Synthetic peptide
HCANFNVLFSTVFPP

SEQ ID NO: 230 - protein, 15 aa, Artificial Sequence
  Synthetic peptide
NVLFSTVFPPTSFGP

SEQ ID NO: 231 - protein, 15 aa, Artificial Sequence
  Synthetic peptide
YPKCDRAMPNMLRIM

SEQ ID NO: 232 - protein, 15 aa, Artificial Sequence
  Synthetic peptide
RAMPNMLRIMASLVL

SEQ ID NO: 233 - protein, 15 aa, Artificial Sequence
  Synthetic peptide
AVTANVNALLSTDGN

SEQ ID NO: 234 - protein, 15 aa, Artificial Sequence
  Synthetic peptide
KTDGTLMIERFVSLA

SEQ ID NO: 235 - protein, 15 aa, Artificial Sequence
  Synthetic peptide
DVFHLYLQYIRKLHD

SEQ ID NO: 236 - protein, 15 aa, Artificial Sequence
  Synthetic peptide
PQNQRNAPRITFGGP

SEQ ID NO: 237 - protein, 15 aa, Artificial Sequence
  Synthetic peptide
NAPRITFGGPSDSTG

SEQ ID NO: 238 - protein, 15 aa, Artificial Sequence
  Synthetic peptide
TFGGPSDSTGSNQNG

SEQ ID NO: 239 - protein, 15 aa, Artificial Sequence
  Synthetic peptide
QDALFAYTKRNVIPT

SEQ ID NO: 240 - protein, 15 aa, Artificial Sequence
  Synthetic peptide
IAATRGATVVIGTSK

SEQ ID NO: 241 - protein, 15 aa, Artificial Sequence
  Synthetic peptide
SHRFYRLANECAQVL

SEQ ID NO: 242 - protein, 15 aa, Artificial Sequence
  Synthetic peptide
SEMVMCGGSLYVKPG

SEQ ID NO: 243 - protein, 15 aa, Artificial Sequence
  Synthetic peptide
FNICQAVTANVNALL

SEQ ID NO: 244 - protein, 15 aa, Artificial Sequence
  Synthetic peptide
KHFYWFFSNYLKRRV

SEQ ID NO: 245 - protein, 15 aa, Artificial Sequence
  Synthetic peptide
DDQIGYYRRATRRIR

SEQ ID NO: 246 - protein, 15 aa, Artificial Sequence
  Synthetic peptide
DAALALLLLDRLNQL

SEQ ID NO: 247 - protein, 15 aa, Artificial Sequence
  Synthetic peptide
LLLLDRLNQLESKMS

SEQ ID NO: 248 - protein, 15 aa, Artificial Sequence
  Synthetic peptide
PSGTWLTYTGAIKLD

SEQ ID NO: 249 - protein, 15 aa, Artificial Sequence
  Synthetic peptide
ALLAVFQSASKIITL

SEQ ID NO: 250 - protein, 15 aa, Artificial Sequence
  Synthetic peptide
KKRWQLALSKGVHFV

SEQ ID NO: 251 - protein, 15 aa, Artificial Sequence
  Synthetic peptide
LVYFLQSINFVRIIM

SEQ ID NO: 252 - protein, 15 aa, Artificial Sequence
  Synthetic peptide
QSINFVRIIMRLWLC

SEQ ID NO: 253 - protein, 15 aa, Artificial Sequence
  Synthetic peptide
KNPLLYDANYFLCWH

SEQ ID NO: 254 - protein, 15 aa, Artificial Sequence
  Synthetic peptide
FLGIITTVAAFHQEC

SEQ ID NO: 255 - protein, 15 aa, Artificial Sequence
  Synthetic peptide
FYSKWYIRVGARKSA

SEQ ID NO: 256 - protein, 15 aa, Artificial Sequence
  Synthetic peptide
YIRVGARKSAPLIEL

SEQ ID NO: 257 - protein, 15 aa, Artificial Sequence
  Synthetic peptide
KVTFFPDLNGDVVAI

SEQ ID NO: 258 - protein, 15 aa, Artificial Sequence
  Synthetic peptide
KLLKSIAATRGATVV

SEQ ID NO: 259 - protein, 15 aa, Artificial Sequence
  Synthetic peptide
LMIERFVSLAIDAYP

SEQ ID NO: 260 - protein, 15 aa, Artificial Sequence
  Synthetic peptide
SLLIVNNATNVVIKV

SEQ ID NO: 261 - protein, 15 aa, Artificial Sequence
  Synthetic peptide
CEFQFCNDPFLGVYY

SEQ ID NO: 262 - protein, 15 aa, Artificial Sequence
  Synthetic peptide
CTFEYVSQPFLMDLE

SEQ ID NO: 263 - protein, 15 aa, Artificial Sequence
  Synthetic peptide
IGINITRFQTLLALH

SEQ ID NO: 264 - protein, 15 aa, Artificial Sequence
  Synthetic peptide
TRFQTLLALHRSYLT

SEQ ID NO: 265 - protein, 15 aa, Artificial Sequence
  Synthetic peptide
FTVEKGIYQTSNFRV

SEQ ID NO: 266 - protein, 15 aa, Artificial Sequence
  Synthetic peptide
SNFRVQPTESIVRFP

SEQ ID NO: 267 - protein, 15 aa, Artificial Sequence
  Synthetic peptide
IVRFPNITNLCPFGE

SEQ ID NO: 268 - protein, 15 aa, Artificial Sequence
  Synthetic peptide
CPFGEVFNATRFASV

SEQ ID NO: 269 - protein, 15 aa, Artificial Sequence
  Synthetic peptide
AYSNNSIAIPTNFTI

SEQ ID NO: 270 - protein, 15 aa, Artificial Sequence
  Synthetic peptide
NLLLQYGSFCTQLNR

SEQ ID NO: 271 - protein, 15 aa, Artificial Sequence
  Synthetic peptide
TQLNRALTGIAVEQD

SEQ ID NO: 272 - protein, 15 aa, Artificial Sequence
  Synthetic peptide
VFAQVKQIYKTPPIK

SEQ ID NO: 273 - protein, 15 aa, Artificial Sequence
  Synthetic peptide
NFSQILPDPSKPSKR

SEQ ID NO: 274 - protein, 15 aa, Artificial Sequence
  Synthetic peptide
KPSKRSFIEDLLFNK

SEQ ID NO: 275 - protein, 15 aa, Artificial Sequence
  Synthetic peptide
SFIEDLLFNKVTLAD

SEQ ID NO: 276 - protein, 15 aa, Artificial Sequence
  Synthetic peptide
CAQKFNGLTVLPPLL

SEQ ID NO: 277 - protein, 15 aa, Artificial Sequence
  Synthetic peptide
AQYTSALLAGTITSG

SEQ ID NO: 278 - protein, 15 aa, Artificial Sequence
  Synthetic peptide
APHGVVFLHVTYVPA

SEQ ID NO: 279 - protein, 15 aa, Artificial Sequence
  Synthetic peptide
ELDKYFKNHTSPDVD

SEQ ID NO: 280 - protein, 15 aa, Artificial Sequence
  Synthetic peptide
GINASVVNIQKEIDR

SEQ ID NO: 281 - protein, 11 aa, Artificial Sequence
  Synthetic peptide
HTTDPSFLGRY

SEQ ID NO: 282 - protein, 11 aa, Artificial Sequence
  Synthetic peptide
TTDPSFLGRYM

SEQ ID NO: 283 - protein, 10 aa, Artificial Sequence
  Synthetic peptide
VTPSGTWLTY

SEQ ID NO: 284 - protein, 9 aa, Artificial Sequence
  Synthetic peptide
AVILRGHLR

SEQ ID NO: 285 - protein, 9 aa, Artificial Sequence
  Synthetic peptide
NSFTRGVYY

SEQ ID NO: 286 - protein, 9 aa, Artificial Sequence
  Synthetic peptide
HVSGTNGTK

SEQ ID NO: 287 - protein, 9 aa, Artificial Sequence
  Synthetic peptide
QIAPGQTGK

SEQ ID NO: 288 - protein, 10 aa, Artificial Sequence
  Synthetic peptide
QGLPNNTASW

SEQ ID NO: 289 - protein, 9 aa, Artificial Sequence
  Synthetic peptide
KMKDLSPRW

SEQ ID NO: 290 - protein, 9 aa, Artificial Sequence
  Synthetic peptide
ATIPIQASL

SEQ ID NO: 291 - protein, 9 aa, Artificial Sequence
  Synthetic peptide
ITFDNLKTL

SEQ ID NO: 292 - protein, 11 aa, Artificial Sequence
  Synthetic peptide
YYVGYLQPRTF

SEQ ID NO: 293 - protein, 12 aa, Artificial Sequence
  Synthetic peptide
VYDPLQPELDSF

SEQ ID NO: 294 - protein, 9 aa, Artificial Sequence
  Synthetic peptide
WTAGAAAYY

SEQ ID NO: 295 - protein, 10 aa, Artificial Sequence
  Synthetic peptide
SVASQSIIAY

SEQ ID NO: 296 - protein, 9 aa, Artificial Sequence
  Synthetic peptide
VASQSIIAY

SEQ ID NO: 297 - protein, 8 aa, Artificial Sequence
  Synthetic peptide
TDNYITTY

SEQ ID NO: 298 - protein, 10 aa, Artificial Sequence
  Synthetic peptide
KTFPPTEPKK

SEQ ID NO: 299 - protein, 9 aa, Artificial Sequence
  Synthetic peptide
SPRWYFYYL

SEQ ID NO: 300 - protein, 10 aa, Artificial Sequence
  Synthetic peptide
RVYSTGSNVF

SEQ ID NO: 301 - protein, 9 aa, Artificial Sequence
  Synthetic peptide
IPIQASLPF

SEQ ID NO: 302 - protein, 8 aa, Artificial Sequence
  Synthetic peptide
TPSKLIEY

SEQ ID NO: 303 - protein, 9 aa, Artificial Sequence
  Synthetic peptide
VPFWITIAY

SEQ ID NO: 304 - protein, 10 aa, Artificial Sequence
  Synthetic peptide
QELIRQGTDY

SEQ ID NO: 305 - protein, 8 aa, Artificial Sequence
  Synthetic peptide
QPYRVVVL

SEQ ID NO: 306 - protein, 10 aa, Artificial Sequence
  Synthetic peptide
TECSNLLLQY

SEQ ID NO: 307 - protein, 9 aa, Artificial Sequence
  Synthetic peptide
KVFRSSVLH

SEQ ID NO: 308 - protein, 10 aa, Artificial Sequence
  Synthetic peptide
VYSSANNCTF

SEQ ID NO: 309 - protein, 9 aa, Artificial Sequence
  Synthetic peptide
IYQTSNFRV

SEQ ID NO: 310 - protein, 9 aa, Artificial Sequence
  Synthetic peptide
FTISVTTEI

SEQ ID NO: 311 - protein, 9 aa, Artificial Sequence
  Synthetic peptide
SANNCTFEY

SEQ ID NO: 312 - protein, 9 aa, Artificial Sequence
  Synthetic peptide
FVFKNIDGY

SEQ ID NO: 313 - protein, 9 aa, Artificial Sequence
  Synthetic peptide
CVADYSVLY

SEQ ID NO: 314 - protein, 9 aa, Artificial Sequence
  Synthetic peptide
VGGNYNYLY

SEQ ID NO: 315 - protein, 8 aa, Artificial Sequence
  Synthetic peptide
RPLLESEL

SEQ ID NO: 316 - protein, 9 aa, Artificial Sequence
  Synthetic peptide
LPKEITVAT

SEQ ID NO: 317 - protein, 14 aa, Artificial Sequence
  Synthetic peptide
FDNPVLPFNDGVYF

SEQ ID NO: 318 - protein, 10 aa, Artificial Sequence
  Synthetic peptide
QIPFAMQMAY

SEQ ID NO: 319 - protein, 9 aa, Artificial Sequence
  Synthetic peptide
DAMRNAGIV

SEQ ID NO: 320 - protein, 10 aa, Artificial Sequence
  Synthetic peptide
YLPYPDPSRI

SEQ ID NO: 321 - protein, 11 aa, Artificial Sequence
  Synthetic peptide
KPFERDISTEI

SEQ ID NO: 322 - protein, 9 aa, Artificial Sequence
  Synthetic peptide
FPQSAPHGV

SEQ ID NO: 323 - protein, 8 aa, Artificial Sequence
  Synthetic peptide
LPQGFSAL

SEQ ID NO: 324 - protein, 9 aa, Artificial Sequence
  Synthetic peptide
MIAQYTSAL

SEQ ID NO: 325 - protein, 10 aa, Artificial Sequence
  Synthetic peptide
MEVTPSGTWL

SEQ ID NO: 326 - protein, 10 aa, Artificial Sequence
  Synthetic peptide
FEYVSQPFLM

SEQ ID NO: 327 - protein, 13 aa, Artificial Sequence
  Synthetic peptide
TESIVRFPNITNL

SEQ ID NO: 328 - protein, 10 aa, Artificial Sequence
  Synthetic peptide
FDEDDSEPVL

SEQ ID NO: 329 - protein, 10 aa, Artificial Sequence
  Synthetic peptide
SEPVLKGVKL

SEQ ID NO: 330 - protein, 10 aa, Artificial Sequence
  Synthetic peptide
SETKCTLKSF

SEQ ID NO: 331 - protein, 13 aa, Artificial Sequence
  Synthetic peptide
TESNKKFLPFQQF

SEQ ID NO: 332 - protein, 8 aa, Artificial Sequence
  Synthetic peptide
AEHVNNSY

SEQ ID NO: 333 - protein, 9 aa, Artificial Sequence
  Synthetic peptide
FVLAAVYRI

SEQ ID NO: 334 - protein, 9 aa, Artificial Sequence
  Synthetic peptide
SPDDQIGYY

SEQ ID NO: 335 - protein, 9 aa, Artificial Sequence
  Synthetic peptide
KAYNVTQAF

SEQ ID NO: 336 - protein, 9 aa, Artificial Sequence
  Synthetic peptide
FAPSASAFF

SEQ ID NO: 337 - protein, 9 aa, Artificial Sequence
  Synthetic peptide
TPSGTWLTY

SEQ ID NO: 338 - protein, 9 aa, Artificial Sequence
  Synthetic peptide
LPAADLDDF

SEQ ID NO: 339 - protein, 9 aa, Artificial Sequence
  Synthetic peptide
KLLEQWNLV

SEQ ID NO: 340 - protein, 9 aa, Artificial Sequence
  Synthetic peptide
SMWSFNPET

SEQ ID NO: 341 - protein, 9 aa, Artificial Sequence
  Synthetic peptide
KPRQKRTAT

SEQ ID NO: 342 - protein, 8 aa, Artificial Sequence
  Synthetic peptide
QPYRVVVL

SEQ ID NO: 343 - protein, 11 aa, Artificial Sequence
  Synthetic peptide
YENQKLIANQF

SEQ ID NO: 344 - protein, 9 aa, Artificial Sequence
  Synthetic peptide
FPRGQGVPI

SEQ ID NO: 345 - protein, 9 aa, Artificial Sequence
  Synthetic peptide
WFFSNYLKR

SEQ ID NO: 346 - protein, 12 aa, Artificial Sequence
  Synthetic peptide
GVAPGTAVLRQW

SEQ ID NO: 347 - protein, 9 aa, Artificial Sequence
  Synthetic peptide
ASAFFGMSR

SEQ ID NO: 348 - protein, 9 aa, Artificial Sequence
  Synthetic peptide
MACLVGLMW

SEQ ID NO: 349 - protein, 10 aa, Artificial Sequence
  Synthetic peptide
CATVHTANKW

SEQ ID NO: 350 - protein, 10 aa, Artificial Sequence
  Synthetic peptide
QFAPSASAFF

SEQ ID NO: 351 - protein, 9 aa, Artificial Sequence
  Synthetic peptide
LPLTQYNRY

SEQ ID NO: 352 - protein, 9 aa, Artificial Sequence
  Synthetic peptide
SELVIGAVI

SEQ ID NO: 353 - protein, 9 aa, Artificial Sequence
  Synthetic peptide
AEIRASANL

SEQ ID NO: 354 - protein, 10 aa, Artificial Sequence
  Synthetic peptide
SASAFFGMSR

SEQ ID NO: 355 - protein, 9 aa, Artificial Sequence
  Synthetic peptide
WTAGAAAYY

SEQ ID NO: 356 - protein, 9 aa, Artificial Sequence
  Synthetic peptide
FVLAAVYRI

SEQ ID NO: 357 - protein, 8 aa, Artificial Sequence
  Synthetic peptide
YLITPVHV

SEQ ID NO: 358 - protein, 12 aa, Artificial Sequence
  Synthetic peptide
KPVETSNSFDVL

SEQ ID NO: 359 - protein, 13 aa, Artificial Sequence
  Synthetic peptide
AESHVDTDLTKPY

SEQ ID NO: 360 - protein, 9 aa, Artificial Sequence
  Synthetic peptide
TSNQVAVLY

SEQ ID NO: 361 - protein, 9 aa, Artificial Sequence
  Synthetic peptide
AEVQIDRLI

SEQ ID NO: 362 - protein, 9 aa, Artificial Sequence
  Synthetic peptide
GLEAPFLYL

SEQ ID NO: 363 - protein, 10 aa, Artificial Sequence
  Synthetic peptide
GVFVSNGTHW

SEQ ID NO: 364 - protein, 9 aa, Artificial Sequence
  Synthetic peptide
LSYFIASFR

SEQ ID NO: 365 - protein, 9 aa, Artificial Sequence
  Synthetic peptide
SPRWYFYYL

SEQ ID NO: 366 - protein, 9 aa, Artificial Sequence
  Synthetic peptide
SIIAYTMSL

SEQ ID NO: 367 - protein, 9 aa, Artificial Sequence
  Synthetic peptide
AGDSGFAAY

SEQ ID NO: 368 - protein, 9 aa, Artificial Sequence
  Synthetic peptide
HLMSFPQSA

SEQ ID NO: 369 - protein, 9 aa, Artificial Sequence
  Synthetic peptide
QSASKIITL

SEQ ID NO: 370 - protein, 10 aa, Artificial Sequence
  Synthetic peptide
TTDPSFLGRY

SEQ ID NO: 371 - protein, 9 aa, Artificial Sequence
  Synthetic peptide
LAAVYRINW

SEQ ID NO: 372 - protein, 10 aa, Artificial Sequence
  Synthetic peptide
KSAGFPFNKW

SEQ ID NO: 373 - protein, 9 aa, Artificial Sequence
  Synthetic peptide
LYLYALVYF

SEQ ID NO: 374 - protein, 10 aa, Artificial Sequence
  Synthetic peptide
TVKNGSIHLY

SEQ ID NO: 375 - protein, 14 aa, Artificial Sequence
  Synthetic peptide
GELGDVRETMSYLF

SEQ ID NO: 376 - protein, 11 aa, Artificial Sequence
  Synthetic peptide
RVAGDSGFAAY

SEQ ID NO: 377 - protein, 9 aa, Artificial Sequence
  Synthetic peptide
SFKEELDKY

SEQ ID NO: 378 - protein, 9 aa, Artificial Sequence
  Synthetic peptide
GSVAYESLR

SEQ ID NO: 379 - protein, 10 aa, Artificial Sequence
  Synthetic peptide
ETIQITISSF

SEQ ID NO: 380 - protein, 9 aa, Artificial Sequence
  Synthetic peptide
FVRIIMRLW

SEQ ID NO: 381 - protein, 9 aa, Artificial Sequence
  Synthetic peptide
GAMDTTSYR

SEQ ID NO: 382 - protein, 10 aa, Artificial Sequence
  Synthetic peptide
VENPHLMGWD

SEQ ID NO: 383 - protein, 9 aa, Artificial Sequence
  Synthetic peptide
EYVSQPFLM

SEQ ID NO: 384 - protein, 9 aa, Artificial Sequence
  Synthetic peptide
FVSNGTHWF

SEQ ID NO: 385 - protein, 9 aa, Artificial Sequence
  Synthetic peptide
SIIQFPNTY

SEQ ID NO: 386 - protein, 9 aa, Artificial Sequence
  Synthetic peptide
KPNELSRVL

SEQ ID NO: 387 - protein, 10 aa, Artificial Sequence
  Synthetic peptide
ATSRTLSYYK

SEQ ID NO: 388 - protein, 9 aa, Artificial Sequence
  Synthetic peptide
FPLKLRGTA

SEQ ID NO: 389 - protein, 14 aa, Artificial Sequence
  Synthetic peptide
EVTGDSCNNYMLTY

SEQ ID NO: 390 - protein, 11 aa, Artificial Sequence
  Synthetic peptide
EVGHTDLMAAY

SEQ ID NO: 391 - protein, 14 aa, Artificial Sequence
  Synthetic peptide
ITEEVGHTDLMAAY

SEQ ID NO: 392 - protein, 9 aa, Artificial Sequence
  Synthetic peptide
FEPSTQYEY

SEQ ID NO: 393 - protein, 9 aa, Artificial Sequence
  Synthetic peptide
VPHISRQRL

SEQ ID NO: 394 - protein, 9 aa, Artificial Sequence
  Synthetic peptide
ILFTRFFYV

SEQ ID NO: 395 - protein, 9 aa, Artificial Sequence
  Synthetic peptide
KEIDRLNEV

SEQ ID NO: 396 - protein, 10 aa, Artificial Sequence
  Synthetic peptide
KQEILGTVSW

SEQ ID NO: 397 - protein, 10 aa, Artificial Sequence
  Synthetic peptide
YLTNDVSFLA

SEQ ID NO: 398 - protein, 10 aa, Artificial Sequence
  Synthetic peptide
QPYRVVVLSF

SEQ ID NO: 399 - protein, 10 aa, Artificial Sequence
  Synthetic peptide
YTNSFTRGVY

SEQ ID NO: 400 - protein, 10 aa, Artificial Sequence
  Synthetic peptide
LTNDNTSRYW

SEQ ID NO: 401 - protein, 9 aa, Artificial Sequence
  Synthetic peptide
KSNLKPFER

SEQ ID NO: 402 - protein, 9 aa, Artificial Sequence
  Synthetic peptide
YLQPRTFLL

SEQ ID NO: 403 - protein, 9 aa, Artificial Sequence
  Synthetic peptide
QELGKYEQY

SEQ ID NO: 404 - protein, 10 aa, Artificial Sequence
  Synthetic peptide
YTTTIKPVTY

SEQ ID NO: 405 - protein, 9 aa, Artificial Sequence
  Synthetic peptide
WVLNNDYYR

SEQ ID NO: 406 - protein, 9 aa, Artificial Sequence
  Synthetic peptide
SFYYVWKSY

SEQ ID NO: 407 - protein, 9 aa, Artificial Sequence
  Synthetic peptide
LAYYFMRFR

SEQ ID NO: 408 - protein, 9 aa, Artificial Sequence
  Synthetic peptide
ITLKKRWQL

SEQ ID NO: 409 - protein, 10 aa, Artificial Sequence
  Synthetic peptide
NSTNVTIATY

SEQ ID NO: 410 - protein, 9 aa, Artificial Sequence
  Synthetic peptide
YVNTFSSTF

SEQ ID NO: 411 - protein, 9 aa, Artificial Sequence
  Synthetic peptide
VLKGVKLHY

SEQ ID NO: 412 - protein, 8 aa, Artificial Sequence
  Synthetic peptide
FLPGVYSV

SEQ ID NO: 413 - protein, 9 aa, Artificial Sequence
  Synthetic peptide
LPNNTASWF

SEQ ID NO: 414 - protein, 9 aa, Artificial Sequence
  Synthetic peptide
NYNYLYRLF

SEQ ID NO: 415 - protein, 10 aa, Artificial Sequence
  Synthetic peptide
YVYANGGKGF

SEQ ID NO: 416 - protein, 9 aa, Artificial Sequence
  Synthetic peptide
IPFAMQMAY

SEQ ID NO: 417 - protein, 10 aa, Artificial Sequence
  Synthetic peptide
EVAVKMFDAY

SEQ ID NO: 418 - protein, 10 aa, Artificial Sequence
  Synthetic peptide
SETKCTLKSF

SEQ ID NO: 419 - protein, 9 aa, Artificial Sequence
  Synthetic peptide
NSIAIPTNF

SEQ ID NO: 420 - protein, 9 aa, Artificial Sequence
  Synthetic peptide
DVLLPLTQY

SEQ ID NO: 421 - protein, 12 aa, Artificial Sequence
  Synthetic peptide
EVFNATRFASVY

SEQ ID NO: 422 - protein, 9 aa, Artificial Sequence
  Synthetic peptide
HWFVTQRNF

SEQ ID NO: 423 - protein, 9 aa, Artificial Sequence
  Synthetic peptide
NELSRVLGL

SEQ ID NO: 424 - protein, 9 aa, Artificial Sequence
  Synthetic peptide
RPDTRYVLM

SEQ ID NO: 425 - protein, 9 aa, Artificial Sequence
  Synthetic peptide
VENMTPRDL

SEQ ID NO: 426 - protein, 12 aa, Artificial Sequence
  Synthetic peptide
SEYKGPITDVFY

SEQ ID NO: 427 - protein, 11 aa, Artificial Sequence
  Synthetic peptide
RFPNITNLCPF

SEQ ID NO: 428 - protein, 11 aa, Artificial Sequence
  Synthetic peptide
FPQSAPHGVVF

SEQ ID NO: 429 - protein, 9 aa, Artificial Sequence
  Synthetic peptide
RSVASQSII

SEQ ID NO: 430 - protein, 10 aa, Artificial Sequence
  Synthetic peptide
DTVIEVQGYK

SEQ ID NO: 431 - protein, 10 aa, Artificial Sequence
  Synthetic peptide
DVVAIDYKHY

SEQ ID NO: 432 - protein, 9 aa, Artificial Sequence
  Synthetic peptide
NSFTRGVYY

SEQ ID NO: 433 - protein, 14 aa, Artificial Sequence
  Synthetic peptide
RVAGDSGFAAYSRY

SEQ ID NO: 434 - protein, 9 aa, Artificial Sequence
  Synthetic peptide
IPIQASLPF

SEQ ID NO: 435 - protein, 9 aa, Artificial Sequence
  Synthetic peptide
YEQYIKWPW

SEQ ID NO: 436 - protein, 9 aa, Artificial Sequence
  Synthetic peptide
MSALNHTKK

SEQ ID NO: 437 - protein, 9 aa, Artificial Sequence
  Synthetic peptide
GEVFNATRF

SEQ ID NO: 438 - protein, 9 aa, Artificial Sequence
  Synthetic peptide
VFKNIDGYF

SEQ ID NO: 439 - protein, 10 aa, Artificial Sequence
  Synthetic peptide
VFVSNGTHWF

SEQ ID NO: 440 - protein, 9 aa, Artificial Sequence
  Synthetic peptide
IEVNSFSGY

SEQ ID NO: 441 - protein, 10 aa, Artificial Sequence
  Synthetic peptide
SVPWDTIANY

SEQ ID NO: 442 - protein, 9 aa, Artificial Sequence
  Synthetic peptide
AEWFLAYIL

SEQ ID NO: 443 - protein, 10 aa, Artificial Sequence
  Synthetic peptide
TEKSNIIRGW

SEQ ID NO: 444 - protein, 10 aa, Artificial Sequence
  Synthetic peptide
NPIQLSSYSL

SEQ ID NO: 445 - protein, 9 aa, Artificial Sequence
  Synthetic peptide
DSAEVAVKM

SEQ ID NO: 446 - protein, 9 aa, Artificial Sequence
  Synthetic peptide
FLPRVFSAV

SEQ ID NO: 447 - protein, 9 aa, Artificial Sequence
  Synthetic peptide
NTVKSVGKF

SEQ ID NO: 448 - protein, 9 aa, Artificial Sequence
  Synthetic peptide
MSNLGMPSY

SEQ ID NO: 449 - protein, 9 aa, Artificial Sequence
  Synthetic peptide
VFAQVKQIY

SEQ ID NO: 450 - protein, 10 aa, Artificial Sequence
  Synthetic peptide
LPIGINITRF

SEQ ID NO: 451 - protein, 10 aa, Artificial Sequence
  Synthetic peptide
YGANKDGIIW

SEQ ID NO: 452 - protein, 9 aa, Artificial Sequence
  Synthetic peptide
FVSNGTHWF

SEQ ID NO: 453 - protein, 9 aa, Artificial Sequence
  Synthetic peptide
VTYVPAQEK

SEQ ID NO: 454 - protein, 9 aa, Artificial Sequence
  Synthetic peptide
VVSTGYHFR

SEQ ID NO: 455 - protein, 9 aa, Artificial Sequence
  Synthetic peptide
TISLAGSYK

SEQ ID NO: 456 - protein, 10 aa, Artificial Sequence
  Synthetic peptide
YTNSFTRGVY

SEQ ID NO: 457 - protein, 9 aa, Artificial Sequence
  Synthetic peptide
RVYSTGSNV

SEQ ID NO: 458 - protein, 9 aa, Artificial Sequence
  Synthetic peptide
ELIRQGTDY

SEQ ID NO: 459 - protein, 10 aa, Artificial Sequence
  Synthetic peptide
ISLAGSYKDW

SEQ ID NO: 460 - protein, 9 aa, Artificial Sequence
  Synthetic peptide
EIDPKLDNY

SEQ ID NO: 461 - protein, 9 aa, Artificial Sequence
  Synthetic peptide
AELAKNVSL

SEQ ID NO: 462 - protein, 10 aa, Artificial Sequence
  Synthetic peptide
DSKVGGNYNY

SEQ ID NO: 463 - protein, 12 aa, Artificial Sequence
  Synthetic peptide
SEYTGNYQCGHY

SEQ ID NO: 464 - protein, 9 aa, Artificial Sequence
  Synthetic peptide
TPINLVRDL

SEQ ID NO: 465 - protein, 10 aa, Artificial Sequence
  Synthetic peptide
TPCSFGGVSV

SEQ ID NO: 466 - protein, 9 aa, Artificial Sequence
  Synthetic peptide
YLQPRTFLL

SEQ ID NO: 467 - protein, 9 aa, Artificial Sequence
  Synthetic peptide
INITRFQTL

SEQ ID NO: 468 - protein, 9 aa, Artificial Sequence
  Synthetic peptide
TLADAGFIK

SEQ ID NO: 469 - protein, 9 aa, Artificial Sequence
  Synthetic peptide
LVAEWFLAY

SEQ ID NO: 470 - protein, 9 aa, Artificial Sequence
  Synthetic peptide
LTDEMIAQY

SEQ ID NO: 471 - protein, 9 aa, Artificial Sequence
  Synthetic peptide
IAIPTNFTI

SEQ ID NO: 472 - protein, 14 aa, Artificial Sequence
  Synthetic peptide
KENSYTTTIKPVTY

SEQ ID NO: 473 - protein, 9 aa, Artificial Sequence
  Synthetic peptide
RIKASMPTT

SEQ ID NO: 474 - protein, 9 aa, Artificial Sequence
  Synthetic peptide
ETKCTLKSF

SEQ ID NO: 475 - protein, 10 aa, Artificial Sequence
  Synthetic peptide
LTAFGLVAEW

SEQ ID NO: 476 - protein, 9 aa, Artificial Sequence
  Synthetic peptide
KASMPTTIA

SEQ ID NO: 477 - protein, 9 aa, Artificial Sequence
  Synthetic peptide
RNRFLYIIK

SEQ ID NO: 478 - protein, 10 aa, Artificial Sequence
  Synthetic peptide
GYLQPRTFLL

SEQ ID NO: 479 - protein, 9 aa, Artificial Sequence
  Synthetic peptide
VASQSIIAY

SEQ ID NO: 480 - protein, 9 aa, Artificial Sequence
  Synthetic peptide
STAALGVLM

SEQ ID NO: 481 - protein, 9 aa, Artificial Sequence
  Synthetic peptide
VEVQPQLEM

SEQ ID NO: 482 - protein, 9 aa, Artificial Sequence
  Synthetic peptide
VVFLHVTYV

SEQ ID NO: 483 - protein, 9 aa, Artificial Sequence
  Synthetic peptide
RFDNPVLPF

SEQ ID NO: 484 - protein, 9 aa, Artificial Sequence
  Synthetic peptide
FVFKNIDGY

SEQ ID NO: 485 - protein, 9 aa, Artificial Sequence
  Synthetic peptide
CVADYSVLY

SEQ ID NO: 486 - protein, 13 aa, Artificial Sequence
  Synthetic peptide
NTNSSPDDQIGYY

SEQ ID NO: 487 - protein, 9 aa, Artificial Sequence
  Synthetic peptide
YERHSLSHF

SEQ ID NO: 488 - protein, 12 aa, Artificial Sequence
  Synthetic peptide
YSDVENPHLMGW

SEQ ID NO: 489 - protein, 9 aa, Artificial Sequence
  Synthetic peptide
IAIPTNFTI

SEQ ID NO: 490 - protein, 9 aa, Artificial Sequence
  Synthetic peptide
DTFCAGSTF

SEQ ID NO: 491 - protein, 11 aa, Artificial Sequence
  Synthetic peptide
EESSAKSASVY

SEQ ID NO: 492 - protein, 10 aa, Artificial Sequence
  Synthetic peptide
RVYSTGSNVF

SEQ ID NO: 493 - protein, 9 aa, Artificial Sequence
  Synthetic peptide
YYHKNNKSW

SEQ ID NO: 494 - protein, 9 aa, Artificial Sequence
  Synthetic peptide
IAATRGATV

SEQ ID NO: 495 - protein, 9 aa, Artificial Sequence
  Synthetic peptide
GVYFASTEK

SEQ ID NO: 496 - protein, 9 aa, Artificial Sequence
  Synthetic peptide
QELGKYEQY

SEQ ID NO: 497 - protein, 9 aa, Artificial Sequence
  Synthetic peptide
SSSDNIALL

SEQ ID NO: 498 - protein, 9 aa, Artificial Sequence
  Synthetic peptide
ATSRTLSYY

SEQ ID NO: 499 - protein, 10 aa, Artificial Sequence
  Synthetic peptide
EVFAQVKQIY

SEQ ID NO: 500 - protein, 10 aa, Artificial Sequence
  Synthetic peptide
LPYPDPSRIL

SEQ ID NO: 501 - protein, 8 aa, Artificial Sequence
  Synthetic peptide
VPLHGTIL

SEQ ID NO: 502 - protein, 11 aa, Artificial Sequence
  Synthetic peptide
QEVFAQVKQIY

SEQ ID NO: 503 - protein, 10 aa, Artificial Sequence
  Synthetic peptide
YAISAKNRAR

SEQ ID NO: 504 - protein, 11 aa, Artificial Sequence
  Synthetic peptide
FELDERIDKVL

SEQ ID NO: 505 - protein, 10 aa, Artificial Sequence
  Synthetic peptide
ELVIGAVILR

SEQ ID NO: 506 - protein, 9 aa, Artificial Sequence
  Synthetic peptide
ASMPTTIAK

SEQ ID NO: 507 - protein, 10 aa, Artificial Sequence
  Synthetic peptide
TEKSNIIRGW

SEQ ID NO: 508 - protein, 9 aa, Artificial Sequence
  Synthetic peptide
NASVVNIQK

SEQ ID NO: 509 - protein, 9 aa, Artificial Sequence
  Synthetic peptide
QVVDMSMTY

SEQ ID NO: 510 - protein, 9 aa, Artificial Sequence
  Synthetic peptide
MLAHAEETR

SEQ ID NO: 511 - protein, 9 aa, Artificial Sequence
  Synthetic peptide
DVFYKENSY

SEQ ID NO: 512 - protein, 10 aa, Artificial Sequence
  Synthetic peptide
GVYYPDKVFR

SEQ ID NO: 513 - protein, 9 aa, Artificial Sequence
  Synthetic peptide
NSFTRGVYY

SEQ ID NO: 514 - protein, 9 aa, Artificial Sequence
  Synthetic peptide
EPVLKGVKL

SEQ ID NO: 515 - protein, 10 aa, Artificial Sequence
  Synthetic peptide
HTTDPSFLGR

SEQ ID NO: 516 - protein, 8 aa, Artificial Sequence
  Synthetic peptide
NITRFQTL

SEQ ID NO: 517 - protein, 9 aa, Artificial Sequence
  Synthetic peptide
AEAELAKNV

SEQ ID NO: 518 - protein, 9 aa, Artificial Sequence
  Synthetic peptide
VENPDILRV

SEQ ID NO: 519 - protein, 9 aa, Artificial Sequence
  Synthetic peptide
YENFNQHEV

SEQ ID NO: 520 - protein, 9 aa, Artificial Sequence
  Synthetic peptide
EAKKVKPTV

SEQ ID NO: 521 - protein, 9 aa, Artificial Sequence
  Synthetic peptide
IANQFNSAI

SEQ ID NO: 522 - protein, 10 aa, Artificial Sequence
  Synthetic peptide
NTSNQVAVLY

SEQ ID NO: 523 - protein, 10 aa, Artificial Sequence
  Synthetic peptide
EILDITPCSF

SEQ ID NO: 524 - protein, 9 aa, Artificial Sequence
  Synthetic peptide
GTITVEELK

SEQ ID NO: 525 - protein, 9 aa, Artificial Sequence
  Synthetic peptide
GVVFLHVTY

SEQ ID NO: 526 - protein, 9 aa, Artificial Sequence
  Synthetic peptide
LPFNDGVYF

SEQ ID NO: 527 - protein, 12 aa, Artificial Sequence
  Synthetic peptide
TETDLTKGPHEF

SEQ ID NO: 528 - protein, 12 aa, Artificial Sequence
  Synthetic peptide
REGVFVSNGTHW

SEQ ID NO: 529 - protein, 9 aa, Artificial Sequence
  Synthetic peptide
KIYSKHTPI

SEQ ID NO: 530 - protein, 9 aa, Artificial Sequence
  Synthetic peptide
TLDSKTQSL

SEQ ID NO: 531 - protein, 9 aa, Artificial Sequence
  Synthetic peptide
LSYFIASFR

SEQ ID NO: 532 - protein, 9 aa, Artificial Sequence
  Synthetic peptide
LTDEMIAQY

SEQ ID NO: 533 - protein, 9 aa, Artificial Sequence
  Synthetic peptide
LVSDIDITF

SEQ ID NO: 534 - protein, 9 aa, Artificial Sequence
  Synthetic peptide
GEVFNATRF

SEQ ID NO: 535 - protein, 12 aa, Artificial Sequence
  Synthetic peptide
AAGLEAPFLYLY

SEQ ID NO: 536 - protein, 8 aa, Artificial Sequence
  Synthetic peptide
EPLVDLPI

SEQ ID NO: 537 - protein, 8 aa, Artificial Sequence
  Synthetic peptide
YAWNRKRI

SEQ ID NO: 538 - protein, 9 aa, Artificial Sequence
  Synthetic peptide
LVIGAVILR

SEQ ID NO: 539 - protein, 9 aa, Artificial Sequence
  Synthetic peptide
GEVFNATRF

SEQ ID NO: 540 - protein, 10 aa, Artificial Sequence
  Synthetic peptide
AAISDYDYYR

SEQ ID NO: 541 - protein, 9 aa, Artificial Sequence
  Synthetic peptide
KEIDRLNEV

SEQ ID NO: 542 - protein, 8 aa, Artificial Sequence
  Synthetic peptide
AEVAVKMF

SEQ ID NO: 543 - protein, 10 aa, Artificial Sequence
  Synthetic peptide
TECSNLLLQY

SEQ ID NO: 544 - protein, 10 aa, Artificial Sequence
  Synthetic peptide
KQEILGTVSW

SEQ ID NO: 545 - protein, 9 aa, Artificial Sequence
  Synthetic peptide
ASRELKVTF

SEQ ID NO: 546 - protein, 12 aa, Artificial Sequence
  Synthetic peptide
EEVGHTDLMAAY

SEQ ID NO: 547 - protein, 8 aa, Artificial Sequence
  Synthetic peptide
SPRRARSV

SEQ ID NO: 548 - protein, 9 aa, Artificial Sequence
  Synthetic peptide
QLTPTWRVY

SEQ ID NO: 549 - protein, 9 aa, Artificial Sequence
  Synthetic peptide
EVFAQVKQI

SEQ ID NO: 550 - protein, 9 aa, Artificial Sequence
  Synthetic peptide
AYSNNSIAI

SEQ ID NO: 551 - protein, 9 aa, Artificial Sequence
  Synthetic peptide
TSNQVAVLY

SEQ ID NO: 552 - protein, 10 aa, Artificial Sequence
  Synthetic peptide
EVARDLSLQF

SEQ ID NO: 553 - protein, 9 aa, Artificial Sequence
  Synthetic peptide
VAAGLEAPF

SEQ ID NO: 554 - protein, 9 aa, Artificial Sequence
  Synthetic peptide
GADPIHSLR

SEQ ID NO: 555 - protein, 9 aa, Artificial Sequence
  Synthetic peptide
LTAVVIPTK

SEQ ID NO: 556 - protein, 9 aa, Artificial Sequence
  Synthetic peptide
VVVNAANVY

SEQ ID NO: 557 - protein, 9 aa, Artificial Sequence
  Synthetic peptide
TVYSHLLLV

SEQ ID NO: 558 - protein, 10 aa, Artificial Sequence
  Synthetic peptide
ATEGALNTPK

SEQ ID NO: 559 - protein, 9 aa, Artificial Sequence
  Synthetic peptide
LPFNDGVYF

SEQ ID NO: 560 - protein, 9 aa, Artificial Sequence
  Synthetic peptide
GVYYHKNNK

SEQ ID NO: 561 - protein, 9 aa, Artificial Sequence
  Synthetic peptide
RYRIGNYKL

SEQ ID NO: 562 - protein, 9 aa, Artificial Sequence
  Synthetic peptide
FVIRGDEVR

SEQ ID NO: 563 - protein, 10 aa, Artificial Sequence
  Synthetic peptide
APSASAFFGM

SEQ ID NO: 564 - protein, 10 aa, Artificial Sequence
  Synthetic peptide
RASANLAATK

SEQ ID NO: 565 - protein, 9 aa, Artificial Sequence
  Synthetic peptide
FTIGTVTLK

SEQ ID NO: 566 - protein, 9 aa, Artificial Sequence
  Synthetic peptide
LLLDRLNQL

SEQ ID NO: 567 - protein, 9 aa, Artificial Sequence
  Synthetic peptide
GTRNPANNA

SEQ ID NO: 568 - protein, 9 aa, Artificial Sequence
  Synthetic peptide
ADAGFIKQY

SEQ ID NO: 569 - protein, 9 aa, Artificial Sequence
  Synthetic peptide
RLQSLQTYV

SEQ ID NO: 570 - protein, 9 aa, Artificial Sequence
  Synthetic peptide
LPPLLTDEM

SEQ ID NO: 571 - protein, 9 aa, Artificial Sequence
  Synthetic peptide
LEMELTPVV

SEQ ID NO: 572 - protein, 9 aa, Artificial Sequence
  Synthetic peptide
QVVNVVTTK

SEQ ID NO: 573 - protein, 9 aa, Artificial Sequence
  Synthetic peptide
MASLVLARK

SEQ ID NO: 574 - protein, 13 aa, Artificial Sequence
  Synthetic peptide
GPQNQRNAPRITF

SEQ ID NO: 575 - protein, 9 aa, Artificial Sequence
  Synthetic peptide
AMYTPHTVL

SEQ ID NO: 576 - protein, 10 aa, Artificial Sequence
  Synthetic peptide
RPQGLPNNTA

SEQ ID NO: 577 - protein, 10 aa, Artificial Sequence
  Synthetic peptide
DTIANYAKPF

SEQ ID NO: 578 - protein, 9 aa, Artificial Sequence
  Synthetic peptide
KEIDRLNEV

SEQ ID NO: 579 - protein, 10 aa, Artificial Sequence
  Synthetic peptide
KTFPPTEPKK

SEQ ID NO: 580 - protein, 9 aa, Artificial Sequence
  Synthetic peptide
FAVSKGFFK

SEQ ID NO: 581 - protein, 9 aa, Artificial Sequence
  Synthetic peptide
IPFAMQMAY

SEQ ID NO: 582 - protein, 9 aa, Artificial Sequence
  Synthetic peptide
IPTNFTISV

SEQ ID NO: 583 - protein, 9 aa, Artificial Sequence
  Synthetic peptide
YANRNRFLY

SEQ ID NO: 584 - protein, 9 aa, Artificial Sequence
  Synthetic peptide
EILPVSMTK

SEQ ID NO: 585 - protein, 10 aa, Artificial Sequence
  Synthetic peptide
KVGGNYNYLY

SEQ ID NO: 586 - protein, 9 aa, Artificial Sequence
  Synthetic peptide
HVTFFIYNK

SEQ ID NO: 587 - protein, 9 aa, Artificial Sequence
  Synthetic peptide
WEPEFYEAM

SEQ ID NO: 588 - protein, 10 aa, Artificial Sequence
  Synthetic peptide
HVTYVPAQEK

SEQ ID NO: 589 - protein, 10 aa, Artificial Sequence
  Synthetic peptide
QTVKPGNFNK

SEQ ID NO: 590 - protein, 10 aa, Artificial Sequence
  Synthetic peptide
VELGTEVNEF

SEQ ID NO: 591 - protein, 9 aa, Artificial Sequence
  Synthetic peptide
SELLTPLGI

SEQ ID NO: 592 - protein, 12 aa, Artificial Sequence
  Synthetic peptide
IPYNSVTSSIVI

SEQ ID NO: 593 - protein, 9 aa, Artificial Sequence
  Synthetic peptide
EGYLNSTNV

SEQ ID NO: 594 - protein, 10 aa, Artificial Sequence
  Synthetic peptide
LPYGANKDGI

SEQ ID NO: 595 - protein, 10 aa, Artificial Sequence
  Synthetic peptide
NKATYKPNTW

SEQ ID NO: 596 - protein, 9 aa, Artificial Sequence
  Synthetic peptide
YQDVNCTEV

SEQ ID NO: 597 - protein, 9 aa, Artificial Sequence
  Synthetic peptide
WTAGAAAYY

SEQ ID NO: 598 - protein, 13 aa, Artificial Sequence
  Synthetic peptide
DYVYLPYPDPSRI

SEQ ID NO: 599 - protein, 9 aa, Artificial Sequence
  Synthetic peptide
KTFPPTEPK

SEQ ID NO: 600 - protein, 8 aa, Artificial Sequence
  Synthetic peptide
AAVYRINW

SEQ ID NO: 601 - protein, 9 aa, Artificial Sequence
  Synthetic peptide
RLFARTRSM

SEQ ID NO: 602 - protein, 13 aa, Artificial Sequence
  Synthetic peptide
DFYDFAVSKGFFK

SEQ ID NO: 603 - protein, 9 aa, Artificial Sequence
  Synthetic peptide
APYIVGDVV

SEQ ID NO: 604 - protein, 9 aa, Artificial Sequence
  Synthetic peptide
FAMQMAYRF

SEQ ID NO: 605 - protein, 9 aa, Artificial Sequence
  Synthetic peptide
VTYVPAQEK

SEQ ID NO: 606 - protein, 12 aa, Artificial Sequence
  Synthetic peptide
SFPQSAPHGVVF

SEQ ID NO: 607 - protein, 9 aa, Artificial Sequence
  Synthetic peptide
RLFRKSNLK

SEQ ID NO: 608 - protein, 9 aa, Artificial Sequence
  Synthetic peptide
LPPAYTNSF

SEQ ID NO: 609 - protein, 10 aa, Artificial Sequence
  Synthetic peptide
AYVNTFSSTF

SEQ ID NO: 610 - protein, 9 aa, Artificial Sequence
  Synthetic peptide
GAAAYYVGY

SEQ ID NO: 611 - protein, 9 aa, Artificial Sequence
  Synthetic peptide
QEILGTVSW

SEQ ID NO: 612 - protein, 9 aa, Artificial Sequence
  Synthetic peptide
ESKPSVEQR

SEQ ID NO: 613 - protein, 8 aa, Artificial Sequence
  Synthetic peptide
LPFGWLIV

SEQ ID NO: 614 - protein, 10 aa, Artificial Sequence
  Synthetic peptide
MPTTIAKNTV

SEQ ID NO: 615 - protein, 9 aa, Artificial Sequence
  Synthetic peptide
TLDSKTQSL

SEQ ID NO: 616 - protein, 9 aa, Artificial Sequence
  Synthetic peptide
SEIIGYKAI

SEQ ID NO: 617 - protein, 14 aa, Artificial Sequence
  Synthetic peptide
TVYDPLQPELDSFK

SEQ ID NO: 618 - protein, 10 aa, Artificial Sequence
  Synthetic peptide
SWMESEFRVY

SEQ ID NO: 619 - protein, 9 aa, Artificial Sequence
  Synthetic peptide
AEAELAKNV

SEQ ID NO: 620 - protein, 9 aa, Artificial Sequence
  Synthetic peptide
KVFRSSVLH

SEQ ID NO: 621 - protein, 9 aa, Artificial Sequence
  Synthetic peptide
VPMEKLKTL

SEQ ID NO: 622 - protein, 9 aa, Artificial Sequence
  Synthetic peptide
FAYANRNRF

SEQ ID NO: 623 - protein, 11 aa, Artificial Sequence
  Synthetic peptide
VYLPYPDPSRI

SEQ ID NO: 624 - protein, 9 aa, Artificial Sequence
  Synthetic peptide
MFTPLVPFW

SEQ ID NO: 625 - protein, 9 aa, Artificial Sequence
  Synthetic peptide
FAYTKRNVI

SEQ ID NO: 626 - protein, 9 aa, Artificial Sequence
  Synthetic peptide
RTNVYLAVF

SEQ ID NO: 627 - protein, 10 aa, Artificial Sequence
  Synthetic peptide
FVVSTGYHFR

SEQ ID NO: 628 - protein, 9 aa, Artificial Sequence
  Synthetic peptide
IMASLVLAR

SEQ ID NO: 629 - protein, 10 aa, Artificial Sequence
  Synthetic peptide
SEDAQGMDNL

SEQ ID NO: 630 - protein, 10 aa, Artificial Sequence
  Synthetic peptide
AKSHNIALIW

SEQ ID NO: 631 - protein, 9 aa, Artificial Sequence
  Synthetic peptide
WFVTQRNFY

SEQ ID NO: 632 - protein, 9 aa, Artificial Sequence
  Synthetic peptide
SICSTMTNR

SEQ ID NO: 633 - protein, 9 aa, Artificial Sequence
  Synthetic peptide
QYIKWPWYI

SEQ ID NO: 634 - protein, 10 aa, Artificial Sequence
  Synthetic peptide
NGTITVEELK

SEQ ID NO: 635 - protein, 9 aa, Artificial Sequence
  Synthetic peptide
FLAHIQWMV

SEQ ID NO: 636 - protein, 9 aa, Artificial Sequence
  Synthetic peptide
NYMPYFFTL

SEQ ID NO: 637 - protein, 9 aa, Artificial Sequence
  Synthetic peptide
TLKEILVTY

SEQ ID NO: 638 - protein, 9 aa, Artificial Sequence
  Synthetic peptide
YGFQPTNGV

SEQ ID NO: 639 - protein, 9 aa, Artificial Sequence
  Synthetic peptide
TPHTVLQAV

SEQ ID NO: 640 - protein, 10 aa, Artificial Sequence
  Synthetic peptide
DGVYFASTEK

SEQ ID NO: 641 - protein, 9 aa, Artificial Sequence
  Synthetic peptide
YVMHANYIF

SEQ ID NO: 642 - protein, 10 aa, Artificial Sequence
  Synthetic peptide
FERDISTEIY

SEQ ID NO: 643 - protein, 9 aa, Artificial Sequence
  Synthetic peptide
EEVVENPTI

SEQ ID NO: 644 - protein, 9 aa, Artificial Sequence
  Synthetic peptide
KAYNVTQAF

SEQ ID NO: 645 - protein, 9 aa, Artificial Sequence
  Synthetic peptide
WLDMVDTSL

SEQ ID NO: 646 - protein, 9 aa, Artificial Sequence
  Synthetic peptide
AGLPYGANK

SEQ ID NO: 647 - protein, 9 aa, Artificial Sequence
  Synthetic peptide
IASFRLFAR

SEQ ID NO: 648 - protein, 10 aa, Artificial Sequence
  Synthetic peptide
KVAGFAKFLK

SEQ ID NO: 649 - protein, 9 aa, Artificial Sequence
  Synthetic peptide
QTNSPRRAR

SEQ ID NO: 650 - protein, 8 aa, Artificial Sequence
  Synthetic peptide
AEHVNNSY

SEQ ID NO: 651 - protein, 10 aa, Artificial Sequence
  Synthetic peptide
AYVDNSSLTI

SEQ ID NO: 652 - protein, 9 aa, Artificial Sequence
  Synthetic peptide
YIATNGPLK

SEQ ID NO: 653 - protein, 10 aa, Artificial Sequence
  Synthetic peptide
RTTNGDFLHF

SEQ ID NO: 654 - protein, 10 aa, Artificial Sequence
  Synthetic peptide
QYIKWPWYIW

SEQ ID NO: 655 - protein, 9 aa, Artificial Sequence
  Synthetic peptide
QPTESIVRF

SEQ ID NO: 656 - protein, 11 aa, Artificial Sequence
  Synthetic peptide
ITDVFYKENSY

SEQ ID NO: 657 - protein, 9 aa, Artificial Sequence
  Synthetic peptide
SANNCTFEY

SEQ ID NO: 658 - protein, 9 aa, Artificial Sequence
  Synthetic peptide
SVYAWNRKR

SEQ ID NO: 659 - protein, 9 aa, Artificial Sequence
  Synthetic peptide
YMSALNHTK

SEQ ID NO: 660 - protein, 10 aa, Artificial Sequence
  Synthetic peptide
AEWFLAYILF

SEQ ID NO: 661 - protein, 9 aa, Artificial Sequence
  Synthetic peptide
IPTNFTISV

SEQ ID NO: 662 - protein, 8 aa, Artificial Sequence
  Synthetic peptide
LPQGFSAL

SEQ ID NO: 663 - protein, 9 aa, Artificial Sequence
  Synthetic peptide
LPFNDGVYF

SEQ ID NO: 664 - protein, 9 aa, Artificial Sequence
  Synthetic peptide
MSNLGMPSY

SEQ ID NO: 665 - protein, 10 aa, Artificial Sequence
  Synthetic peptide
MELTPVVQTI

SEQ ID NO: 666 - protein, 9 aa, Artificial Sequence
  Synthetic peptide
DSGFAAYSR

SEQ ID NO: 667 - protein, 9 aa, Artificial Sequence
  Synthetic peptide
AGLEAPFLY

SEQ ID NO: 668 - protein, 10 aa, Artificial Sequence
  Synthetic peptide
LPIGINITRF

SEQ ID NO: 669 - protein, 9 aa, Artificial Sequence
  Synthetic peptide
ATVVIGTSK

SEQ ID NO: 670 - protein, 10 aa, Artificial Sequence
  Synthetic peptide
VENPDILRVY

SEQ ID NO: 671 - protein, 10 aa, Artificial Sequence
  Synthetic peptide
LEFGATSAAL

SEQ ID NO: 672 - protein, 9 aa, Artificial Sequence
  Synthetic peptide
NELSRVLGL

SEQ ID NO: 673 - protein, 9 aa, Artificial Sequence
  Synthetic peptide
LPPAYTNSF

SEQ ID NO: 674 - protein, 9 aa, Artificial Sequence
  Synthetic peptide
LVAEWFLAY

SEQ ID NO: 675 - protein, 9 aa, Artificial Sequence
  Synthetic peptide
LPLVSSQCV

SEQ ID NO: 676 - protein, 9 aa, Artificial Sequence
  Synthetic peptide
SALNHTKKW

SEQ ID NO: 677 - protein, 9 aa, Artificial Sequence
  Synthetic peptide
VVFLHVTYV

SEQ ID NO: 678 - protein, 9 aa, Artificial Sequence
  Synthetic peptide
AISDYDYYR

SEQ ID NO: 679 - protein, 8 aa, Artificial Sequence
  Synthetic peptide
MPYFFTLL

SEQ ID NO: 680 - protein, 9 aa, Artificial Sequence
  Synthetic peptide
NPLLYDANY

SEQ ID NO: 681 - protein, 9 aa, Artificial Sequence
  Synthetic peptide
VARDLSLQF

SEQ ID NO: 682 - protein, 9 aa, Artificial Sequence
  Synthetic peptide
FVVPGLPGT

SEQ ID NO: 683 - protein, 9 aa, Artificial Sequence
  Synthetic peptide
YNYLYRLFR

SEQ ID NO: 684 - protein, 9 aa, Artificial Sequence
  Synthetic peptide
VATSRTLSY

SEQ ID NO: 685 - protein, 10 aa, Artificial Sequence
  Synthetic peptide
YPQVNGLTSI

SEQ ID NO: 686 - protein, 11 aa, Artificial Sequence
  Synthetic peptide
TEIDPKLDNYY

SEQ ID NO: 687 - protein, 8 aa, Artificial Sequence
  Synthetic peptide
VYYPDKVF

SEQ ID NO: 688 - protein, 9 aa, Artificial Sequence
  Synthetic peptide
IAAVITREV

SEQ ID NO: 689 - protein, 9 aa, Artificial Sequence
  Synthetic peptide
KCYGVSPTK

SEQ ID NO: 690 - protein, 10 aa, Artificial Sequence
  Synthetic peptide
EVKPFITESK

SEQ ID NO: 691 - protein, 9 aa, Artificial Sequence
  Synthetic peptide
SIIAYTMSL

SEQ ID NO: 692 - protein, 10 aa, Artificial Sequence
  Synthetic peptide
NESLIDLQEL

SEQ ID NO: 693 - protein, 9 aa, Artificial Sequence
  Synthetic peptide
GTHWFVTQR

SEQ ID NO: 694 - protein, 9 aa, Artificial Sequence
  Synthetic peptide
FASVYAWNR

SEQ ID NO: 695 - protein, 9 aa, Artificial Sequence
  Synthetic peptide
LPPAYTNSF

SEQ ID NO: 696 - protein, 11 aa, Artificial Sequence
  Synthetic peptide
QEVFAQVKQIY

SEQ ID NO: 697 - protein, 10 aa, Artificial Sequence
  Synthetic peptide
FLPFFSNVTW

SEQ ID NO: 698 - protein, 9 aa, Artificial Sequence
  Synthetic peptide
NATRFASVY

SEQ ID NO: 699 - protein, 9 aa, Artificial Sequence
  Synthetic peptide
QPTESIVRF

SEQ ID NO: 700 - protein, 9 aa, Artificial Sequence
  Synthetic peptide
ASRELKVTF

SEQ ID NO: 701 - protein, 9 aa, Artificial Sequence
  Synthetic peptide
ASFDNFKFV

SEQ ID NO: 702 - protein, 10 aa, Artificial Sequence
  Synthetic peptide
VSSPDAVTAY

SEQ ID NO: 703 - protein, 9 aa, Artificial Sequence
  Synthetic peptide
YANRNRFLY

SEQ ID NO: 704 - protein, 13 aa, Artificial Sequence
  Synthetic peptide
REQIDGYVMHANY

SEQ ID NO: 705 - protein, 9 aa, Artificial Sequence
  Synthetic peptide
DAVRDPQTL

SEQ ID NO: 706 - protein, 8 aa, Artificial Sequence
  Synthetic peptide
FPREGVFV

SEQ ID NO: 707 - protein, 9 aa, Artificial Sequence
  Synthetic peptide
VYMPASWVM

SEQ ID NO: 708 - protein, 12 aa, Artificial Sequence
  Synthetic peptide
VAIHADQLTPTW

SEQ ID NO: 709 - protein, 9 aa, Artificial Sequence
  Synthetic peptide
FLYENAFLP

SEQ ID NO: 710 - protein, 10 aa, Artificial Sequence
  Synthetic peptide
RFASVYAWNR

SEQ ID NO: 711 - protein, 9 aa, Artificial Sequence
  Synthetic peptide
FERDISTEI

SEQ ID NO: 712 - protein, 9 aa, Artificial Sequence
  Synthetic peptide
AVHFISNSW

SEQ ID NO: 713 - protein, 9 aa, Artificial Sequence
  Synthetic peptide
YIFFASFYY

SEQ ID NO: 714 - protein, 9 aa, Artificial Sequence
  Synthetic peptide
GLTVLPPLL

SEQ ID NO: 715 - protein, 9 aa, Artificial Sequence
  Synthetic peptide
KSAAEASKK

SEQ ID NO: 716 - protein, 9 aa, Artificial Sequence
  Synthetic peptide
IYLYLTFYL

SEQ ID NO: 717 - protein, 9 aa, Artificial Sequence
  Synthetic peptide
VVTTKIALK

SEQ ID NO: 718 - protein, 9 aa, Artificial Sequence
  Synthetic peptide
DYKHYTPSF

SEQ ID NO: 719 - protein, 9 aa, Artificial Sequence
  Synthetic peptide
FTSDYYQLY

SEQ ID NO: 720 - protein, 10 aa, Artificial Sequence
  Synthetic peptide
FVENPDILRV

SEQ ID NO: 721 - protein, 9 aa, Artificial Sequence
  Synthetic peptide
YYKKDNSYF

SEQ ID NO: 722 - protein, 8 aa, Artificial Sequence
  Synthetic peptide
AYILFTRF

SEQ ID NO: 723 - protein, 9 aa, Artificial Sequence
  Synthetic peptide
VVAIDYKHY

SEQ ID NO: 724 - protein, 10 aa, Artificial Sequence
  Synthetic peptide
MSALNHTKKW

SEQ ID NO: 725 - protein, 9 aa, Artificial Sequence
  Synthetic peptide
TTIAKNTVK

SEQ ID NO: 726 - protein, 9 aa, Artificial Sequence
  Synthetic peptide
ATSRTLSYY

SEQ ID NO: 727 - protein, 9 aa, Artificial Sequence
  Synthetic peptide
SVLNDILSR

SEQ ID NO: 728 - protein, 8 aa, Artificial Sequence
  Synthetic peptide
RLQSLQTY

SEQ ID NO: 729 - protein, 10 aa, Artificial Sequence
  Synthetic peptide
YFTSDYYQLY

SEQ ID NO: 730 - protein, 12 aa, Artificial Sequence
  Synthetic peptide
SASKIITLKKRW

SEQ ID NO: 731 - protein, 9 aa, Artificial Sequence
  Synthetic peptide
ITLKKRWQL

SEQ ID NO: 732 - protein, 10 aa, Artificial Sequence
  Synthetic peptide
YKTFPPTEPK

SEQ ID NO: 733 - protein, 9 aa, Artificial Sequence
  Synthetic peptide
AEIRASANL

SEQ ID NO: 734 - protein, 9 aa, Artificial Sequence
  Synthetic peptide
MSMTYGQQF

SEQ ID NO: 735 - protein, 9 aa, Artificial Sequence
  Synthetic peptide
VVDYGARFY

SEQ ID NO: 736 - protein, 10 aa, Artificial Sequence
  Synthetic peptide
QEYADVFHLY

SEQ ID NO: 737 - protein, 9 aa, Artificial Sequence
  Synthetic peptide
SAKSASVYY

SEQ ID NO: 738 - protein, 9 aa, Artificial Sequence
  Synthetic peptide
MPASWVMRI

SEQ ID NO: 739 - protein, 10 aa, Artificial Sequence
  Synthetic peptide
STGSNVFQTR

SEQ ID NO: 740 - protein, 9 aa, Artificial Sequence
  Synthetic peptide
GMSRIGMEV

SEQ ID NO: 741 - protein, 10 aa, Artificial Sequence
  Synthetic peptide
KMFDAYVNTF

SEQ ID NO: 742 - protein, 11 aa, Artificial Sequence
  Synthetic peptide
FADDLNQLTGY

SEQ ID NO: 743 - protein, 9 aa, Artificial Sequence
  Synthetic peptide
SLIYSTAAL

SEQ ID NO: 744 - protein, 9 aa, Artificial Sequence
  Synthetic peptide
KSAGFPFNK

SEQ ID NO: 745 - protein, 11 aa, Artificial Sequence
  Synthetic peptide
VTVKNGSIHLY

SEQ ID NO: 746 - protein, 9 aa, Artificial Sequence
  Synthetic peptide
SLDNVLSTF

SEQ ID NO: 747 - protein, 10 aa, Artificial Sequence
  Synthetic peptide
NPANNAAIVL

SEQ ID NO: 748 - protein, 8 aa, Artificial Sequence
  Synthetic peptide
ISTKHFYW

SEQ ID NO: 749 - protein, 9 aa, Artificial Sequence
  Synthetic peptide
STNVTIATY

SEQ ID NO: 750 - protein, 9 aa, Artificial Sequence
  Synthetic peptide
RLFRKSNLK

SEQ ID NO: 751 - protein, 10 aa, Artificial Sequence
  Synthetic peptide
STGSNVFQTR

SEQ ID NO: 752 - protein, 10 aa, Artificial Sequence
  Synthetic peptide
KFCLEASFNY

SEQ ID NO: 753 - protein, 9 aa, Artificial Sequence
  Synthetic peptide
TSNPTTFHL

SEQ ID NO: 754 - protein, 9 aa, Artificial Sequence
  Synthetic peptide
LGAENSVAY

SEQ ID NO: 755 - protein, 9 aa, Artificial Sequence
  Synthetic peptide
VMYMGTLSY

SEQ ID NO: 756 - protein, 8 aa, Artificial Sequence
  Synthetic peptide
LPRVFSAV

SEQ ID NO: 757 - protein, 8 aa, Artificial Sequence
  Synthetic peptide
LPGVYSVI

SEQ ID NO: 758 - protein, 14 aa, Artificial Sequence
  Synthetic peptide
QSDIEVTGDSCNNY

SEQ ID NO: 759 - protein, 9 aa, Artificial Sequence
  Synthetic peptide
ALDPLSETK

SEQ ID NO: 760 - protein, 10 aa, Artificial Sequence
  Synthetic peptide
FGEYSHVVAF

SEQ ID NO: 761 - protein, 14 aa, Artificial Sequence
  Synthetic peptide
KTFPPTEPKKDKKK

SEQ ID NO: 762 - protein, 10 aa, Artificial Sequence
  Synthetic peptide
IHADQLTPTW

SEQ ID NO: 763 - protein, 9 aa, Artificial Sequence
  Synthetic peptide
AIVSTIQRK

SEQ ID NO: 764 - protein, 10 aa, Artificial Sequence
  Synthetic peptide
YSRYRIGNYK

SEQ ID NO: 765 - protein, 9 aa, Artificial Sequence
  Synthetic peptide
KLMGHFAWW

SEQ ID NO: 766 - protein, 11 aa, Artificial Sequence
  Synthetic peptide
RVAGDSGFAAY

SEQ ID NO: 767 - protein, 9 aa, Artificial Sequence
  Synthetic peptide
EILPVSMTK

SEQ ID NO: 768 - protein, 9 aa, Artificial Sequence
  Synthetic peptide
SSAKSASVY

SEQ ID NO: 769 - protein, 9 aa, Artificial Sequence
  Synthetic peptide
YLATALLTL

SEQ ID NO: 770 - protein, 10 aa, Artificial Sequence
  Synthetic peptide
KFLPFQQFGR

SEQ ID NO: 771 - protein, 9 aa, Artificial Sequence
  Synthetic peptide
STNVTIATY

SEQ ID NO: 772 - protein, 10 aa, Artificial Sequence
  Synthetic peptide
YVYANGGKGF

SEQ ID NO: 773 - protein, 9 aa, Artificial Sequence
  Synthetic peptide
STVFPPTSF

SEQ ID NO: 774 - protein, 9 aa, Artificial Sequence
  Synthetic peptide
KLINIIIWF

SEQ ID NO: 775 - protein, 9 aa, Artificial Sequence
  Synthetic peptide
HSYFTSDYY

SEQ ID NO: 776 - protein, 10 aa, Artificial Sequence
  Synthetic peptide
TNVLEGSVAY

SEQ ID NO: 777 - protein, 9 aa, Artificial Sequence
  Synthetic peptide
EIDPKLDNY

SEQ ID NO: 778 - protein, 10 aa, Artificial Sequence
  Synthetic peptide
GVYYPDKVFR

SEQ ID NO: 779 - protein, 11 aa, Artificial Sequence
  Synthetic peptide
EVTPSGTWLTY

SEQ ID NO: 780 - protein, 9 aa, Artificial Sequence
  Synthetic peptide
KVAGFAKFL

SEQ ID NO: 781 - protein, 9 aa, Artificial Sequence
  Synthetic peptide
DASGKPVPY

SEQ ID NO: 782 - protein, 9 aa, Artificial Sequence
  Synthetic peptide
AEIRASANL

SEQ ID NO: 783 - protein, 9 aa, Artificial Sequence
  Synthetic peptide
LYDKLVSSF

SEQ ID NO: 784 - protein, 9 aa, Artificial Sequence
  Synthetic peptide
LEAPFLYLY

SEQ ID NO: 785 - protein, 9 aa, Artificial Sequence
  Synthetic peptide
NSASFSTFK

SEQ ID NO: 786 - protein, 9 aa, Artificial Sequence
  Synthetic peptide
WTFGAGAAL

SEQ ID NO: 787 - protein, 9 aa, Artificial Sequence
  Synthetic peptide
YYQLYSTQL

SEQ ID NO: 788 - protein, 9 aa, Artificial Sequence
  Synthetic peptide
TSNQVAVLY

SEQ ID NO: 789 - protein, 8 aa, Artificial Sequence
  Synthetic peptide
RTRSMWSF

SEQ ID NO: 790 - protein, 10 aa, Artificial Sequence
  Synthetic peptide
FVNEFYAYLR

SEQ ID NO: 791 - protein, 10 aa, Artificial Sequence
  Synthetic peptide
NVYADSFVIR

SEQ ID NO: 792 - protein, 9 aa, Artificial Sequence
  Synthetic peptide
DAVNLLTNM

SEQ ID NO: 793 - protein, 9 aa, Artificial Sequence
  Synthetic peptide
VAMPNLYKM

SEQ ID NO: 794 - protein, 10 aa, Artificial Sequence
  Synthetic peptide
SVASQSIIAY

SEQ ID NO: 795 - protein, 10 aa, Artificial Sequence
  Synthetic peptide
LPFAMGIIAM

SEQ ID NO: 796 - protein, 9 aa, Artificial Sequence
  Synthetic peptide
RIAGHHLGR

SEQ ID NO: 797 - protein, 13 aa, Artificial Sequence
  Synthetic peptide
YTMSLGAENSVAY

SEQ ID NO: 798 - protein, 10 aa, Artificial Sequence
  Synthetic peptide
FELDERIDKV

SEQ ID NO: 799 - protein, 9 aa, Artificial Sequence
  Synthetic peptide
AEVQIDRLI

SEQ ID NO: 800 - protein, 9 aa, Artificial Sequence
  Synthetic peptide
GTITSGWTF

SEQ ID NO: 801 - protein, 9 aa, Artificial Sequence
  Synthetic peptide
FPQSAPHGV

SEQ ID NO: 802 - protein, 11 aa, Artificial Sequence
  Synthetic peptide
YECDIPIGAGI

SEQ ID NO: 803 - protein, 8 aa, Artificial Sequence
  Synthetic peptide
AEVQIDRL

SEQ ID NO: 804 - protein, 15 aa, Artificial Sequence
  Synthetic peptide
NHNFLVQAGNVQLRV

SEQ ID NO: 805 - protein, 15 aa, Artificial Sequence
  Synthetic peptide
QNCVLKLKVDTANPK

SEQ ID NO: 806 - protein, 15 aa, Artificial Sequence
  Synthetic peptide
SEETGTLIVNSVLLF

SEQ ID NO: 807 - protein, 15 aa, Artificial Sequence
  Synthetic peptide
LAILTALRLCAYCCN

SEQ ID NO: 808 - protein, 15 aa, Artificial Sequence
  Synthetic peptide
TSHKLVLSVNPYVCN

SEQ ID NO: 809 - protein, 15 aa, Artificial Sequence
  Synthetic peptide
ISPYNSQNAVASKIL

SEQ ID NO: 810 - protein, 15 aa, Artificial Sequence
  Synthetic peptide
NVNRFNVAITRAKVG

SEQ ID NO: 811 - protein, 15 aa, Artificial Sequence
  Synthetic peptide
QPYVFIKRSDARTAP

SEQ ID NO: 812 - protein, 15 aa, Artificial Sequence
  Synthetic peptide
VLSFCAFAVDAAKAY

SEQ ID NO: 813 - protein, 15 aa, Artificial Sequence
  Synthetic peptide
NMFITREEAIRHVRA

SEQ ID NO: 814 - protein, 15 aa, Artificial Sequence
  Synthetic peptide
REEAIRHVRAWIGFD

SEQ ID NO: 815 - protein, 15 aa, Artificial Sequence
  Synthetic peptide
PLMYKGLPWNVVRIK

SEQ ID NO: 816 - protein, 15 aa, Artificial Sequence
  Synthetic peptide
EIIKSQDLSVVSKVV

SEQ ID NO: 817 - protein, 15 aa, Artificial Sequence
  Synthetic peptide
TQLCQYLNTLTLAVP

SEQ ID NO: 818 - protein, 15 aa, Artificial Sequence
  Synthetic peptide
QIDGYVMHANYIFWR

SEQ ID NO: 819 - protein, 15 aa, Artificial Sequence
  Synthetic peptide
PLNSIIKTIQPRVEK

SEQ ID NO: 820 - protein, 15 aa, Artificial Sequence
  Synthetic peptide
EEIAIILASFSASTS

SEQ ID NO: 821 - protein, 15 aa, Artificial Sequence
  Synthetic peptide
SPLYAFASEAARVVR

SEQ ID NO: 822 - protein, 15 aa, Artificial Sequence
  Synthetic peptide
AITILDGISQYSLRL

SEQ ID NO: 823 - protein, 15 aa, Artificial Sequence
  Synthetic peptide
QTFFKLVNKFLALCA

SEQ ID NO: 824 - protein, 15 aa, Artificial Sequence
  Synthetic peptide
GETFVTHSKGLYRKC

SEQ ID NO: 825 - protein, 15 aa, Artificial Sequence
  Synthetic peptide
KGGKIVNNWLKQLIK

SEQ ID NO: 826 - protein, 15 aa, Artificial Sequence
  Synthetic peptide
LFVAAIFYLITPVHV

SEQ ID NO: 827 - protein, 15 aa, Artificial Sequence
  Synthetic peptide
DTRYVLMDGSIIQFP

SEQ ID NO: 828 - protein, 15 aa, Artificial Sequence
  Synthetic peptide
FGEYSHVVAFNTLLF

SEQ ID NO: 829 - protein, 15 aa, Artificial Sequence
  Synthetic peptide
NTLLFLMSFTVLCLT

SEQ ID NO: 830 - protein, 15 aa, Artificial Sequence
  Synthetic peptide
TIAYIICISTKHFYW

SEQ ID NO: 831 - protein, 15 aa, Artificial Sequence
  Synthetic peptide
CTFLLNKEMYLKLRS

SEQ ID NO: 832 - protein, 15 aa, Artificial Sequence
  Synthetic peptide
LLVLVQSTQWSLFFF

SEQ ID NO: 833 - protein, 15 aa, Artificial Sequence
  Synthetic peptide
SLFFFLYENAFLPFA

SEQ ID NO: 834 - protein, 15 aa, Artificial Sequence
  Synthetic peptide
LCLFLLPSLATVAYF

SEQ ID NO: 835 - protein, 15 aa, Artificial Sequence
  Synthetic peptide
TLVYKVYYGNALDQA

SEQ ID NO: 836 - protein, 15 aa, Artificial Sequence
  Synthetic peptide
NRYFRLTLGVYDYLV

SEQ ID NO: 837 - protein, 15 aa, Artificial Sequence
  Synthetic peptide
DAFKLNIKLLGVGGK

SEQ ID NO: 838 - protein, 15 aa, Artificial Sequence
  Synthetic peptide
VLKKLKKSLNVAKSE

SEQ ID NO: 839 - protein, 15 aa, Artificial Sequence
  Synthetic peptide
LIVTALRANSAVKLQ

SEQ ID NO: 840 - protein, 15 aa, Artificial Sequence
  Synthetic peptide
SDFVRATATIPIQAS

SEQ ID NO: 841 - protein, 15 aa, Artificial Sequence
  Synthetic peptide
MFHLVDFQVTIAEIL

SEQ ID NO: 842 - protein, 15 aa, Artificial Sequence
  Synthetic peptide
IAEILLIIMRTFKVS

SEQ ID NO: 843 - protein, 15 aa, Artificial Sequence
  Synthetic peptide
LIIMRTFKVSIWNLD

SEQ ID NO: 844 - protein, 15 aa, Artificial Sequence
  Synthetic peptide
TFKVSIWNLDYIINL

SEQ ID NO: 845 - protein, 15 aa, Artificial Sequence
  Synthetic peptide
IWNLDYIINLIIKNL

SEQ ID NO: 846 - protein, 15 aa, Artificial Sequence
  Synthetic peptide
YIINLIIKNLSKSLT

SEQ ID NO: 847 - protein, 15 aa, Artificial Sequence
  Synthetic peptide
MKIILFLALITLATC

SEQ ID NO: 848 - protein, 15 aa, Artificial Sequence
  Synthetic peptide
VKHVYQLRARSVSPK

SEQ ID NO: 849 - protein, 15 aa, Artificial Sequence
  Synthetic peptide
LYSPIFLIVAAIVFI

SEQ ID NO: 850 - protein, 15 aa, Artificial Sequence
  Synthetic peptide
ENLLLYIDINGNLHP

SEQ ID NO: 851 - protein, 15 aa, Artificial Sequence
  Synthetic peptide
EAARYMRSLKVPATV

SEQ ID NO: 852 - protein, 15 aa, Artificial Sequence
  Synthetic peptide
DNFKFVCDNIKFADD

SEQ ID NO: 853 - protein, 15 aa, Artificial Sequence
  Synthetic peptide
NELSRVLGLKTLATH

SEQ ID NO: 854 - protein, 15 aa, Artificial Sequence
  Synthetic peptide
LEASFNYLKSPNFSK

SEQ ID NO: 855 - protein, 15 aa, Artificial Sequence
  Synthetic peptide
GSLIYSTAALGVLMS

SEQ ID NO: 856 - protein, 15 aa, Artificial Sequence
  Synthetic peptide
ISSFKWDLTAFGLVA

SEQ ID NO: 857 - protein, 15 aa, Artificial Sequence
  Synthetic peptide
FDAYVNTFSSTFNVP

SEQ ID NO: 858 - protein, 15 aa, Artificial Sequence
  Synthetic peptide
SHNIALIWNVKDFMS

SEQ ID NO: 859 - protein, 15 aa, Artificial Sequence
  Synthetic peptide
PDILRVYANLGERVR

SEQ ID NO: 860 - protein, 15 aa, Artificial Sequence
  Synthetic peptide
SLLMPILTLTRALTA

SEQ ID NO: 861 - protein, 15 aa, Artificial Sequence
  Synthetic peptide
EFYAYLRKHFSMMIL

SEQ ID NO: 862 - protein, 15 aa, Artificial Sequence
  Synthetic peptide
LRKHFSMMILSDDAV

SEQ ID NO: 863 - protein, 15 aa, Artificial Sequence
  Synthetic peptide
GLVASIKNFKSVLYY

SEQ ID NO: 864 - protein, 15 aa, Artificial Sequence
  Synthetic peptide
NNATNVVIKVCEFQF

SEQ ID NO: 865 - protein, 15 aa, Artificial Sequence
  Synthetic peptide
QPTESIVRFPNITNL

SEQ ID NO: 866 - protein, 15 aa, Artificial Sequence
  Synthetic peptide
VFNATRFASVYAWNR

SEQ ID NO: 867 - protein, 15 aa, Artificial Sequence
  Synthetic peptide
SVASQSIIAYTMSLG

SEQ ID NO: 868 - protein, 15 aa, Artificial Sequence
  Synthetic peptide
WTFGAGAALQIPFAM

SEQ ID NO: 869 - protein, 15 aa, Artificial Sequence
  Synthetic peptide
AQALNTLVKQLSSNF

SEQ ID NO: 870 - protein, 15 aa, Artificial Sequence
  Synthetic peptide
GAISSVLNDILSRLD

SEQ ID NO: 871 - protein, 15 aa, Artificial Sequence
  Synthetic peptide
VQIDRLITGRLQSLQ

SEQ ID NO: 872 - protein, 15 aa, Artificial Sequence
  Synthetic peptide
LNEVAKNLNESLIDL

SEQ ID NO: 873 - protein, 15 aa, Artificial Sequence
  Synthetic peptide
YEQYIKWPWYIWLGF

SEQ ID NO: 874 - protein, 15 aa, Artificial Sequence
  Synthetic peptide
NSLFRMPLGVYNYKI

SEQ ID NO: 875 - protein, 15 aa, Artificial Sequence
  Synthetic peptide
NHAFWVFSYCRKLGT

SEQ ID NO: 876 - protein, 15 aa, Artificial Sequence
  Synthetic peptide
PLLENIDYFNMRRAK

SEQ ID NO: 877 - protein, 15 aa, Artificial Sequence
  Synthetic peptide
EYYEFLNKHFSMMIL

SEQ ID NO: 878 - protein, 15 aa, Artificial Sequence
  Synthetic peptide
NDVAFVSTFNVLQDV

SEQ ID NO: 879 - protein, 15 aa, Artificial Sequence
  Synthetic peptide
KEEAVKRVRAWVGFD

SEQ ID NO: 880 - protein, 15 aa, Artificial Sequence
  Synthetic peptide
YQKVFRVYLAYIKKL

SEQ ID NO: 881 - protein, 15 aa, Artificial Sequence
  Synthetic peptide
LNKHFSMMILSDDGV

SEQ ID NO: 882 - protein, 15 aa, Artificial Sequence
  Synthetic peptide
VPLNAIPSLAANTLN

SEQ ID NO: 883 - protein, 15 aa, Artificial Sequence
  Synthetic peptide
MRFYIIIASFIKLFS

SEQ ID NO: 884 - protein, 15 aa, Artificial Sequence
  Synthetic peptide
QLKQLEKACNIAKSA

SEQ ID NO: 885 - protein, 15 aa, Artificial Sequence
  Synthetic peptide
ISPYNSQNFAAKRVL

SEQ ID NO: 886 - protein, 15 aa, Artificial Sequence
  Synthetic peptide
TDHKYVLSVSPYVCN

SEQ ID NO: 887 - protein, 15 aa, Artificial Sequence
  Synthetic peptide
KITEWPTATGDVVLA

SEQ ID NO: 888 - protein, 15 aa, Artificial Sequence
  Synthetic peptide
QYSFKLVMNGLVFGL

SEQ ID NO: 889 - protein, 15 aa, Artificial Sequence
  Synthetic peptide
TDLTVTSAGQPCVAS

SEQ ID NO: 890 - protein, 15 aa, Artificial Sequence
  Synthetic peptide
ALIATAHSSIKQGTQ

SEQ ID NO: 891 - protein, 15 aa, Artificial Sequence
  Synthetic peptide
LQSAATIRSVAYVAN

SEQ ID NO: 892 - protein, 15 aa, Artificial Sequence
  Synthetic peptide
YENKLKAKNESSSLC

SEQ ID NO: 893 - protein, 15 aa, Artificial Sequence
  Synthetic peptide
MPFLLDDLVPRAYYL

SEQ ID NO: 894 - protein, 15 aa, Artificial Sequence
  Synthetic peptide
NSSILSLCAFSVDPK

SEQ ID NO: 895 - protein, 15 aa, Artificial Sequence
  Synthetic peptide
SPCKELEGVGAKVSA

SEQ ID NO: 896 - protein, 15 aa, Artificial Sequence
  Synthetic peptide
DLALKLKGLDAMFFY

SEQ ID NO: 897 - protein, 15 aa, Artificial Sequence
  Synthetic peptide
KDVYELRYNGAIRFD

SEQ ID NO: 898 - protein, 15 aa, Artificial Sequence
  Synthetic peptide
SLFVDYSNLLHSKVK

SEQ ID NO: 899 - protein, 15 aa, Artificial Sequence
  Synthetic peptide
NRFFKCTMGVYDFKV

SEQ ID NO: 900 - protein, 15 aa, Artificial Sequence
  Synthetic peptide
GHFNEEFYNFLRLRG

SEQ ID NO: 901 - protein, 15 aa, Artificial Sequence
  Synthetic peptide
LLSSLTLTVKFVVES

SEQ ID NO: 902 - protein, 15 aa, Artificial Sequence
  Synthetic peptide
VLLERYVSLAIDAYP

SEQ ID NO: 903 - protein, 15 aa, Artificial Sequence
  Synthetic peptide
DYYGYLRKHFSMMIL

SEQ ID NO: 904 - protein, 15 aa, Artificial Sequence
  Synthetic peptide
LHNFSVSHNGVFLGV

SEQ ID NO: 905 - protein, 15 aa, Artificial Sequence
  Synthetic peptide
RDFAIRNVRGWLGMD

SEQ ID NO: 906 - protein, 15 aa, Artificial Sequence
  Synthetic peptide
LFTNSILMLDKQGQL

SEQ ID NO: 907 - protein, 15 aa, Artificial Sequence
  Synthetic peptide
LRKHFSMMILSDDGV

SEQ ID NO: 908 - protein, 15 aa, Artificial Sequence
  Synthetic peptide
SDLSTLAVTAIVVVG

SEQ ID NO: 909 - protein, 15 aa, Artificial Sequence
  Synthetic peptide
KAINNIVASFSSVND

SEQ ID NO: 910 - protein, 15 aa, Artificial Sequence
  Synthetic peptide
LIKQLKRAMNIAKSE

SEQ ID NO: 911 - protein, 15 aa, Artificial Sequence
  Synthetic peptide
TQLCQYLNSTTMCVP

SEQ ID NO: 912 - protein, 15 aa, Artificial Sequence
  Synthetic peptide
ISPYNSQNYVASRFL

SEQ ID NO: 913 - protein, 15 aa, Artificial Sequence
  Synthetic peptide
KDGFFTYLNGVIREK

SEQ ID NO: 914 - protein, 15 aa, Artificial Sequence
  Synthetic peptide
VPFDVLCNEFLATFI

SEQ ID NO: 915 - protein, 15 aa, Artificial Sequence
  Synthetic peptide
KHLKSIVNTRNATVV

SEQ ID NO: 916 - protein, 15 aa, Artificial Sequence
  Synthetic peptide
FIAGNTVHANYIFWR

SEQ ID NO: 917 - protein, 15 aa, Artificial Sequence
  Synthetic peptide
GVFVQDPAPIDIDAF

SEQ ID NO: 918 - protein, 15 aa, Artificial Sequence
  Synthetic peptide
STILQAAGLCVVCGS

SEQ ID NO: 919 - protein, 15 aa, Artificial Sequence
  Synthetic peptide
YVTNEIGLNASVTLK

SEQ ID NO: 920 - protein, 15 aa, Artificial Sequence
  Synthetic peptide
MDLLLDDFVTILKSL

SEQ ID NO: 921 - protein, 15 aa, Artificial Sequence
  Synthetic peptide
VDISYLNRARGSSAA

SEQ ID NO: 922 - protein, 15 aa, Artificial Sequence
  Synthetic peptide
ANGFFYIDVGNHRSA

SEQ ID NO: 923 - protein, 15 aa, Artificial Sequence
  Synthetic peptide
IIAVELLLLDFKTAV

SEQ ID NO: 924 - protein, 15 aa, Artificial Sequence
  Synthetic peptide
PSVAVRTYSEAAAQG

SEQ ID NO: 925 - protein, 15 aa, Artificial Sequence
  Synthetic peptide
LNLSSELKQLEAKTA

SEQ ID NO: 926 - protein, 15 aa, Artificial Sequence
  Synthetic peptide
LNKHFSMIILSDDGV

SEQ ID NO: 927 - protein, 15 aa, Artificial Sequence
  Synthetic peptide
LNKHFNMMILSDDGV

SEQ ID NO: 928 - protein, 15 aa, Artificial Sequence
  Synthetic peptide
TQLCQYLSTTTLAVP

SEQ ID NO: 929 - protein, 15 aa, Artificial Sequence
  Synthetic peptide
NVNRFNVAITRARKG

SEQ ID NO: 930 - protein, 15 aa, Artificial Sequence
  Synthetic peptide
LFFHFSMMILSDDGV

SEQ ID NO: 931 - protein, 15 aa, Artificial Sequence
  Synthetic peptide
TQLCQYLSTTTIAVP

SEQ ID NO: 932 - protein, 15 aa, Artificial Sequence
  Synthetic peptide
EIDGSVMHANYLFWR

SEQ ID NO: 933 - protein, 15 aa, Artificial Sequence
  Synthetic peptide
LLIKRFVSLAIHAYP

SEQ ID NO: 934 - protein, 15 aa, Artificial Sequence
  Synthetic peptide
EFYEFLNKHFSMMIL

SEQ ID NO: 935 - protein, 15 aa, Artificial Sequence
  Synthetic peptide
KCLKSIAATRGVSVV

SEQ ID NO: 936 - protein, 15 aa, Artificial Sequence
  Synthetic peptide
RDEAIRRVRAWVGFD

SEQ ID NO: 937 - protein, 15 aa, Artificial Sequence
  Synthetic peptide
NVNRFNLAITRAKKG

SEQ ID NO: 938 - protein, 15 aa, Artificial Sequence
  Synthetic peptide
FNKHFSMMILSDDGV

SEQ ID NO: 939 - protein, 15 aa, Artificial Sequence
  Synthetic peptide
TQLCQYLNTTTIAVP

SEQ ID NO: 940 - protein, 15 aa, Artificial Sequence
  Synthetic peptide
ISPYNSQNYVAKRIL

SEQ ID NO: 941 - protein, 15 aa, Artificial Sequence
  Synthetic peptide
TDHKYVLSVAPYVCN

SEQ ID NO: 942 - protein, 15 aa, Artificial Sequence
  Synthetic peptide
DIDGNVMHANYLFWR

SEQ ID NO: 943 - protein, 15 aa, Artificial Sequence
  Synthetic peptide
NSVFRMPMGVYNYKI

SEQ ID NO: 944 - protein, 15 aa, Artificial Sequence
  Syntjetic peptide
NHVLWLFSYCRKIGV

SEQ ID NO: 945 - protein, 15 aa, Artificial Sequence
  Synthetic peptide
PSNSIVCRFDTRVLN

SEQ ID NO: 946 - protein, 15 aa, Artificial Sequence
  Synthetic peptide
LLIERFVSLAIDAYP

SEQ ID NO: 947 - protein, 15 aa, Artificial Sequence
  Synthetic peptide
EYYEFLCKHFSMMIL

SEQ ID NO: 948 - protein, 15 aa, Artificial Sequence
  Synthetic peptide
FYGPYRDAQVVQLPV

SEQ ID NO: 949 - protein, 15 aa, Artificial Sequence
  Synthetic peptide
KDEAIKRVRGWVGFD

SEQ ID NO: 950 - protein, 15 aa, Artificial Sequence
  Synthetic peptide
LERVSLWNYGKPINL

SEQ ID NO: 951 - protein, 15 aa, Artificial Sequence
  Synthetic peptide
NVNRFNVAITRAKKG

SEQ ID NO: 952 - protein, 15 aa, Artificial Sequence
  Synthetic peptide
LCKHFSMMILSDDGV

SEQ ID NO: 953 - protein, 15 aa, Artificial Sequence
  Synthetic peptide
SKLFITKDEAIKRVR

SEQ ID NO: 954 - protein, 15 aa, Artificial Sequence
  Synthetic peptide
LEYPIISNEVSINTS

SEQ ID NO: 955 - protein, 15 aa, Artificial Sequence
  Synthetic peptide
QIKQLEKACNIAKSV

SEQ ID NO: 956 - protein, 15 aa, Artificial Sequence
  Synthetic peptide
TQLCQYLNTTTLAVP

SEQ ID NO: 957 - protein, 15 aa, Artificial Sequence
  Synthetic peptide
ISPYNSQNYVAKRVL

SEQ ID NO: 958 - protein, 15 aa, Artificial Sequence
  Synthetic peptide
TNHKYVLSVSPYVCN

SEQ ID NO: 959 - protein, 15 aa, Artificial Sequence
  Synthetic peptide
KVTVWPVATGDVVLA

SEQ ID NO: 960 - protein, 15 aa, Artificial Sequence
  Synthetic peptide
ETFGKPVIWFCHDEA

SEQ ID NO: 961 - protein, 15 aa, Artificial Sequence
  Synthetic peptide
ETFGKPVIWFCHDEA

SEQ ID NO: 962 - protein, 15 aa, Artificial Sequence
  Synthetic peptide
EIDGNVMHANYLFWR

SEQ ID NO: 963 - protein, 15 aa, Artificial Sequence
  Synthetic peptide
SRFVMRLQTIATICG

SEQ ID NO: 964 - protein, 15 aa, Artificial Sequence
  Synthetic peptide
VLLTVDGVNFKSISL

SEQ ID NO: 965 - protein, 15 aa, Artificial Sequence
  Synthetic peptide
LALLYRNLKCSYVLN

SEQ ID NO: 966 - protein, 15 aa, Artificial Sequence
  Synthetic peptide
KIEDLSIRNLQKRLY

SEQ ID NO: 967 - protein, 15 aa, Artificial Sequence
  Synthetic peptide
MHWLIRFIVFVANML

SEQ ID NO: 968 - protein, 15 aa, Artificial Sequence
  Synthetic peptide
VDNVYVTYAGSVWHI

SEQ ID NO: 969 - protein, 15 aa, Artificial Sequence
  Synthetic peptide
TRSMTYCRVGACEYA

SEQ ID NO: 970 - protein, 15 aa, Artificial Sequence
  Synthetic peptide
APATGWLLYQLLNGL

SEQ ID NO: 971 - protein, 15 aa, Artificial Sequence
  Synthetic peptide
PGNTFITVEAAIELS

SEQ ID NO: 972 - protein, 15 aa, Artificial Sequence
  Synthetic peptide
WLLPDAAEELASPMK

SEQ ID NO: 973 - protein, 15 aa, Artificial Sequence
  Synthetic peptide
NRFCKCTLGVYDFCV

SEQ ID NO: 974 - protein, 15 aa, Artificial Sequence
  Synthetic peptide
KSFSTFESAYMPIAD

SEQ ID NO: 975 - protein, 15 aa, Artificial Sequence
  Synthetic peptide
PCPSILKVIDGGKIW

SEQ ID NO: 976 - protein, 15 aa, Artificial Sequence
  Synthetic peptide
ILLERYVSLAIDAYP

SEQ ID NO: 977 - protein, 15 aa, Artificial Sequence
  Synthetic peptide
DFYGYLQKHFSMMIL

SEQ ID NO: 978 - protein, 15 aa, Artificial Sequence
  Synthetic peptide
LHNFSIISGTAFLGV

SEQ ID NO: 979 - protein, 15 aa, Artificial Sequence
  Synthetic peptide
RDFAMRHVRGWLGMD

SEQ ID NO: 980 - protein, 15 aa, Artificial Sequence
  Synthetic peptide
NDKITEFQLDYSIDV

SEQ ID NO: 981 - protein, 15 aa, Artificial Sequence
  Synthetic peptide
NANRFNVAITRAKKG

SEQ ID NO: 982 - protein, 15 aa, Artificial Sequence
  Synthetic peptide
LQKHFSMMILSDDSV

SEQ ID NO: 983 - protein, 15 aa, Artificial Sequence
  Synthetic peptide
VDHSAFAYESAVVNG

SEQ ID NO: 984 - protein, 15 aa, Artificial Sequence
  Synthetic peptide
NLVFNILSMFSSSFS

SEQ ID NO: 985 - protein, 15 aa, Artificial Sequence
  Synthetic peptide
IIKQLKKAMNVAKAE

SEQ ID NO: 986 - protein, 15 aa, Artificial Sequence
  Synthetic peptide
TQLCQYFNSTTLCVP

SEQ ID NO: 987 - protein, 15 aa, Artificial Sequence
  Synthetic peptide
ISPYNSQNYVAARLL

SEQ ID NO: 988 - protein, 15 aa, Artificial Sequence
  Synthetic peptide
TDHKFILAITPYVCN

SEQ ID NO: 989 - protein, 15 aa, Artificial Sequence
  Synthetic peptide
QEATLPDIAEDVVDQ

SEQ ID NO: 990 - protein, 15 aa, Artificial Sequence
  Synthetic peptide
QTLFCNIMKFSDRPF

SEQ ID NO: 991 - protein, 15 aa, Artificial Sequence
  Synthetic peptide
KCLKSIVATRNATVV

SEQ ID NO: 992 - protein, 15 aa, Artificial Sequence
  Synthetic peptide
FIDGNIIHANYVFWR

SEQ ID NO: 993 - protein, 15 aa, Artificial Sequence
  Synthetic peptide
SAVAVVGGTIQILAS

SEQ ID NO: 994 - protein, 15 aa, Artificial Sequence
  Synthetic peptide
ALLAFFLSKHSDFGL

SEQ ID NO: 995 - protein, 15 aa, Artificial Sequence
  Synthetic peptide
LAKFTKLLLLIYTLY

SEQ ID NO: 996 - protein, 15 aa, Artificial Sequence
  Synthetic peptide
VIDNEIIVKPNISLC

SEQ ID NO: 997 - protein, 15 aa, Artificial Sequence
  Synthetic peptide
NVHLKDVTKENQEIL

SEQ ID NO: 998 - protein, 15 aa, Artificial Sequence
  Synthetic peptide
IKNVNSVRDWLKSLK

SEQ ID NO: 999 - protein, 15 aa, Artificial Sequence
  Synthetic peptide
FISVLDITDAAVKAA

SEQ ID NO: 1000 - protein, 15 aa, Artificial Sequence
  Synthetic peptide
DAGHSLTWLWLLCGL

SEQ ID NO: 1001 - protein, 15 aa, Artificial Sequence
  Synthetic peptide
PEGCVLTNTGSVVKP

SEQ ID NO: 1002 - protein, 15 aa, Artificial Sequence
  Synthetic peptide
ELLLALLAFFLSKHS

SEQ ID NO: 1003 - protein, 15 aa, Artificial Sequence
  Synthetic peptide
ALDKLYKVFGSPVMT

SEQ ID NO: 1004 - protein, 15 aa, Artificial Sequence
  Synthetic peptide
APEGLVFLHTVLLPT

SEQ ID NO: 1005 - protein, 15 aa, Artificial Sequence
  Synthetic peptide
AWYFLAMLTGLLPSL

SEQ ID NO: 1006 - protein, 15 aa, Artificial Sequence
  Synthetic peptide
CAQYYNGIMVLPGVA

SEQ ID NO: 1007 - protein, 15 aa, Artificial Sequence
  Synthetic peptide
CVEMHNKINLCDDPE

SEQ ID NO: 1008 - protein, 15 aa, Artificial Sequence
  Synthetic peptide
EEIEYVHGDALHTLR

SEQ ID NO: 1009 - protein, 15 aa, Artificial Sequence
  Synthetic peptide
ELLQFVTDPTLIVAS

SEQ ID NO: 1010 - protein, 15 aa, Artificial Sequence
  Synthetic peptide
ESAYMPIADPTHFDI

SEQ ID NO: 1011 - protein, 15 aa, Artificial Sequence
  Synthetic peptide
FAVESGGYIPSDFAF

SEQ ID NO: 1012 - protein, 15 aa, Artificial Sequence
  Synthetic peptide
FSFGKVNNFVKFGSV

SEQ ID NO: 1013 - protein, 15 aa, Artificial Sequence
  Synthetic peptide
GLNKVKYATVVVGST

SEQ ID NO: 1014 - protein, 15 aa, Artificial Sequence
  Synthetic peptide
GNQTLFCNIMKFSDR

SEQ ID NO: 1015 - protein, 15 aa, Artificial Sequence
  Synthetic peptide
GNSLNHLTSQLRQNF

SEQ ID NO: 1016 - protein, 15 aa, Artificial Sequence
  Synthetic peptide
LLLWESGKAKPPLNR

SEQ ID NO: 1017 - protein, 15 aa, Artificial Sequence
  Synthetic peptide
NGSNILEAFTKPVFI

SEQ ID NO: 1018 - protein, 15 aa, Artificial Sequence
  Synthetic peptide
NPVSFVVKPVCSSIF

SEQ ID NO: 1019 - protein, 15 aa, Artificial Sequence
  Synthetic peptide
QAISSSIQAIYDRLD

SEQ ID NO: 1020 - protein, 15 aa, Artificial Sequence
  Synthetic peptide
QPVEGVSSFMNVTLD

SEQ ID NO: 1021 - protein, 15 aa, Artificial Sequence
  Synthetic peptide
QQVDRLITGRLAALN

SEQ ID NO: 1022 - protein, 15 aa, Artificial Sequence
  Synthetic peptide
RLHNFSIISGTAFLG

SEQ ID NO: 1023 - protein, 15 aa, Artificial Sequence
  Synthetic peptide
RVAGRSAIEDILFSK

SEQ ID NO: 1024 - protein, 15 aa, Artificial Sequence
  Synthetic peptide
SAIEDILFSKLVTSG

SEQ ID NO: 1025 - protein, 15 aa, Artificial Sequence
  Synthetic peptide
SNSNYLLEEFDVVFG

SEQ ID NO: 1026 - protein, 15 aa, Artificial Sequence
  Synthetic peptide
TGVNDAITQTSQALQ

SEQ ID NO: 1027 - protein, 15 aa, Artificial Sequence
  Synthetic peptide
TPFMILLVALSLCLT

SEQ ID NO: 1028 - protein, 15 aa, Artificial Sequence
  Synthetic peptide
TTQQAGAGIKYFCGM

SEQ ID NO: 1029 - protein, 15 aa, Artificial Sequence
  Synthetic peptide
VETYIKWPWWVWLCI

SEQ ID NO: 1030 - protein, 15 aa, Artificial Sequence
  Synthetic peptide
VMLQIQLTGILDGDY

SEQ ID NO: 1031 - protein, 15 aa, Artificial Sequence
  Synthetic peptide
VVGGTIQILASVPEK

SEQ ID NO: 1032 - protein, 15 aa, Artificial Sequence
  Synthetic peptide
VWHAKDFNSLSAEGR

SEQ ID NO: 1033 - protein, 15 aa, Artificial Sequence
  Synthetic peptide
WNGVIKNVNSVRDWL

SEQ ID NO: 1034 - protein, 15 aa, Artificial Sequence
  Synthetic peptide
APYGLLFMHFSYKPI

SEQ ID NO: 1035 - protein, 15 aa, Artificial Sequence
  Synthetic peptide
AQALNSLLQQLFNKF

SEQ ID NO: 1036 - protein, 15 aa, Artificial Sequence
  Synthetic peptide
AWRFPCAGRKVNFNE

SEQ ID NO: 1037 - protein, 15 aa, Artificial Sequence
  Synthetic peptide
CGIKYVAQPTEDVVD

SEQ ID NO: 1038 - protein, 15 aa, Artificial Sequence
  Synthetic peptide
CKFAVCGDGFVPFLL

SEQ ID NO: 1039 - protein, 15 aa, Artificial Sequence
  Synthetic peptide
CNAQEQQIYFFEGVA

SEQ ID NO: 1040 - protein, 15 aa, Artificial Sequence
  Synthetic peptide
CVQSFNGIKVLPPIL

SEQ ID NO: 1041 - protein, 15 aa, Artificial Sequence
  Synthetic peptide
DLLSEYGTFCDNINS

SEQ ID NO: 1042 - protein, 15 aa, Artificial Sequence
  Synthetic peptide
EDAIIVNDENSSIKV

SEQ ID NO: 1043 - protein, 15 aa, Artificial Sequence
  Synthetic peptide
ELSHWFKNQTSIAPN

SEQ ID NO: 1044 - protein, 15 aa, Artificial Sequence
  Synthetic peptide
ESQGNVVTSVMESQI

SEQ ID NO: 1045 - protein, 15 aa, Artificial Sequence
  Synthetic peptide
FEVEKGVTVDDFVAV

SEQ ID NO: 1046 - protein, 15 aa, Artificial Sequence
  Synthetic peptide
FKFDEPSDATDFIRV

SEQ ID NO: 1047 - protein, 15 aa, Artificial Sequence
  Synthetic peptide
FPKGYVMGLFRSYKT

SEQ ID NO: 1048 - protein, 15 aa, Artificial Sequence
  Synthetic peptide
GAISSSLQEILSRLD

SEQ ID NO: 1049 - protein, 15 aa, Artificial Sequence
  Synthetic peptide
GSSSRSFFEDLLFDK

SEQ ID NO: 1050 - protein, 15 aa, Artificial Sequence
  Synthetic peptide
IFKNNTSFPTNIAVE

SEQ ID NO: 1051 - protein, 15 aa, Artificial Sequence
  Synthetic peptide
IQESIKSLNNSYINL

SEQ ID NO: 1052 - protein, 15 aa, Artificial Sequence
  Synthetic peptide
KLITTACNGISVTQT

SEQ ID NO: 1053 - protein, 15 aa, Artificial Sequence
  Synthetic peptide
LIPRSYYLIQSGIFF

SEQ ID NO: 1054 - protein, 15 aa, Artificial Sequence
  Synthetic peptide
LLCRVTLGDFTIMSG

SEQ ID NO: 1055 - protein, 15 aa, Artificial Sequence
  Synthetic peptide
MLHGGGVAKAIAVAA

SEQ ID NO: 1056 - protein, 15 aa, Artificial Sequence
  Synthetic peptide
NFVALIPDYAKILVN

SEQ ID NO: 1057 - protein, 15 aa, Artificial Sequence
  Synthetic peptide
QPTEDVVDGDVVIRE

SEQ ID NO: 1058 - protein, 15 aa, Artificial Sequence
  Synthetic peptide
SFFEDLLFDKVKLSD

SEQ ID NO: 1059 - protein, 15 aa, Artificial Sequence
  Synthetic peptide
SVATFYIEHYVNRLV

SEQ ID NO: 1060 - protein, 15 aa, Artificial Sequence
  Synthetic peptide
VGGLYEIKIPTNFTI

SEQ ID NO: 1061 - protein, 15 aa, Artificial Sequence
  Synthetic peptide
VQIDRLINGRLTALN

SEQ ID NO: 1062 - protein, 15 aa, Artificial Sequence
  Synthetic peptide
WFNTMLDASAPATGW

SEQ ID NO: 1063 - protein, 15 aa, Artificial Sequence
  Synthetic peptide
YEMYVKWPWYVWLLI

SEQ ID NO: 1064 - protein, 15 aa, Artificial Sequence
  Synthetic peptide
YSFGRCPTSSIIKHP

SEQ ID NO: 1065 - protein, 15 aa, Artificial Sequence
  Synthetic peptide
APDGLLFLHTVLLPT

SEQ ID NO: 1066 - protein, 15 aa, Artificial Sequence
  Synthetic peptide
ASGVFGVNLRTNFTI

SEQ ID NO: 1067 - protein, 15 aa, Artificial Sequence
  Synthetic peptide
CAQYYNGIMVLPGVA

SEQ ID NO: 1068 - protein, 15 aa, Artificial Sequence
  Synthetic peptide
CVDLHNKINLCDDPE

SEQ ID NO: 1069 - protein, 15 aa, Artificial Sequence
  Synthetic peptide
DTCFGVSKPNAIDVE

SEQ ID NO: 1070 - protein, 15 aa, Artificial Sequence
  Synthetic peptide
EFRDYFNNNTDSIVI

SEQ ID NO: 1071 - protein, 15 aa, Artificial Sequence
  Synthetic peptide
FDVVFGHGAGSVVFV

SEQ ID NO: 1072 - protein, 15 aa, Artificial Sequence
  Synthetic peptide
FENYIKWPWWVWLII

SEQ ID NO: 1073 - protein, 15 aa, Artificial Sequence
  Synthetic peptide
FSPFNSLLCGDIVSG

SEQ ID NO: 1074 - protein, 15 aa, Artificial Sequence
  Synthetic peptide
GLNASVTLKICKFSR

SEQ ID NO: 1075 - protein, 15 aa, Artificial Sequence
  Synthetic peptide
GSALNHLTSQLRHNF

SEQ ID NO: 1076 - protein, 15 aa, Artificial Sequence
  Synthetic peptide
GTFESAAAGTFVLDM

SEQ ID NO: 1077 - protein, 15 aa, Artificial Sequence
  Synthetic peptide
GVFGVNLRTNFTIKG

SEQ ID NO: 1078 - protein, 15 aa, Artificial Sequence
  Synthetic peptide
LFVVALFIGVSFIDY

SEQ ID NO: 1079 - protein, 15 aa, Artificial Sequence
  Synthetic peptide
NIAFNVVKKGCFTGV

SEQ ID NO: 1080 - protein, 15 aa, Artificial Sequence
  Synthetic peptide
NLLKQYTSACKTIED

SEQ ID NO: 1081 - protein, 15 aa, Artificial Sequence
  Synthetic peptide
NNTDSIVIGGVTYQL

SEQ ID NO: 1082 - protein, 15 aa, Artificial Sequence
  Synthetic peptide
NQLRLAFLGASVTED

SEQ ID NO: 1083 - protein, 15 aa, Artificial Sequence
  Synthetic peptide
QPFSFSFRDELGVRV

SEQ ID NO: 1084 - protein, 15 aa, Artificial Sequence
  Synthetic peptide
QQVDRLITGRLAALN

SEQ ID NO: 1085 - protein, 15 aa, Artificial Sequence
  Synthetic peptide
RIAGRSALEDLLFSK

SEQ ID NO: 1086 - protein, 15 aa, Artificial Sequence
  Synthetic peptide
SALEDLLFSKVVTSG

SEQ ID NO: 1087 - protein, 15 aa, Artificial Sequence
  Synthetic peptide
SAMHSLLFGMLRRLD

SEQ ID NO: 1088 - protein, 15 aa, Artificial Sequence
  Synthetic peptide
SLWRVTAVHSDGMFV

SEQ ID NO: 1089 - protein, 15 aa, Artificial Sequence
  Synthetic peptide
SSATDAIIAVELLLL

SEQ ID NO: 1090 - protein, 15 aa, Artificial Sequence
  Synthetic peptide
STVVEVKSAIVCASV

SEQ ID NO: 1091 - protein, 15 aa, Artificial Sequence
  Synthetic peptide
TLFKFLLLLYAIYAL

SEQ ID NO: 1092 - protein, 15 aa, Artificial Sequence
  Synthetic peptide
VMNNIVLFLTWLLSM

SEQ ID NO: 1093 - protein, 15 aa, Artificial Sequence
  Synthetic peptide
VTEDVKFAASTGVID

SEQ ID NO: 1094 - protein, 15 aa, Artificial Sequence
  Synthetic peptide
WFCANQSTSVYSANG

SEQ ID NO: 1095 - protein, 15 aa, Artificial Sequence
  Synthetic peptide
YFSQLLCEPIKLVNS

SEQ ID NO: 1096 - protein, 15 aa, Artificial Sequence
  Synthetic peptide
AEALNNLLQQLSNRF

SEQ ID NO: 1097 - protein, 15 aa, Artificial Sequence
  Synthetic peptide
APYGLYFIHFSYVPT

SEQ ID NO: 1098 - protein, 15 aa, Artificial Sequence
  Synthetic peptide
AQIDRLINGRLTALN

SEQ ID NO: 1099 - protein, 15 aa, Artificial Sequence
  Synthetic peptide
CQFKDKNLQDLWVLY

SEQ ID NO: 1100 - protein, 15 aa, Artificial Sequence
  Synthetic peptide
CVQSYKGIKVLPPLL

SEQ ID NO: 1101 - protein, 15 aa, Artificial Sequence
  Synthetic peptide
ELDQWFKNQTSVAPD

SEQ ID NO: 1102 - protein, 15 aa, Artificial Sequence
  Synthetic peptide
ETFTVCADGFMPFLL

SEQ ID NO: 1103 - protein, 15 aa, Artificial Sequence
  Synthetic peptide
GAISASLQEILSRLD

SEQ ID NO: 1104 - protein, 15 aa, Artificial Sequence
  Synthetic peptide
GIFAKVKNTKVIKDR

SEQ ID NO: 1105 - protein, 15 aa, Artificial Sequence
  Synthetic peptide
ICSASDMTNPDYTNL

SEQ ID NO: 1106 - protein, 15 aa, Artificial Sequence
  Synthetic peptide
IFINNTTYPTNVAVE

SEQ ID NO: 1107 - protein, 15 aa, Artificial Sequence
  Synthetic peptide
ISSTVRLQAGTATEY

SEQ ID NO: 1108 - protein, 15 aa, Artificial Sequence
  Synthetic peptide
KASSRSAIEDLLFDK

SEQ ID NO: 1109 - protein, 15 aa, Artificial Sequence
  Synthetic peptide
KRGEKGAYNKDHGRG

SEQ ID NO: 1110 - protein, 15 aa, Artificial Sequence
  Synthetic peptide
LIREIVMNASPYDLE

SEQ ID NO: 1111 - protein, 15 aa, Artificial Sequence
  Synthetic peptide
LLFDVIVAWHVVRDP

SEQ ID NO: 1112 - protein, 15 aa, Artificial Sequence
  Synthetic peptide
LQEAIKVLNQSYINL

SEQ ID NO: 1113 - protein, 15 aa, Artificial Sequence
  Synthetic peptide
LVPQENYSSIRFASV

SEQ ID NO: 1114 - protein, 15 aa, Artificial Sequence
  Synthetic peptide
NIEAWLNDKSVPSPL

SEQ ID NO: 1115 - protein, 15 aa, Artificial Sequence
  Synthetic peptide
NPPTNVVSHVNGDWF

SEQ ID NO: 1116 - protein, 15 aa, Artificial Sequence
  Synthetic peptide
SAIEDLLFDKVKLSD

SEQ ID NO: 1117 - protein, 15 aa, Artificial Sequence
  Synthetic peptide
SLGFYNPPTNVVSHV

SEQ ID NO: 1118 - protein, 15 aa, Artificial Sequence
  Synthetic peptide
SQLVEYGSFCDNINA

SEQ ID NO: 1119 - protein, 15 aa, Artificial Sequence
  Synthetic peptide
SVKSYDSLVYTGVLG

SEQ ID NO: 1120 - protein, 15 aa, Artificial Sequence
  Synthetic peptide
SVVEVVTTSLTPCGY

SEQ ID NO: 1121 - protein, 15 aa, Artificial Sequence
  Synthetic peptide
TIFNCVYALNNVYLG

SEQ ID NO: 1122 - protein, 15 aa, Artificial Sequence
  Synthetic peptide
TILNTACGVFEVDDT

SEQ ID NO: 1123 - protein, 15 aa, Artificial Sequence
  Synthetic peptide
VGGLYEIQIPSEFTI

SEQ ID NO: 1124 - protein, 15 aa, Artificial Sequence
  Synthetic peptide
VKNIPRYASAVAQAF

SEQ ID NO: 1125 - protein, 15 aa, Artificial Sequence
  Synthetic peptide
VSATVLQNNELMPAK

SEQ ID NO: 1126 - protein, 15 aa, Artificial Sequence
  Synthetic peptide
YEYYVKWPWYVWLLI

SEQ ID NO: 1127 - protein, 266 aa, Artificial Sequence
  Synthetic peptide
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNGGSGGGSGYEQYIKWPWYIWLGFIAGLIAIVMVTIMLCCMTSCCSCLK

SEQ ID NO: 1128 - protein, 13 aa, Artificial Sequence
  Synthetic peptide
AKFVAAWTLKAAA

SEQ ID NO: 1129 - protein, 287 aa, Artificial Sequence
  Synthetic peptide
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNGGSGGGSGAKFVAAWTLKAAAGGSGGGSGYEQYIKWPWYIWLGFIAGLIAIVMVTIMLCCMTSCCSCLK

SEQ ID NO: 1130 - protein, 1258 aa, Artificial Sequence
  Synthetic peptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGGSGGSNHNFLVQAGNVQLRV

SEQ ID NO: 1131 - protein, 1237 aa, Artificial Sequence
  Synthetic peptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSAACKSQLVEYGSFCDNINRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNSLLQQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFL

SEQ ID NO: 1132 - protein, 256 aa, Artificial Sequence
  Synthetic peptide
SDFVRATATIPIQASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNGGSGGGSGNHNFLVQAGNVQLRV

SEQ ID NO: 1133 - protein, 218 aa, Artificial Sequence
  Synthetic peptide
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYRIDTTATSCQIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKN

SEQ ID NO: 1134 - protein, 14 aa, Artificial Sequence
  Synthetic peptide
MFVFLVLLPLVSSQ

SEQ ID NO: 1135 - protein, 11 aa, Artificial Sequence
  Synthetic peptide
GGSGHHHHHHC

SEQ ID NO: 1136 - protein, 12 aa, Artificial Sequence
  Synthetic peptide
AKFVAAWTLKAA

SEQ ID NO: 1137 - protein, 222 aa, Artificial Sequence
  Synthetic peptide
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 1138 - protein, 419 aa, Artificial Sequence
  Synthetic peptide
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 1139 - protein, 290 aa, Artificial Sequence
  Synthetic peptide
SAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 1140 - protein, 275 aa, Artificial Sequence
  Synthetic peptide
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 1141 - protein, 225 aa, Artificial Sequence
  Synthetic peptide
MSNDNCTGDIVTHLKNWNFGWNVILTIFIVILQFGHYKYSRLFYGLKMLVLWLLWPLVLALSIFDTWANWDSNWAFVAFSFFMAVSTLVMWVMYFANSFRLFRRARTFWAWNPEVNAITVTTVLGQTYYQPIQQAPTGITVTLLSGVLYVDGHRLASGVQVHNLPEYMTVAVPSTTIIYSRVGRSVNSQNSTGWVFYVRVKHGDFSAVSSPMSNMTENERLLHFF

SEQ ID NO: 1142 - protein, 223 aa, Artificial Sequence
  Synthetic peptide
MNKSFLPQFTSDQAVTFLKEWNFSLGVILLFITIILQFGYTSRSMFVYLIKMIILWLMWPLTITLTIFNCFYALNNAFLAFSIVFTIISIVIWILYFVNSIRLFIRTGSWWSFNPETNNLMCIDMKGKMFVRPVIEDYHTLTATVIRGHLYIQGVKLGTGYTLSDLPVYVTVAKVQVLCTYKRAFLDKLDVNSGFAVFVKSKVGNYRLPSSKPSGMDTALLRA

SEQ ID NO: 1143 - protein, 226 aa, Artificial Sequence
  Synthetic peptide
MSNSSVPLLEVYVHLRNWNFSWNLILTLFIVVLQYGHYKYSRLLYGLKMSVLWCLWPLVLALSIFDCFVNFNVDWVFFGFSILMSIITLCLWVMYFVNSFRLWRRVKTFWAFNPETNAIISLQVYGHNYYLPVMAAPTGVTLTLLSGVLLVDGHKIATRVQVGQLPKYVIVATPSTTIVCDRVGRSVNETSQTGWAFYVRAKHGDFSGVASQEGVLSEREKLLHLI

SEQ ID NO: 1144 - protein, 230 aa, Artificial Sequence
  Synthetic peptide
MSSKTTPAPVYIWTADEAIKFLKEWNFSLGIILLFITIILQFGYTSRSMFVYVIKMIILWLMWPLTIILTIFNCVYALNNVYLGLSIVFTIVAIIMWIVYFVNSIRLFIRTGSFWSFNPETNNLMCIDMKGTMYVRPIIEDYHTLTVTIIRGHLYIQGIKLGTGYSLADLPAYMTVAKVTHLCTYKRGFLDRISDTSGFAVYVKSKVGNYRLPSTQKGSGMDTALLRNNI

SEQ ID NO: 1145 - protein, 389 aa, Artificial Sequence
  Synthetic peptide
MATVKWADASEPQRGRQGRIPYSLYSPLLVDSEQPWKVIPRNLVPINKKDKNKLIGYWNVQKRFRTRKGKRVDLSPKLHFYYLGTGPHKDAKFRERVEGVVWVAVDGAKTEPTGYGVRRKNSEPEIPHFNQKLPNGVTVVEEPDSRAPSRSQSRSQSRGRGESKPQSRNPSSDRNHNSQDDIMKAVAAALKSLGFDKPQEKDKKSAKTGTPKPSRNQSPASSQTSAKSLARSQSSETKEQKHEMQKPRWKRQPNDDVTSNVTQCFGPRDLDHNFGSAGVVANGVKAKGYPQFAELVPSTAAMLFDSHIVSKESGNTVVLTFTTRVTVPKDHPHLGKFLEELNAFTREMQQHPLLNPSALEFNPSQTSPATAEPVRDEVSIETDIIDEVN

SEQ ID NO: 1146 - protein, 441 aa, Artificial Sequence
  Synthetic peptide
MSYTPGHYAGSRSSSGNRSGILKKTSWADQSERNYQTFNRGRKTQPKFTVSTQPQGNTIPHYSWFSGITQFQKGRDFKFSDGQGVPIAFGVPPSEAKGYWYRHSRRSFKTADGQQKQLLPRWYFYYLGTGPYANASYGESLEGVFWVANHQADTSTPSDVSSRDPTTQEAIPTRFPPGTILPQGYYVEGSGRSASNSRPGSRSQSRGPNNRSLSRSNSNFRHSDSIVKPDMADEIANLVLAKLGKDSKPQQVTKQNAKEIRHKILTKPRQKRTPNKHCNVQQCFGKRGPSQNFGNAEMLKLGTNDPQFPILAELAPTPGAFFFGSKLDLVKRDSEADSPVKDVFELHYSGSIRFDSTLPGFETIMKVLEENLNAYVNSNQNTDSDSLSSKPQRKRGVKQLPEQFDSLNLSAGTQHISNDFTPEDHSLLATLDDPYVEDSVA

SEQ ID NO: 1147 - protein, 377 aa, Artificial Sequence
  Synthetic peptide
MASVNWADDRAARKKFPPPSFYMPLLVSSDKAPYRVIPRNLVPIGKGNKDEQIGYWNVQERWRMRRGQRVDLPPKVHFYYLGTGPHKDLKFRQRSDGVVWVAKEGAKTVNTSLGNRKRNQKPLEPKFSIALPPELSVVEFEDRSNNSSRASSRSSTRNNSRDSSRSTSRQQSRTRSDSNQSSSDLVAAVTLALKNLGFDNQSKSPSSSGTSTPKKPNKPLSQPRADKPSQLKKPRWKRVPTREENVIQCFGPRDFNHNMGDSDLVQNGVDAKGFPQLAELIPNQAALFFDSEVSTDEVGDNVQITYTYKMLVAKDNKNLPKFIEQISAFTKPSSIKEMQSQSSHVAQNTVLNASIPESKPLADDDSAIIEIVNEVLH

SEQ ID NO: 1148 - protein, 448 aa, Artificial Sequence
  Synthetic peptide
MSFTPGKQSSSRASSGNRSGNGILKWADQSDQFRNVQTRGRRAQPKQTATSQQPSGGNVVPYYSWFSGITQFQKGKEFEFVEGQGVPIAPGVPATEAKGYWYRHNRRSFKTADGNQRQLLPRWYFYYLGTGPHAKDQYGTDIDGVYWVASNQADVNTPADIVDRDPSSDEAIPTRFPPGTVLPQGYYIEGSGRSAPNSRSTSRTSSRASSAGSRSRANSGNRTPTSGVTPDMADQIASLVLAKLGKDATKPQQVTKHTAKEVRQKILNKPRQKRSPNKQCTVQQCFGKRGPNQNFGGGEMLKLGTSDPQFPILAELAPTAGAFFFGSRLELAKVQNLSGNPDEPQKDVYELRYNGAIRFDSTLSGFETIMKVLNENLNAYQQQDGMMNMSPKPQRQRGHKNGQGENDNISVAVPKSRVQQNKSRELTAEDISLLKKMDEPYTEDTSEI

SEQ ID NO: 1149 - protein, 279 aa, Artificial Sequence
  Synthetic peptide
SGKTTSMFKSISLFAGFFVMFWAELFVYTTTIWVNPGFLTPFMILLVALSLCLTFVVKHKVLFLQVFLLPSIIVAAIQNCAWDYHVTKVLAEKFDYNVSVMQMDIQGFVNIFICLFVALLHTWRFAKERCTHWCTYLFSLIAVLYTALYSYDYVSLLVMLLCAISNEWYIGAIIFRICRFGVAFLPVEYVSYFDGVKTVLLFYMLLGFVSCMYYGLLYWINRFCKCTLGVYDFCVSPAEFKYMVANGLNAPNGPFDALFLSFKLMGIGGPRTIKVSTVQ

SEQ ID NO: 1150 - protein, 287 aa, Artificial Sequence
  Synthetic peptide
SKTKRFIKETIYWILISTFLFSCIISAFVKWTIFMYINTHMIGVTLCVLCFVSFMMLLVKHKHFYLTMYIIPVLCTLFYVNYLVVYKEGFRGFTYVWLSYFVPAVNFTYVYEVFYGCILCVFAIFITMHSINHDIFSLMFLVGRIVTLISMWYFGSNLEEDVLLFITAFLGTYTWTTILSLAIAKIVANWLSVNIFYFTDVPYIKLILLSYLFIGYILSCYWGFFSLLNSVFRMPMGVYNYKISVQELRYMNANGLRPPRNSFEAILLNLKLLGIGGVPVIEVSQIQ

SEQ ID NO: 1151 - protein, 279 aa, Artificial Sequence
  Synthetic peptide
SGKVIFGLKTMFLFSVFFTMFWAELFIYTNTIWINPVILTPIFCLLLFLSLVLTMFLKHKFLFLQVFLLPTVIATALYNCVLDYYIVKFLADHFNYNVSVLQMDVQGLVNVLVCLFVVFLHTWRFSKERFTHWFTYVCSLIAVAYTYFYSGDFLSLLVMFLCAISSDWYIGAIVFRLSRLIVFFSPESVFSVFGDVKLTLVVYLICGYLVCTYWGILYWFNRFFKCTMGVYDFKVSAAEFKYMVANGLHAPHGPFDALWLSFKLLGIGGDRCIKISTVQ

SEQ ID NO: 1152 - protein, 110 aa, Artificial Sequence
  Synthetic peptide
NNELMPAKLKIQVVNSGPDQTCNTPTQCYYNNSNNGKIVYAILSDVDGLKYTKILKDDGNFVVLELDPPCKFTVQDAKGLKIKYLYFVKGCNTLARGWVVGTISSTVRLQ

SEQ ID NO: 1153 - protein, 133 aa, Artificial Sequence
  Synthetic peptide
MALGLFTLQLVSAVNQSLSNAKVSAEVSRQVIQDVKDGTVTFNLLAYTLMSLFVVYFALFKARSHRGRAALIVFKILILFVYVPLLYWSQAYIYATLIAVILLGRFFHTAWHCWLYKTWDFIVFNVTTLCYAR

SEQ ID NO: 1154 - protein, 225 aa, Artificial Sequence
  Synthetic peptide
MPFGGLFQLTLESTINKSVANLKLPPHDVTVLRDNLKPVTTLSTITAYLLVSLFVTYFALFKPLTARGRVACFVLKLLTLFVYVPLLVLFGMYLDSFIIFSTLLFRFIHVGYYAYLYKNFSFVLFNVTKLCFVSGKCWYLEQSFYENRFAAIYGGDHYVVLGGETITFVSFDDLYVAIRGSCEKNLQLMRKVDLYNGAVIYIFAEEPVVGIVYSSQLYEDVPSIN